(19) United States
(12) Patent Application Publication
(10) Pub. No.: US 2004/0214232 A1
(43) Pub. Date: Oct. 28, 2004

(54) Generation of skeletal diversity within a combinatorial library

(76) Inventors: Martin D. Burke, Cambridge, MA (US); Eric M. Berger, Concord, MA (US); Ohyun Kwon, Los Angeles, CA (US); Seung Bum Park, Arlington, MA (US); Stuart L. Schreiber, Boston, MA (US)

Correspondence Address: Patent Department, C. Hunter Baker, M.D., Ph.D., Choate, Hall & Stewart, Exchange Place, 53 State Street, Boston, MA, 02129, US

(21) Appl. No.: 10/640,834

(22) Filed: Aug. 14, 2003

Related U.S. Application Data
(60) Provisional application No. 60/404,204, filed on Aug. 16, 2002.

(51) Int. Cl. G01N 33/53; G01N 33/543

(52) U.S. Cl. 435/7.1; 436/518

(57) ABSTRACT

The present invention provides a method of synthesizing a library of chemical compounds with skeletal diversity. Two approaches are used to create skeletal diversity within a library of chemical compounds: (1) the “branching pathways” (or reagent-based) approach; and (2) the “folding pathways” (or substrate-based) approach. Upon exposure to certain reaction conditions the members of the library undergo unique transformations into a diverse collection of molecular skeletons, which can be functionalized and derivatized further to generate a large collection of unique, natural product-like compounds. A furan-based library synthesized using the folding pathways approach is provided, and a polycyclic library created using the braching pathways approach is also provided. The invention also provides materials, reagents, intermediates, and kits useful in the practice of the inventive method as well as method for screening the inventive compounds.

### RELATED APPLICATIONS

The present application claims priority to provisional application U.S. Ser. No. 60/404,204, filed Aug. 16, 2002, entitled “Generation of Skeletal Diversity within a Combinatorial Library”, the entire contents of which is incorporated herein by reference.

### BACKGROUND OF THE INVENTION

Nature has played a key role in the search for new pharmacological agents. The diversity of small molecule chemical compounds produced by Nature and commonly known as natural products has aided chemists and biologists in the discovery of new pharmacological agents to treat and prevent human disease. Natural products have been used as a pharmacological agent themselves (e.g., penicillin), or the natural product may be a lead compound which is further modified and/or studied to yield a pharmacological agent (Newman et al. Nat. Prod. Rep. 17:215-234, 2000; incorporated herein by reference). Natural products have also played a key role as probes of biological function (Schreiber Chem. and Eng. News 22-32, Oct. 26, 1992; incorporated herein by reference). Natural products have been found to alter biological function and thereby these molecules are useful in elucidating signal transduction pathways, cell trafficking pathways, protein function, etc. (Schreiber et al. J. Am. Chem. Soc. 112:5583, 1990; Mitchison Chem. and Biol. 1:3, 1994; each of which is incorporated herein by reference).

More recently chemists have begun to synthesize their own collections of diverse chemical compounds rather than relying exclusively on Mother Nature. Using combinatorial chemistry, chemists have created arrays of thousands to millions of chemical compounds quickly and efficiently and in large enough quantities to assay for biological activities (Hall et al. J. Comb. Chem. 3(2):125-150, 2001; Nicolaou et al. Angew. Chem. Int. Ed. Engl. 36:2097-2103, 1997; Nicolaou et al. J. Am. Chem. Soc. 120:10814-10826, 1998; Lee et al. Org. Lett. 1:1859-1862, 1999; Xu et al. J. Am. Chem. Soc. 121:4898-4899, 1999; Wipf et al. J. Am. Chem. Soc. 122:9391-9395, 2000; Boger et al. J. Am. Chem. Soc. 122:6382-6394, 2000; Nicolaou et al. J. Am. Chem. Soc. 122:9968-9976, 2000; each of which is incorporated herein by reference). In simple terms, combinatorial chemistry subjects a template with a variety of sites for functionalization to various reagents to produce an array of chemical compounds. Each site on a template is reacted with one of many different possible reagents to create diversity in the library. However, the resulting compounds are generally related structurally since all are derived from the same core structure of the template. The compounds having a common molecular skeleton display chemical information similarly in three-dimensional space, thus limiting the pool of potential binding partners to only those macromolecules with a complementary three-dimensional surface.

Combinatorial libraries with greater diversity would potentially lead to more hits from the library in any one screen. In some cases, the libraries synthesized from one core template may never be able to produce compounds with a certain biological activity given the constraints of the core structure used to generate the library. A library with diversity in the molecular skeleton would allow for greater diversity with a greater potential of creating compounds with the desired biological activity.

### SUMMARY OF THE INVENTION

The present invention provides a system for preparing a collection of chemical compounds based on a template which undergoes a transformation leading to skeletal diversity within the collection. The skeletal diversity within the collection of compounds can be accomplished in one of two ways: (1) by subjecting the template to different reaction conditions the template will undergo different reactions to yield different molecular skeletons (the “branching pathway” approach or reagent-based approach); and (2) by subjecting templates with information encoded in the precursor molecules to the same reaction condition thereby yielding different molecular skeletons (the “folding pathways” approach or substrate-based approach). The present invention provides methods, strategies, compositions, reagents, intermediates, and kits useful in the generation of combinatorial libraries using the above two approaches. The invention alse provides libraries of chemical compounds. The resulting combinatorial libraries are more diverse in terms of chemical structures of the members and populate chemical space with small molecules having complex and diverse molecular skeletons. Whereas libraries having a common molecular skeleton display chemical information similarly with respect to three-dimensional space, libraries of the present invention display chemical information in three-dimensional space in many different configurations depending on the molecular skeletons created.

In the “branching pathway” (reagent-based) approach, the precursor molecules are split up into groups and each group is subjected to a different set of reaction conditions designed to yield a certain molecular skeleton. The resulting molecular skeletons can then be further functionalized to produce a large collection of diverse chemical compounds. One of the advantages to this approach is that all the chemical compounds do not have the same underlying molecular skeleton. Instead, there are many different molecular skeletons in the library thereby expanding the chemical diversity of the library.

In the “folding pathways” (substrate-based) approach, the precursor molecules have encoded within them information leading to different molecular skeletons when exposed to a common set of reaction conditions. This approach is analogous to the folding pathway of proteins in which the primary amino acid sequence of a protein encodes how a protein will fold into a 3-D structure. For example, in generating a combinatorial library using the “folding pathways” approach, the precursor template may have certain functional groups at certain sites which allow for certain reactions such as cyclization, isomerization, and ring opening to take place when exposed to the common reaction conditions. In certain embodiments, more than one site on the precursor template may affect the molecular skeleton produced. Any chemical compounds may be used as a precursor template for the “folding pathways” approach. In certain embodiments, the precursor template undergoes a rearrangement or restructuring (e.g., isomerization, ring opening, and ring closing reactions) reaction when exposed to certain reaction conditions. The final molecular skeleton derived from the precursor template results from the structure of the precursor template. Some useful reactions which can be used in the “folding pathways” approach to generate skeletal diversity include oxidation, reduction, cyclopropanation, epoxidation, olefination, ring closing reactions, ring opening reactions, etc.

Libraries of chemical compounds synthesized using the “branching pathways” or “folding pathways” approach may be further derivatized or functionalized before and/or after the molecular skeleton is generated from the precursor template. As would be appreciated by one of skill in the art, any methods known in the art can be used to derivatize or functionalize the members of the library during the production process. In certain embodiments, split-pool synthetic methods are used. In certain embodiments, the reactions are done on compounds attached to a solid support using solid phase chemistry. Preferably the reactions are high yielding with only one product resulting. The reaction sequence that a member of the library is subjected to may be encoded using tags attached to the solid support the actual member is attached to when solid phase methods are used.

One example of an inventive combinatorial library is one based on furan derivatives. The functionalization of the furan derivatives allows for different reactions to occur thereby generating skeletal diversity. In this way, the furan derivatives encode information leading to the molecular skeleton that will be formed upon exposure to certain common reaction conditions. Different furan derivatives will lead to different molecular skeletons. These molecular skeletons once produced can be further functionalized to generate a diverse set of chemical compounds. The compounds of the furan library may be used in studies in chemical genetics where small molecules are used to perturb and thereby study protein function. These compounds are useful as pharmacological agents or lead compounds in the development of pharmacological agents. Examples of chemical compounds of the inventive library includes those of the general formulae:

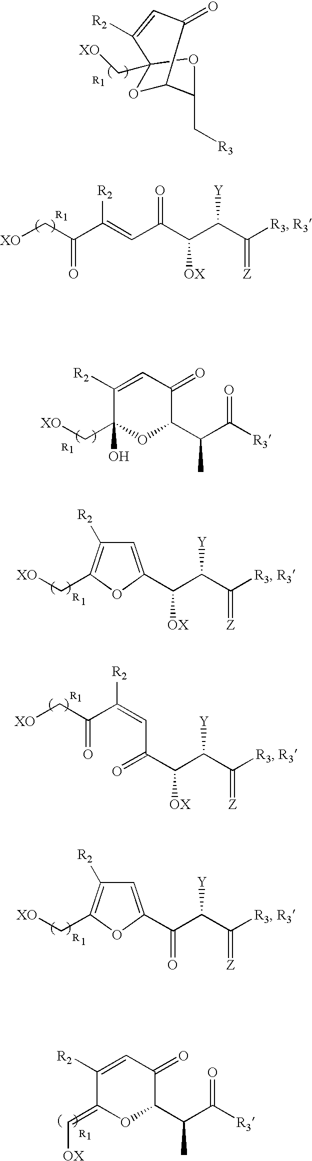

wherein exemplary R₁, R₂, R₃, and R₃′ groups are shown in the figures and in the claims; however, these groups, as would be appreciated by one of skill in the art, are only exemplary and other groups could be used in their place as long as the rules of chemistry are not violated.

The present invention also provides for kits useful in the preparation of combinatorial libraries based on the “branching pathway” or “folding pathways” approach to generate skeletal or chemical diversity. These kits may include solid supports, template precursors, template precursors attached to solid supports, reagents, catalysts, reagents for cleavage from the solid supports, instructions, solvents, acids, bases, encoding tags, etc. The kits may also contain materials, reagents, cells, proteins, protocols, etc. useful in the assaying of the newly synthesized chemical compounds for certain biological activities.

### Definitions

This invention provides a new family of compounds with a range of biological properties. Compounds of this invention have biological activities relevant for the treatment of diseases including proliferative diseases such as cancer, wound healing, and bacterial infections to name a few. Compounds of this invention include those specifically set forth above and described herein, and are illustrated in part by the various classes, subgenera and species disclosed elsewhere herein.

It will be appreciated by one of ordinary skill in the art that asymmetric centers may exist in the compounds of the present invention. Thus, inventive compounds and pharmaceutical compositions thereof may be in the form of an individual enantiomer, diastereomer, or geometric isomer, or may be in the form of a mixture of stereoisomers. In certain embodiments, the compounds of the invention are enantiopure compounds. In certain other embodiments, mixtures of stereoisomers or diastereomers are provided.

Additionally, the present invention provides pharmaceutically acceptable derivatives of the inventive compounds, and methods of treating a subject using these compounds, pharmaceutical compositions thereof, or either of these in combination with one or more additional therapeutic agents. The phrase, “pharmaceutically acceptable derivative”, as used herein, denotes any pharmaceutically acceptable salt, ester, or salt of such ester, of such compound, or any other adduct or derivative which, upon administration to a patient, provides (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety that is susceptible to removal in vivo, yielding the parent molecule as the pharmacologically active species. An example of a pro-drug is an ester which is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group”, has used herein, it is meant that a particular functional moiety, e.g., O, S, carbonyl, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative disorders, cancer, wound healing, infectious diseases, and immunological diseases. Preferably, the substituent is small than the compound or core structure of the compound. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes both straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 aliphatic carbon atoms. Illustrative aliphatic groups thus include, but are not limited to, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alkoxy”, or “thioalkyl” as used herein refers to an alkyl group, as previously defined, attached to the parent molecular moiety through an oxygen atom or through a sulfur atom. In certain embodiments, the alkyl group contains 1-20 alipahtic carbon atoms. In certain other embodiments, the alkyl group contains 1-10 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. In certain embodiments, the alkyl group contains 1-20 aliphatic carbon atoms. In certain other embodiments, the alkyl group contains 1-10 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like. Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(R)₂; —OC(O)Rₓ; —OCO₂Rₓ; 13 OCON(Rₓ)₂; —N(Rₓ)₂; —S(O)₂Rₓ; wherein each occurrence of R, independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples which are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiments of the present invention, the term “heteroaryl”, as used herein, refers to a cyclic aromatic radical having from five to ten ring atoms of which one ring atom is selected from S, O, and N; zero, one or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one, two or three of the hydrogen atoms thereon independently with any one or more of the following moieties including, but not limited to: aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃—; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or hetercyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

The term “heteroaliphatic”, as used herein, refers to aliphatic moieties which contain one or more oxygen, sulfur, nitrogen, phosphorous or silicon atoms, e.g., in place of carbon atoms. Heteroaliphatic moieties may be branched, unbranched or cyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6- or 7- membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quatemized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

The term “solid support”, as used herein, refers to a material having a rigid or semi-rigid surface. Such materials will preferably take the form of small beads, pellets, disks, chips, dishes, multi-well plates, glass slides, wafers, or the like, although other forms may be used. In some embodiments, at least one surface of the substrate will be substantially flat. The term “surface” refers to any generally two-dimensional structure on a solid substrate and may have steps, ridges, kinks, terraces, and the like without ceasing to be a surface. The material of the solid support may be glass, metal, polymeric, or crystalline in nature.

The term “polymeric support”, as used herein, refers to a soluble or insoluble polymer to which an amino acid or other chemical moiety can be covalently bonded by reaction with a functional group of the polymeric support. Many suitable polymeric supports are known, and include soluble polymers such as polyethylene glycols or polyvinyl alcohols, as well as insoluble polymers such as polystyrene resins. A suitable polymeric support includes functional groups such as those described below. A polymeric support is termed “soluble” if a polymer, or a polymer-supported compound, is soluble under the conditions employed. However, in general, a soluble polymer can be rendered insoluble under defined conditions. Accordingly, a polymeric support can be soluble under certain conditions and insoluble under other conditions.

The term “linker”, as used herein, refers to a chemical moiety utilized to attach a compound of interest to a solid support to facilitate synthesis of inventive compounds. Exemplary linkers are described in Example 2, as described herein. It will be appreciated that other linkers (including silicon-based linkers and other linkers) that are known in the art can also be employed for the synthesis of the compounds of the invention.

Unless indicated otherwise, the terms defined below have the following meanings:

“Combinatorial libraries”: A combinatorial library is any collection of chemical compounds created using combinatorial chemistry. In general, diversity in the library is created by using a diverse set of chemically similar reagents at each step of the synthesis of the library's members. For example, a library constructed in three steps with 10 different reagents used in each step would result in 1,000 discrete library members. These compounds may then be screened for useful biological or chemical properties. The chemical compounds of the combinatorial library may be small molecules, organic compounds, organometallic compounds, polymers, polynucleotides, peptides, proteins, etc. In certain embodiments, the chemical compounds are small molecules or organic compounds. The library may contain at least 50, 100, 500, 1000, 10000, 100000, or 1 million members. The combinatorial library may be created through split and pool synthetic techniques. In certain embodiments, the library is created on a solid phase or polymeric support.

“Compound”: The term “compound” or “chemical compound” as used herein can include organometallic compounds, organic compounds, metals, transitional metal complexes, and small molecules. In certain preferred embodiments, polynucleotides are excluded from the definition of compounds. In other preferred embodiments, polynucleotides and peptides are excluded from the definition of compounds. In a particularly preferred embodiment, the term compounds refers to small molecules (e.g., preferably, non-peptidic and non-oligomeric) and excludes peptides, polynucleotides, transition metal complexes, metals, and organometallic compounds.

“Core structure” refers to the underlying structure of the precursor templates which are exposed to reaction condition such that the core structure along with other functional groups of the precursor template undergo a transformation to form the molecular skeletons of the member of the final library. These core structures may have additional functional groups and structures off them which affect the transformation into molecular skeletons. The transformation may involve rearrangements (e.g., hydrogen shifts, methyl shifts), ring opening, ring closings, migrations (e.g., double bond migration), or any combination thereof. In certain embodiments, there will be a core structure common to all or many of the precursor templates in a library. Core structures may includes carbocyclic systems (e.g., cyclohexane, cyclopropane, cyclobutane), heterocyclic systems (e.g., epoxides, aziridines), aromatic carbocyclic systems (e.g., phenyl, substituted phenyls), aromatic heterocyclic systems (e.g., furans, imidazoles, purines, pyrimidines, oxazoles, thiazoles), oxygen-containing heterocycles, nitrogen-containing heterocycles, sulfur-containing heterocycles, polycyclic systems, unsaturated systems, polyunsaturated systems (e.g., alpha,beta-unsaturated ketones, isoprenoids), conjugated polyunsaturated systems (e.g., dienes), alkene-containing systems, alkyne-containing systems, etc. Preferably, the core structure includes some degree of unsaturation.

“Libraries”: Libraries refer to any collection of chemical compounds. Any type of chemical compound may be member of a library including small molecules, organic compounds, organometallic compounds, polymers, polynucleotides, peptides, proteins, sugars, carbohydrates, etc. In certain embodiments, the chemical compounds are small molecules. Libraries may include random collections of compounds such as those found in a historical collection of a pharmaceutical company. A library in certain embodiments is a combinatorial library as defined supra.

“Molecular skeleton”: Molecular skeleton, as used herein, refers to the underlying structure of a chemical compound once the precursor template has been reacted under certain reaction conditions. A molecular skeleton may be a core structure giving a molecule its shape. For example, a molecular skeleton may be the core structure to which appendages, functional groups, building blocks, or other moieties are covalently linked. In certain embodiments, the molecular skeleton may be a combination of rigidifying elements in the form of bonding and/or non-bonding interactions. The molecular skeleton provides sites for functionalization, derivatization, and diversification. In certain embodiments, the molecular skeleton is a cyclic structure. In certain embodiments, the molecular skeleton may contain more than one cyclic structure. These cyclic structure may be linked in any way that does not defy the law of chemistry, for example, spiro linked, fused, bridging, etc. In certain other embodiments, the molecular skeleton is a linear structure containing no cyclic structures.

“Natural Product-Like Compound”: As used herein, the term “natural product-like compound” refers to compounds that are similar to complex natural products which nature has selected through evolution. Typically, these compounds contain one or more stereocenters, a high density and diversity of functionality, and a diverse selection of atoms within one structure. In this context, diversity of functionality can be defined as varying the topology, charge, size, hydrophilicity, hydrophobicity, and reactivity to name a few, of the functional groups present in the compounds. The term, “high density of functionality”, as used herein, can preferably be used to define any molecule that contains preferably three or more latent or active diversifiable functional moieties. These structural characteristics may additionally render the inventive compounds functionally reminiscent of complex natural products, in that they may interact specifically with a particular biological receptor, and thus may also be functionally natural product-like.

“Peptide” of “protein”: According to the present invention, a “peptide” or “protein” comprises a string of at least three amino acids linked together by peptide bonds. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a famesyl group, an isofarnesyl group, a fatty acid group, a linker for conjugation, functionalization, or other modification, etc.

“Polynucleotide” or “oligonucleotide”: Polynucleotide or oligonucleotide refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Skeletal diversity”: The term “skeletal diversity” as applied to a collection of chemical compounds such as small molecules refers to the degree of different molecular skeletons within the collection. A collection with many different molecular skeletons would have more skeletal diversity than a collection derived from one molecular skeleton as is found in traditional combinatorial libraries, and thereby display chemical information more differently in three-dimensional space. A library with a high degree of skeletal diversity allows different functional groups off the molecular skeletons to occupy different regions of chemical space as compared to other members of the same library. For example, in earlier combinatorial libaries the functional groups are arranged in a two-dimensional, approximately circular area around the molecular skeleton. In libaries of the invention with skeletal diversity, the functional groups are arranged in a three-dimensional, spherical volume around the molecule's center.

“Small Molecule”: As used herein, the term “small molecule” refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 1500, although this characterization is not intended to be limiting for the purposes of the present invention. Examples of “small molecules” that occur in nature include, but are not limited to, taxol, dynemicin, and rapamycin. Examples of “small molecules” that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J. Am. Chem. Soc. 120:8565, 1998; incorporated herein by reference). In certain other preferred embodiments, natural-product-like small molecules are utilized.

“Template precursor”: Template precursor as used herein refers to a chemical compound that when subjected to certain reaction conditions will undergo a rearrangement or restructuring to create a molecular skeleton. In certain embodiments, the sites of functionalization on the template precursor may affect the molecular skeleton created when the precursor is subjected to certain reaction conditions. The template precursor may be cyclic or acyclic.

### DESCRIPTION OF THE DRAWING

FIG. 1 shows the analogy between the folding pathways approach and Nature's encoding of structural information in the primary amino acid sequence of proteins.

FIG. 2 shows the reaction mechanism of the Achmatowicz reaction.

FIG. 3 shows the use of furan oxidation in target-oriented synthesis to generate different molecular skeletons.

FIG. 4 shows a general split-pool synthetic plan for a folding pathway to generage skeletal diversity. The diamond-filled arrow is introduced to represent split-pool step(s) in diversity-oriented synthesis pathways.

FIG. 5 depicts a reaction network that converts a common, macrobead-bound furfural precursor 16 into furan derivatives 23-26 containing different linear side chains. These furan derivatives are then transformed into distinct molecular skeletons 27-30 under a common set of reaction conditions. Conditions: (a) (EtO)₂POCH₂CO₂CH₂CHCH₂, LiOH, THF, rt. (b) Pd(PPh₃)₄, thiosalicylic acid, THF, rt. (c) ClCOCH₂CH(CH₃)₂, iPr₂NEt, THF, 4° C.; LiBH₄, iPr₂NEt, THF, 4° C. (d) PhNCO, Pyr, CH₂Cl₂, rt. (e) OSO₄, (DHQD)₂PHAL, NMO, TEAAT, Acetone/H₂O (10:1), 4° C. (f) (CH₃O)₂C(CH₃)₂, CSA, CH₂Cl₂, rt. (g) (4S,5R)-(−)-4-methyl-5-phenyl-3-propionyl-2-oxazolidinone, nBu₂BOTf, Et₃N, CH₂Cl₂, −78° C. to 0° C.; H₂O₂, pH 7 buffer, MeOH, 4° C. (h) C₄H₃OCO₂H, DIC, iPr₂Net, DMAP, DMF/CH₂Cl₂ (1:1), rt. (i) NBS, NaHCO₃, NaOAc, THF/H₂O (5:1), 4° C. (j) CSA, CH₂Cl₂, 55° C.

FIG. 6 shows how distinct molecular skeletons could be generated in one pot on a diverse collection of macrobead-bound furan derivatives. Conditions: (a) (EtO)₂POCH₂CO₂CH₂CHCH₂, LiOH, THF, rt. (b) Pd(PPh₃)₄, thiosalicylic acid, THF, rt. (c) ClCOCH₂CH(CH₃)₂, iPr₂NEt, THF, 4° C.; LiBH₄, iPr₂NEt, THF, 4° C. (d) RNCO, Pyr, CH₂Cl₂, rt. (e) OSO₄, (DHQD)₂PHAL, NMO, TEAAT, Acetone/H₂O (10:1), 4° C. (f) (CH₃O)₂C(CH₃)₂, CSA, CH₂Cl₂, rt. (g) NBS, NaHCO₃, NaOAc, THF/H₂O (5:1), 4° C. (h) CSA, CH₂Cl₂, 55° C.

FIG. 7 shows a pathway to generate furfural derivatives compatible with split-pool synthesis.

FIG. 8 is a general split-pool synthetic plan using the folding pathway approach to generate three unique molecular skeletons under a common set of reaction conditions. “R” represents building blocks that are attached using split-pool synthesis.

FIG. 9 shows an encoded library with 4000 unique, spatially segregated compounds with three distinct molecular skeletons, all generated under a final, common set of reaction conditions.

FIG. 10 shows the orthogonal functionalization of commercially available 4,5-dibromofurfural via an iterative sequence of regioselective Suzuki reactions.

FIG. 11 shows a reaction network that generates a collection (Library #1) of macrobead-bound furfural precursors 4 and converts them into furan derivatives 5-8containing different linear side chains. These furan derivatives are then transformed into distinct molecular skeletons 9, 10/11, and 12 under a common set of reaction conditions. Conditions: (a) (EtO)₂POCH₂CO₂CH₂CHCH₂, LiOH, THF, rt. (b) Pd(PPh₃)₄, thiosalicylic acid, THF, rt. (c) ClCOCH₂CH(CH₃)₂, iPr₂NEt, THF, 4° C.; LiBH₄, iPr₂NEt, THF, 4° C. (d) PhNCO, Pyr, CH₂Cl₂, rt. (e) OSO₄, (DHQD)₂PHAL or (DHQD)₂PHAL, NMO, TEAAT, Acetone/H₂O (10:1), 4° C. (f) (CH₃O)₂C(CH₃)₂, CSA, CH₂Cl₂, rt. (g) (4S,5R)-(−)-4-methyl-5-phenyl-3-propionyl-2-oxazolidinone, nBu₂BOTf, Et₃N, CH₂Cl₂, −78° C. to 0° C.; H₂O₂, pH 7 buffer, MeOH, 4° C. (h) C₄H₃OCO₂H, DIC, iPr₂Net, DMAP, DMF/CH₂Cl_{2 (}1:1), rt. (i) NBS, NaHCO₃, NaOAc, THF/H₂O (5:1), 4° C. (j) CSA, CH₂Cl₂, 55° C.

FIG. 12 shows the building blocks of Library #1.

FIG. 13 shows a synthetic scheme for Library #2. In Library #2, more that one site of variability is impacting on the skeleton resulting in a combinatorial matrix (3×2=6>3+2=5) of molecular skeletons.

FIG. 14 shows synthetic strategies for generating building block diversity and skeletal diversity in diversity-oriented synthesis. (A) Schematic representation of the one synthesis-one skeleton approach for generating building block diversity combinatorially (the diamond-filled arrow is used in DOS to represent a split-pool step). (B) Schematic representation of the σ-element based synthesis strategy: transforming substrates having different σ-elements, i.e., appendages that pre-encode skeletal diversity, into products having different skeletons using common reaction conditions. With this approach, split-pool synthesis can be used to pre-encode skeletal diversity combinatorially, thereby generating small molecules having diverse skeletons very efficiently. (C) Schematic of a hybrid synthesis strategy for generating a collection of compounds representing a set of complete, overlapping matrices of building block and skeletal diversity elements, i.e., a complete combinatorial matrix of molecular skeletons, each derivatized with a complete combinatorial matrix of building blocks (the equivalent of several different collections of compounds synthesized individually using the one synthesis-one skeleton approach). (D) Forward-synthetic plan for a σ-element-based DOS pathway that uses a common set of reaction conditions to transform a collection of three furan-derived substrates into a collection of three products having distinct molecular skeletons. The number of nucleophilic hydroxyl groups (two, one, or zero) on the two methylene carbons flanking the furan ring represents a skeletal information unit (indicated by the symbol “σ”). (E) A common set of reagents was used to transform three substrates 5, 6, and 7, having different skeletal information pre-encoded in their structures, into three products 8, 9, and 10 having distinct molecular skeletons. Conditions: (a) trifluoromethanesulfonic acid, CH₂Cl₂ rt, 20 min; 5-hexen-1-ol, 2,6-lutidine, CH₂Cl₂, rt, 12 h; (b) 9-BBN, THF, rt, 5 h; 5-bromofuraldehyde, PdCl₂dppf, NaOH, THF/H₂O (5/1), 65° C., 20 h, 0.679 meq./g; (c) allyldiethylphosphonoacetate, LiOH, THF, rt, 25 h; (d) Pd(PPh₃)₄, thiosalicylic acid, THF, rt, 24 h; (e) isobutylchloroformate, 4-methylmorpholine, i-Pr₂NEt, THF, 0° C., 2 h; LiBH₄, i-Pr₂NEt, THF, 4° C., 24 h, purity 68%; (f) phenyl isocyanate, pyridine, CH₂Cl₂, rt, 24 h; (g) OSO₄, (DHQD)₂PHAL, 4-methylmorpholine N-oxide, TEAAT, Acetone/H₂O (10/1), 4° C., 48 h, purity >90%; (h) (S)-(+)-4-benzyl-3-propionyl-2-oxazolidinone, n-Bu₂BOTf, Et₃N, CH₂Cl₂, 72 h, −78° C. to 0° C.; 30% aq. H₂O₂, pH7 buffer, MeOH 4° C., 12 h, purity >90%; (i) acetic anhydride, i-Pr₂NEt, DMAP, CH₂Cl₂, rt, 28 h, purity >90%; (j) N-bromosuccinimide, NaHCO₃, NaOAc, THF/H₂O (4/1), rt, 1 h; PPTS, CH₂Cl₂, 40-45° C., 20 h, 8: purity 64% (22), 9: purity 86%, 10: purity >90%. Purities were determined by LCMS analysis (UV detection at λ₂₁₄) following HF-mediated cleavage of compounds from macrobeads.

FIG. 15 shows the generation of skeletal diversity combinatorially. (A) Substitutions at the 4-position of a common α-alkoxy furan core were found to also effect the formation of distinct molecular skeletons. X=(S)-(+)-4-benzyl-2-oxazolidinone, Ar=m-methylphenyl. Conditions: (a) 9-BBN, THF, rt, 5 h; 4,5-dibromofuraldehyde, PdCl₂dppf, NaOH, THF/H₂O (5/1), 65° C., 18 h, 0.188 meq./g; (b) 9-BBN, THF, rt, 5 h; 4-m-MePh-5-bromofuraldehyde, PdCl₂dppf, NaOH, THF/H₂O (5/1), 65° C., 22 h, 0.545 meq./g; (c) (S)-(+)-4-benzyl-3-propionyl-2-oxazolidinone, n-Bu₂BOTf, Et₃N, CH₂Cl₂, 72 h, −78° C. to 0° C.; 30% aq. H₂O₂, pH7 buffer, MeOH, 4° C., 12 h, 13: purity >90%, 15: purity >90%; (d) acetic anhydride, i-Pr₂NEt, DMAP, CH₂Cl₂, rt, 28 h, 14: purity >90%, 16: purity >90%; (e) N-bromosuccinimide, NaHCO₃, NaOAc, THF/H₂O (4/1), rt, 1 h; PPTS, CH₂Cl₂, CH₂Cl₂, 40-45° C., 20 h, 17: purity 90%, 14′: purity >90%, 18: purity 72%, 19: purity 66%. Purities were determined by LCMS analysis (UV detection at λ₂₁₄) following HF-mediated cleavage of compounds from macrobeads. (B) A complete, (3×2=6) combinatorial matrix of skeletal information units (—H, —Br, or —Ar at the 4-position of furan combined with —OH or —OAc on the α-methylene) resulted in a complete matrix of distinct skeletal outcomes under a common set of reaction conditions. (C) The missing bonds in both the substrates and products in FIG. 2B represent potential attachment sites to which building blocks could be appended. The six ‘substrates,’ having a (3×2) matrix of different appendages attached to a common α-alkoxy furan skeleton resemble the types of compounds typically derived from the one synthesis-one skeleton approach. Alternatively, the six ‘products’ represent six distinct molecular skeletons generated combinatorially using the σ-element-based synthesis strategy. Comparing and contrasting these two collections (which are almost constitutionally isomeric) can provide a metric for the skeletal diversity generated in this one reaction using a common set of reagents (25) (see supporting information for details). By replacing each of the missing bonds in the twelve structures shown in FIG. 2B with methyl groups (or a methylene group for the ‘left side’ of structure 9), we were able to generate a collection of simplified structures which all share in common the seven contiguous carbon atoms labeled C₁-C₇. Determination of equilibrium conformer and equilibrium geometry using semiempirical AM1 and Hartree-Fock (6-31G*) calculations, respectively, produced two collections of three-dimensional structures, from which the following geometrical parameters could be derived (each parameter is meant to provide unique information regarding the relative positions of the building block attachment sites, C₁ and C₇, in three-dimensional space): 1. the distance (in angstroms) between the two attachment sites C₁ and C₇, 2. the angle between C₁, the midpoint between C₃ and C₅, and C₇, and 3. the dihedral angle between C₁, C₃, C₅, and C₇ (every other carbon). As shown in FIG. 2C, when these three parameters are plotted, the six substrates create a very dense cluster (the two lobes of this dense cluster represent the acetylated and non-acetylated substrates). In stark contrast, the six products distribute much more broadly (both plots are drawn to the same scale) consistent with a diverse display of chemical information in three-dimensional space.

FIG. 16 shows the parallel synthesis of a complete combinatorial matrix of molecular skeletons, each derivatized with a complete combinatorial matrix of building blocks. (A) Four sets of appendages attached to a common α-alkoxy furan skeleton, two that do (skeletal information units σ₁ and σ₂), and two that do not (building blocks BB₁ and BB₂) influence the formation of distinct molecular skeletons upon exposure of a collection of substrates to a common set of reagents. For the 36 substrates 20a-jj 36/36 (100%) of the predicted structures were confirmed by ¹H NMR and HRMS, and 35/36 (97%) of these compounds were determined to be ≧70% pure by LCMS analysis. (B) Transformation of this collection of 36 substrates 20a-jj into a collection of 36 products representing two complete, overlapping matrices of building blocks and molecular skeletons. Conditions: N-bromosuccinimide, NaHCO₃, NaOAc, THF/H₂O (4/1), rt, 1 h; PPTS, CH₂Cl₂, 40-45 C, 20 h. ¹H NMR, LCMS, and HRMS were consistent with the formation of the anticipated functionalized skeleton in 36/36 cases (100%), and 26/36 (72%) of these products were determined to be >70% pure by LCMS analysis.

FIG. 17 illustrates the split-pool synthesis of ˜1260 compounds 55 representing a complete, combinatorial (3×2=6) matrix of molecular skeletons, each derivatized with a combinatorial (7×15=105) matrix of building blocks in both enantiomeric/diastereomeric forms (6×105×2=1260) (see supporting information for experimental details). LCMS analysis of compounds cleaved from 120 of these macrobeads confirmed the structure encoded by the orthogonally cleaved chemical tags in 120 out of 120 cases (100%). Moreover, 84/120 (70%) of these compounds were determined to be ≧70% pure by LCMS analysis.

FIG. 18 illustrates the branched pathway (reagent-based) approach to the synthesis of a library. Hydroxyl-substituted aromatic aldehydes (most frequently, phenolic aldehydes; vanillin is illustrated) were loaded onto high capacity macrobeads (denoted by the asterisk-within-a-circle symbol), converted to trienes, and reacted with dienophiles. The degree of substitution on the dienophiles determines whether they participate in the second cycloaddition (see text for details). The diamond inserted in the arrow denotes a split-and-pool step.

FIG. 19 shows 40 hydroxyaldehydes (top), 41 disubstituted dienophiles (middle), and 22 tri- or tetrasubstituted dienophiles (bottom) as building blocks used in the branched diversity-oriented synthesis pathway.

FIG. 20 shows products derived from the intermediates in FIG. 18 and several related products characterized by X-ray crystallography. Ring B on each skeleton is highlighted in black in the Chem 3D images derived from X-ray coordinates. Compounds 7′, 12, and 13 were produced through solution-phase synthesis during the pathway development phase of this research.

FIG. 21 illustrates how the branched (reagent-based) diversity-oriented synthesis pathway leads to compounds having ten distinct skeletons.

FIG. 22 illustrates skeletal diversity displayed by 10 discrete core structures and their 3-dimensional illustrations. The ring B on each skeleton is highlighted in black (perpendicular to the page) for comparison.

FIG. 23 shows the distribution of library members in molecular descriptor space. Two molecular descriptors (molecular weight and calculated logP value) are shown.

### DESCRIPTION OF CERTAIN PREFERRED EMBODIMENTS

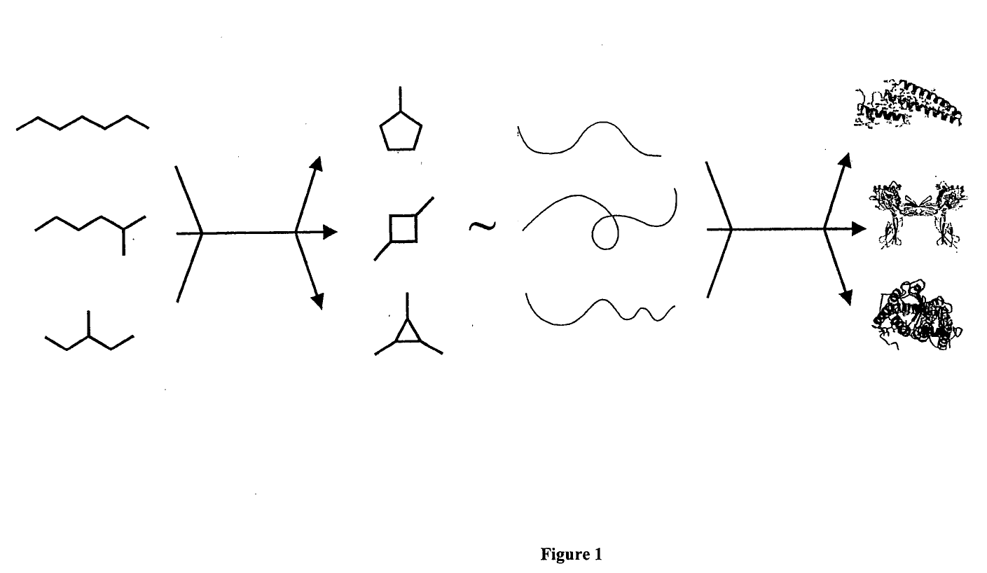

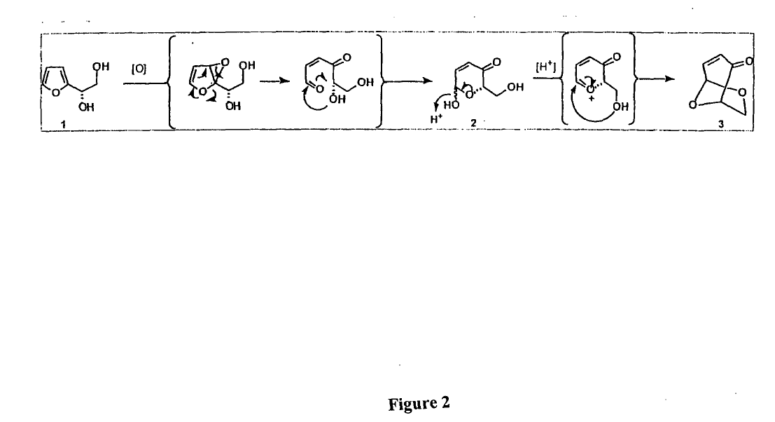

Skeletal diversity has been a long sought goal in the synthesis of combinatorial libraries of natural-product-like small molecules. Achieving skeletal diversity in a combinatorial library would allow for greater chemical diversity within a library rather than having all the chemical compounds of the library having a common core or molecular skeleton. One strategy for achieving skeletal diversity is the inventive branching reaction pathways approach (also known as the reagent-based approach), in which a single precursor is exposed to different reaction conditions to effect unique transformations into alternative skeletons (Schreiber, Science 287:1964-1969, 2000; incorporated herein by reference). A second inventive strategy (substrate-based approach) is to generate a diverse collection of template precursors that when exposed to a common set of reaction conditions will undergo unique transformations to yield a diverse collection of molecular skeletons, analogous to the way Nature encodes folding information in the string of amino acids making up a protein (Anfinsen, Science 181:223-230, 1973; incorporated herein by reference) (FIG. 1). This second strategy has been termed the “folding pathways” approach or substrate-based approach in recognition of this conceptual similarity.

Any chemical compound may be used as a template precursor in the construction of a combinatorial library with skeletal diversity using the above approaches. In certain embodiment the chemical compound undergoes a rearrangement or restructuring when subjected to certain reaction conditions. In certain embodiments, there may be a common set of reaction to generate molecular skeletons. In other embodiments, different reaction conditions generate a different molecular skeleton. These rearrangements or restructuring may involve a ring opening, ring closing, isomerization, carbon-carbon bond formation, carbon-carbon bond breaking, etc. In certain embodiment the template precursor is linear in nature with no cyclic structures. In other embodiments, the template precursor possesses cyclic structures.

The template precursor may be modified using split-pool synthesis or other techniques to create different functional groups at various sites of the molecule. In the folding pathways (substrate-based) approach, the identity and location of these functional groups will determine the eventual molecular skeleton of the library members. For example, a hydroxyl group may lead to a cyclization reaction whereas a protected hydroxyl group may not allow such a cyclization. By contrast, in the branching pathway (reagent-based) approach, the template precursors, preferably attached to a solid support such as a macrobead, are exposed to different sets of reaction conditions to effect the change in the molecular skeleton. In certain embodiments, the reaction conditions used in the folding pathways approach are oxidative. In other embodiments, the conditions are reductive. The change in molecular skeleton may be catalyzed by acid or base. In certain embodiments, an organometallic reagent is used to effect the change. In generating the molecular skeletons of the library, one or more ring systems may be opened, one or more rings systems may be formed, stereocenters may be epimerized, double bonds may be isomerized, unsaturated functional groups may shift, carbon-carbon bonds may be broken or formed, rings may be aromatized, hydrogens may migrate, etc. Preferably, the molecular skeletons generated in the library allow for the display of the functional groups in a variety of ways in three-dimensional space.

After the molecular skeleton of the members has been formed, the molecules may be further modified using any of the techniques of combinatorial chemistry including split-pool synthesis. For example, various functional groups may be added, removed, or modified. Protecting groups may be removed. In certain embodiments, the synthesis of each member of the library involves less than 10 steps, preferably less than 7, and more preferably between 3 and 5 steps. After the final products have been prepared, the molecules may be removed from the solid support and characterized. In certain embodiments, the reaction sequence or structure of the molecule is determined by decoding a set of tags placed on the solid support during the synthesis of the molecule. In certain embodiments, the tags are polyhalogenated aryl compounds. The molecule may also be characterized by more traditional methods such as NMR (e.g., ¹H, ¹³C), IR, UV, rotation, mass spectroscopy, chromatography (e.g., HPLC, TLC), melting point, etc. The members of the library may be screened using any assays known to identify compounds with a particular ability or characteristic as described herein.

As would be appreciated by one of skill in this art, the production of a combinatorial library with skeletal diversity may include the use any of the techniques or methods known in the area of combinatorial chemistry or synthetic organic chemistry. Certain useful methods and techniques include split-pool synthesis, tagging, solid phase synthesis, purification techniques, etc. These techniques may be used before, after, or during the reactions creating skeletal diversity within the library. Various techniques are described in the literature (please see Bunin et al. J. Am. Chem. Soc. 114:10997 (1992); DeWitt et al. Proc. Natl. Acad. Sci. U.S.A. 90:6909 (1993); Houghten et al. Nature 354:84 (1991); Lam et al. Nature 354:82 (1991); Dolle J. Comb. Chem. 4:369 (2002); Tan et al. J. Am. Chem. Soc. 120:8565 (1998); U.S. Pat. No. 6,573,110; U.S. Pat. No. 6,518,017; U.S. Pat. No. 6,489,093; U.S. Pat. No. 6,468,806; U.S. Pat. No. 6,440,667; U.S. Pat. No. 6,417,010; U.S. Pat. No. 6,413,724; U.S. Pat. No. 6,377,895; U.S. Pat. No. 6,355,490; U.S. Pat. No. 6,310,244; U.S. Pat. No. 6,274,716; U.S. Pat. No. 6,274,385; U.S. Pat. No. 6,255,120; U.S. Pat. No. 6,224,832; U.S. Pat. No. 6,207,820; U.S. Pat. No. 6,168,912; U.S. Pat. No. 6,114,309; U.S. Pat. No. 6,087,186; U.S. Pat. No. 6,075,166; U.S. Pat. No. 6,061,636; U.S. Pat. No. 6,045,755; U.S. Pat. No. 6,025,371; U.S. Pat. No. 6,017,768; U.S. Pat. No. 5,980,704; U.S. Pat. No. 5,962,337; U.S. Pat. No. 5,958,702; U.S. Pat. No. 9,945,070; U.S. Pat. No. 5,919,955; U.S. Pat. No. 5,880,972; U.S. Pat. No. 5,856,496; U.S. Pat. No. 5,821,130; U.S. Pat. No. 5,792,431; U.S. Pat. No. 5,785,927; U.S. Pat. No. 5,753,187; U.S. Pat. No. 5,741,713; U.S. Pat. No. 5,712,146; U.S. Pat. No. 5,698,685; U.S. Pat. No. 5,688,997; U.S. Pat. No. 5,618,825; U.S. Pat. No. 5,603,351; U.S. Pat. No. 5,506,337; US Published Patent Application 2003/0144260; US 2003/0142713; US 2003/0142704; US 2003/0139322; US 2003/0138788; US 2003/0130804; US 2003/0124599; US 2003/0120066; 2003/0119059; US 2003/0113800; US 2003/0108946; 2003/0104481; 2003/0100018; 2003/0082830; US 2003/0082630; US 2003/0077760; US 2003/0077707; US 2003/0059847; US 2003/0059826; US 2003/0049619; US 2003/0038941; US 2003/0035756; US 2003/0003489; US 2003/0032205; US 2002/0193563; US 2002/0183371; US 2002/0182735; US 2002/0182714; US 2002/0172970; US 2002/0164275; US 2002/0161028; US 2002/0160527; US 2002/0160413; US 2002/0160380; US 2002/0146744; US 2002/0143476; US 2002/0143144; US 2002/0135753; US 2002/0127608; US 2002/0127599; US 2002/0127598; US 2002/01155106; US 2002/0102611; US 2002/0102608; US 2002/0098598; US 2002/0098513; US 2002/0094541; US 2002/0085063; US 2002/0077491; US 2002/0072594; US 2002/0067120; US 2002/0061598; US 2002/0061258; US 2002/0052003; US 2002/0045991; US 2002/0037534; US 2002/0029114; US 2002/0025535; US 2002/0022626; US 2002/0022243; US 2002/0022237; US 2002/0019013; US 2002/0017617; US 2002/0012948; US 2002/0012912; US 2002/0009627; US 2002/0006672; US 2002/0001541; US 2001/0053555; US 2001/0053530; US 2001/0051349; US 2001/0029028; US 2001/0025084; each of which is incorporated herein by reference).

Furan-based Libraries

In one embodiment of the “folding pathways” (substrate based) approach, the chemistry of fiuran derivatives with its skeletal diversity-generating potential was used to generate furan-based libraries. It is known in the art that the oxidation of furan derivatives leads to highly reactive ene-dione intermediates. As shown in FIG. 2, Achmatowicz and coworkers first demonstrated that treatment of a furan 1,2-diol (e.g., 1) with an oxidant such as mCPBA effects oxidative opening of the furan ring to yield an ene-dione intermediate that is trapped intramolecularly by the alpha hydroxyl group to give an enone-containing cyclic hemiacetal (2) (Achmatowicz Tetrahedron 27:1973-1996, 1971; incorporated herein by reference). Subsequent treatment of this hemiacetal intermediate with catalytic amounts of a Bronsted acid effects formation of an oxocarbenium ion, which is then trapped intramolecularly by the beta hydroxyl group to yield a [3.2.1] bicyclic ketal structure containing a bridging enone (3).

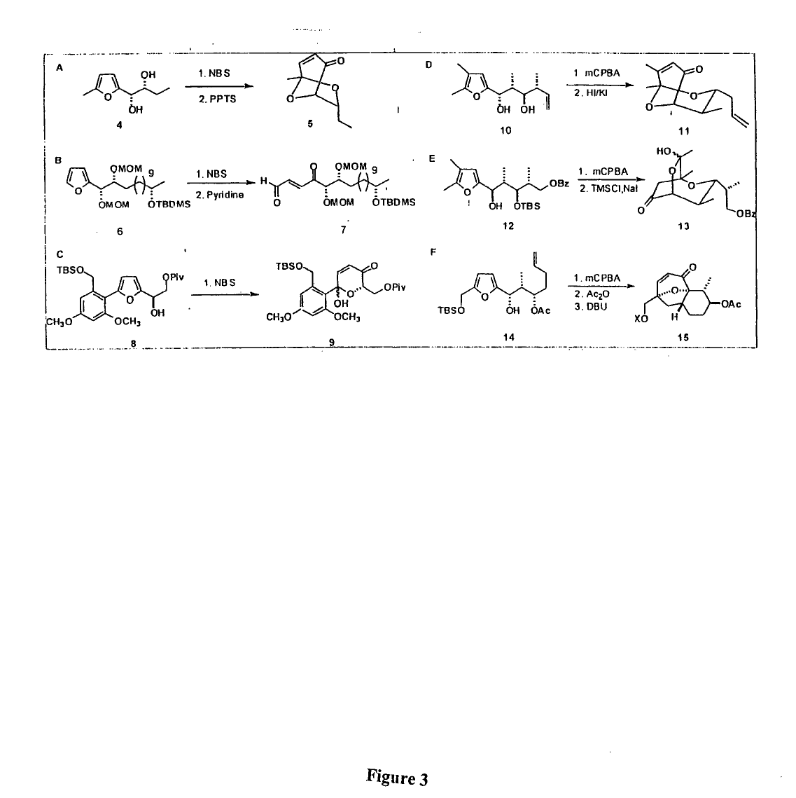

The Achmatowicz reaction has been used extensively in the context of target-oriented synthesis (e.g., see FIG. 3A) (Ogasawara Chem. Commun. 1477-1478, 1996; incorporated herein by reference). In addition, there are reports demonstrating the potential of this furan oxidation chemistry to generate skeletons different from the [3.2.1] bicycle when the furan is flanked by alternative linear side-chains (FIG. 3B-F). For example, Kobayashi and coworkers have shown that the bis protected 1,2 diol 6 undergoes oxidative furan cleavage followed by pyridine-mediated cis to trans isomerization of the cis ene-dione intermediate to yield a trans ene-dione skeleton (FIG. 3B) (Kobayashi J. Org. Chem. 63:7505-7515, 1998; incorporated herein by reference). In addition, Doherty and coworkers have demonstrated that a highly functionalized furan derivative 8 containing a nucleophilic hydroxyl group only in the alpha position can undergo oxidative ring expansion to yield an epimeric mixture of cyclic hemiacetals 9 that lack a second nucleophilic center (FIG. 3C) (O'Doherty Org. Lett. 2(25):4033-4036, 2000; incorporated herein by reference). All three of these transformations may be used under a common set of reaction conditions, i.e., oxidation to effect furan cleavage followed by acid-mediated bicycloketalization, cis to trans isomerization, or epimerization of an anomeric center yield three distinct molecular skeletons.

The chemical compounds of the library may be further functionalized, for example using the techniques of combinatorial chemistry, to further diversify the compounds in the library. In one embodiment, the compounds are functionalized using split-pool chemistry. To give but a few examples of reactions that may be performed on the resulting molecular templates, hydroxyl group or other nucleophiles may be alkylated, oxidized, acylated, protected, reduced, reacted with electrophiles, etc.; ene-diones may be alkylated or reduced; olefins may be oxidatively cleaved, reduced, hydroxylated, isomerized, oxidized to form an epoxide or aziridine, alkylated, etc.; carbonyls may be reduced, used to form olefins in Wittig-type reactions, used as electrophiles in reactions such as the Aldol reaction; oxidized, etc.; protected hydroxyl groups may be deprotected and then further reacted. As would be appreciated by one of skill in the art, the functional groups that result from one set of reactions may be further reacted in subsequent reactions. In addition, the reactions may be carried out in an enantioselective or diastereoseletive manner. In certain embodiments, the reactions have been shown to give high yields in the solid phase.

In using the split-pool synthetic methodology, tags may be attached to the solid support to which the members of the library are attached so as to identify the synthetic history of the compounds on the solid support. Upon the selection of a compound that needs to be identified, the tags attached to the solid support may be decoded to elucidate the structure of the chemical compound attached to the solid support.

In certain embodiments, the furan derivatives used in preparing a library are of the formula:

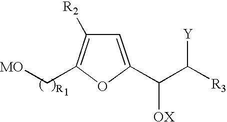

wherein R₁ is an aliphatic or heteroaliphatic linker; preferably, alkyl containing 1-20 carbons;

R₂ is hydrogen, halogen, lower alkyl, aliphatic, heteroaliphatic, aryl, heteroaryl, alkylaryl, or alkylheteroaryl; preferably, hydrogen, fluorine, bromine, chlorine, iodine, aryl, heteroaryl;

R₃ is alkyl, alkenyl, alkynyl, aliphatic, heteroaliphatic, aryl, heteroaryl;

M is a solid support or hydrogen;

X is a hydrogen, protecting group, acetyl, lower alkyl group, or lower acyl group; and

Y is hydrogen, halogen, hydroxyl, protected hydroxyl, amino, alkylamino, dialkylamino, lower alkyl, aliphatic, heteroaliphatic, aryl, or heteroaryl; preferably, hydrogen, hydroxyl, protected hydroxyl, methyl, ethyl, propyl, butyl, pentyl, or hexyl.

In certain other embodiments, the furan derivative is of the formula:

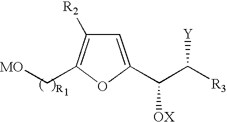

wherein R₁, R₂, R₃, M, X and Y are as defined above.

In yet other embodiments, the furan derivative is of the formula:

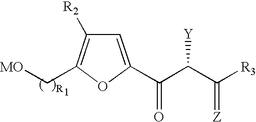

wherein R₁, R₂, R₃, M, X, and Y are as defined above;

Z is oxygen, sulfur, or NR_{J}, wherein R_{J} is hydrogen, lower alkyl, or hydroxyl.

In certain embodiments, R1 is selected from the following group:

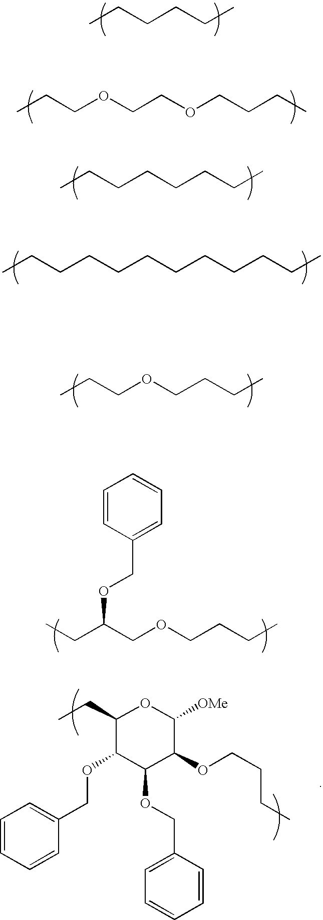

In certain embodiments, R₂ is hydrogen, bromine, phenyl, or substituted phenyl.

In certain embodiments, R₃ is selected from the following:

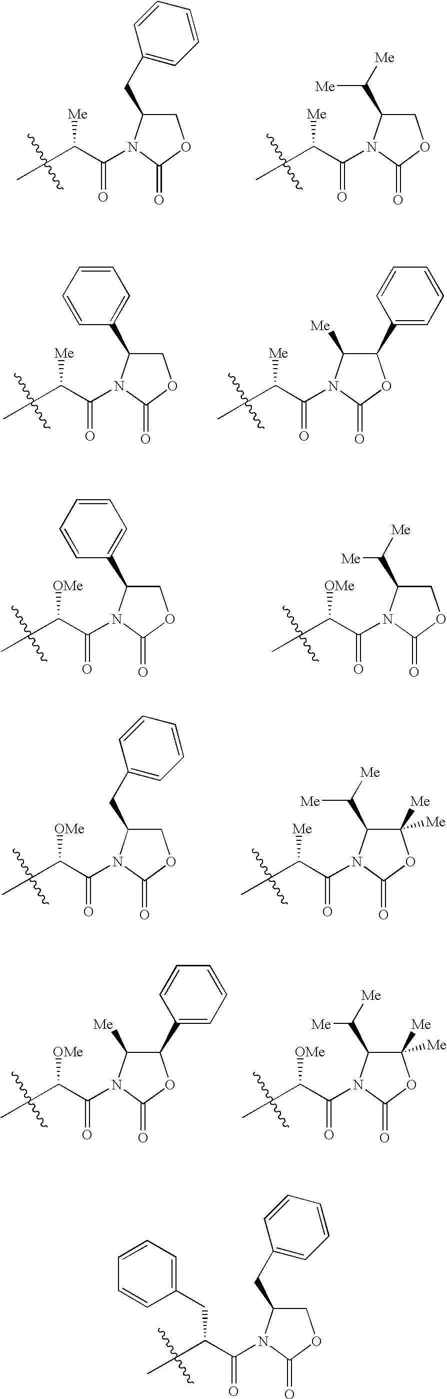

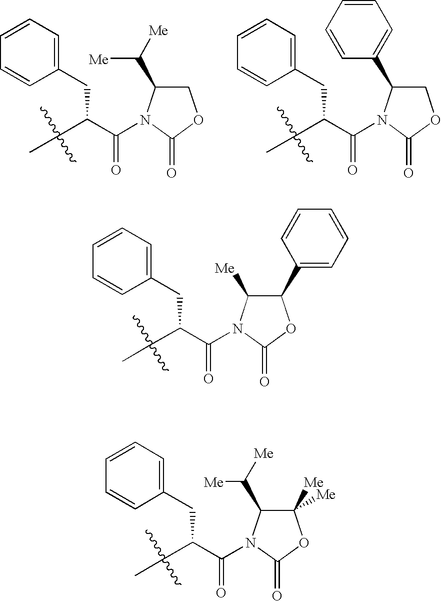

In certain embodiments, X is hydrogen or acetyl.

The furan derivatives may be treated to oxidative and acidic conditions (e.g., NBS, PPTS) resulting in library members with various molecular skeletons upon which various functional groups are displayed. The following basic molecular skeletons may be created by this method:

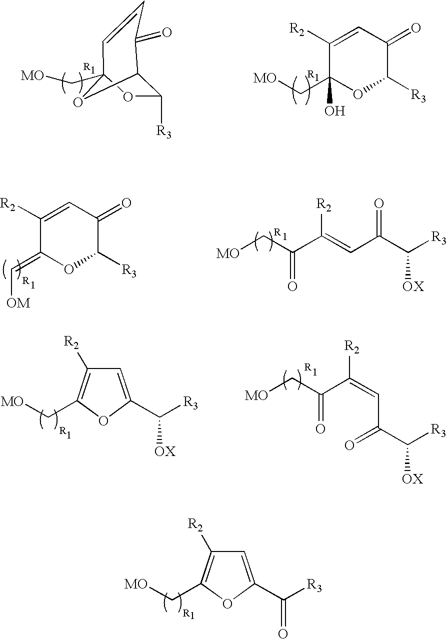

wherein R₁, R₂, R₃, M, and X are as defined supra.

Compounds of the invention include any of the following:

[image]

Libraries of Polycyclic Compounds Based on Reagent-based Approach

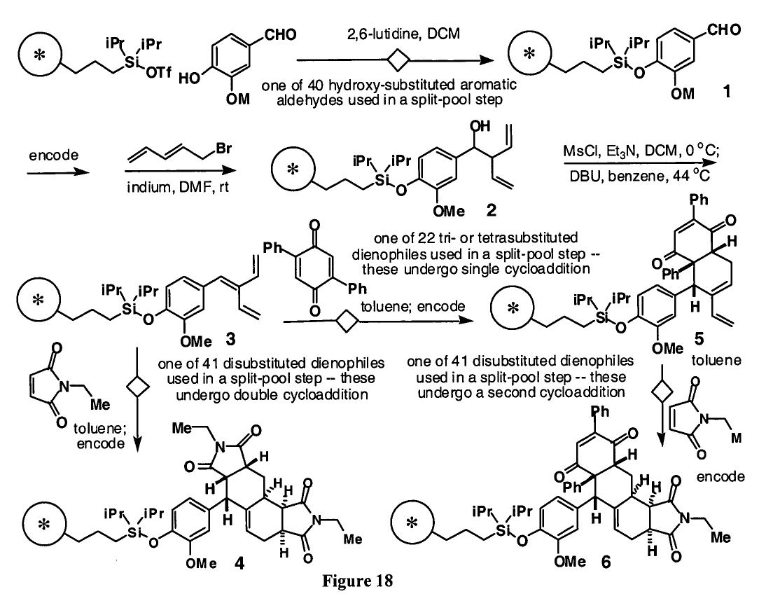

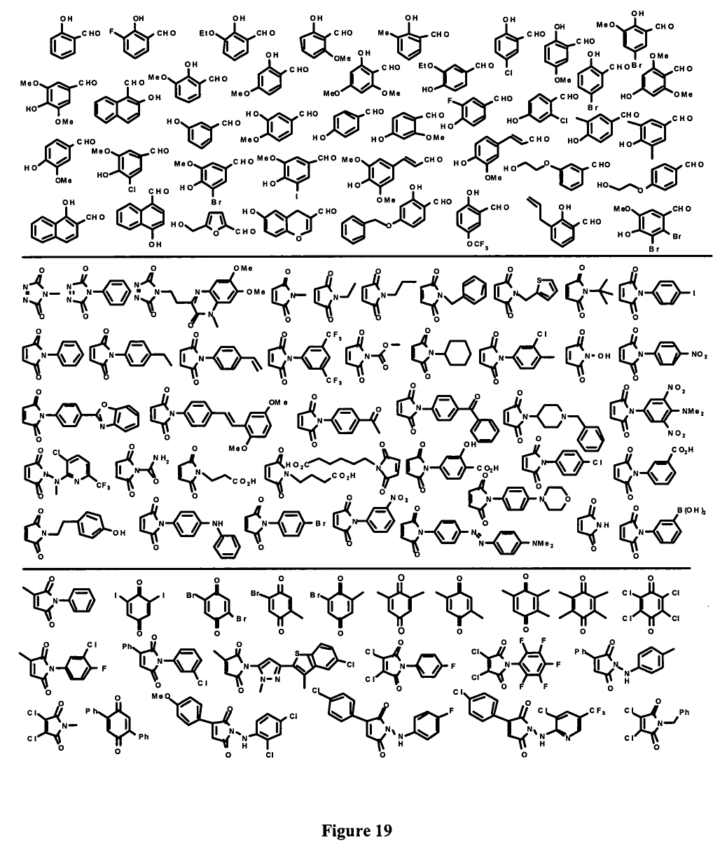

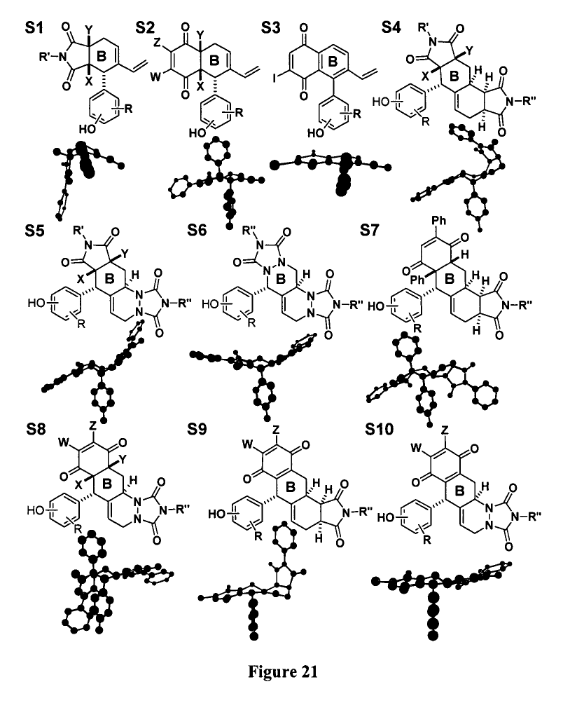

In one aspect, the invention provides the compounds of a library of 29,400 discrete, polycyclic compounds created using the reagent-based approach. These compounds are created by consecutive Diels-Alder cycloaddition reactions using a variety of dienophiles (see FIG. 19). Forty hydroxy aldehydes as shown at the top of FIG. 19 are attached to macrobeads. These aromatic aldehydes were then reacted to form cross-conjugates trienes susceptible to various cycloaddition reactions (see FIG. 18). The resulting trienes were reacted with various substituted dienophiles (see middle and bottom of FIG. 19) to achieve compounds having ten distinct molecular skeletons (see FIG. 21). As would be appreciated by one of skill in the art other hydroxyaldehydes and dienophiles could be used to produce greater diversity in the library or to product analogous libraries. In addition, a diene other than 5-bromo-1,3-pentadiene may be used to create further diversity in the library. The final compounds may be cleaved from the macrobead and assayed, characterized, or purified using any of the techniques known in the art.

Compounds of the invention include any compound of a formula selected from the group below:

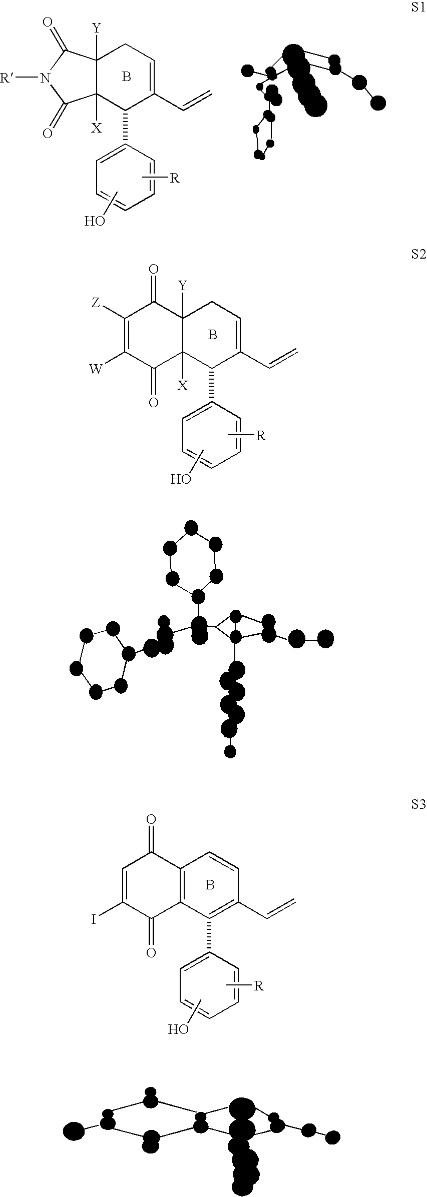

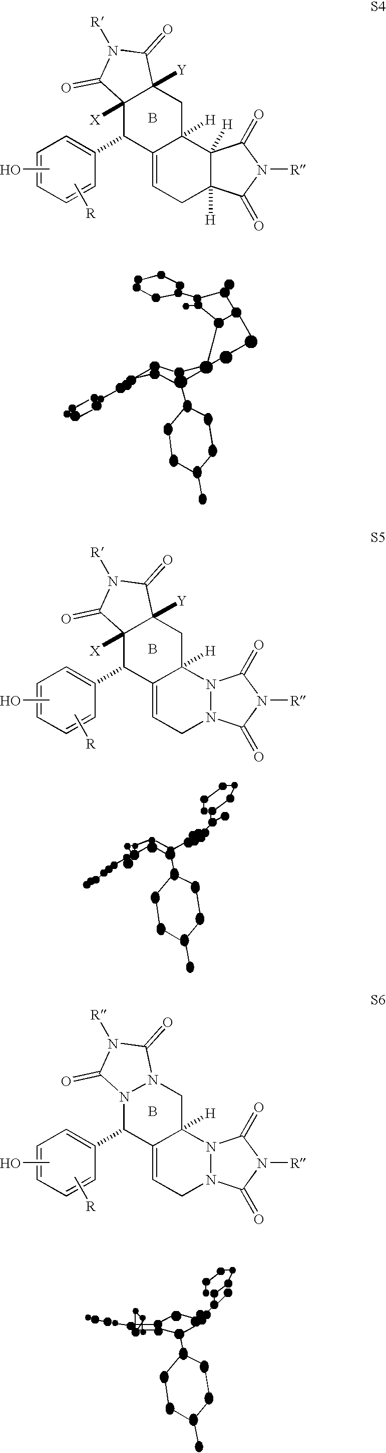

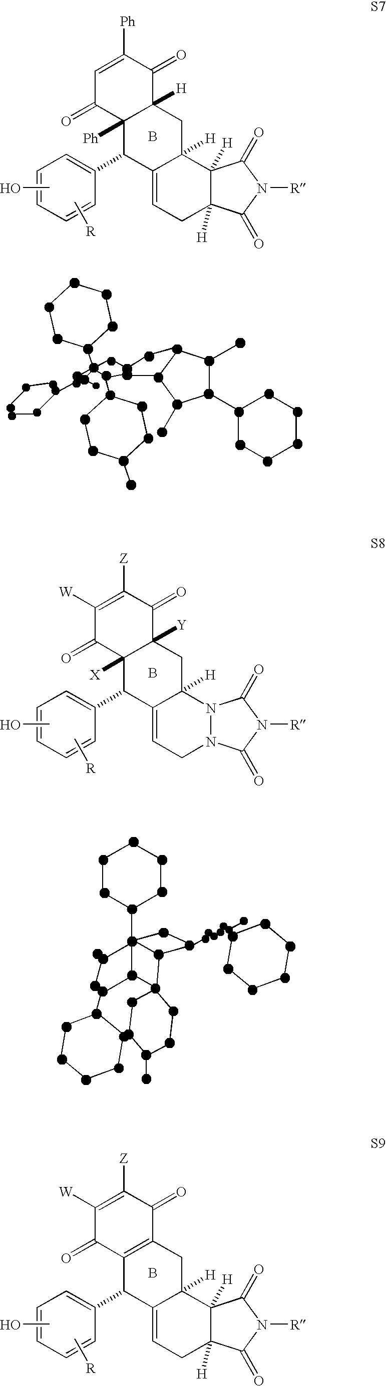

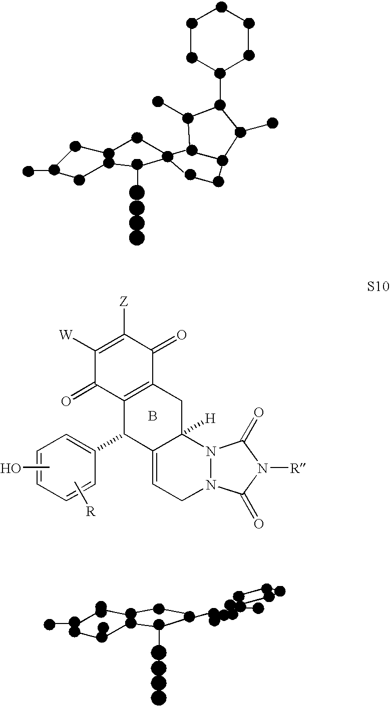

wherein wherein R is hydrogen, halogen, lower alkyl, lower alkoxy, or hydroxy; preferably each occurrence of R is independently selected from the group consisting of fluorine, chlorine, bromine, iodine, methoxy, ethoxy, benxyloxy, methyl, ethyl, propyl, and allyl;

n is an integer between 1 and 4;

R′ and R″ are independently hydrogen, aryl, substituted aryl, heteroaryl, substituted heteroaryl, carobcyclic, heterocyclic, acyl, hydroxyl, lower alkyl, or lower alkenyl;preferably, R′ is selected from the group consisting of hydrogen, methyl, ethyl, propyl, tert-butyl, arylalkyl,benzyl, phenyl, substituted phenyl, acyl, cyclohexyl, hydroxy, amino, alkylamino, and dialkylamino; and R″ is selected from the group consisting of hydrogen, methyl, phenyl, arylalkyl, and heteroarylalkyl; and

W, X, Y, and Z are independently hydrogen, lower alkyl, aryl, substituted aryl, heteroaryl, substituted heteroaryl, or halogen; preferably, W, X, Y, and Z are each independently selected from the group consisting of hydrogen, methyl, ethyl, propyl, fluorine, bromine, chlorine, iodine, phenyl, and substituted phenyl.

Uses

The chemical compounds produced using the inventive method may be used in any chemical application. Examples of uses of the compounds include catalysts, ligands for catalysts, pharmaceutical agents, research tools, materials, polymers for materials, etc.

In certain embodiments, the compounds of the library are used in known biological assays to identify compounds with a certain biological activity. For example, the compounds may be used in certain cell based assays to identify compounds with anti-neoplastic activity or with the ability to affect the cell cycle. Other assays may be used to identify compounds with antibiotic activity against certain organisms. Once a compound with a given activity has been identified, it may be used as a pharmacological agent or as a lead compound in the pursuit of a pharmacological agent.

In other embodiments, the compounds are used in the field of chemical genetics as research tools to study cell functioning. For example, the chemical compounds may be used to perturb and thereby understand protein function.

In certain embodiments, the compounds of the invention are provided as pharmaceutical compositions. These pharmaceutical composition may also include excipients useful in the pharmaceutical arts. The compound of the invention in such pharmaceutical compositions may be provided as a particular stereoisomer in pure, substantially pure, or racemic form. The compound may be provided as a particular crystalline polymorph. In certain embodiments, the pharmaceutical composition is provided in a form (e.g., tablet, capsule, solution, suspension, etc.) convenient for administration to an animal in need of treatment, preferably a mammal, more preferably a human.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### A Diversity-oriented Synthetic Pathway Analogous to Protein Folding used to Generate Skeletal Diversity within a Novel Library of 8,000 Unique Compound Derived from Furan Oxidation Chemistry

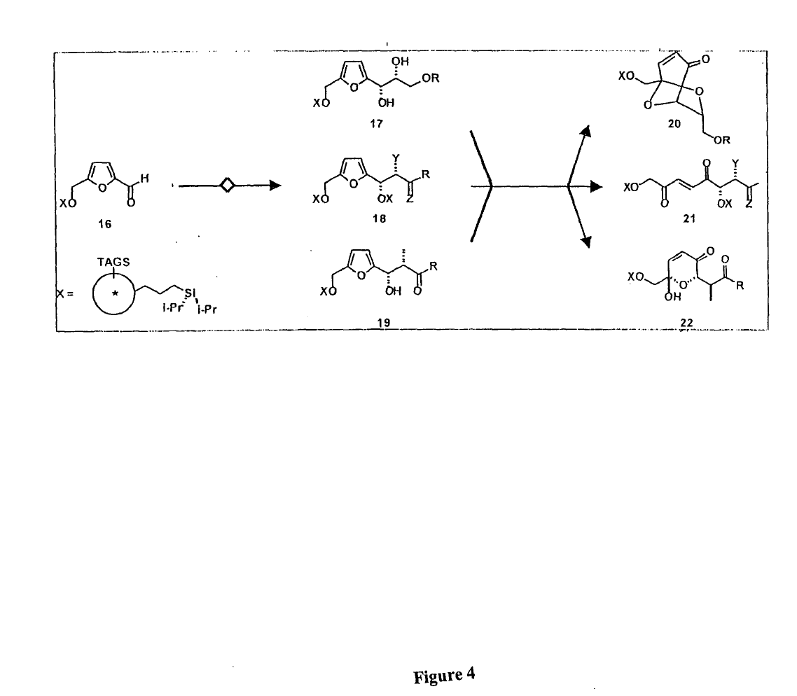

As shown in FIG. 4, guided by the folding pathways strategy, an encoded, split-pool synthetic plan has been developed to generate a diverse collection of macrobead-bound furan derivatives that contain three different linear side chains similar to those shown in FIG. 3A-C. The chemical information contained in the different side chains is transformed into skeletal diversity under a common set of furan oxidation/acid catalysis conditions.

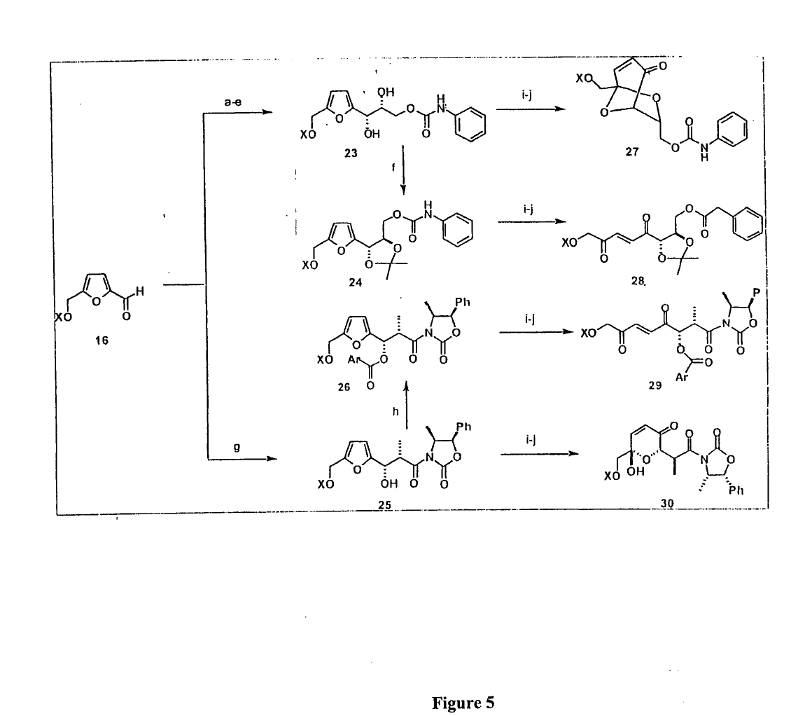

As shown in FIG. 5, a reaction network was developed that converts a common, macrobead-bound furfural precursor into the desired furan derivatives. Specifically, a Horner-Wadsworth-Emmons olefination of macrobead-bound 5-hydroxymethylfurfural 16 with allyl diethylphosphonoacetate/LiOH effected quantitative conversion into the desired trans-α,βunsaturated allylester (E/Z>95/5, conversion and stereoselectivity were determined by cleavage of the compound from the macrobeads with HP-pyridine followed by ¹H NMR and LCMS analysis of the crude product residue). Palladium/thiosalicylic acid-mediated deallylation proceeded smoothly to generate the desired trans-α,β-unsaturated acid, which was then cleanly reduced to the corresponding allylic alcohol in a two-step procedure involving activation with isobutylchloroformate followed by LiBH₄ mediated reduction of the mixed anhydride. Functionalization of the allylic alcohol with phenyl isocyanate, followed by a Sharpless asymmetric dihydroxylation resulted in quantitative conversion to the desired syn α,β-furan diol 23. The enantiomeric excess (e.e.) for this reaction was >90% in a solution-phase model system. The e.e. for the reaction on a solid-phase support has not yet been determined. Treatment of diol 23 with 2,2-dimethoxypropane in the presence of camphorsulfonic acid was found to effect clean conversion into 24, in which the two nucleophilic hydroxyls have been tied up in a ketal ring. In addition, a route was developed to convert the same macrobead-bound furfural starting material into derivatives containing only a single hydroxyl group in the a position. Specifically, the Evans' aldol reaction preformed on the macrobead-bound aldehyde 16 effected quantitative conversion into the desired aldol adduct 25 with >95% d.e. Moreover, it was found that the nucleophilic hydroxyl group in 25 could acylated under mild conditions, and the product 26 contains a linear side chain that is functionally equivalent to that found in compound 24. Finally, a set of common reaction conditions (i.e., NBS oxidation followed by treatment with camphorsulfonic acid) was found that effect conversion of 23, 24/26, and 25 into the enone-bridged bicyclic ketal 27, the trans ene-diones 28/29, and the monocyclic hemiacetal 30 respectively.

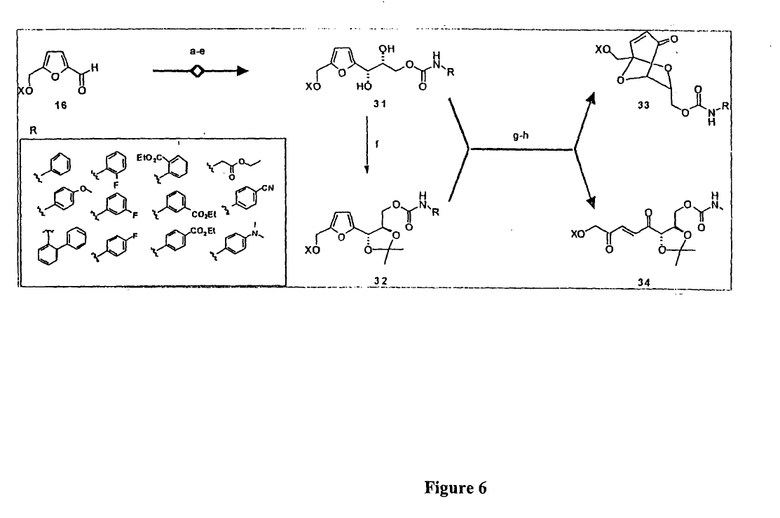

Distinct molecular skeletons were then generated in one pot based on macrobead-bound furan derivatives with a diverse set of building blocks attached to the right-hand portion of the compounds. As shown in FIG. 6, a series of commercially-available isocyanate building blocks were screened, and a set of twelve were found to undergo efficient coupling and subsequent Sharpless asymmetric dihydroxylation to yield a collection of twelve unique furan diols 31. Some N-oxide formation was observed for the dimethylamino-containing building block. Twelve individual beads with each containing a unique furan diol were then isolated and subjected to ketalization conditions en masse. The resulting twelve macrobeads 32 that each contained a unique “protected” furan diol were then combined with twelve macrobeads 31 that each contained a unique unprotected furma diol in one pot and subjected to NBS followed by CSA. The twenty-four individual product macrobeads were then segregated and cleaved with HF-pyridine, and the crude residue from each cleavage reaction was subjected to LCMS analysis. The results showed that both the [3.2.1] bicyclic ketal 33 and the trans ene-dione skeletons 34 were generated in one pot under a common set of reaction conditions. For nineteen out of the twenty-four individual beads the mass of the major product corresponded to one these two molecular skeletons (scaffolds). In this experiment, twelve out of the twenty-four beads released products that were estimated to be 80-90% pure, this after seven or eight steps on the solid-phase without the benefit of purification of the various synthetic intermediates.

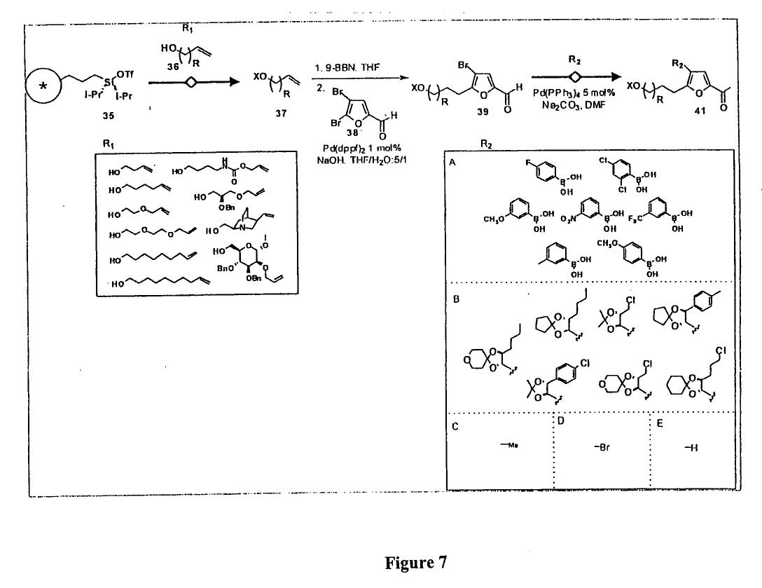

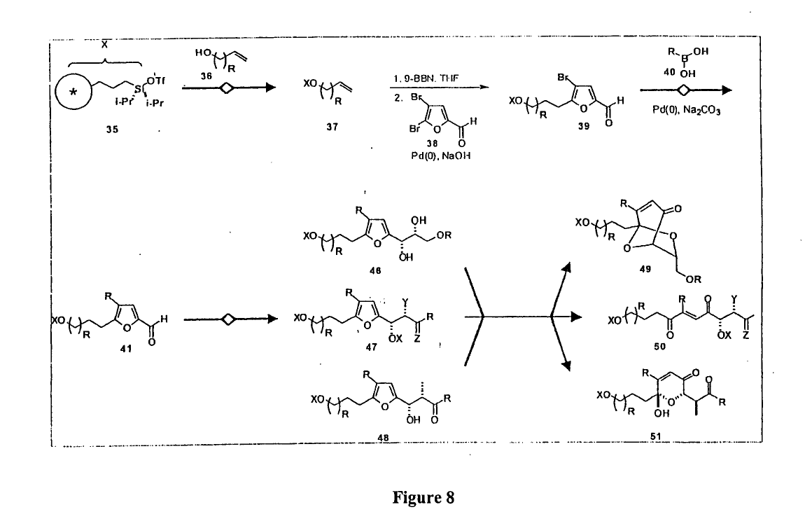

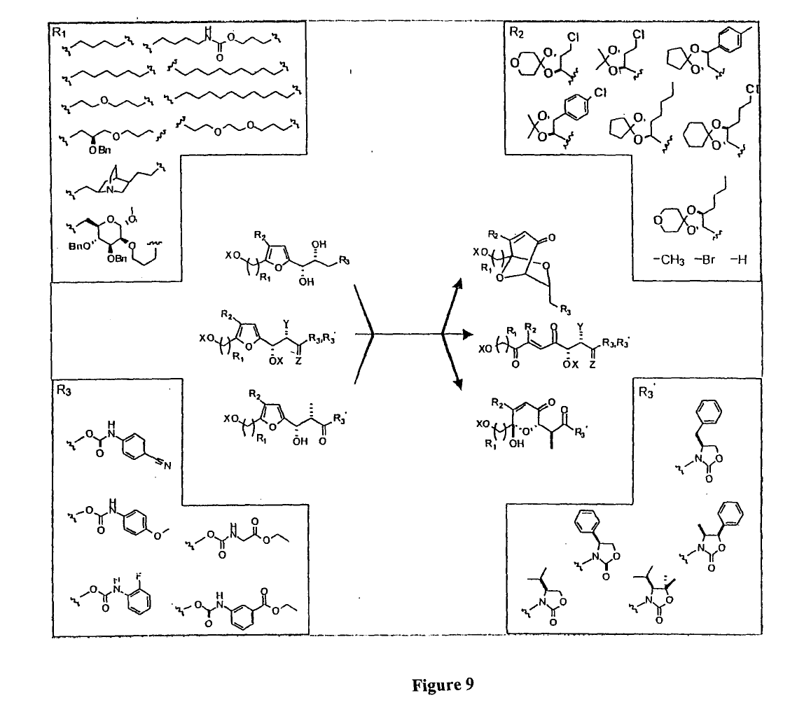

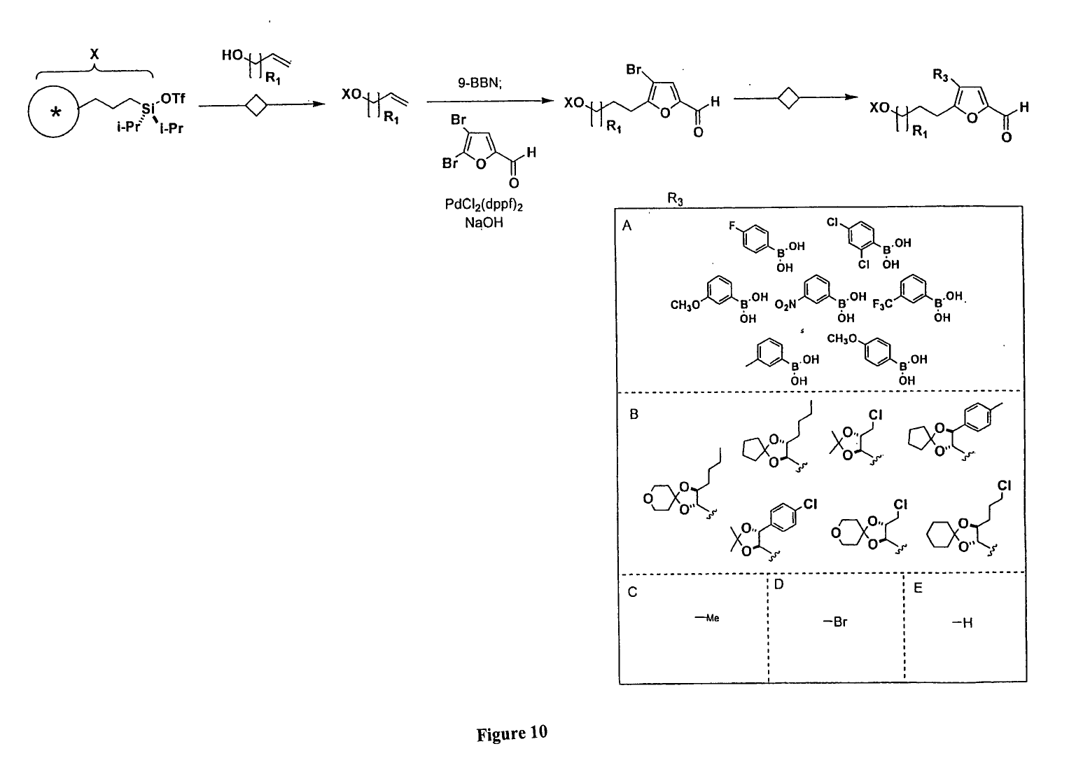

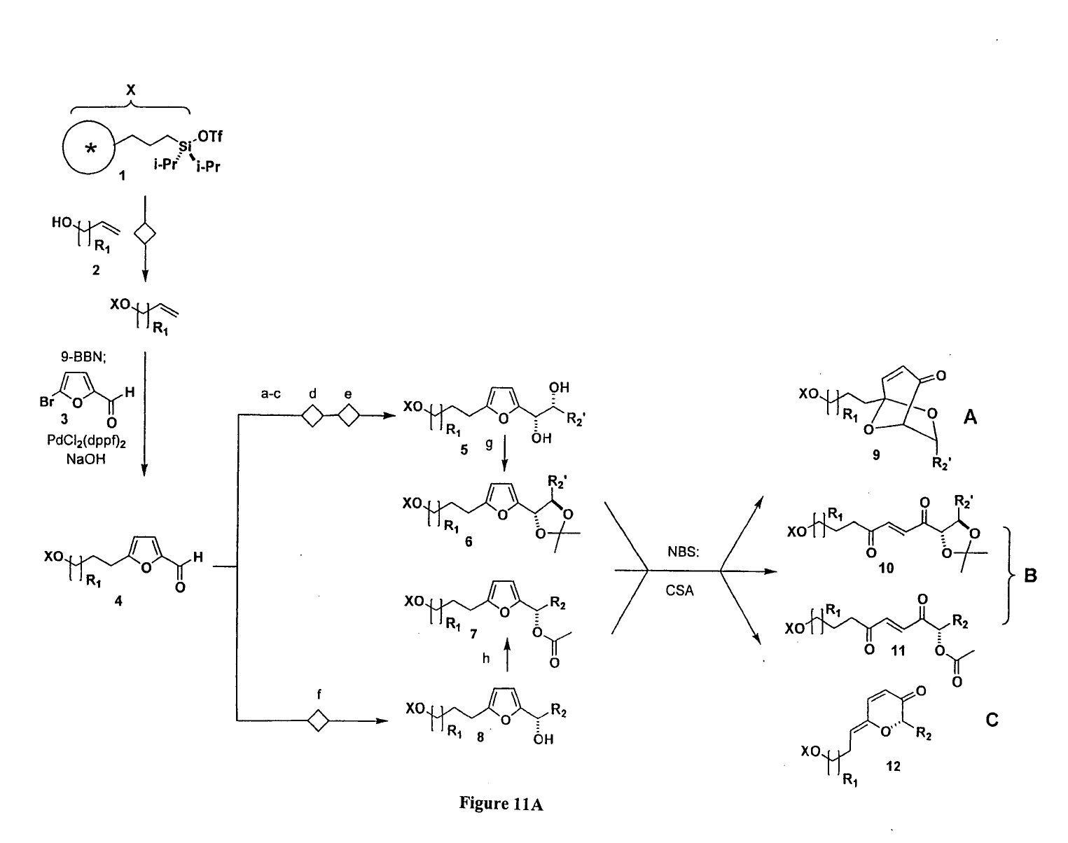

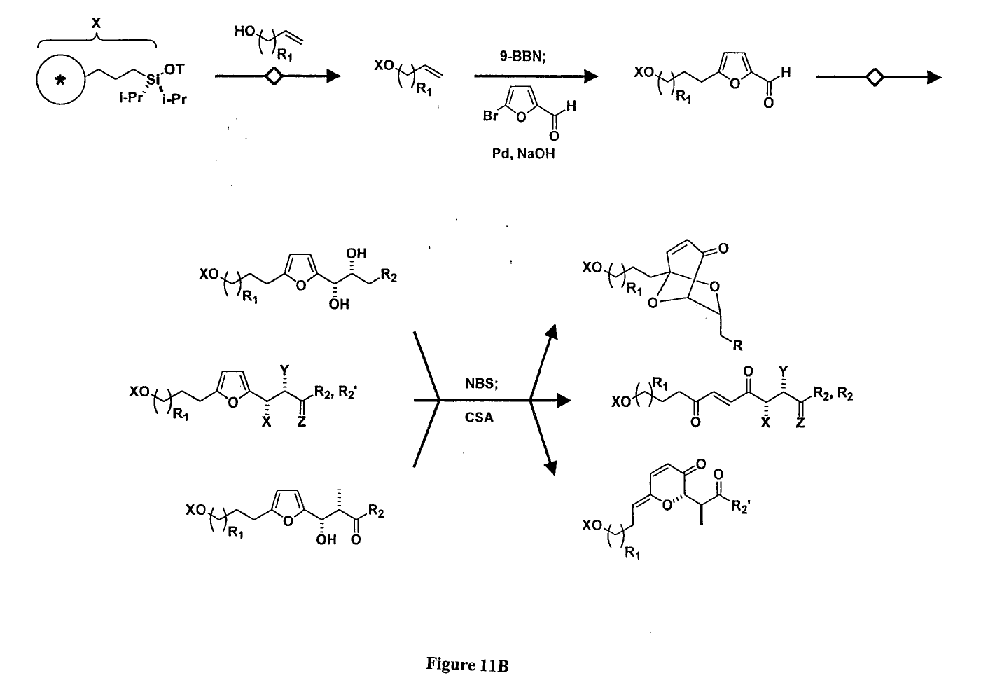

To enhance the building block diversity of the final library a split-pool sequence was developed to combinatorially generate macrobead-bound furfural derivatives to serve as substrates in this pathway. The applicability of the folding pathways strategy to a collection of highly diverse, combinatorially generated linear precursors was tested by pursuing the orthogonal functionalization of commercially available 4,5-dibromofurfural via an iterative sequence of regioselective Suzuki reactions (for a related regioselective Sonogashira coupling, see Bach, T. Eur. J. Org. Chem. 2045-2057, 1999; incorporated herein by reference). As shown in FIG. 7, this iterative Suzuki reaction sequence proceeds very cleanly on macrobeads and building block testing suggests that this route could prove to be highly general in the context of a split-pool synthesis.

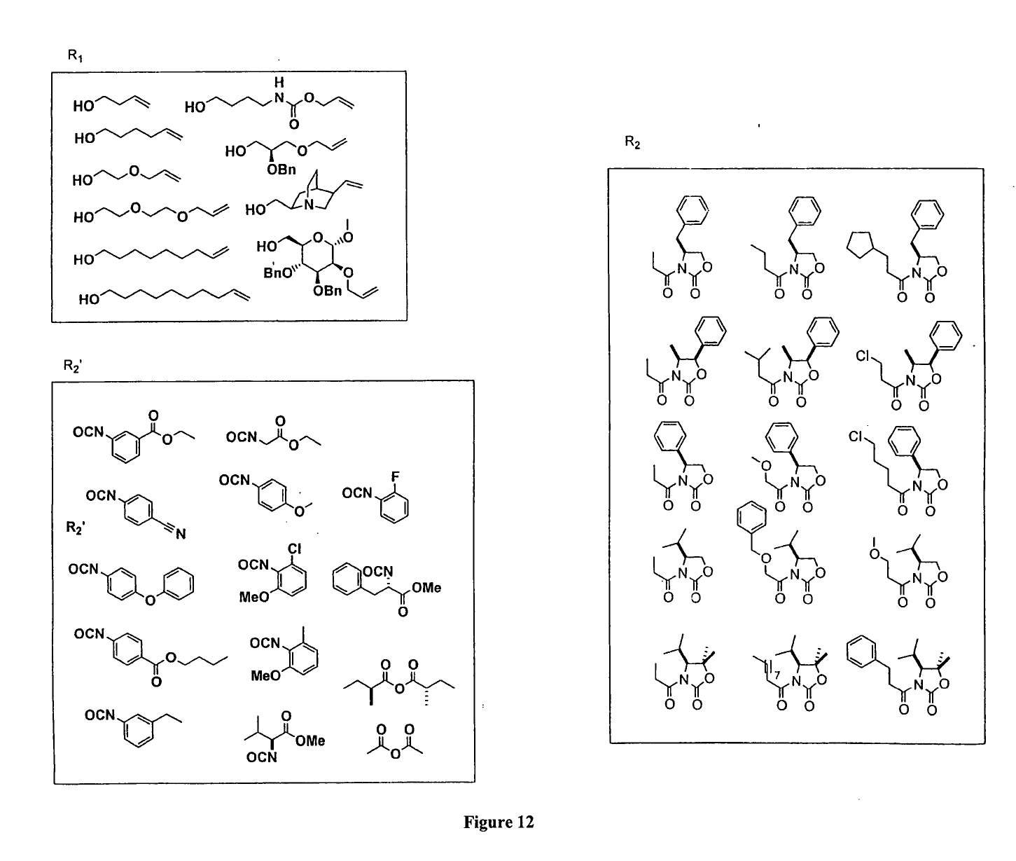

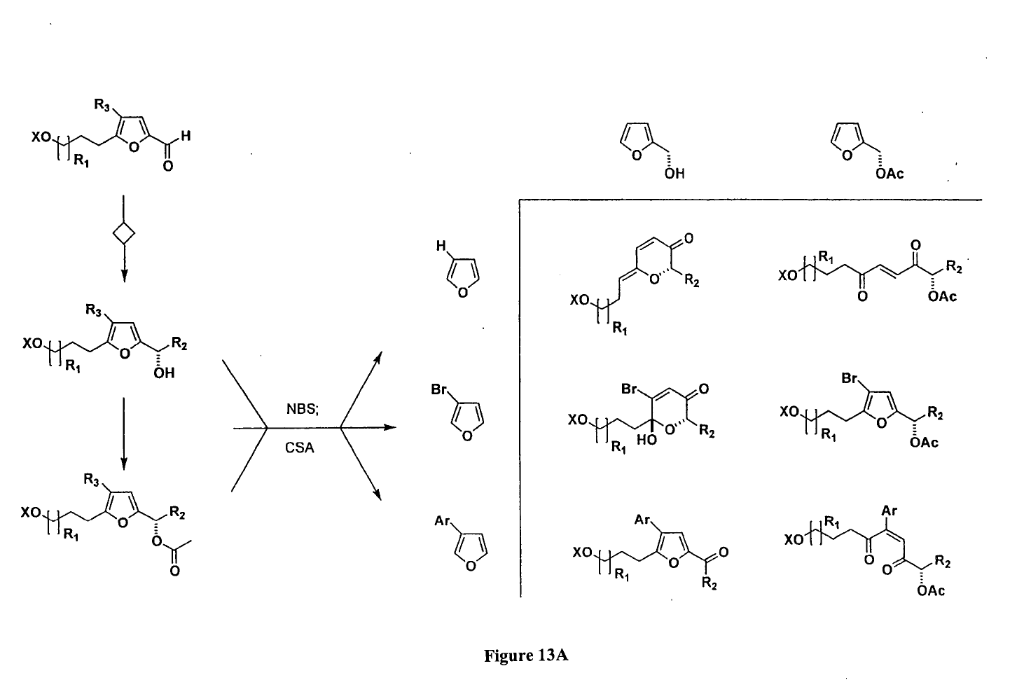

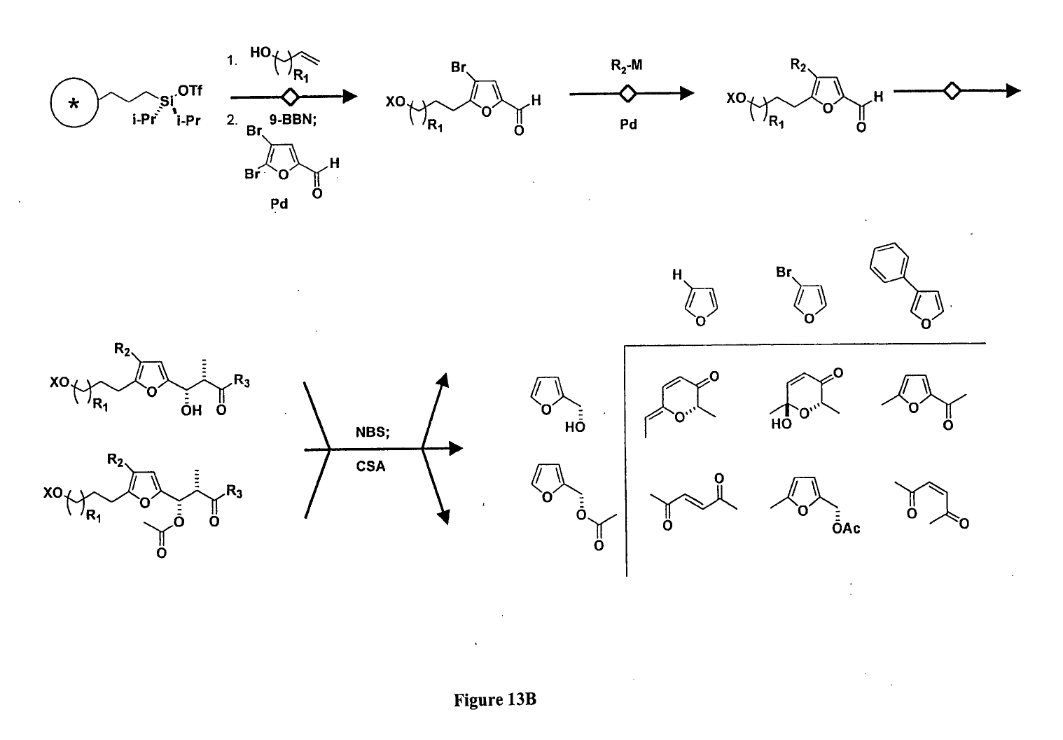

Guided by the folding pathways strategy, the furan oxidation chemistry was used to create an encoded library that includes unique, highly functionalized molecular skeletons under a common set of reaction conditions (see Example 2). The iterative Suzuki reaction sequence shown in FIG. 7 in a split-pool format can be used to generate a collection of macrobead-bound furfural derivatives containing all possible combinations of two sets of building blocks. The chemistry developed for the simpler model system can be used to convert a pooled set of bis-functionalized furfural derivatives into three distinct sets of linear structures, each containing a third building block on the right hand portion of the compounds (see FIG. 12 for Evans' aldol reactions, five different oxazolidinones—each commercially available in both enantiomeric forms-will serve as both a chiral auxilliary and a third building block). Finally, this pooled collection of furan derivatives is be exposed to a common set of oxidation/acid catalyst conditions to effect transformation into highly functionalized [3.2.1 ] bicyclic ketal, trans ene-dione, cis ene-dione, and monocyclic hemiacetal skeletons.

As shown in FIG. 7, some of the building blocks in building block set #1 are chiral and commercially available in non-racemic form. The Sharpless asymmetric dihydroxylation and the Evans asymmetric aldol reaction can be used to convert the combinatorially derived furfural precursors into both possible stereoisomers of the desired furan linear side chains. Therefore, for substrates that contain one of the chiral building blocks, these steps in the reaction network represent diastereoselective reactions in which it will be necessary for the chiral reagent (i.e., the Sharpless dihydroxylation catalyst or the Evans' aldol auxillary) to override any inherent bias of the macrobead-bound chiral substrate to yield a diastereomerically enriched product. Both enantiomers of these chiral reagents are readily available for both reactions, and all possible combinations of the anticipated diastereomeric products can be generated. The availability of powerful chiral reagents to override substrate bias and deliver pure, diastereomeric products is critical to the generation of high levels of stereochemical diversity in diversity-oriented synthesis. The generation of high levels of stereochemical diversity in this manner has not yet been accomplished in the context of a solid phase, split-pool library synthesis; however, the Sharpless asymmetric dihydroxylation and the Evans aldol reaction have proven to be able to override substrate bias and deliver diastereomerically enriched products on individual substrates in the context of target-oriented synthesis. FIG. 9 summarizes the synthesis of a representative library.

The strategy for synthesizing libraries with skeletal diversity is of great value to scientists pursuing studies in chemical genetics, in which small molecules are used to perturb and thereby understand protein function. These natural product-like compounds generated using the folding pathway strategy may be valuable as potential pharmacological agents themselves or as lead compounds in the search for pharmacological agents for the promotion and/or restoration of human health.

### Example 2

### Combinatorial Synthesis Strategy for Generating Diverse Skeletons of Small Molecules

The macromolecules that carry out the many functions required for life have enormous structural diversity, and this suggests that complementary levels of structural diversity will be needed in collections of candidate small molecules in order to find specific modulators for each of those functions. Diversity-oriented synthesis (DOS) is being used by organic chemists with the aim of populating chemical space efficiently with small molecules having complex and diverse molecular skeletons (S. L. Schreiber, Science 287, 1964 (2000); S. L. Schreiber, Chem. Eng. News 81, 51 (2003); each of which is incorporated herein by reference). Efficient access to skeletal complexity can be achieved in DOS using pairs of complexity-generating reactions, where the product of one is the substrate for another (Schreiber, Science 287, 1964 (2000); S. L. Schreiber, Chem. Eng. News 81, 51 (2003); D. Lee, J. K. Sello, S. L. Schreiber, Org Lett. 2, 709 (2000); each of which is incorporated herein by reference). Gaining efficient access to skeletal diversity, however, has proven to be much more challenging. Achieving this goal in a format amenable to screening in biological assays stands to impact the field of chemical genetics, where small molecules are used in a systematic way to perturb and thereby understand protein function (S. L. Schreiber, Chem. Eng. News 81, 51 (2003); incorporated herein by reference), and may also find use in the pharmaceutical industry, where small molecule-mediated modulation of protein function is used to promote and restore human health.

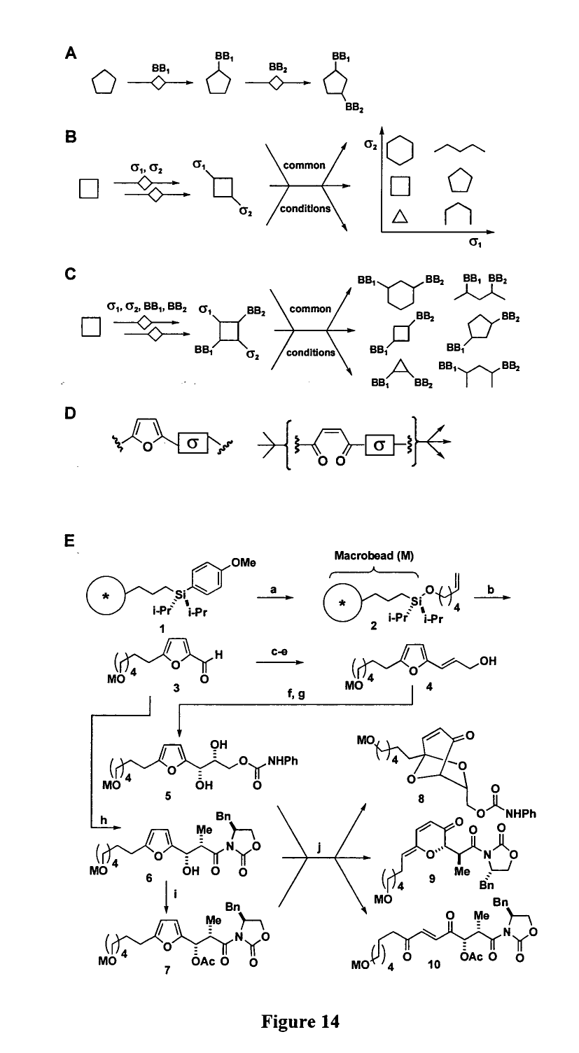

The synthesis strategy most commonly used to access diverse collections of small molecules involves appending different sets of building blocks to a common molecular skeleton (B. A. Bunin and J. A. Ellman, J. Am. Chem. Soc. 114, 10997 (1992); S. H. DeWitt, J. S. Kiely, C. J. Stankovic, M. C. Schroeder, D. M. Reynolds Cody, M. R. Pavia, Proc. Natl. Acad. Soc. US.A. 90, 6909 (1993); each of which is incorporated herein by reference). If this molecular skeleton has multiple reactive sites with potential for orthogonal functionalization, the technique of split-pool synthesis (A. Furka, F. Sebestyén, M. Asgedom, G. Dibó, in Highlights of Modern Biochemistry, Proceedings of the 14^{th} International Congress of Biochemistry, Prague, Czechoslovakia, (1988) (VSP, Utrecht, Netherlands, 1988), vol. 13, p. 47; R. A. Houghten, C. Pinilla, S. E. Blondelle, J. R. Appel, C. T. Dooley, J. H. Cuervo, Nature 354, 84 (1991); K. S. Lam, S. E. Salmon, E. M. Hersh, V. J. Hruby, W. M. Kazmierski, R. J. Knapp, Nature 354, 82 (1991); each of which is incorporated herein by reference) can be used to harness the power of combinatorics (a multiplicative increase in the number of products with an additive increase in the number of reaction conditions), and thereby generate all possible combinations of building blocks (i.e., the complete matrix) very efficiently (FIG. 14A). This one synthesis-one skeleton strategy has proven to be general, and capable of generating hundreds, thousands, or even millions of distinct small molecules in just three-five steps (R. E. Dolle, J. Comb. Chem. 4, 369 (2002); D. S. Tan, M. A. Foley, M. D. Shair, S. L. Schreiber, J. Am. Chem. Soc. 120, 8565 (1998); each of which is incorporated herein by reference). However, although this approach is highly efficient, its impact in the academic and pharmaceutical realms has been very limited (R. Breinbauer, I. R. Vetter, H. Waldmann. Angew. Chem. Int. Ed. 41, 2878 (2002); incorporated herein by reference). This is likely because compounds having a common molecular skeleton display chemical information similarly in three-dimensional space, thus limiting the pool of potential binding partners to only those macromolecules with a complementary three-dimensional binding surface.

We therefore set as our aim the development of an alternative synthesis strategy having the potential to generate collections of compounds representing many different molecular skeletons as efficiently as the one synthesis-one skeleton approach generates collections of compounds representing a single molecular skeleton decorated with many combinations of building blocks. Achieving this goal requires the ability to generate skeletal diversity, rather than building block diversity, combinatorially. Toward this end, we envisioned replacing building blocks with skeletal information elements (σ-elements), which we define as appendages that pre-encode skeletal diversity such that substrates having different a-elements can be transformed into products having different skeletons using a common set of reaction conditions (FIG. 14B). As demonstrated in this report, sets of σ-elements can be identified that act in combination, i.e., a complete matrix of σ-elements can pre-encode a complete matrix of skeletal outcomes, thus making it possible to generate skeletal diversity combinatorially. Moreover, we demonstrate the use of encoded split-pool synthesis to generate a collection of ˜1260 compounds representing both a matrix of σ-elements and a matrix of building blocks appended to the same skeleton, followed by the transformation of these pooled substrates into ˜1260 products representing a complete, combinatorial matrix of molecular skeletons, each derivatized with a combinatorial matrix of building blocks in both enantiomeric/diastereomeric forms (FIG. 14C).

To realize the potential of this σ-element-based strategy for generating diverse skeletons combinatorially in the context of split-pool synthesis, the transformation of substrates having different σ-elements into products having different skeletons using common reaction conditions is a requirement because split-pool synthesis involves the pooling of resin-bound reaction products after each step and it is not possible to ad-hoc optimize reaction conditions for each compound in subsequent transformations. Such a transformation can be planned by first identifying a relatively unreactive core structure that can be transformed under mild conditions into a more reactive intermediate. If different σ-elements having complementary reactivity with this latent intermediate are appended to this common core, then, in theory, these mild conditions can be used to liberate the latent reactive intermediate and allow this complementary reactivity to be realized, resulting in the formation of different skeletons (i.e., diverse displays of chemical information in three-dimensional space).

For example, as shown in FIG. 14D, the aromatic furan ring is a relatively unreactive core structure that can be transformed into a more reactive, electrophilic cis-enedione intermediate under mild oxidative reaction conditions (N. Clauson-Kaas, P. Dietrich, J. T. Nielson, Acta Chem. Scand. 7, 845 (1953); N. Elming, in Advances in Org. Chem. 2, 67 (1960); O. Achmatowicz Jr., P. Bukowski, B. Szechner, Z. Zwierzchowska, A. Zamojski, Tetrahedron 27, 1973 (1971); each of which is incorporated herein by reference). We anticipated that a collection of three substrates having appended to a common furan ring distinct σ-elements in the form of bis-methylene side chains containing two, one, or zero nucleophilic hydroxyl groups could be transformed into a collection of products having distinct molecular skeletons (N. Clauson-Kaas, P. Dietrich, J. T. Nielson, Acta Chem. Scand. 7, 845 (1953); N. Elming, in Advances in Org. Chem. 2, 67 (1960); O. Achmatowicz Jr., P. Bukowski, B. Szechner, Z. Zwierzchowska, A. Zamojski, Tetrahedron 27, 1973 (1971); T. Taniguchi, M. Takeuchi, K. Ogasawara, Tetrahedron Asymmetry 9, 1451 (1998); J. M. Harris, G. A. O'Doherty, Tet. Lett. 41, 183 (2000); Y. Kobayashi, M. Nakano, G. B. Kumar, K. Kishihara, J. Org. Chem. 63, 7505 (1998); each of which is incorporated herein by reference) using an identical set of oxidative and acidic reaction conditions. To explore this possibility, we developed the reaction pathway shown in FIG. 14E. A palladium-catalyzed B-alkyl Suzuki reaction was used to generate macrobead-bound (H. E. Blackwell, L. Pérez, R. A. Stavenger, J. A. Tallarico, E. C. Eatough, M. Foley, S. L. Schreiber, Chem. Biol. 8, 1167 (2001); incorporated herein by reference) furaldehyde 3 from the terminal olefin 2. This furaldehyde precursor was then converted into three different products 5, 6, and 7 containing bis-methylene side-chains with two, one, and zero nucleophilic hydroxyl groups, respectively. Specifically, a sequence of Horner-Wadsworth-Emmons olefination, deallylation, reduction, carbamate formation, and Sharpless asymmetric dihydroxylation converted 3 into the diol 5. Alternatively, the Evans aldol reaction (D. A. Evans, J. Bartroli, T. L. Shih, J. Am. Chem. Soc. 103, 2127 (1981); incorporated herein by reference) with or without subsequent acetylation of the hydroxyl group of the resulting aldol adduct transformed the same furaldehyde precursor 3 into the two products 6 and 7.

After screening a variety of oxidative and acidic reaction conditions, we were successful in identifying a common set, N-bromosuccinimide (NBS) in THF/H₂O:4/1 and pyridiniump-toluene sulfonate (PPTS) in CH₂Cl₂, that were effective in transforming these three substrates 5, 6, and 7 having distinct σ-elements appended to a common furan core into the three products 8, 9, and 10, each having a distinct molecular skeleton. The diol 5 underwent NBS-mediated oxidative ring expansion and subsequent bicycloketalization (O. Achmatowicz Jr., P. Bukowski, B. Szechner, Z. Zwierzchowska, A. Zamojski, Tetrahedron 27, 1973 (1971); T. Taniguchi, M. Takeuchi, K. Ogasawara, Tetrahedron Asymmetry 9, 1451 (1998); each of which is incorporated herein by reference) to yield the [3.2.1] bicycle 8. The aldol adduct 6 containing one flanking hydroxyl group underwent initial NBS-mediated oxidative ring expansion to yield an isolable, intermediate cyclic hemiketal (O. Achmatowicz Jr., P. Bukowski, B. Szechner, Z. Zwierzchowska, A. Zamojski, Tetrahedron 27, 1973 (1971); J. M. Harris, G. A. O'Doherty, Tet. Lett. 41, 183 (2000); each of which is incorporated herein by reference) followed by an unanticipated, PPTS-catalyzed dehydration to yield the alkylidene pyran-3-one 9 as a single geometric isomer. Finally, the acetylated aldol adduct 7 underwent oxidative furan ring opening followed by olefin isomerization (N. Elming, in Advances in Org Chem. 2, 67 (1960); Y. Kobayashi, M. Nakano, G. B. Kumar, K. Kishihara, J. Org. Chem. 63, 7505 (1998); each of which is incorporated herein by reference) to yield the trans-enedione 10.

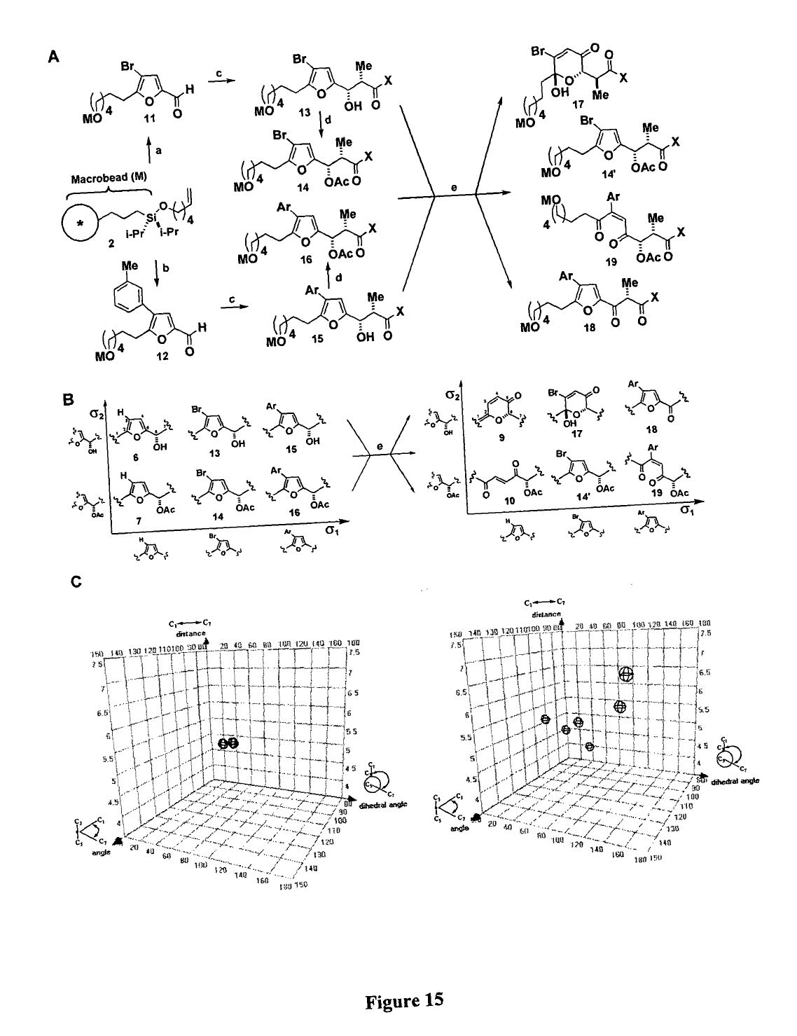

Having demonstrated the transformation of substrates having different σ-elements into products having different skeletons using common reaction conditions, we set out to determine if this approach could generate skeletal diversity combinatorially. To do so requires the identification of at least two sets of σ-elements that can be appended at different sites and function in combination to pre-encode a matrix of distinct skeletal outcomes (see FIG. 14B). Toward this end, it was determined during the course of further studies with this furan-based system that different appendages at the 4-position of the furan core can also effect a variety of unique skeletal outcomes, i.e. appendages at this position function as a second σ-element. Specifically, as shown in FIG. 15A, we varied the 5-bromofuraldehyde unit used in the Suzuki reaction to generate both the 4-bromo (for a related regioselective Pd-mediated coupling with 4,5-dibromofuraldehyde see: T. Bach, L. Krüger, Eur. J. Org. Chem. 2045 (1999); incorporated herein by reference) and 4-aryl derivatives 11 and 12. These 4-substituted furaldehydes were excellent substrates for the Evans aldol reaction and subsequent acetylation resulting in formation of products 13-16. When exposed to the same oxidative (NBS) and acidic (PPTS) conditions used previously, each substrate 13-16 was transformed into a product with a distinct molecular skeleton. The 4-bromo-α-hydroxy furan 13 underwent oxidative ring expansion without subsequent acid-catalyzed dehydration to yield the cyclic hemiketal 17 as a >9:1 mixture of epimers (The unusually high diastereoselectivity observed in the oxidative ring expansion of this substrate is likely a function of both the methyl-bearing stereogenic β-carbon and the chiral auxiliary. The stereochemistry at the anomeric carbon has been tentatively assigned. The assigned structures of products 9, 10, 14′, 18, and 19 are consistent with two-dimensional homonuclear (¹H-¹H) and heteronuclear (¹H-¹³C) correlation NMR experiments, and one-dimensional ¹H NMR NOE experiments.). The 4-bromo-α-acetoxyfuran 14, having two electron-withdrawing appendages, proved completely resistant to oxidation and remained unchanged upon exposure to these reaction conditions. Treatment of the 4-aryl-α-hydroxy furan 15 with NBS resulted in initial oxidative ring expansion to yield an isolable, aryl-substituted cyclic hemiketal similar to 17, which upon exposure to PPTS underwent an unanticipated ring contraction, dehydration, and rearomatization reaction to yield the α-keto furan 18 (For related acid-mediated rearrangements of sugars into α-ketofurans see: F. H. Newth. Advan. Carbohydrate Chem. 6, 83 (1951). Epimerization of the potentially labile stereogenic center in 18 was not observed. Similar stability in related compounds has been attributed to A-1,3-strain with the Evans' auxiliary.). Finally, upon exposure to the same NBS/PPTS conditions, the 4-aryl-α-acetoxyfuran 16 underwent oxidative furan cleavage without subsequent olefin isomerization to yield the cis-enedione 19.

Combining results from FIG. 14E and FIG. 15A, it was possible to assemble a collection of six macrobead-bound substrates 6, 7, and 13-16 (one substrate per macrobead, see FIG. 15B) representing a complete, (3×2=6) combinatorial matrix of σ-elements (—H, —Br, or —Ar at the 4-position of furan combined with —OH or —OAc on the α-methylene). These six individual macrobeads were placed in the same reaction vessel and collectively exposed to the same oxidative and acidic reaction conditions described above, resulting in a complete, non-redundant combinatorial matrix of distinct skeletal outcomes, i.e., a multiplicative increase in skeletons with an additive increase in σ-elements, in the form of products 9, 10, 14′, and 17-19 (FIG. 15B). A metric for evaluating the diversity of the display of chemical information in three-dimensional space generated in this collective transformation is presented in FIG. 15C and in the corresponding supporting information.

These results demonstrate the potential of this σ-element-based strategy to harness the power of combinatorics and thereby generate a complete matrix of distinct molecular skeletons (as opposed to a complete matrix of building blocks) very efficiently. We next set out to determine if this combinatorial matrix of σ-elements could prove to be general and effectively pre-encode the same matrix of distinct skeletal outcomes when a complete, combinatorial matrix of building blocks was also appended to the same common core (see FIG. 14C). If successful, this strategy would provide a highly efficient mechanism to access a collection of compounds representing a set of complete, overlapping matrices of these diversity elements, i.e., a complete combinatorial matrix of molecular skeletons, each derivatized with a complete combinatorial matrix of building blocks (the equivalent of several different collections of compounds synthesized individually using the one synthesis-one skeleton approach).

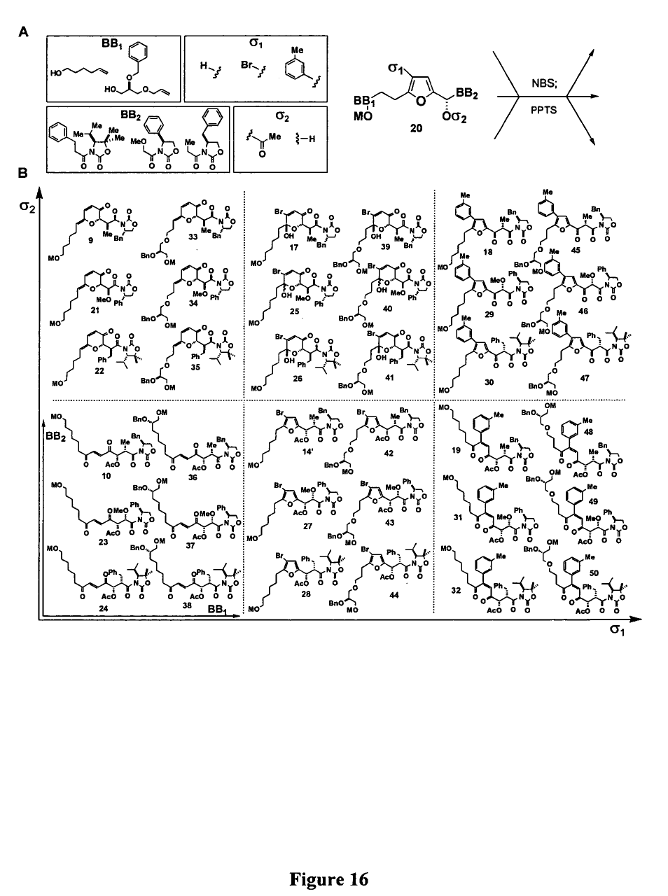

To explore this potential, we first identified two sets of candidate building blocks, BB₁ and BB₂ by determining that structurally diverse coupling partners could be used in the Suzuki and Evans aldol reactions (FIG. 16A). We then prepared a collection of 36 compounds 20a-jj representing all possible combinations of a (2×3) matrix of these candidate building blocks and the (3×2) matrix of σ-elements described above. After cleaving each of these 36 substrates from macrobeads (˜5 mg of macrobeads were cleaved yielding ˜0.5 mg of each compound), 36/36 (100%) of the predicted structures were confirmed by ¹H NMR and HRMS (error <5 ppm), and 35/36 (97%) of these compounds were determined to be ≧70% pure by LCMS analysis (UV detection at λ₂₁₄). We then exposed these 36 substrates to the same set of oxidative and acidic reaction conditions described above and characterized all of the resulting products (after cleaving from macrobeads) using ¹H NMR, LCMS, and HRMS. As shown in FIG. 16B, all three forms of characterization were consistent with the formation of the anticipated functionalized skeleton in 36/36 cases (100%, HRMS error <5 ppm), and 26/36 (72%) of these products were determined to be ≧70% pure by LCMS analysis. These results demonstrate that a common set of reaction conditions were effective in transforming these 36 substrates, representing all possible combinations of σ-elements and building blocks appended to a common α-alkoxy furan core, into a collection of 36 products representing a complete (3×2=6) combinatorial matrix of molecular skeletons, each derivatized with a complete (2×3=6) combinatorial matrix of building blocks.

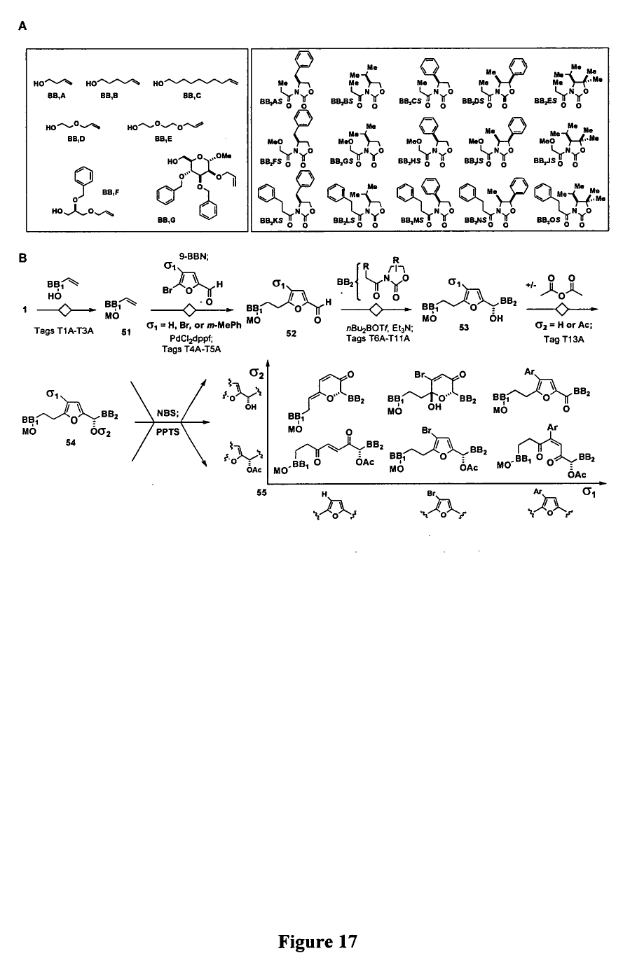

Finally, we set out to realize the demonstrated potential of this σ-element-based strategy to generate overlapping, combinatorial matrices of molecular skeletons and appended building blocks in the context of a highly efficient, five-step, fully-encoded split-pool synthesis pathway (FIG. 17). Toward this end, we first expanded our collections of candidate building blocks to include the diverse set of seven commercially available, terminal olefin-containing primary alcohols (BB₁A-BB₁G) and 15 acyl oxazolidinone coupling partners shown in FIG. 17A (BB₂AS-BB₂OS—a complete matrix of five commercially available, non-racemic, chiral oxazolidinones and three different acyl side chains). The 15 enantiomeric acyl oxazolidinones (BB₂AR-BB₂OR) were also prepared, allowing us to take advantage of reagent-based stereocontrol to generate both sets of possible enantiomeric or diastereomeric (when BB₁ is chiral) aldol adducts. We then confirmed that our synthesis pathway was compatible with the Still chemical encoding technology (H. P. Nestler, P. A. Bartlett, W. C. Still, J: Org Chem. 59, 4723 (1994); H. E. Blackwell, L. Pérez, R. A. Stavenger, J. A. Tallarico, E. C. Eatough, M. Foley, S. L. Schreiber, Chem. Biol. 8, 1167 (2001); incorporated herein by reference), and carried out a fully-encoded split-pool synthesis (FIG. 17B).

Specifically, a series of four consecutive split-pool steps were used to generate very efficiently a collection of ˜1260 compounds 54 representing a set of overlapping matrices of σ-elements (σ₁×σ₂) and building blocks (BB₁×BB₂) appended to a common α-alkoxy-furan skeleton in both enantiomeric/diastereomeric forms. The compound and chemical tags were cleaved (H. E. Blackwell, L. Përez, R. A. Stavenger, J. A. Tallarico, E. C. Eatough, M. Foley, S. L. Schreiber, Chem. Biol. 8, 1167 (2001); incorporated herein by reference) from 60 individual macrobeads 54 and analyzed by LCMS and GC, respectively. These data were found to be consistent for 60/60 (100%) of these macrobeads, and the compounds cleaved from 55/60 (92%) of these macrobeads were determined to be ≧70% pure by LCMS analysis. We then placed this pooled collection of ˜1260 macrobead-bound substrates (˜4410 macrobeads, multiplicative factor=3.5 (Statistical calculations and computer simulations suggest that a multiplicative factor of 3.1 is required to provide 99% confidence of achieving 95% coverage of the complete, theoretical combinatorial matrix for a split-pool synthesis involving four split-pool cycles with ten pools per cycle. K. Burgess, A. I. Liaw, and N. Wang. J. Med. Chem. 37, 2985 (1994), incorporated herein by reference)) in a singleflask and exposed them to the same oxidative and acidic reaction conditions described above to yield a collection of ˜1260 products 55 representing a complete, combinatorial (3×2=6) matrix of molecular skeletons, each derivatized with a combinatorial (7×15=105) matrix of building blocks in both enantiomeric/diastereomeric forms (6×105×2=1260). LCMS analysis of compounds cleaved from 120 of these macrobeads was consistent with the structure encoded by the corresponding chemical tags in 120 out of 120 cases (100%). Moreover, 84/120 (70%) of these compounds were determined to be >70% pure by LCMS analysis.

In this report, we have described and implemented a synthesis strategy that involves transforming substrates having different σ-elements, i.e., appendages that pre-encode skeletal diversity, into products having different skeletons using common reaction conditions. A major advantage of this σ-element-based approach is that skeletal diversity can be pre-encoded into substrates combinatorially using split-pool synthesis, thus making it possible to generate a complete matrix of molecular skeletons in a highly efficient manner. In addition, forming diverse skeletons late in the synthesis pathway (in contrast to forming a skeleton first as it typical with the one synthesis-one skeleton approach) facilitates the generation of functionalized skeletons that might otherwise be difficult to access, such as those having building blocks coupled via carbon-carbon bonds at stereogenic quaternary carbon centers (e.g 17 and related products) and those having potentially unstable structural elements (e.g., enediones 10 and 19 and related products). Also, the maintenance of structural similarity and therefore common reactivity until late in the synthesis pathway facilitates the realization of this strategy using the highly efficient, split-pool technique. Moreover, with this approach, split-pool synthesis can be used to generate a collection of compounds representing overlapping matrices of molecular skeletons and appended building blocks in both enantiomeric/diastereomeric forms (e.g. 55). To the best of our knowledge, such a collection of non-oligomeric small molecules potentially representing all possible combinations of building block, stereochemical, and skeletal diversity elements is unprecedented. Systematic screening of this collection of compounds in the form of small molecule microarrays (G. MacBeath, A. N. Koehier, S. L. Schreiber J. Am. Chem. Soc. 121, 7967 (1999); incorporated herein by reference) in protein binding assays and in the form of stock solutions in cell-based phenotypic assays may advance our fundamental understanding of the roles these three diversity elements play in small molecule-protein interactions.

The σ-element-based diversity-oriented synthesis strategy demonstrated in this report has potential for general application in the planning of efficient synthesis pathways that generate collections of small molecules having skeletal diversity. Future directions include exploring this potential generality, increasing the size and dimensionality of σ-element matrices (even semi-redundant matrices should prove to be highly valuable), and incorporating the pairwise use of complexity-generating reactions into σ-element-based skeletal diversity-generating pathways. The σ-element-based approach is rich with potential for discovering and utilizing unanticipated skeletal outcomes. It may be possible to realize this potential efficiently by using encoded split-pool synthesis to append a complete matrix of candidate σ-elements to a common core having latent reactivity, exposing this collection to common conditions, and then searching among the products for distinct skeletons of suitable purity.

### Experimentals

### I. General Methods

Materials. Commercially available reagents were obtained from Aldrich Chemical Co. (Milwaukee, Wis.), Fluka Chemical Corp. (Milwaukee, Wis.), Bachem (Bubendorf, Switzerland), and MoscowMedChemLabs (Moscow, Russia) and used without further purification unless otherwise noted. All solvents were dispensed from a solvent purification system that passes solvents through packed columns (THF, Et₂O, CH₃CN, and CH₂Cl₂: dry neutral alumina; hexane, benzene, and toluene: dry neutral alumina and Q5 reactant; DMF: activated molecular sieves). Water was double distilled. Triethylamine, diisopropylethylamine, and 2,6-lutidine were distilled under nitrogen from CaH₂. Macrobeads 1 were prepared by Max Narovlyansky at Harvard's ICCB: Longwood as previously described (John A. Tallarico et al. J. Comb Chem 2001, 3, 312-318; incorporated herein by reference).

Solution phase reactions. All solution-phase reactions were performed in oven- or flame-dried glassware under positive argon pressure unless otherwise indicated. Reactions were monitored by analytical thin-layer chromatography performed using indicated solvent on E. Merck silica gel 60 F₂₅₄ plates (0.25 mm). Compounds were visualized with a UV lamp (λ₂₅₄) and/or staining with cerium ammonium molybdate.

Solid phase reactions. Solid-phase reaction were performed in oven- or flame-dried glassware (I-Chem vials or Wheaton vials, fitted with Teflon-coated caps) with gentle mixing provided by Thermoline Vari-Mix shaker or a Vortex Genie-2 vortexer (VWR 58815-178, setting V1-V2) fitted with a 60 microtube insert. After reactions were completed, resin was isolated by filtration in 10 ml Amersham columns on a Vac-Man laboratory Vacuum Manifold (Promega A723 1) fitted with nylon 3-way stopcocks (Biorad 732-8107). Resin was then washed as indicated and solvent was removed in vacuo. All compounds were cleaved from the solid-support resin using the following standard procedure: To resin in a polypropylene eppendorf tube at rt under ambient was added a freshly prepared solution of 5% HF/Pyr in THF (10-100 μL per mg of resin). The resulting mixture was then agitated at rt for 2 h. The reaction was then quenched with the addition of neat methoxytrimethylsilane or ethoxytrimethylsilane (2/1 v/v relative to 5% HF/Pyr in THF solution). The resulting mixture was then agitated at rt for 10 minutes, and the solution was then transferred to a Wheaton vial. Resin was washed twice with THF. The combined reaction solution and wash solutions were concentrated in vacuo and the cleaved product was then analyzed as indicated.

Purification and analysis. Flash chromatography was performed using the indicated solvent on E. Merck silica gel 60. All yields refer to compounds cleaved from 75-85 mg of macrobeads and purified by flash chromatography. Infrared spectra were recorded as a thin film on NaCl plates on a Nicolet 5PC FT-IR spectrometer with internal referencing. Absorption maxima (νₘₐₓ) are reported in wavenumbers (cm⁻¹). ¹H NMR spectra were recorded on Varian Unity/Inova500 (500 MHz) spectrometer. ¹³C NMR spectra were recorded on Varian Unity/Inova400 (400 MHz) spectrometer. Chemical shifts (δ) are reported in ppm and referenced to CDCl₃. (¹H-NMR, 7.26; ¹³C-NMR, 77.0, center line). Nanotube solid-phase MAS ¹HNMR were obtained in CD₂Cl₂ on a Varian Inova 600 instrument fitted with a magic-angle spinning nanoprobe. Reverse-phase LCMS data was obtained with a Gilson/Finnigan LCMS system. LCMS chromatography was performed on a SymmetryShield™ RP_{8, 3.5} uM, 4.6×100 mm column (Waters Corporation, Milford, Mass., Batch #111) using a flow rate of 1 ml/min and a 10 min gradient of 20-80% CH₃CN in water, constant 0.1% formic acid, with UV detection at 214 and 280 nm. MS analysis was performed with a Finnigan Aqa MS detector with ES+ ionization. Chiral LC was performed on a Gilson LC system using a Chiralpak® AS™ 250×4.6 mm column (Amylose tris-[(S)-α-methylbenzyl carbamate] coated on 10 μm silica-gel substrate, Chiral Technologies Inc., Exton, Pa.) using a flow rate of 1 ml/min and an eluent of 4% IPA in hexanes. High resolution mass spectra were obtained at the mass spectrometry facility at Harvard University using a Micromass LCT (ES) spectrometer.

### II.

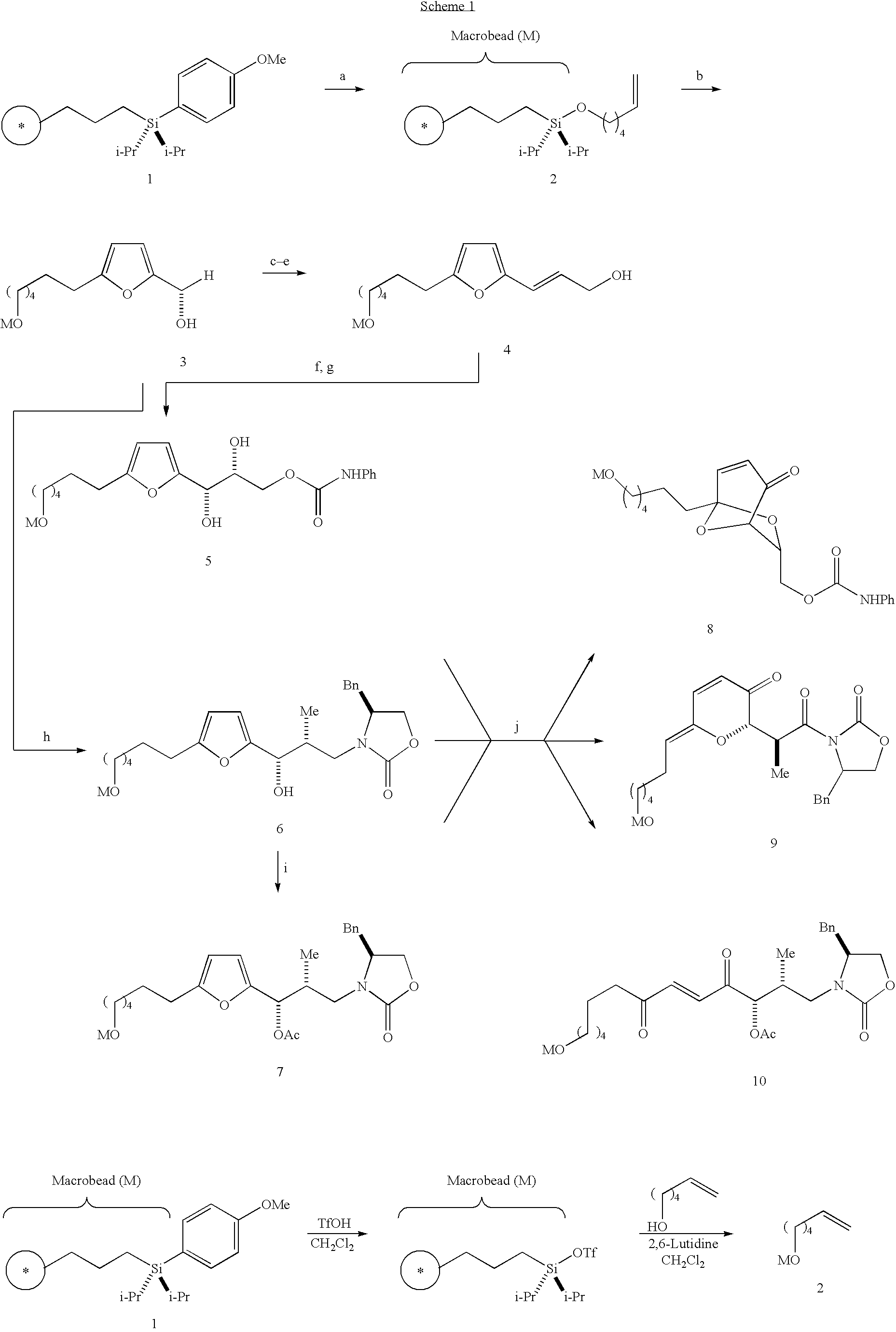

Macrobead-bound-5-hexen-1-ol (2). 3-[Diisopropyl(p-methoxyphenyl)silyl]propyl functionalized macrobeads 1 (400 mg, estimated loading ˜1.3 meq Si/g, ˜0.52 mmol) in a 20 mL polypropylene tube at rt under Ar were allowed to swell in CH₂Cl₂ (15 ml) for 10 min. The colorless beads were then filtered and again washed with CH₂Cl₂ (15 mL×10 min.), and then resuspended in a 2.5% (v/v) solution of TMSCl in CH₂Cl₂ (15 mL) for 30 min. The beads were again filtered and washed thrice with CH₂Cl₂ (5 min each) and then suspended in a 3% (v/v) solution of trifluoromethanesulfonic acid in CH₂Cl₂ (9.2 mL, 3.12 mmol) for 20 min during which the reaction tube was shaken periodically and the beads turned orange. After filtration, the orange-colored beads were again thrice washed with CH₂Cl₂ and then resuspended in a minimum volume of CH₂Cl₂ (1 mL). Freshly distilled 2,6-lutidine was then added (485 uL, 4.2 mmol) resulting in bead discoloration followed by 5-hexen-1-ol (500 uL, 4.2 mmol). The resulting colorless reaction mixture was then shaken manually and let stand at rt for 12 h. The beads were then filtered, washed with CH₂Cl₂ (5×15 mL×5 min. each), and the solvent was removed under Ar flow followed by residual solvent removal in vacuo to yield resin 2 (372 mg) loaded with 5-hexen-1-ol. MAS ¹H NMR (600 MHz, CD₂Cl₂) selected peaks δ 5.81 (br s), 5.00 (d, J=17.0 Hz), 4.93 (d, J=8 Hz), 3.65 (br s).

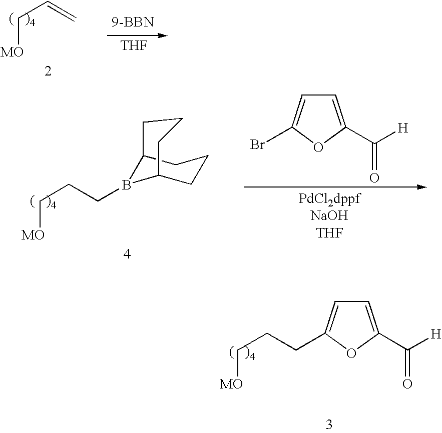

Macrobead-bound-5-(6-hydroxy-hexyl)-furan-2-carbaldehyde (3). Colorless beads 1 (500 mg, max theoretical loading 1.3 meq/g, 0.65 mmol) were washed with THF (2×10 mL×10 min each) at rt and then resuspended in 15 mL THF. A 0.5M solution of 9-BBN in THF (10 mL, 5.0 mmol) was then added and the resulting mixture was manually agitated and let stand at rt for 5 h. The reaction solution was then removed via cannula and the colorless resin was washed thoroughly with THF (5×15 mL×10 min each). To the resin was then added solid PdCl₂dppf (6.1 mg, 0.0075 mmol), 5-bromofuraldehyde (438 mg, 2.5 mmol) via cannula as a solution in THF (6.25 mL), and a 1M aq. solution of NaOH (1.25 mL, 1.25 mmol). The resulting orange reaction mixture was sealed under a cloud of Ar and heated at 65° C. with periodic manual agitation for 18 h (reaction mixture turned dark brown). The yellow/orange resin was then isolated by filtration and washed as follows, 4×(5×THF, 5×H₂O, 5×THF, THF/H₂O: 3/1×30 min), 5×THF, THF×30 min, 5×CH₂Cl₂, CH₂Cl₂×30 min, 5×anh. CH₂Cl₂, anh. CH₂Cl₂×30 min, and then the solvent was removed in vacuo to yield 535 mg of yellow/orange product resin 3. 5 mg of this resin was then treated with HF/Pyridine cleavage conditions (see General Methods) to yield crude product with LCMS purity >85% (λ₂₁₄), t_{R} 4.82 min. 75 mg of this resin was then treated with HF/Pyridine cleavage conditions and the crude product was purified by flash chromatography (SiO₂, hexane/EtOAc: 1/2) to afford a yellow oil (10.0 mg, 0.679 meq./g, 58% over two steps based on estimated meq. Si/g). R_{f}=0.27 (hexane/EtOAc:1/2); FTIR (film, cm⁻¹) 3426, 2932, 2859, 1674, 1518, 1399, 1024; ¹H NMR (500 MHz, CDCl₃) δ 9.51 (s, 1H), 7.17 (d, J=3.5 Hz, 1H), 6.23 (d, J=4.0 Hz, 1H), 3.64 (t, J=7.0 Hz, 2H), 2.73 (t, J=7.5Hz, 2H), 1.72 (m, 2H), 1.57 (m, 2H), 1.39 (m, 4H); ¹³C NMR (100 MHz, CDCl₃) δ 176.9, 163.9, 151.8, 123.6, 108.7, 62.8, 32.5, 28.9, 28.3, 27.5, 25.3; HRMS (ES⁺) calculated for C₁₁H₁₆O₃ (M+H)⁺: 197.1177, Found: 197.1177.

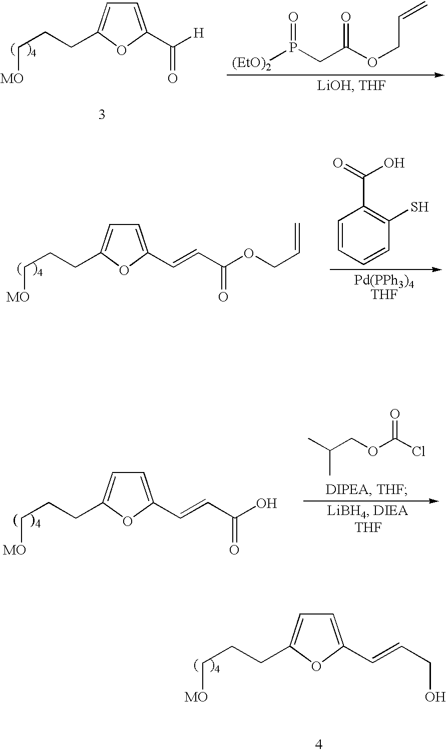

Macrobead-bound-trans-3-[5-(6-hydroxy-hexyl)-furan-2-yl]-prop-2-en-1-ol (4). To a stirred solution of allyldiethylphosphonoacetate (0.664 mL, 3.15 mmol) in THF (10.5 mL) at rt was added solid LiOH (151 mg. 6.29 mmol). The resulting mixture was stirred vigorously at rt for 4 h. The stir bar was then removed and resin 3 (315 mg, 0.679 meq/g, 0.214 mmol) was added. The resulting reaction mixture was sealed under a cloud of Ar and tumbled at rt for 25 h. Resin was then isolated by filtration and washed as follows: 5×THF, 5×H₂O, 5×THF, THF/dilute aq. NH₄Cl (sat. aq. NH₄Cl/H₂O: 1/2): 1/1×1 h. 5×THF, 5×H₂O, 5×THF, THF/H₂O: 3/1×1 h, 5×THF, THF×30 min, 5×CH₂Cl₂, CH₂Cl₂×20 min, 5×anhydrous CH₂Cl₂, anhydrous CH₂Cl₂×10 min. Solvent was then removed in vacuo to yield 336 mg of yellow-orange resin. This resin (331 mg) was then added to a mixture of Pd(PPh₃)₄ (382 mg, 0.331 mmol) in THF (7.2 mL). To this mixture was then added solid thiosalicylic acid (510 mg, 3.31 mmol) and the resulting dark red mixture was sealed under a cloud of Ar, covered with aluminum foil, and tumbled at rt for 24 h. Resin was then isolated by filtration and washed as follows: 5×(5×THF, THF×1 h), 5×CH₂Cl₂, CH₂Cl₂×20 min, 5×anhydrous CH₂Cl₂, anhydrous CH₂Cl₂×10 min. Solvent was then removed in vacuo to yield 322 mg of yellow-orange resin. This resin (317 mg) was then washed twice with anhydrous THF and then resuspended in THF (29 mL). Diisopropylethylamine (2.75 mL, 15.8 mmol) was then added and the resulting mixture was cooled to 0° C. To this mixture was added 4-methylmorpholine (0.035 mL, 0.317 mmol) and isobutylchloroformate (0.411 mL, 3.17 mmol). The resulting mixture was maintained at 0° C. for 2 h, with periodic manual agitation every 30 minutes. The reaction solution was removed via cannula and the resin was washed with 8.6% (v/v) diisopropylethylamine in THF (3×15 mL×5 min each) at 0° C. Resin was then resuspended in a solution of 8.6% (v/v) diisopropylethylamine in THF (40 mL) at 0° C., and to this mixture was added solid LiBH₄ (21 mg, 0.95 mmol). The resulting mixture was sealed under a cloud of Ar and tumbled at 4° C. for 24 h. The resin was then isolated by filtration at rt and washed as follows: 5×THF, 5×H₂O, 5×THF, THF/dilute aq. NH₄Cl (sat. aq. NH₄Cl/H₂O: 1/2): 1/1×1 h. 5×THF, 5×H₂O, 5×THF, THF/H₂O: 3/1×1 h, 5×THF, 5×H₂O, 5×THF, THF×1 h, 5×CH₂Cl₂, CH₂Cl₂×30 min, 5×anhydrous CH₂Cl₂, anhydrous CH₂Cl₂×30 min and then solvent was removed in vacuo to yield light yellow product resin 4 (320 mg). 5 mg of this product resin was then treated with HF/Pyridine cleavage conditions (see General Methods) to yield crude product with LCMS purity 68% (λ₂₁₄), t_{R} 5.80 min. 75 mg of this resin was then treated with HF/Pyridine cleavage conditions and the crude product was purified by flash chromatography (SiO₂, hexane/EtOAc: 1/2) to afford the desired allylic alcohol as a colorless solid [8.1 mg, 0.482 meq./g, Theoretical yield 0.637 meq./g, 76% from 3, E/Z : >20/1 (¹H NMR)]. R_{f}=0.24 hexane/EtOAc: 1/2); FTIR (film, cm⁻¹) 3349, 2928, 2857, 1661, 1588, 1532, 1463, 1380, 1254; ¹H NMR (500 MHz, CDCl₃) δ 6.37 (dd, J=16 Hz, 1 Hz, 1H), 6.21 (dt, J=16 Hz, 6 Hz, 1H), 6.13 (d, J=3.5 Hz, 1H), 5.95 (d, J=3.5 Hz, 1H), 4.27 (d, J=5.5 Hz, 2H), 3.64 (t, J=7 Hz, 2H) 2.61 (t, J=7.5 Hz, 2H), 1.66 (m, 2H), 1.58 (m, 2H), 1.39 (m, 4H); ¹³C NMR (100 MHz, CDCl₃) δ 156.4, 150.6, 125.5, 119.8, 109.1, 106.6, 63.5, 62.9, 32.6, 28.9, 28.0, 27.9, 25.4; HRMS (ES⁺) calculated for C₁₃H₂O₃ (M−H)⁻: 223.1334, Found: 223.1333. LiOH-promoted HWE-olefination: F. Bonadies, A. Cardilli, A. Lattanzi, L.R. Orelli, and A. Scettri. Tet. Lett. 35, 3383 (1994).

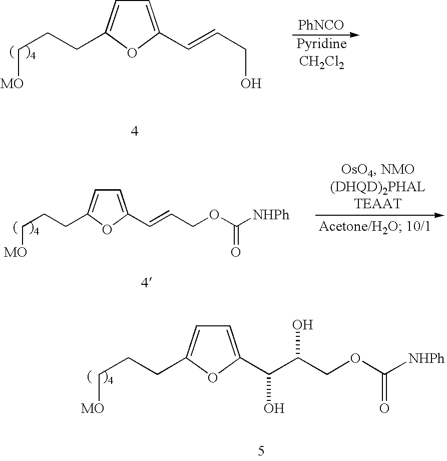

Macrobead-bound-(2R,3S)-phenyl-carbamic acid 2,3-dihydroxy-3-[5-(6-hydroxy-hexyl)-furan-2-yl]-propyl ester (5). Light yellow beads 4 (220 mg, 0.482 meq./g, 0.106 mmol) were washed with CH₂Cl₂ (2×10 mL×10 min each) at rt and then resuspended in CH₂Cl₂ (11 mL). To this mixture at rt was added pyridine (0.356 mL, 4.41 mmol) and phenyl isocyanate (0.239 mL, 2.20 mmol). The resulting mixture was sealed under a cloud of Ar and tumbled at rt for 24 h. Resin was then isolated by filtration and washed as follows: 5×THF, 5×H₂O, 5×THF, THF/dil. aq. NaHCO₃ (sat. aq. NaHCO₃/H₂O: 1/2): 1/1×1 h, 5×THF, 5×H₂O, 5×THF, THF/dilute aq. NH₄Cl (sat. aq. NH₄Cl/H₂O: 1/2): 1/1×1 h, 5×THF, 5×H₂O, 5×THF, THF/H₂O: 3/1×1 h, 5×THF, THF×1 h, 5×CH₂Cl₂, CH₂Cl₂×30 min, and then residual solvent was removed in vacuo to yield 245 mg of yellow resin. A separate vessel was then charged with (DHQD)₂PHAL (10.6 mg, 0.0135 mmol), tetraethylammonium acetate tetrahydrate (113 mg, 0.433 mmol) and 4-methylmorpholine N-oxide (76.2 mg, 0.650 mmol). This solid mixture was dissolved in a solution of acetone/water: 10/1 at rt under ambient and to this clear, slightly yellow-tinted solution was added OSO₄ as a 2.5 wt% solution in tert-butyl alcohol (0.060 ml, 0.00542 mmol). The resulting clear, yellow-tinted solution was let stand at rt with periodic manual agitation for 15 min and then cooled to 0° C. The resin (217 mg) was then added and the resulting mixture was sealed and tumbled at 4° C. for 48 h. The reaction solution was then removed via syringe and quenched with excess sodium metabisulfite, and the resin was washed with acetone/water: 10/1 (1×5 mL×10 min, 1×15 mL×30 min) at 4° C., and then isolated by filtration and washed as follows: 5×THF, 10% pyridine in THF×1 h, 5×THF, 10% pyridine in THF×12 h, 5×THF, 10% pyridine in THF×4 h, 5×THF, 10% pyridine in THF×4 h, 5×THF, 5×H₂O, 5×THF, THF/dil. aq. NaHCO₃ (sat. aq. NaHCO₃/H₂O: 1/2): 1/1×45 min, 5×THF, 5×H₂O, 5×THF, THF/dilute aq. NH₄Cl (sat. aq. NH₄Cl/H₂O: 1/2): 1/1×45 min, 5×THF, 5×H₂O, 5×THF, THF/H₂O: 3/1×1 h, 5×THF, THF×45 min, 5×DMF, DMF×45 min, 5×THF, THF×45 min, 5×anh. THF, anh. THF×30 min, and then solvent was removed under Ar flow followed by residual solvent removal in vacuo. 5.2 mg of this resin was then treated with HF/Pyridine cleavage conditions (see General Methods) to yield crude product with LCMS purity >90% (λ₂₁₄), t_{R} 5.92 min. 75.2 mg of this resin was then treated with HF/Pyridine cleavage conditions and the crude product was purified by flash chromatography (SiO₂, EtOAc/MeOH: 100/0→90/10) to afford a yellow oil [8.3 mg, 0.292 meq./g, Theoretical yield 0.449 meq./g, 65% from 4. The enantioselectivity obtained in this reaction was determined after converting 5→8 (vide infra). R_{f}=0.33 (EtOAc/MeOH: 99/1); FTIR (film, cm⁻¹) 3322, 2932, 2858, 1711, 1601, 1547, 1501, 1445, 1315, 1224, 1055; ¹HNMR (500 MHz, CDCl₃) δ 7.38-7.28 (m, 4H), 7.07 (t, J=7 Hz, 1H), 6.96 (br s, 1H), 6.27 (d, J=3 Hz, 1H), 5.94 (d, J=3 Hz, 1H), 4.65 (d, J=5.5 Hz, 1H), 4.27 (dd, J=11.5, 3.5 Hz, 1H), 4.22-4.13 (m, 2H), 3.62 (t, J=6.5 Hz, 2H), 3.26 (br s, 1H), 2.97 (br s, 1H), 2.60 (t, J=7 Hz, 2H), 1.63 (m, 2H), 1.55 (m, 2H), 1.35 (m, 4H); ¹³C NMR (100 MHz, CDCl₃) δ 156.7, 153.7, 150.8, 137.5, 129.1, 123.7, 118.7, 108.9, 105.7, 72.0, 68.0, 66.0, 62.8, 32.4, 28.7, 27.8, 27.7, 25.3; HRMS (ES⁺) calculated for C₂₀H₂₇NO₆ (M+Na)⁺: 400.1736, Found: 400.1737.

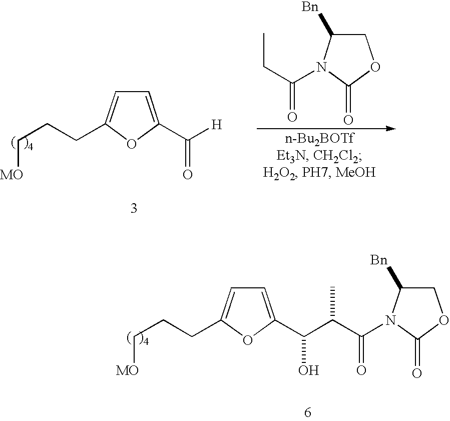

Macrobead-bound-(4S)-4-benzyl-3-{(3S,2S)-3-hydroxy-3-[5-(6-hydroxy-hexyl)-furan-2-yl]-2methyl-propionyl}-oxazolidin-2-one (6). Yellow-orange beads 3 (365 mg, 0.679 meq./ g, 0.248 mmol) were washed with CH₂Cl₂ (2×15 mL×10 min each) at rt, and then cooled to −78° C. In a separate vessel, to a stirred solution of (S)-(+)-4-benzyl-3-propionyl-2-oxazolidinone (426 mg, 1.83 mmol) in CH₂Cl₂ (7.3 mL) at 0° C. was added a 1M solution of dibutylboron triflate in CH₂Cl₂ (1.92 mL, 1.92 mmol, (nBu₂BOTf solution was obtained from Aldrich chemical company and stored at −26° C. upon delivery. Best results were obtained when this reagent was used within 2 weeks of shipping date) followed by triethylamine (0.305 mL, 2.19 mmol). The resulting enolate solution was cooled to −78° C. and then transferred rapidly via cannula to the vessel containing 3. The resulting mixture was sealed under a cloud of Ar and maintained at −78° C. for 48 h, −26° C. for 24 h, and 0° C. for 1 h (with periodic manual agitation about once every 8 h). The reaction was then quenched with the addition of pH7 phosphate buffer (7 mL), MeOH (7 mL), and 30% aq. H₂O₂ (4.7 mL), and the resulting mixture was tumbled at 4° C. for 12 h. Resin was then isolated by filtration and washed as follows: 5×CH₂Cl₂, 5×DMF, 5×THF, 5×CH₂Cl₂, CH₂Cl₂×1 h, 5×DMF, DMF×1 h, 5×THF, THF×1 h, 5×CH₂Cl₂, CH₂Cl₂×30 min, 5×anhydrous CH₂Cl₂, anhydrous CH₂Cl₂×30 min, and residual solvent was removed in vacuo to yield 6 as light yellow beads (431 mg). 5.2 mg of this resin was then treated with HF/Pyridine cleavage conditions (see General Methods) to yield crude product with LCMS purity >90% (λ₂₁₄), t_{R} 7.74 min. 75.2 mg of this resin was then treated with HF/Pyridine cleavage conditions and the crude product was purified by flash chromatography (SiO₂, Hexanes/EtOAc: 1/1→1/2) to afford a yellow oil [18.2 mg, 0.0424 mmol, 0.563 meq./g, Theoretical yield 0.586 meq./g, 96% from 3, dr >20:1 (¹H NMR)]. R_{f}=0.30 hexane/EtOAc:1/2); FTIR (film, cm⁻) 3442, 2933, 2859, 1781, 1696, 1454, 1387, 1210, 1108, 1012; ¹H NMR (500 MHz, CDCl₃) δ 7.35-7.27 (m, 3H), 7.19 (app d, J=6.5 Hz, 2H), 6.17 (d, J=3 Hz, 1H), 5.90 (d, J=3 Hz, 1H), 5.01 (m, 1H), 4.62 (m, 1H), 4.16 (m, 3H), 3.62 (t, J=6 Hz, 2H), 3.24 (dd, J=13 Hz, 3 Hz, 1H), 2.99 (br d, J=4 Hz, 1H), 2.78 (dd, J=13 Hz, 9 Hz, 1H), 2.58 (t, J=7.5 Hz, 2H), 1.62 (m, 2H), 1.56 (m, 2H), 1.36 (m, 7H); ¹³C NMR (100 MHz, CDCl₃) δ 176.2, 156.0, 152.8, 152.1, 135.0, 129.4, 128.9, 127.4, 107.3, 105.3, 68.7, 66.2, 62.8, 55.2, 42.5, 37.8, 32.5, 28.7, 27.8 (2 carbons), 25.3, 12.2; HRMS (ES⁺) calculated for C₂₄H₃₁NO₆(M+NH₄)⁺: 447.2495, Found: 447.2497.

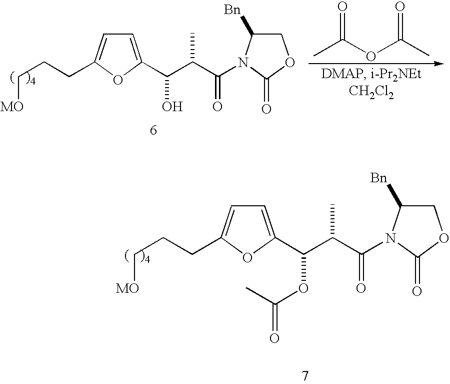

Macrobead-bound-(4S)-4-benzyl-3-{(3S,2S)-3-acetoxy-3-[5-(6-hydroxy-hexyl)-furan-2-yl]-2-methyl-propionyl}-oxazolidin-2-one (7). Light yellow beads 6 (0.180 g, 0.563 meq./g, 0.101 mmol) were washed with CH₂Cl₂ (2×9 mL×5 min each) at rt and then resuspended in 9 mL CH₂Cl₂. To this mixture at rt was added diisopropylethylamine (0.627 mL, 3.6 mmol), DMAP (22 mg, 0.18 mmol), and acetic anhydride (0.170 mL, 1.8 mmol). The resulting mixture was sealed under a cloud of Ar and tumbled at rt for 28 h. Resin was then isolated by filtration and washed as follows: 5×CH₂Cl₂, 5×THF, 5×CH₂Cl₂, CH₂Cl₂×45 min, 5×THF, THF×45 min, 5×CH₂Cl₂, CH₂Cl₂×45 min, 5×anh. CH₂Cl₂, anh. CH₂Cl₂×20 min. Solvent was then removed in vacuo to yield 13 as light yellow beads. 5.0 mg of this resin was then treated with HF/Pyridine cleavage conditions (see General Methods) to yield crude product with LCMS purity >90% (p214), t_{R} 8.83 min. 75.2 mg of this resin was then treated with HF/Pyridine cleavage conditions and the crude product was purified by flash chromatography (SiO₂, Hexanes/EtOAc: 1/1→1/2) to afford a yellow oil [17.1 mg, 0.0363 mmol, 0.482 meq./g, Theoretical yield 0.550 meq./g, 88% from 6). R_{f} =0.17 hexane/EtOAc:1/1); FTIR (film, cm⁻¹) 3545, 2933, 2859, 1782, 1744, 1700, 1455, 1387, 1225, 1108, 1016; ¹H NMR (500 MHz, CDCl₃) δ 7.34-7.25 (m, 3H), 7.18 (d, J=7 Hz, 2H), 6.22 (d, J=3.5 Hz, 1H), 6.14 (d, J=7 Hz, 1H), 5.89 (d, J=3 Hz, 1H), 4.51 (m, 2H), 4.13 (m, 2H), 3.62 (t, J=6 Hz, 2H), 3.23 (dd, J=13, 3 Hz, 1H), 2.75 (dd, J=13, 9.5 Hz, 1H), 2.56 (t, J=7.5 Hz, 2H), 2.09 (s, 3H), 1.60 (m, 2H), 1.56 (m, 2H), 1.39-1.30 (m, 4H), 1.33 (d, J=7 Hz, 3H); ¹³C NMR (100 MHz, CDCl₃) 173.4, 170.1, 156.6, 153.1, 149.1, 135.1, 129.4, 128.9, 127.4, 109.6, 105.5, 69.2, 66.2, 62.8, 55.4, 40.8, 37.8, 32.5, 28.8, 27.8, 27.7, 25.3, 21.0, 13.3; HRMS (ES⁺) calculated for C₂₆H₃₃NO₇ (M+Na)⁺: 494.2155, Found: 494.2169.

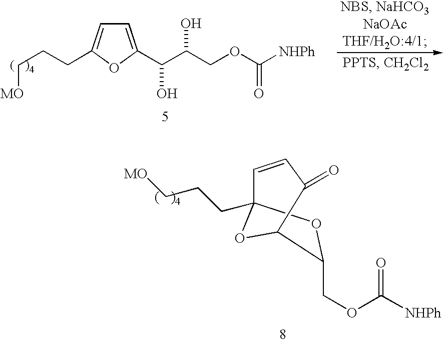

Macrobead-bound-phenyl-carbamic acid (1S,5S,7R)-5-(6-hydroxy-hexyl)-2-oxo-6,8-dioxabi-cyclo[3.2.11]-oct-3-en-7-ylmethyl ester (8). To a mixture of light yellow beads 5 (0.090 g, 0.292 meq./g, 0.026 mmol) in THF/water: 4/1 at rt under ambient was added NaHCO₃ (227 mg, 2.70 mmol), NaOAc (111 mg, 1.35 mmol), and N-bromosuccinimide (160 mg, 0.90 mmol). The resulting mixture was sealed, wrapped in foil, and tumbled at rt for 1 h. Resin was then isolated by filtration and washed as follows: 5×THF, 5×H₂O, 5×THF, THF/water: 3/1×1 h, 5×THF, THF×1 h, 5×CH₂Cl₂, CH₂Cl₂×30 min, 5×anh. CH₂Cl₂, anh. CH₂Cl₂×30 min, and then transfer to a separate vessel containing a 0.00075M solution of pyridiniump-toluenesulfonate in CH₂Cl₂ (20 mL). The resulting mixture was sealed under a cloud of argon and maintained at 40-45° C. (oil bath) for 20 h. Resin was then isolated by filtration and washed as follows: 5×THF, 5×H₂O, 5×THF, THF/dil. aq. NaHCO₃ (sat. aq. NaHCO₃/H₂O: 1/2): 1/1×1 h, 5×THF, 5×H₂O, 5×THF, THF/dilute aq. NH₄Cl (sat. aq. NH₄Cl/H₂O: 1/2): 1/1×1 h, 5×THF, 5×H₂O, 5×THF, THF/H₂O: 3/1×1 h, 5×THF, THF×1 h, 5×CH₂Cl₂, CH₂Cl₂×30 min, 5×anh. CH₂Cl₂, anh. CH₂Cl₂×30 min. Solvent was then removed in vacuo to yield 8 as light yellow beads. 5.4 mg of this resin was then treated with HF/Pyridine cleavage conditions (see General Methods) to yield crude product with LCMS purity 64%, t_{R} 7.04 min (an impurity at t_{R}=8.20 min which was ¹H NMR-silent and had an MS isotope pattern consistent with an osmium-containing substance was not included in purity calculation for this product; for additional purity information, see 1H NMR of crude and purified product 8). 80.8 mg of the product resin was then treated with HF/Pyridine cleavage conditions and the crude product was purified by flash chromatography (SiO₂, Hexanes/EtOAc: 1/1→1/2) to afford a yellow oil [2.9 mg, 0.00773 mmol, 0.0.096 meq./g, Theoretical yield 0.0.292 meq./g, 33% from 5). R_{f}=0.31 (hexane/EtOAc: 1/2); FTIR (film, cm⁻¹) 3323, 2932, 2859, 1705, 1599, 1537, 1491, 1445, 1400, 1309, 1220, 1075; ¹H NMR (500 MHz, CDCl₃) δ 7.44-7.26 (m, 4H), 7.08 (t, J=7 Hz, 1H), 7.02 (d, J=10 Hz, 1H), 6.82 (br m, 1H), 6.08 (dd, J=10 Hz, 1 Hz, 1H), 4.59 (app d, J=6.5 Hz, 1H), 4.30-4.26 (m, 2H), 4.06 (app q, J=5 Hz, 1H), 3.64 (t, J=6 Hz, 2H), 2.02-1.91 (m, 2H), 1.60-1.48 (m, 4H), 1.44-1.35 (m, 4H); ¹³C NMR (100 MHz, CDCl₃) δ 194.0, 150.7 (2C), 132.1, 129.1, 126.5, 123.7, 120.2, 106.0, 94.4, 82.3, 73.2, 62.8, 34.5,32.4,29.1,25.4, 22.3; HRMS (ES⁺) calculated for C₂₀H₂₅NO₆(M+Na)⁺: 398.1580, Found: 398.1570. To determine the enantioselectivity achieved in the asymmetric dihydroxylation reaction (4′→5), this reaction was repeated using the pseudoenantiomeric ligand (DHQ)₂PHAL, and the enantiomeric diol was subjected to the same conditions described above. A ˜1/1 mixture of the two purified, enantiomeric bicyclic ketals was then prepared, and separation was achieved on a Chiralpak® AS™ 250×4.6 mm column (Amylose tris-[(S)-α-methylbenzyl carbamate] coated on 10 μm silica-gel substrate, Chiral Technologies Inc., Exton, Pa.) using a flow rate of 1 mmin and an eluent of 4% IPA in hexanes [t_{R}(⁸)=3.13 min., t_{R}(enantiomeric 8)=4.09 min). Using this LC method, the enantiomeric ratio achieved in the transformation of 4′→5 was determined to be major:minor 83:17, and the stereochemistry of the major isomer was assigned using the Sharpless mnemonic.

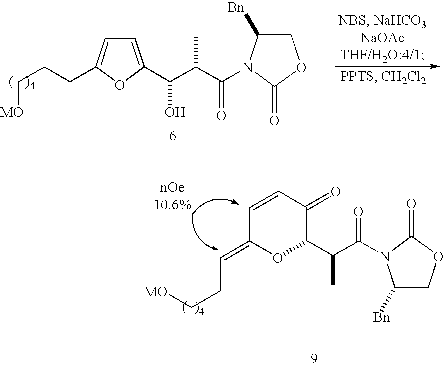

Macrobead-bound-(4S)-4-benzyl-3-{((2S)-2-[(2S)-6-(6-hydroxy-hexylidene)-3-oxo-3,6-dihydro-2H-pyran-2-yl]-propionyl}-oxazolidin-2-one (9). Light yellow beads 6 (0.090 g, 0.563 meq./g, 0.051 mmol) were treated with the same reaction conditions and washing protocols described above for the transformation of 5→8. Solvent was then removed in vacuo to yield 9 as light yellow beads. 5.2 mg of this resin was then treated with HF/Pyridine cleavage conditions (see General Methods) to yield crude product with LCMS purity 86% (λ₂₁₄), t_{R} 8.15 min. 84.6 mg of this resin was then treated with HF/Pyridine cleavage conditions and the crude product was purified by flash chromatography (SiO₂, Hexanes/EtOAc: 1/1→1/2) to afford a yellow oil [7.2 mg, 0.0168 mmol, 0.199 meq./g, Theoretical yield 0.564 meq./g, 35% from 6). R_{f}=0.24 (hexane/EtOAc:1/2); FTIR (film, cm⁻¹) 3432, 2932, 2859, 1780, 1695, 1455, 1391, 1354, 1213, 1112, 1051; ¹HNMR(500 MHz, CDCl₃) δ 7.35-7.26 (m, 3H), 7.21 (app d, J=7 Hz, 2H), 6.93 (d, J=10 Hz, 1H), 5.94 (d, J=10.5 Hz, 1H), 5.22 (t, J=8 Hz, 1H), 4.77-4.70 (m, 1H), 4.73 (d, J=8.5 Hz, 1H), 4.32-4.26 (m, 2H), 4.18 (dd, J=8.5 Hz, 2.5 Hz, 1H), 3.66 (t, J=6.5 Hz, 2H), 3.28 (dd, J=13 Hz, 3.5 Hz, 1H), 2.81 (dd, J=13 Hz, 10 Hz, 1H), 2.32-2.26 (m, 2H), 1.62-1.40 (m, 6H), 1.41 (d, J=6.5 Hz, 3H); ¹³C NMR (100 MHz, CDCl₃) δ 193.4, 173.9, 153.3, 146.9, 141.9, 135.2, 129.4, 128.9, 127.3, 122.1, 121.6, 81.0, 66.3, 62.8, 55.5, 39.8, 38.0, 32.5, 28.7, 27.5, 25.5, 13.7; HRMS (ES⁺) calculated for C₂₄H₂₉NO₆ (M+H)⁺: 428.2073, Found: 428.2061.

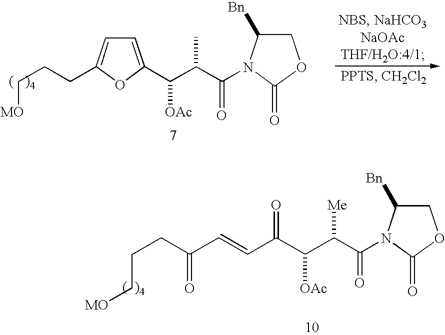

Macrobead-bound-1-((4S)-4-Benzyl-2-oxo-oxazolidin-3-yl)-(2S,3S)-3-acetoxy-13-hydroxy-2-methyl-tridec-5-ene-1,4,7-trione (10). Light yellow beads 7 (0.090 g, 0.482 meq./g, 0.043 mmol) were treated with the same reaction conditions and washing protocols described above for the transformation of 5→8. Solvent was then removed in vacuo to yield 10 as light yellow beads. 5.2 mg of this resin was then treated with HF/Pyridine cleavage conditions (see General Methods) to yield crude product with LCMS purity >90% (λ₂₁₄), t_{R} 8.12 min. 83.8 mg of this resin was then treated with HF/Pyridine cleavage conditions and the crude product was purified by flash chromatography (SiO₂, Hexanes/EtOAc: 1/1→1/2) to afford a yellow oil [15.8 mg, 0.0324 mmol, 0.387 meq./g, Theoretical yield 0.478 meq./g, 81% from 7). R_{f}=0.23 (hexane/EtOAc:1/2); FTIR (film, cm⁻¹) 3539, 2934, 2860, 1779, 1746, 1691, 1454, 1390, 1220, 1108, 1047; ¹H NMR (500 MHz, CDCl₃) δ 7.35-7.27 (m, 3H), 7.19 (app d, J=6 Hz, 2H), 7.16 (d, J=15.5 Hz, 1H), 7.03 (d, J=15.5 Hz, 1H), 5.73 (d, J=5 Hz, 1H), 4.67-4.61 (m, 1H), 4.33-4.27 (m, 2H), 4.21 (dd, J=9 Hz, 2.5 Hz, 1H), 3.64 (t, J=6.5 Hz, 2H), 3.24 (dd, J=13 Hz, 3 Hz, 1H), 2.79 (dd, J=13 Hz, 10 Hz, 1H), 2.66 (t, J=7 Hz, 2H), 2.18 (s, 3H), 1.66 (m, 2H), 1.57 (m, 2H), 1.42-1.32 (m, 4H), 1.25 (d, J=7.5 Hz, 3H); ¹³C NMR (100 MHz, CDCl₃) δ 200.0, 194.7, 172.7, 170.2, 153.3, 137.8, 134.9, 132.1, 129.4, 129.0, 127.4, 76.9, 66.5, 62.8, 55.4, 41.8, 39.5, 37.8, 32.5, 28.8, 25.4, 23.5, 20.6, 12.0; HRMS (ES⁺) calculated for C₂₆H₃₃NO₈ (M+H)⁺: 488.2284, Found: 488.2275.

[image]

[image]

[image]

[image]

[image]

[image]

[image]

[image]

[image]

### III

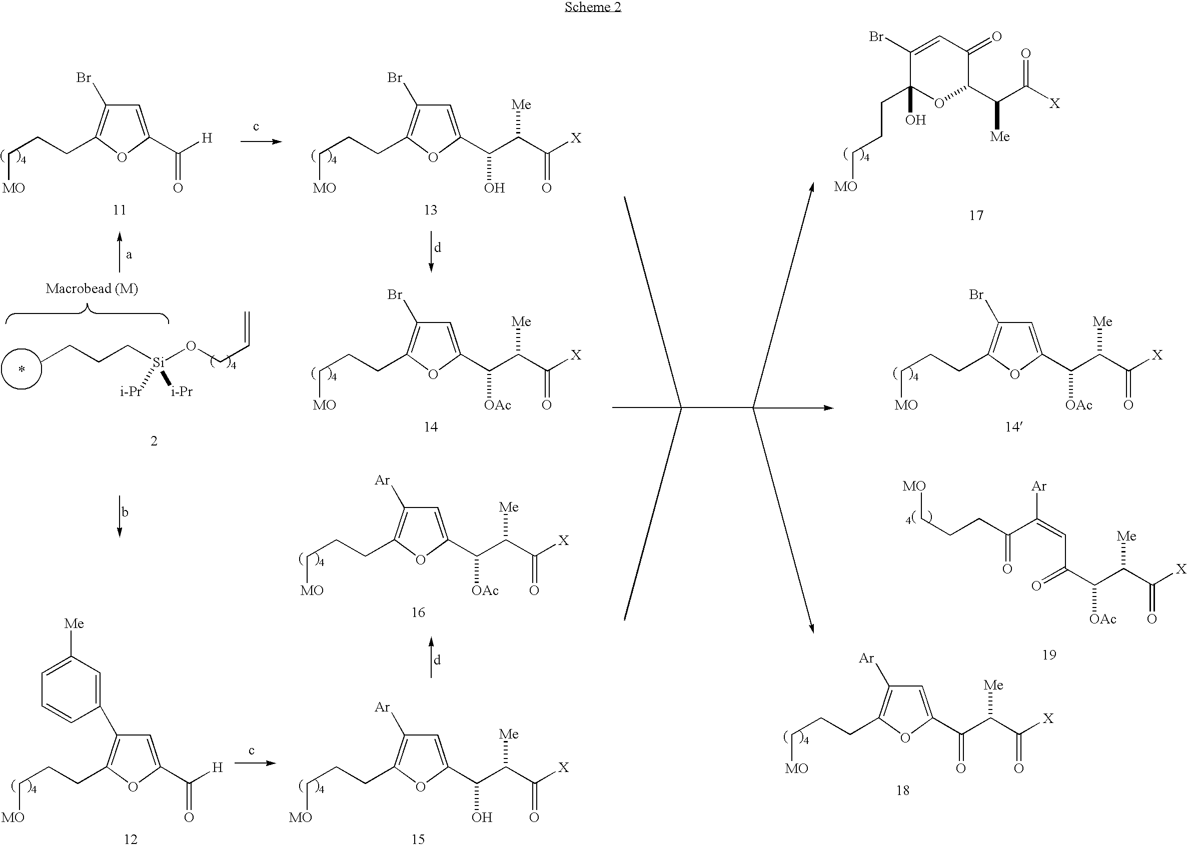

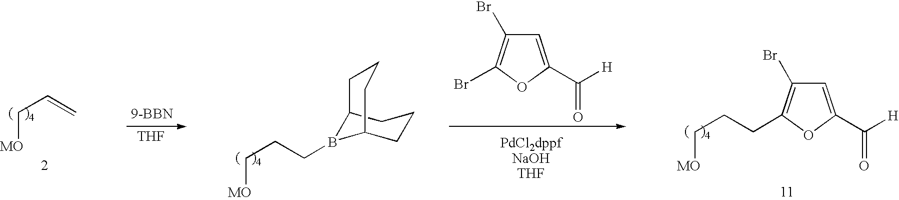

Macrobead-bound-4-Bromo-5-(6-hydroxy-hexyl)-furan-2-carbaldehyde (11) Colorless beads 2 (500 mg) were washed with THF (2×10 mL×10 min each) at rt and then resuspended in 15 mL THF. A 0.5M solution of 9-BBN in THF (10 mL, 5.0 mmol) was then added and the resulting mixture was manually agitated and let stand at rt for 5 h. The reaction solution was then removed via cannula and the colorless resin was washed thoroughly with THF (5×15 mL×10 min each). To the resin was then added solid PdCl₂dppf (10.2 mg, 0.0125 mmol), 4,5-dibromo-2-furaldehyde (635 mg, 2.5 mmol) via cannula as a solution in THF (6.25 mL), and a 1M aq. solution of NaOH (1.25 mL, 1.25 mmol). The resulting orange reaction mixture was sealed under a cloud of Ar and heated at 65° C. with periodic manual agitation for 18 h (reaction mixture turned dark brown). The dark orange resin was then isolated by filtration and washed as follows, 4×(5×THF, 5×H₂O, 5×THF, THF/H₂O: 3/1×30 min), 5×THF, THF×30 min, 5×CH₂Cl₂, CH₂Cl₂×30 min, 5×anh. CH₂Cl₂, anh. CH₂Cl₂×30 min, and then the solvent was removed in vacuo to yield 525 mg of dark orange product resin 11. 5 mg of this resin was then treated with HF/Pyridine cleavage conditions (see General Methods) to yield crude product with LCMS purity 88% (λ₂₁₄), t_{R} 6.40 min. 75.4 mg of this resin was then treated with HF/Pyridine cleavage conditions and the crude product was purified by flash chromatography (SiO₂, hexane/EtOAc: 1/2) to afford a yellow oil (3.9 mg, 0.188 meq./g, 19% over two steps based on estimated meq. Si/g). R_{f}=0.26 (hexanes/EtOAc:1/1); FTIR (film, cm⁻¹) 3401, 2932, 2858, 1683, 1521, 1462, 1393, 1285, 1119; ¹HNMR(500MHz, CDCl₃) δ 9.51 (s, 1H), 7.19 (s, 1H), 3.64 (t, J=7.0 Hz, 2H), 2.76 (t, J=7.5 Hz, 2H), 1.73 (m, 2H), 1.57 (m, 2H), 1.39 (m, 4H); ¹³C NMR (100 MHz, CDCl₃) δ 176.6, 160.3, 150.9, 124.3, 99.3, 62.8, 32.5, 28.8, 27.2, 26.5, 25.3; HRMS (ES⁺) calculated for C₁₁H₁₅BrO₃ (M+H)⁺: 275.0283, Found: 275.0282.

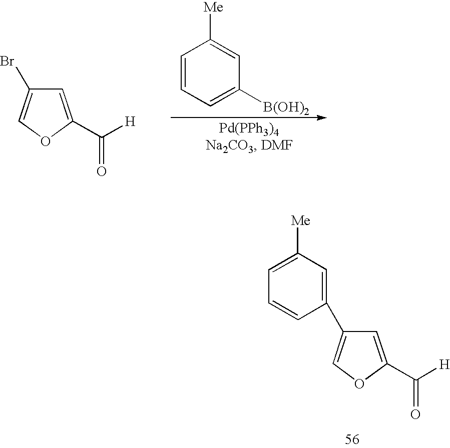

4-m-Tolyl-furan-2-carbaldehyde (56) To a stirred mixture of 4-bromo-2-furaldehyde (ABCR, 5.070 g, 29.0 mmol) and Pd(PPh₃)₄ (0.869 mmol, 1.004 g) in DMF (132 mnL) at rt under Ar was added sodium carbonate (72.4 mmol, 7.68g) as a solution in a minimum amount of water (20 mL), followed by 3-methylbenzeneboronic acid (30.4 mmol, 4.14 g). The resulting light yellow reaction mixture was fitted with a reflux condensor and heated to 105-110° C. with vigorous stirring for 22.5 h (reaction mixture became very dark as reaction progressed). The dark brown reaction mixture was then cooled to rt, filtered over a glass frit, diluted with water (100 mL) and Et₂O (150 mL) and transferred to a separatory funnel. The layers were then separated and the aqueous/DMF layer was extracted with Et₂O (3×100 mL). The combined organic fractions were washed with water (60 mL), brine/water: 1/1 (60 mL), and brine (60 mL), dried over magnesium sulfate, and concentrated in vacuo. Purification by flash chromatography (SiO₂; hexanes/ethyl acetate: 50/1→30/1, column repeated on fractions containing Pd-discoloration) afforded the desired biaryl product 56 as a yellow/orange oil (4.5 g, 24.2 mmol, 83%). R_{f}=0.27 (hexanes/EtOAc:20/1×3 cycles); FTIR (film, cm⁻¹) 3131, 3027, 2920, 2827, 1681, 1613, 1518, 1478, 1349, 1148; ¹HNMR(500MHz, CDCl₃) δ 9.70 (s, 1H), 7.94 (s, 1H), 7.51 (d, J=1 Hz, 1H), 7.31 (m, 3H), 7.16 (m, 1H), 2.40 (s, 3H); ¹³C NMR (100 MHz, CDCl₃) δ 178.0, 153.5, 143.7, 138.8, 130.3, 129.4 ,129.0, 128.9, 126.7, 123.0, 119.0, 21.4; HRMS (ES⁺) calculated for C₁₂H₁₀O₂ (M+H)⁺: 187.0759, Found: 187.0753.

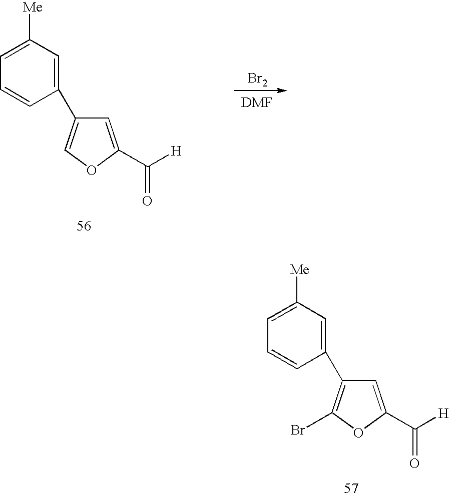

5-Bromo-4-m-tolyl-furan-2-carbaldehyde (57) To DMF (19.3 mL) stirred at −60 to −55° C. under Ar was added bromine (48.3 mmol, 2.48 ml) dropwise over 15 min. The resulting red/orange slurry (solidification occurred upon bromine addition) was warmed to −25° C. over 30 min to yield a bright orange solution (maintained at −25° C.). In a separate flask, 4-m-Tolyl-furan-2-carbaldehyde 56 was dissolved in DMF (19.3 mL) and stirred at rt under Ar. To this solution was added the Br₂/DMF solution dropwise via cannula over 45 min. The resulting dark orange/brown solution was stirred for an additional 15 min and then transferred to a separatory funnel and extracted with 8.5% ethyl acetate/hexanes (5×100 mL, 2×50 mL). The combined extracts were then concentrated in vacuo and the resulting orange DMF solution was dissolved in Et₂O (200 mL) and washed with water (1×40 mL, 1×20 mL) (ethereal layer turned light yellow) and brine (1×20 mL), dried over sodium sulfate, and concentrated in vacuo. The crude product was azeotropically dried (benzene 30 mL, rotary evaporation) to yield 4.3 g of an orange oil, which was purified by flash chromatography (SiO₂, hexanes/ethyl acetate: 100/1→50/1) to yield 3.9 g of the desired product 57 (14.7 mmol, 76%) R_{f}=0.30 (hexanes/EtOAc:20/1×3 cycles); FTIR (film, cm⁻) 3106, 2921, 2824, 1684, 1611, 1578, 1512, 1473, 1370, 1342, 1302, 1167; ¹H NMR (500 MHz, CDCl₃) δ 9.59 (s, 1H), 7.42-7.33 (m, 4H), 7.21 (app d, J=7.5 Hz, 1H), 2.42 (s, 3H); ¹³C NMR (100 MHz, CDCl₃) δ 176.6, 153.6, 138.6, 129.7, 129.2, 128.7, 128.1, 127.9, 127.8, 124.5, 121.8, 21.4; HRMS (ES⁺) calculated for C₁₂H₉BrO₂ (M+H)⁺: 264.9864, Found: 264.9871. For a related transformation see (Sessler and coworkers, J. Org. Chem. 1997, 62, 9251-9260).

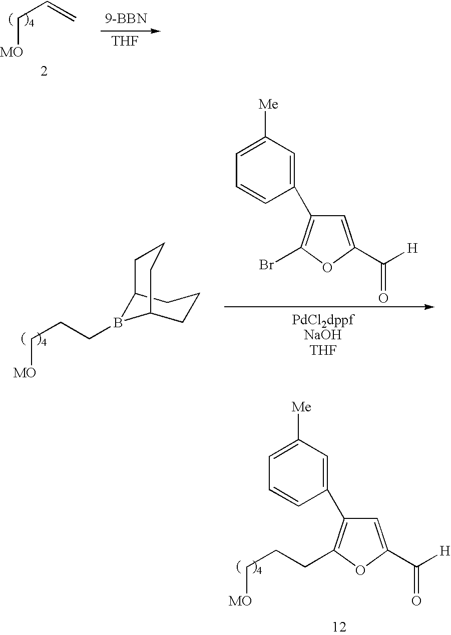

Macrobead-bound-5-(6-Hydroxy-hexyl)-4-m-tolyl-furan-2-carbaldehyde (12) Colorless beads 2 (667 mg, max theoretical loading 1.3 meq/g, 0.867 mmol) were washed with THF (1×30 mL×10 min, 1×20 mL×10 min) at rt and then resuspended in 20.1 mL THF. A 0.5M solution of 9-BBN in THF (13.3 mL, 6.67 mmol) was then added and the resulting mixture was manually agitated and let stand at rt for 5 h. The reaction solution was then removed via cannula and the colorless resin was washed thoroughly with THF (5×15 mL×5-10 min each). To the resin was then added solid PdCl₂dppf (8.2 mg, 0.0075 mmol), 4-m-MePh-5-bromofuraldehyde 57 (884 mg, 3.34 mmol) via cannula as a solution in THF (8.3 mL), and a 1M aq. solution of NaOH (1.67 mL, 1.67 mmol). The resulting orange reaction mixture was sealed under a cloud of Ar and heated at 65° C. with periodic manual agitation for 22 h (reaction mixture turned dark brown). The yellow/orange resin was then isolated by filtration and washed as follows, 5×THF, 5×H₂O, 5×THF, THF/H₂O: 3/1×1 h, 2×(5×THF, THF/H₂O: 3/1×1 h), 5×THF, THF×20 min, 5×CH₂Cl₂, CH₂Cl₂×20 min, 5×anh. CH₂Cl₂, anh. CH₂Cl₂×20 min, and then the solvent was removed in vacuo to yield 761.2 mg of yellow/orange product resin 12. 5.2 mg of this resin was then treated with HF/Pyridine cleavage conditions (see General Methods) to yield crude product with LCMS purity >90% (λ₂₁₄), t_{R} 8.07 min. 75 mg of this resin was then treated with HF/Pyridine cleavage conditions and the crude product was purified by flash chromatography (SiO₂, hexane/EtOAc:1/1) afforded a yellow oil (11.7 mg, 0.545 meq./g loading level). R_{f}=0.29 (hexane/EtOAc:1/1); FTIR (film, cm⁻¹) 3433, 2931, 2858, 1678, 1611, 1526, 1483, 1333, 1122; ¹HNMR (500 MHz, CDCl₃)69.57 (s, 1H), 7.34-7.30 (m, 2H), 7.19-7.14 (m, 3H), 3.61 (t, J=6.5 Hz, 2H), 2.86 (t, J=7.5 Hz, 2H), 2.40 (s, 3H), 1.77 (m, 2H), 1.54 (m, 2H), 1.37 (m, 4H); ¹³C NMR (100 MHz, CDCl₃) δ 177.1, 159.2, 150.9, 138.6, 132.1, 128.7, 128.6, 128.3, 124.9, 124.8, 123.5, 62.8, 32.5, 29.0, 27.9, 27.1, 25.3, 21.5; HRMS (ES⁺) calculated for C₁₈H₂₂O₃ (M+H)⁺: 287.1647, Found: 287.1647.

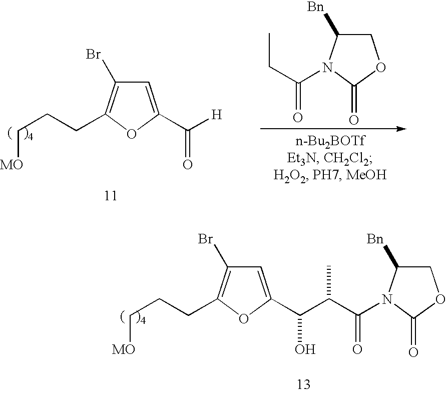

Macrobead-bound-(4S)-4-Benzyl-3-{(3S,2S)-3-[4-bromo-5-(6-hydroxy-hexyl)-furan-2-yl]-3-hydroxy-2-methyl-propionyl}-oxazolidin-2-one (13). Light yellow beads 11 (358 mg, 0.188 meq./g, 0.0673 meq.) were treated with the same reaction conditions used for the transformation of 3→6. After washing, solvent was removed in vacuo to yield 381 mg of light yellow product resin 13. 5.2 mg of this resin was then treated with HF/Pyridine cleavage conditions (see General Methods) to yield crude product with LCMS purity >90% (λ₂₁₄), t_{R} 8.56 min. 75.2 mg of this resin was then treated with HF/Pyridine cleavage conditions and the crude product was purified by flash chromatography (SiO₂, Hexanes/EtOAc: 1/1→1/2) to afford a light yellow oil [8.8 mg, 0.0173 mmol, 0.230 meq./g, Theoretical yield 0.180 meq./g, >95% from 11. R_{f}=0.46 (hexane/EtOAc:1/2); FTIR (film, cm⁻¹) 3446, 2932, 2858, 1781, 1696, 1454, 1386, 1210, 1109, 1014; ¹H NMR (500 MHz, CDCl₃) δ 7.36-7.28 (m, 3H), 7.20 (d, J=7 Hz, 2H), 6.28 (s, 1H), 5.0 (m, 1H), 4.67 (m, 1H), 4.20 (m, 2H), 4.13 (m, 1H), 3.62 (t, J=6 Hz, 2H), 3.24 (dd, J=13.5 Hz, 3 Hz, 1H), 3.12 (br d, J=3.5 Hz, 1H), 2.79 (dd, J=13 Hz, 9 Hz, 1H), 2.61 (t, J=7.5Hz, 2H), 1.62 (m, 2H), 1.56 (m, 2H), 1.4-1.32 (m, 4H), 1.32 (d, J=7 Hz, 3H); ¹³C NMR (100 MHz, CDCl₃) δ 176.2, 152.8, 152.5, 152.3, 134.8, 129.4, 129.0, 127.5, 110.6, 96.4, 68.4, 66.3, 62.8, 55.1, 42.2, 37.8, 32.5, 28.5, 27.4, 25.8, 25.2, 11.9; HRMS (ES⁺) calculated for C₂₄H₃₀BrNO₆ (M+Na)⁺: 530.1154, Found: 530.1169.

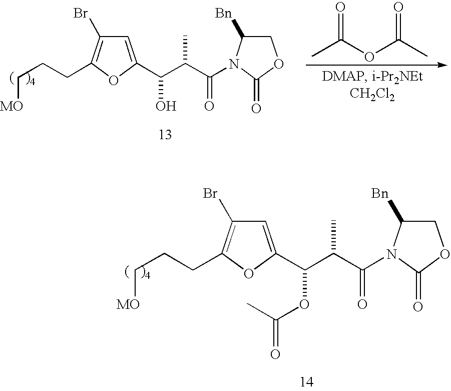

Macrobead-bound-(4S)-4-Benzyl-3-{(3S,2S)-3-[4-bromo-5-(6-hydroxy-hexyl)-furan-2-yl]-3-acetoxy-2-methyl-propionyl}-oxazolidin-2-one (14). Light yellow beads 13 (180 mg, 0.0414 meq.) were treated with the same reaction conditions used for the transformation of 6→7. Solvent was removed in vacuo to yield 183 mg of light yellow product resin 14. 5.0 mg of this resin was then treated with HF/Pyridine cleavage conditions (see General Methods) to yield crude product with LCMS purity >90% (λ₂₁₄), t_{R} 8.94 min. 75.3 mg of this resin was then treated with HF/Pyridine cleavage conditions and the crude product was purified by flash chromatography (SiO₂, Hexanes/EtOAc: 1/1→1/2) to afford a yellow oil (8.5 mg, 0.0154 mmol, 0.205 meq./g, Theoretical yield 0.228 meq./g, 90% from 13). R_{f}=0.21 (hexane/EtOAc:1/1); FTIR (film, cm⁻¹) 3535, 2933, 2859, 1782, 1745, 1698, 1454, 1387, 1223, 1108, 1018; ¹H NMR (500 MHz, CDCl₃) δ 7.34-7.26 (m, 3H), 7.19 (d, J=7.5 Hz, 2H), 6.31 (s, 1H), 6.11 (d, J=7.5 Hz, 1H), 4.55 (m, 1H), 4.47 (m, 1H), 4.16 (m, 2H), 3.62 (t, J=6.5 Hz, 2H), 3.23 (dd, J=13, 3 Hz, 1H), 2.76 (dd, J=15, 9.5 Hz, 1H), 2.60 (t, J=7 Hz, 2H), 2.09 (s, 3H), 1.64-1.53 (m, 4H), 1.40-1.30 (m, 4H), 1.32 (d, J=6.5 Hz, 3H); ¹³C NMR (100 MHz, CDCl₃); δ 173.1, 170.0, 153.1, 153.1, 149.4, 135.0, 129.4, 128.9, 127.4, 112.6, 96.4, 68.7, 66.3, 62.8, 55.3, 40.7, 37.8, 32.5, 28.6, 27.4, 25.9, 25.2, 20.9, 13.2; HRMS (ES⁺) calculated for C₂₆H₃₂BrNO₇ (M+Na)⁺: 572.1260, Found: 572.1277.

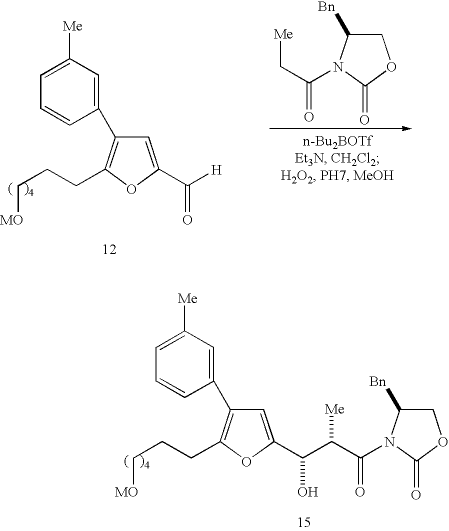

Macrobead-bound-(4S)-4-Benzyl-3-{(3S,2S)-3-hydroxy-3-[5-(6-hydroxy-hexyl)-4-m-tolyl-furan-2-yl]-2-methyl-propionyl}-oxazolidin-2-one (15). Light yellow beads 12 (400 mg, 0.218 meq.) were treated with the same reaction conditions used for the transformation of 3→6. Solvent was removed in vacuo to yield 456 mg of light yellow product resin 15. 5.2 mg of this resin was then treated with HF/Pyridine cleavage conditions (see General Methods) to yield crude product with LCMS purity >90% (λ₂₁₄), t_{R} 9.47 min. 75.2 mg of this resin was then treated with HF/Pyridine cleavage conditions and the crude product was purified by flash chromatography (SiO₂, Hexanes/EtOAc: 1/1→1/2) to afford a yellow oil [18.0 mg, 0.0346 mmol, 0.460 meq./g, Theoretical yield 0.484 meq./g, 95% from 12. R_{f}=0.30 (hexane/EtOAc:1/1); FTIR (film, cm⁻¹) 3446, 2932, 2858, 1782, 1696, 1605, 1455, 1386, 1210, 1109, 1051, 1015; ¹H NMR (500 MHz, CDCl₃) δ 7.36-7.06 (m, 9H), 6.41 (s, 1H), 5.07 (d, J=4 Hz, 1H), 4.65 (m, 1H), 4.25-4.10 (m, 3H), 3.60 (t, J=7 Hz, 2H), 3.25 (dd, J=13.5 Hz, 3 Hz, 1H), 3.10 (br s, 1H), 2.80 (dd, J=13.5 Hz, 9.5 Hz, 1H), 2.75 (t, J=8 Hz, 2H), 2.37 (s, 3H), 1.69 (m, 2H), 1.54 (m, 2H), 1.39 (d, J=7 Hz, 3H, 1.35 (m,4H); ¹³CNMR (100 MHz, CDCl₃);δ 176.3, 152.9, 151.6, 151.2, 138.1, 134.9, 133.9, 129.4, 129.0, 128.4, 128.4, 127.5, 127.2, 124.7, 121.5, 108.5, 68.7, 66.2, 62.8, 55.2, 42.5, 37.8, 32.5, 28.8, 28.1, 26.7, 25.2, 21.5, 12.1; HRMS (ES⁺) calculated for C₃₁H₃₇NO₆ (M+NH₄)⁺: 537.2965, Found: 537.2977.

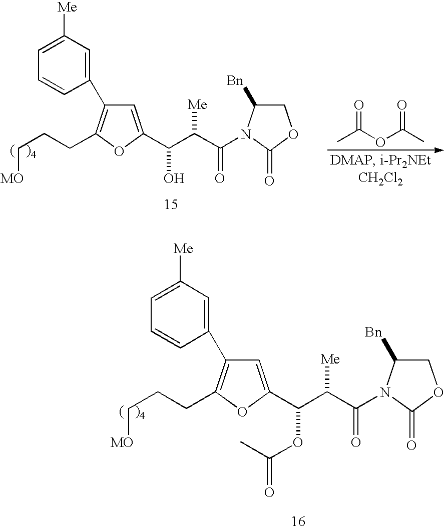

Macrobead-bound-(4S)-4-Benzyl-3-{(3S,2S)-3-acetoxy-3-[5-(6-hydroxy-hexyl)-4-m-tolyl-furan-2-yl]-2-methyl-propionyl}-oxazolidin-2-one (16). Light yellow beads 15 (180 mg, 0.460 meq/g, 0.083 meq.) were treated with the same reaction conditions used for the transformation of 6→7. Solvent was removed in vacuo to yield light yellow product resin 16. 5.2 mg of this resin was then treated with HF/Pyridine cleavage conditions (see General Methods) to yield crude product with LCMS purity >90% (λ₂₁₄), t_{R} 10.55 min. 75.2 mg of this resin was then treated with HF/Pyridine cleavage conditions and the crude product was purified by flash chromatography (SiO₂, Hexanes/EtOAc: 1/1→1/2) to afford a yellow oil (16.0 mg, 0.0285 mmol, 0.379 meq./g, Theoretical yield 0.451 meq./g, 84% from 15). R_{f}=0.26 (hexane/EtOAc:1/1); FTIR (film, cm⁻¹) 3538, 3028, 2932, 2859, 1782, 1744, 1700, 1606, 1455, 1386, 1227, 1108, 1018; ¹H NMR (500 MHz, CDCl₃) δ 7.34-7.06 (m, 9H), 6.44 (s, 1H), 6.19 (d, J=8 Hz, 1H), 4.59-4.50 (m, 2H), 4.16-4.10 (m, 2H), 3.60 (t, J=6.5 Hz, 2H), 3.24 (dd, J=13, 3 Hz, 1H), 2.77 (dd, J=13.5, 10 Hz, 1H), 2.74 (t, J=7.5 Hz, 2H), 2.36 (s, 3H), 2.12 (s, 3H), 1.70-1.64 (m, 2H), 1.54(m, 2H), 1.39-1.32 (m, 4H), 1.36 (d, J=6.5Hz, 3H); ¹³C NMR (100 MHz, CDCl₃);δ 173.4, 170.1, 153.1, 151.7, 148.7, 138.1, 135.1, 133.6, 129.4, 128.9, 128.4, 128.4, 127.4, 127.3, 124.6, 121.6, 110.5, 69.1, 66.2, 62.8, 55.4, 40.8, 37.8, 32.6, 28.9, 28.1, 26.7, 25.3, 21.5, 21.0, 13.2; HRMS (ES⁺) calculated for C₃₃H₃₉NO₇ (M+Na)⁺: 584.2624, Found: 584.2609.

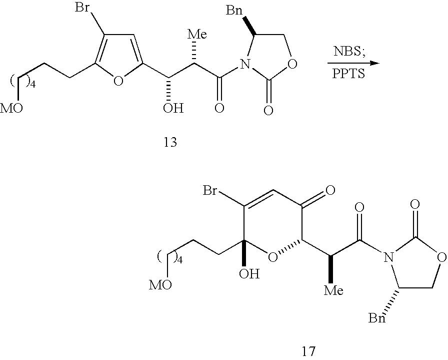

Macrobead-bound-(4S)-4-Benzyl-3-{(2S)-2-[(2S,6R)5-bromo-6-hydroxy-6-(6-hydroxy-hexyl)-3-oxo-3,6-dihydro-2H-pyran-2-yl]-propionyl}-oxazolidin-2-one (17). Light yellow beads 13 (0.090 g, 0.230 meq./g, 0.021 mmol) were treated with the same reaction conditions and washing protocol described above for the transformation of 5→8. Solvent was then removed in vacuo to yield 17 as light yellow beads. 5.2 mg of this resin was then treated with HF/Pyridine cleavage conditions (see General Methods) to yield crude product with LCMS purity 90% (λ₂₁₄), t_{R} 8.14 min, epimeric ratio=9.4:1. 87.8 mg of this resin was then treated with HF/Pyridine cleavage conditions and the crude product was purified by flash chromatography (SiO₂, Hexanes/EtOAc: 1/1→1/2) to afford a yellow oil (8.6 mg, 0.0164 mmol, 0.187 meq./g, Theoretical yield 0.229 meq./g, 82% from 13, the stereochemical assignment at the hemiketal center has been tentatively assigned. R_{f}=0.3 (hexane/EtOAc:1/2); FTIR (film, cm⁻¹) 3452, 2933, 2860, 1781, 1695, 1605, 1455, 1392, 1352, 1208, 1110, 1050; ¹H NMR (500 MHz, CDCl₃) δ 7.35-7.25 (m, 3H), 7.22-7.18 (m, 2H), 6.50 (s, 1H), 4.92 (d, J=8.5 Hz, 1H), 4.74 (m, 1H), 4.30 (app t, J=8.5 Hz, 1H), 4.19 (dd, J=9.5, 2.5 Hz, 1H), 4.12 (dq, J=8, 7 Hz, 1H), 3.65 (t, J=7 Hz, 2H), 3.25 (dd, J=13.5, 3 Hz, 1H), 2.81 (dd, J=13, 10 Hz, 1H), 2.16 (m, 1H), 1.93 (m, 1H), 1.58 (m, 2H), 1.42-1.34 (m, 6H), 1.33 (d, J=7 Hz, 3H); ¹³C NMR (100 MHz, CDCl₃); δ 192.2, 174.3, 153.2, 148.3, 135.1, 131.0, 129.5, 129.0, 127.4, 98.1, 74.5, 66.3, 62.9, 55.3, 40.5, 38.3, 38.0, 32.5, 29.0, 25.4, 23.2, 13.6; HRMS (ES⁺) calculated for C₂₄H₃₀BrNO₇ (M+Na)⁺: 546.1103, Found: 546.1086.

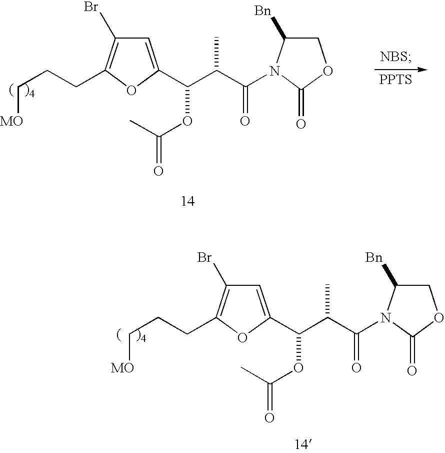

Macrobead-bound-(4S)-4-Benzyl-3-{(3S,2S)-3-[4-bromo-5-(6-hydroxy-hexyl)-furan-2-yl]-3-acetoxy-2-methyl-propionyl}-oxazolidin-2-one (14′). Light yellow beads 14 (0.090 g, 0.205 meq./g, 0.018 mmol) were treated with the same reaction conditions and washing protocols described above for the transformation of 5→8. Solvent was then removed in vacuo to yield unreacted 14′ as light yellow beads. 5.2 mg of this resin was then treated with HF/Pyridine cleavage conditions (see General Methods) to yield crude product with LCMS purity >90% (λ₂₁₄), t_{R} 9.55 min. 84.2 mg of this resin was then treated with HF/Pyridine cleavage conditions and the crude product was purified by flash chromatography (SiO₂, Hexanes/EtOAc: 1/1→1/2) to afford a yellow oil (8.4 mg, 0.053 mmol, 0.181 meq./g, Theoretical yield 0.205 meq./g, 88% from 14).

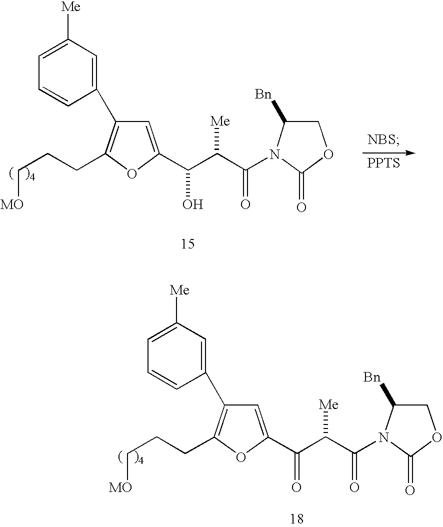

Macrobead-bound-1-((4S)-4-Benzyl-2-oxo-oxazolidin-3-yl)-3-[5-(6-hydroxy-hexyl)-4-m-tolyl-furan-2-yl]-(2S)-2-methyl-propane-1,3-dione (18). Light yellow beads 15 (0.090 g, 0.460 meq./g, 0.041 mmol) were treated with the same reaction conditions and washing protocol described above for the transformation of 5→8. Solvent was then removed in vacuo to yield 18 as light yellow beads. 5.2 mg of this resin was then treated with HF/Pyridine cleavage conditions (see General Methods) to yield crude product with LCMS purity 72% (λ₂₁₄), t_{R} 10.12 min. 86.1 mg of this resin was then treated with HF/Pyridine cleavage conditions and the crude product was purified by flash chromatography (SiO₂, Hexanes/EtOAc: 2/1→1/2) to afford a yellow oil (15.2 mg, 0.0294 mmol, 0.341 meq./g, Theoretical yield 0.460 meq./g, 74% from 15). R_{f}=0.24 (hexane/EtOAc:1/1); FTIR (film, cm⁻¹) 3524, 2933, 2859, 1780, 1706, 1700, 1524, 1482, 1454, 1390, 1358, 1213, 1125, 1014; ¹H NMR (500 MHz, CDCl₃) δ 7.36-7.13 (m, 10H), 5.28 (q, J=7.5 Hz, 1H), 4.77 (m, 1H), 4.25 (app t, J=8.5 Hz, 1H), 4.18 (dd, J=9, 2.5 Hz, 1H), 3.60 (t, J=6.5 Hz, 2H), 3.37 (dd, J=13, 3 Hz, 1H), 2.84 (t, J=7.5 Hz, 2H), 2.79 (dd, J=14, 10 Hz, 1H), 2.39 (s, 3H), 1.76 (m, 2H), 1.58 (d, J=7 Hz, 3H), 1.53 (m, 2H), 1.40-1.34 (m, 4H); ¹³C NMR (100 MHz, CDCl₃) δ 185.7, 170.4, 157.5, 153.9, 149.4, 138.7, 135.4, 132.6, 129.7, 129.2, 129.0, 128.9, 128.4, 127.6, 125.2, 125.0, 120.1, 66.8, 63.0, 55.7, 49.1, 38.2, 32.7, 29.1, 28.1, 27.4, 25.5, 21.7, 14.0; HRMS (ES⁺) calculated for C₃₁H₃₅NO₆ (M+H)⁺: 518.2542, Found: 518.2532.

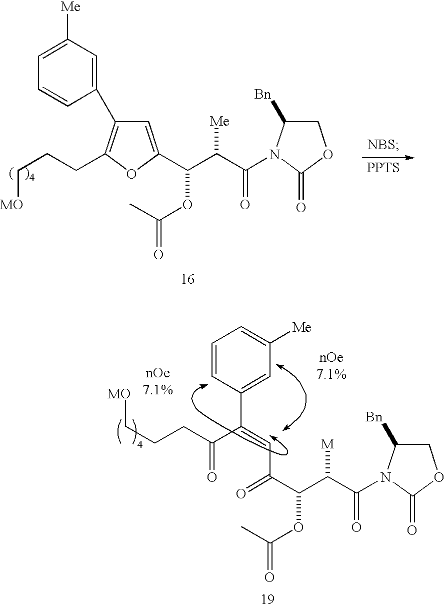

Macrobead-bound-acetic acid (1S)-1-[2-((4S)-4-benzyl-2-oxo-oxazolidin-3-yl)-(1S)-1-methyl-2-oxo-ethyl]-11-hydroxy-2,5-dioxo-4-m-tolyl-undec-3-enyl ester (19). Light yellow beads 16 (0.090 g, 0.379 meq./g, 0.034 mmol) were treated with the same reaction conditions and washing protocol described above for the transformation of 5→8. Solvent was then removed in vacuo to yield 19 as light yellow beads. 5.2 mg of this resin was then treated with HF/Pyridine cleavage conditions (see General Methods) to yield crude product with LCMS purity 66% (λ₂₁₄), t_{R} 9.57 min. 84.4 mg of this resin was then treated with HF/Pyridine cleavage conditions and the crude product was purified by flash chromatography (SiO₂, Hexanes/EtOAc: 2/1→1/2) to afford a yellow oil (13.3 mg, 0.0230 mmol, 0.273 meq./g, Theoretical yield 0.377 meq./g, 72% from 16). R_{f}=0.29 (hexane/EtOAc:1/2); FTIR (film, cm⁻¹) 3537, 2934, 2859, 1779, 1746, 1702, 1577, 1454, 1388, 1223, 1106, 1048; ¹H NMR (500 MHz, CDCl₃) δ 7.35-7.24 (m, 7H), 7.20-7.17 (m, 2H), 6.81 (s, 1H), 5.74 (d, J=5 Hz, 1H), 4.62 (m, 1H), 4.36-4.30 (m, 2H), 4.20 (dd, J=9, 2 Hz, 1H), 3.61 (t, J=7 Hz, 2H), 3.25 (dd, J=13.5, 3 Hz, 1H), 2.80 (dd, J=13.5, 9.5 Hz, 1H), 2.61 (dt, J=18, 7 Hz, 1H), 2.51 (dt, J=18.5, 7.5 Hz, 1H), 2.37 (s, 3H), 2.18 (s, 3H), 1.72 (m, 2H), 1.54 (m, 2H), 1.39-1.33 (m, 4H), 1.23 (d, J=6.5 Hz, 3H); ¹³C NMR (100 MHz, CDCl₃) δ 207.0, 192.9, 172.4, 170.4, 159.5, 153.5, 139.0, 135.0, 132.9, 131.9, 129.4, 129.1, 129.0, 127.7, 127.4, 124.3, 117.9, 77.11, 66.6, 62.8, 55.6, 41.9, 39.2, 37.8, 32.5, 28.4, 25.3, 22.8, 21.4, 20.7, 11.2; HRMS (ES⁺) calculated for C₃₃H₃₉NO₈ (M+NH₄)⁺: 595.3019, Found: 595.3034.

[image]

[image]

[image]

[image]

[image]

[image]

[image]

[image]

[image]

[image]

IV. Combining results from FIG. 14E and FIG. 15A, it was possible to assemble a collection of 6 macrobead-bound substrates 6, 7, and 13-16 representing a complete, 3×2 combinatorial matrix of σ-elements (—H, —Br, or —Ar at the 4-position of furan combined with —OH or —OAc on the α-carbon, FIG. 2B). This collection of 6 individual macrobeads was placed in the same reaction vessel and exposed to the same set of oxidative and acidic reaction conditions described above for the transformation of 5→8, resulting in a complete, combinatorial (3×2=6) matrix of distinct skeletal outcomes, i.e., six unique displays of chemical information in 3-dimensional space, in the form of products 9, 10, 14′, 17, 18, and 19.

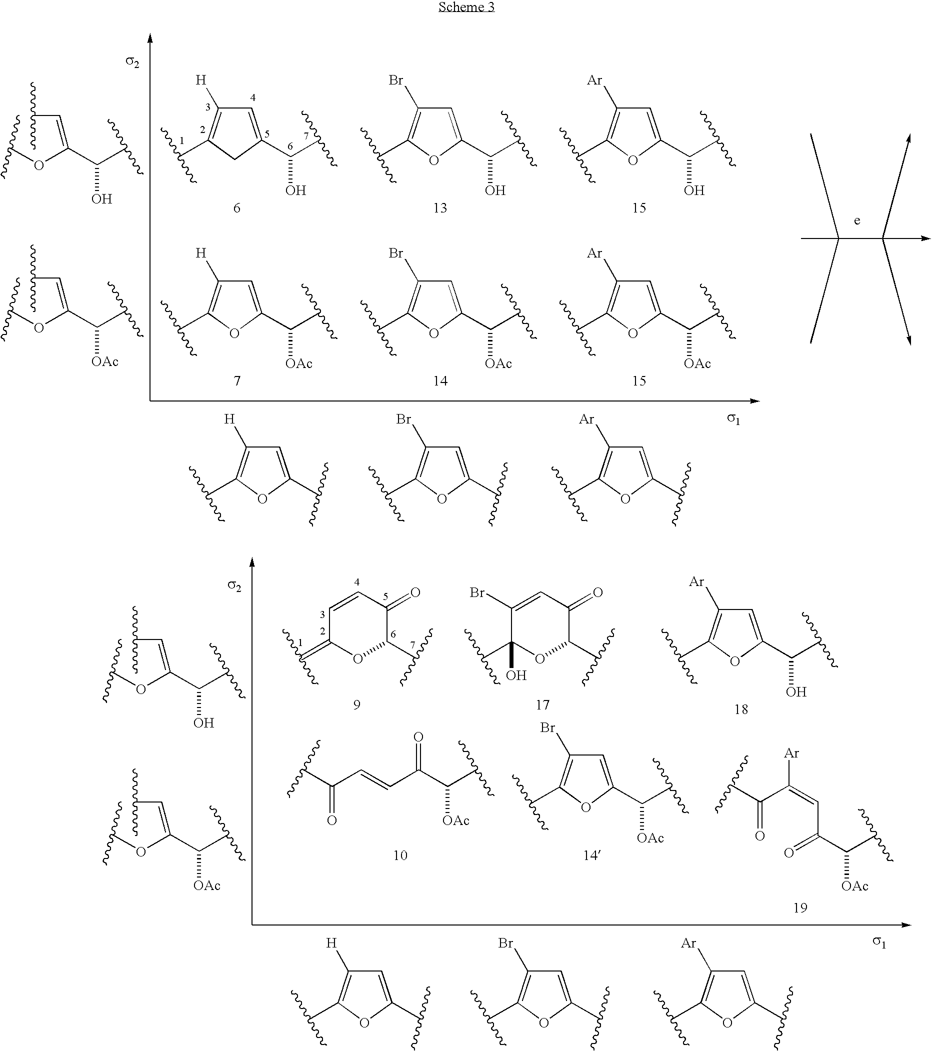

Procedure: (The following experiment was performed in triplicate) A common reaction vessel was charged with 6 individual macrobeads 6, 7, 13, 14, 15, and 16 and to this mixture at rt under ambient was added THF/water: 4/1 (1.5 mL), NaHCO₃ (56.7 mg, 0.675 mmol), NaOAc (27.7 mg, 0.338 mmol), and N-bromosuccinimide (40.0 mg, 0.23 mmol). The resulting mixture was sealed, wrapped in aluminum foil, and tumbled at rt for 1 h. The 6 macrobeads were then isolated from the reaction mixture by filtration and collectively washed as follows: 5×THF, 5×H₂O, 5×THF, THF/water: 3/1×1 h, 5×THF, THF×1 h, 5×CH₂Cl₂, CH₂Cl₂×30 min, 5×anh. CH₂Cl₂, anh. CH₂Cl₂×30 min. After removing the solvent in vacuo, the 6 macrobeads were transferred collectively to a new reaction vessel containing a 0.00075M solution of pyridiniump-toluenesulfonate in CH₂Cl₂ (2 mL). The resulting mixture was sealed under a cloud of argon and maintained at 40-45° C. (oil bath) for 20 h. The 6 macrobeads were then isolated from the reaction mixture by filtration and washed as follows: 5×THF, 5×H₂O, 5×THF, THF/dil. aq. NaHCO₃ (sat. aq. NaHCO₃/H₂O: 1/2): 1/1×1 h, 5×THF, 5×H₂O, 5×THF, THF/dilute aq. NH₄Cl (sat. aq. NH₄Cl/H₂O: 1/2): 1/1×1 h, 5×THF, 5×H20, 5×THF, THF/H₂O: 3/1×1 h, 5×THF, THF×1 h, 5×CH₂Cl₂, CH₂Cl₂×30 min, 5×anh. CH₂Cl₂, anh. CH₂Cl₂×30 min. Solvent was then removed in vacuo to yield 6 product macrobeads, which were segregated into individual polypropylene eppendorf tubes and treated with HF/Pyridine cleavage conditions (see General Methods). The cleaved products were then analyzed by LCMS. In all 3 experiments, 6/6 (100%) of the anticipated compounds were identified as the major product (by t_{R} and mass) cleaved from an individual macrobead.

V. The synthesis of diverse skeletons is critical to achieving diverse displays of chemical information in 3-dimensional space. To provide some form of quantification for this type of diversity found in the set of six skeletons shown in FIG. 2B, we developed a skeletal diversity metric based on the distance, angle, and dihedral angle between common atoms in computationally derived 3-dimensional structures. Specifically, the missing bonds in both the substrates and products in FIG. 2B represent potential attachment sites to which building blocks could be appended. The six substrates, having a 3×2 matrix of different appendages attached to a common cc-alkoxy furan skeleton resemble the types of compounds typically derived from the one synthesis-one skeleton approach. Alternatively, the six products represent six distinct molecular skeletons generated combinatorially using the σ-element-based synthesis strategy. Comparing and contrasting these two collections (which are almost constitutionally isomeric) can provide a metric for the skeletal diversity generated in this one reaction using a common set of reagents. By replacing each of the missing bonds in the 12 structures shown in FIG. 2B with methyl groups (or a methylene group for the ‘left side’ of structure 9), we were able to generate a collection of 12 simplified structures: 6 substrates (6*, 7*, 13*, 14*, 15*, and 16*) and 6 products (9*, 10*, 17*, 14′*, 18*, and 19*), which all share in common the 7 contiguous carbon atoms labeled C₁-C₇. Using the Spartan software package (Spartan '02, Wavefunction, Inc.) and a Gateway PC with an Intel Pentium 4 processor, we then performed the following two-step calculation on all 12 structures: The equilibrium conformer was determined reproducibly using the standard Spartan equilibrium conformer search with semiempirical AM1 calculations, followed by the determination of equilibrium geometry using the Hartree-Fock method with the 6-31G* split-valence basis set.

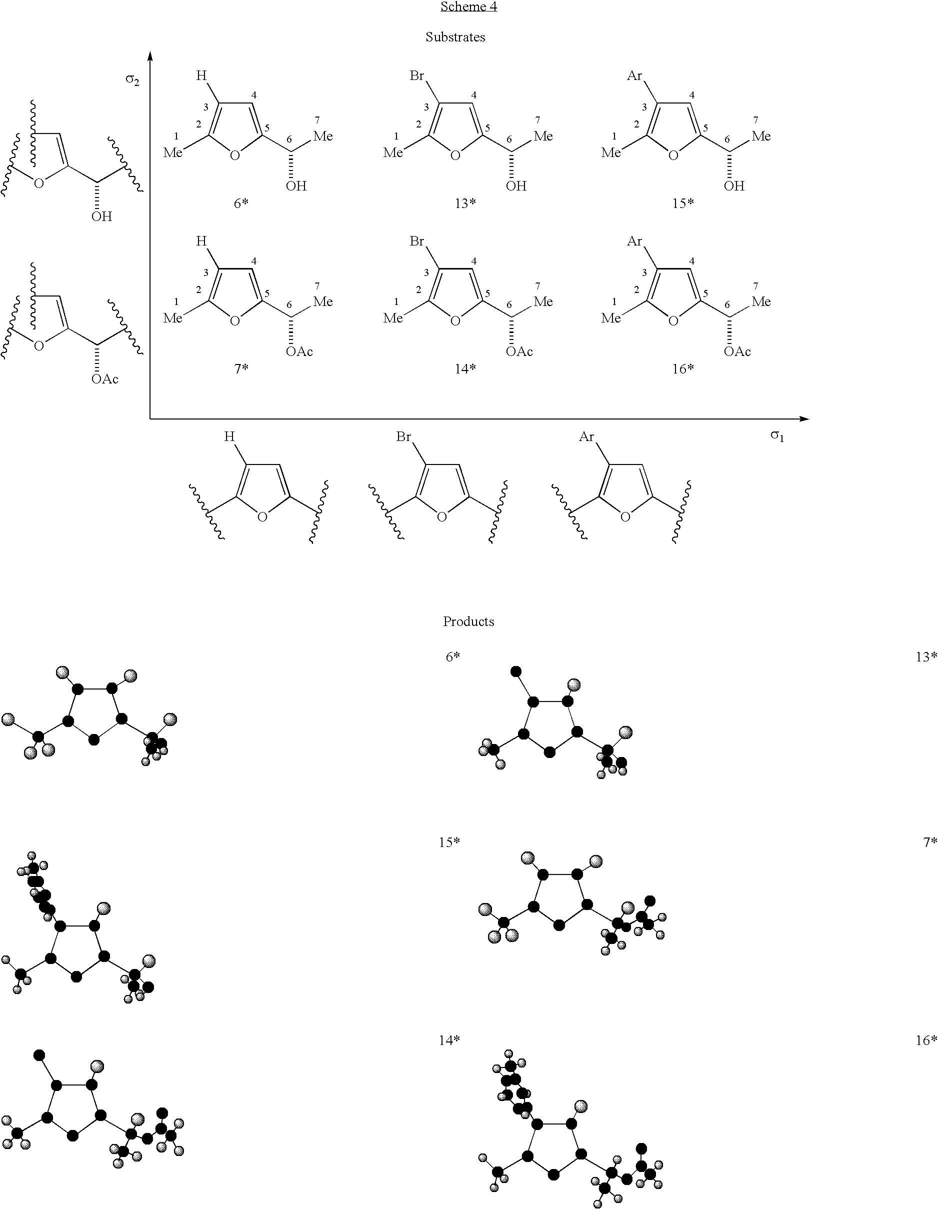

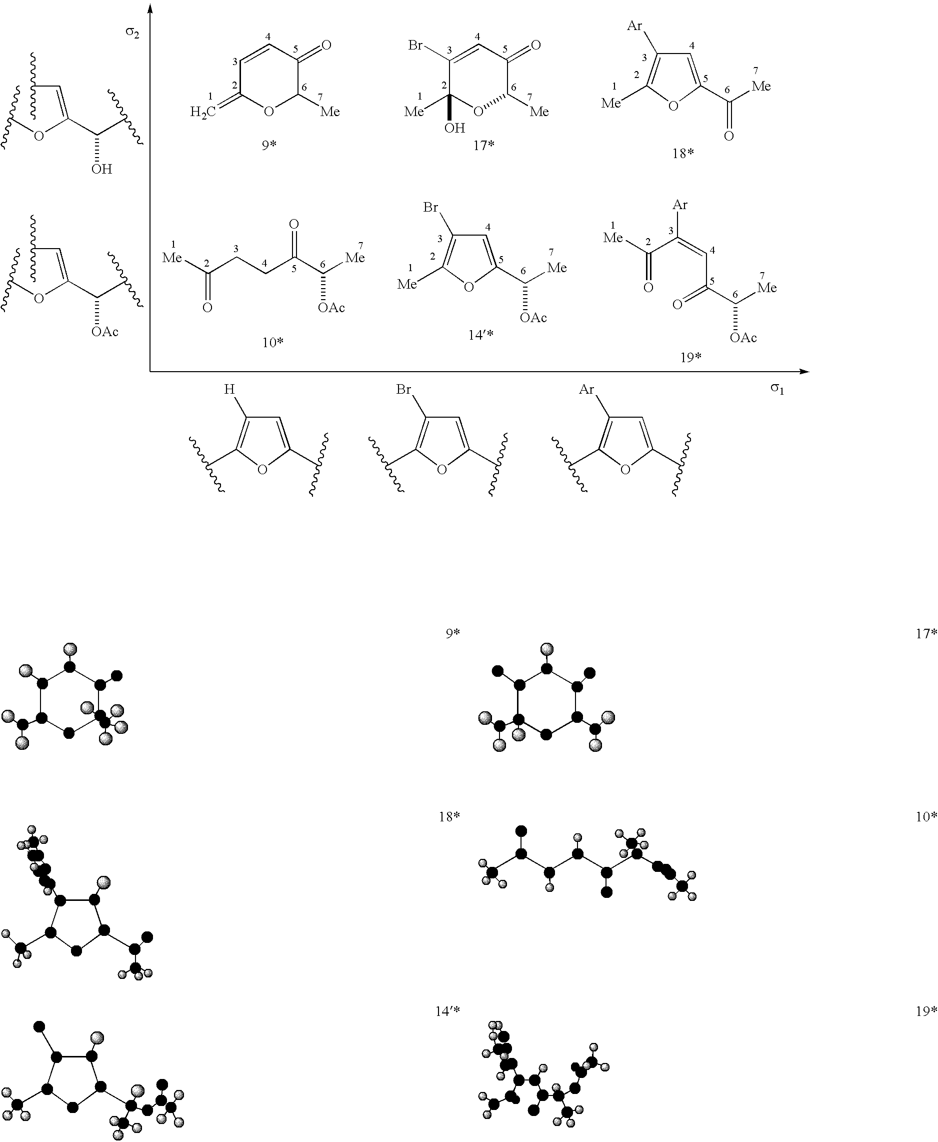

For each of these 12 computationally derived 3-dimensional structures, the positions of every other carbon in the common, contiguous 7-carbon atom stretch were then used to determine the following three parameters (each parameter provides unique information regarding the relative positions of the building block attachment sites, C₁ and C₇, in 3-dimensional space:

1. the distance (in angstroms) between C₁ and C₇

2. the angle C₁—the midpoint between C₃ and C₅-C₇.

3. the dihedral angle comprising C₁, C₃, C₅, and C₇.

This analysis produced the following data

TABLE 1

| | 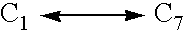 | 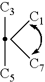 | 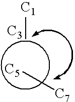 |
|---|---|---|---|
| | distance | angle | dihedral angle |
| | (angstroms) | (degrees) | (degrees) |
| Substrates | | | |
| 6* | 5.30 | 110.6 | 76.5 |
| 7* | 5.17 | 105.9 | 56.8 |
| 13* | 5.30 | 110.6 | 75.2 |
| 14* | 5.16 | 105.8 | 55.7 |
| 15* | 5.27 | 109.6 | 74.9 |
| 16* | 5.14 | 104.9 | 54.2 |
| standard deviation | 0.07 | 2.4 | 10.0 |
| Products | | | |
| 9* | 4.04 | 83.7 | 39.4 |
| 10* | 6.81 | 145.0 | 158.2 |
| 17* | 4.64 | 91.0 | 14.9 |
| 14′* | 5.16 | 105.8 | 55.7 |
| 18* | 5.10 | 102.1 | 2.6 |
| 19* | 5.90 | 123.1 | 128.5 |
| standard deviation | 0.89 | 20.5 | 57.5 |

Plotting these parameters for both substrates and products in a 3-dimensional plot using the Spotfire graphing package produced the 3-D plots shown in FIG. 2C of the text. By this analysis, the 6 substrates, which represent a collection of products having a common skeleton similar to those derived from the one-synthesis-one skeleton approach, create a dense cluster (the two lobes of this dense cluster represent the acetylated and non-acetylated substrates). In contrast, the 6 products, which represent 6 distinct molecular skeletons generated combinatorially using the σ-element-based synthesis strategy, distribute broadly (both plots are drawn to the same scale) consistent with a diverse display of chemical information in 3-dimensional space.

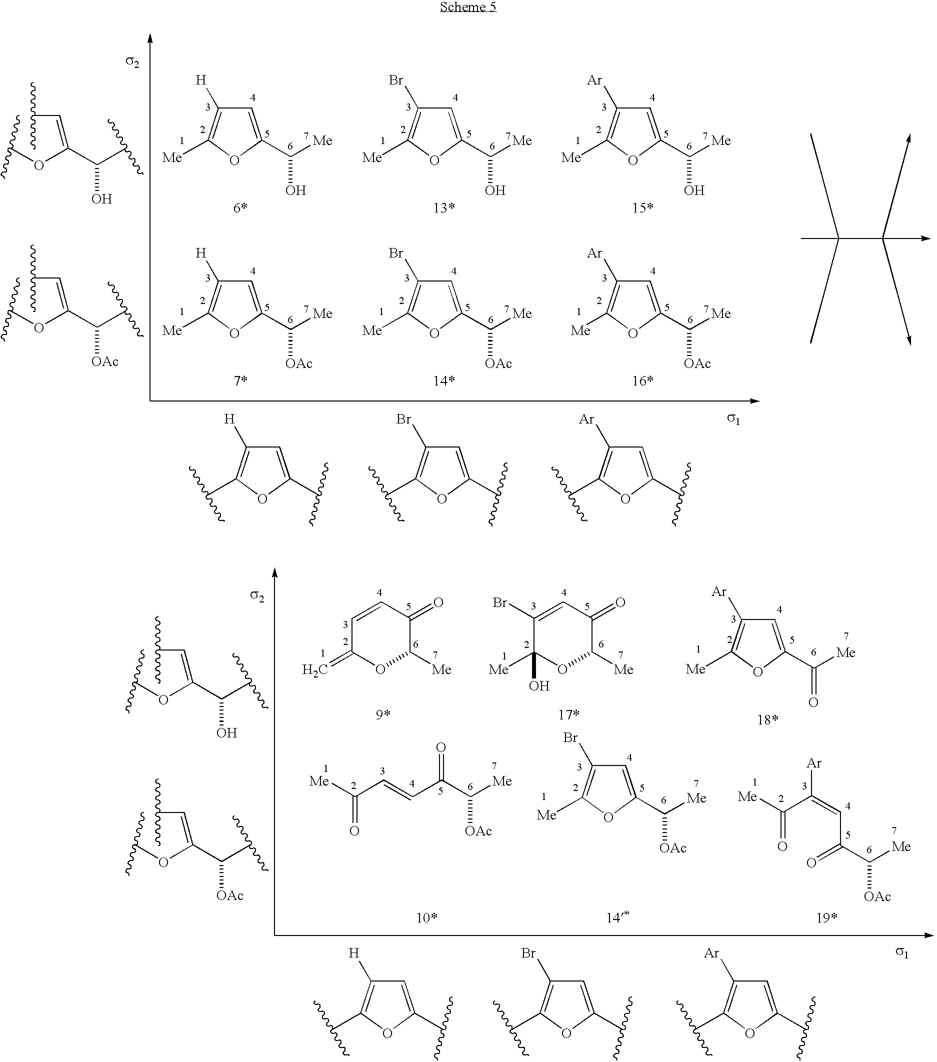

[image]

### VI.

We next set out to determine if this 3×2 combinatorial matrix of σ-elements could effectively pre-encode the same matrix of 6 skeletal outcomes when a complete combinatorial matrix of building blocks was also attached to the same common core (see FIG. 1C). If successful, this strategy would provide a highly efficient mechanism to access a collection of compounds representing a complete set of overlapping matrices of these diversity elements, i.e., a complete matrix of molecular skeletons, each derivatized with a complete matrix of building blocks (the equivalent of 6 different collections of compounds synthesized individually using the one synthesis-one skeleton approach). The 36 substrates 20a-jj were synthesized and exposed to common conditions in parallel as described below.

[image]

Step 1. Loading of Building Block #1 (BB₁).

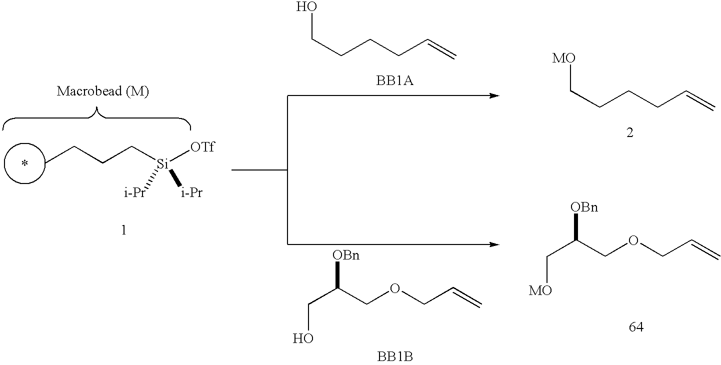

1.2 g of 3-[Diisopropyl(p-methoxyphenyl)silyl]propyl functionalized macrobeads 1 was split into two portions (600 mg each) and each portion was subjected to a unique loading reaction 5 with BB₁A or BB₁B, using the same protocol described previously for the transformation of 1→2 to yield 2 and 64, which were carried on to step 2.

Step 2. Suzuki Coupling of Skeletal Information Unit #1 (σ₁)

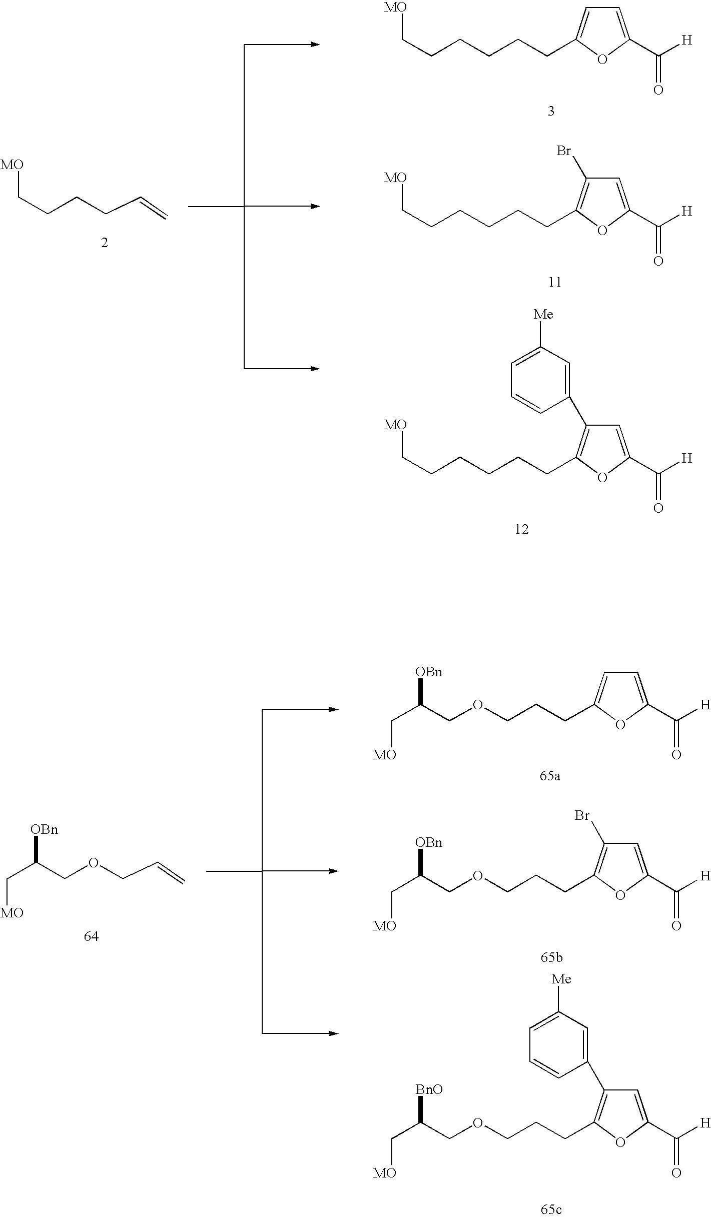

Suzuki coupling of Skeletal Information Unit #1 (σ₁) Colorless beads 2 (555 mg) and 64 (630 mg) were each split evenly by weight into three portions. Each of the three portions of 2 and 64 was then subjected to a B-alkyl Suzuki coupling with a unique 4-substituted-5-bromofuraldehyde (σ₁=H, σ₂=Br, or σ₃=m-MePh, 6 parallel reactions) using the same protocols described previously for the transformation of 2→3, 2→11, and 2→12.

TABLE 2
Results of Step 2

| | | | | | | % Purity | HRMS | | |
|---|---|---|---|---|---|---|---|---|---|
| No. | BB₁ | σ₁ | BB₂ | σ₂ | ¹H NMR | LCMS, 214 nm | Ionization | Calculated | Observed |
| 3 | A | H | — | — | ✓ | >85 | ES+ | (M + H⁺) 197.1177 | 197.1177 |
| 11 | A | Br | — | — | ✓ | >90 | ES+ | (M + H⁺) 275.0283 | 275.0282 |
| 12 | A | m-MePh | — | — | ✓ | >90 | ES+ | (M + H⁺) 287.1647 | 287.1647 |
| 65a | B | H | — | — | ✓ | 88 (280 nm, 92) | ES+ | (M + H⁺) 319.1545 | 319.1536 |
| 65b | B | Br | — | — | ✓ | 71 (280 nm, 94) | ES+ | (M + H⁺) 397.0650 | 397.0645 |
| 65c | B | m-MePh | — | — | ✓ | >90 | ES+ | (M + H⁺) 409.2015 | 409.2015 |

✓: ¹H NMR spectrum consistent with anticipated structure, LCMS purities for 65a and 65b are reported with detection at both λ₂₁₄ and λ₂₈₀; all other LCMS data reported with detection at λ₂₁₄.

Step 3. Evans Aldol Coupling of Building Block #2

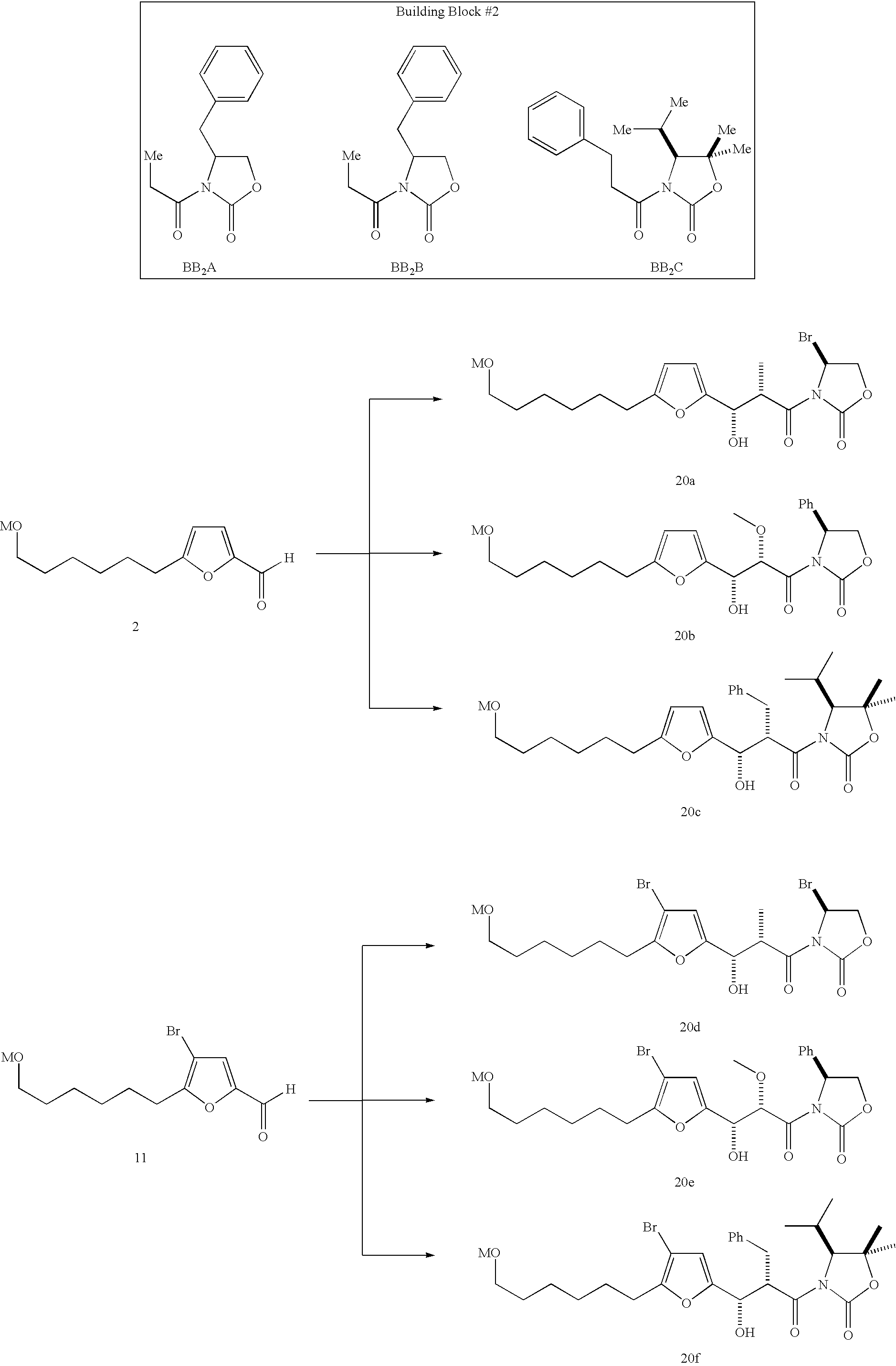

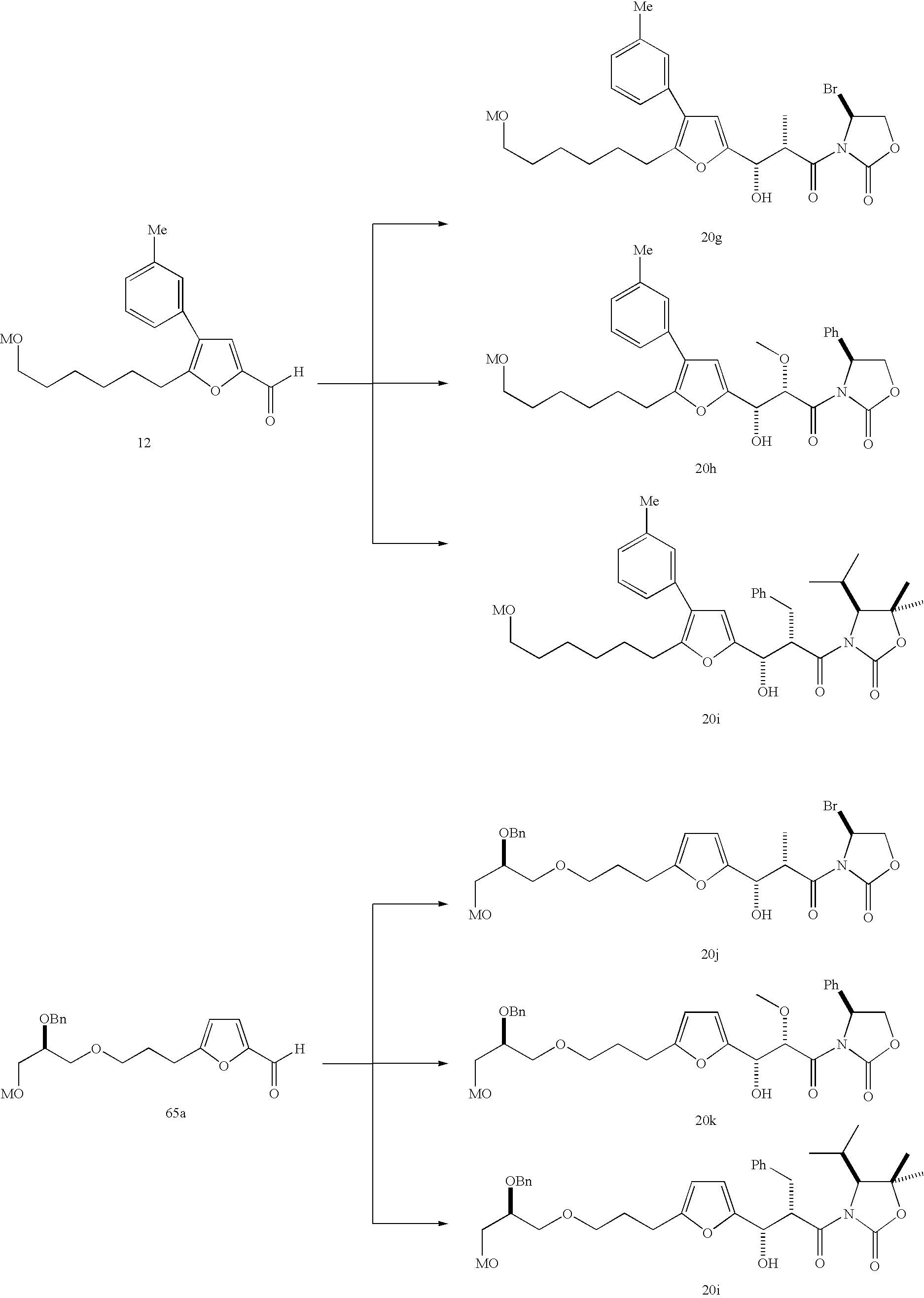

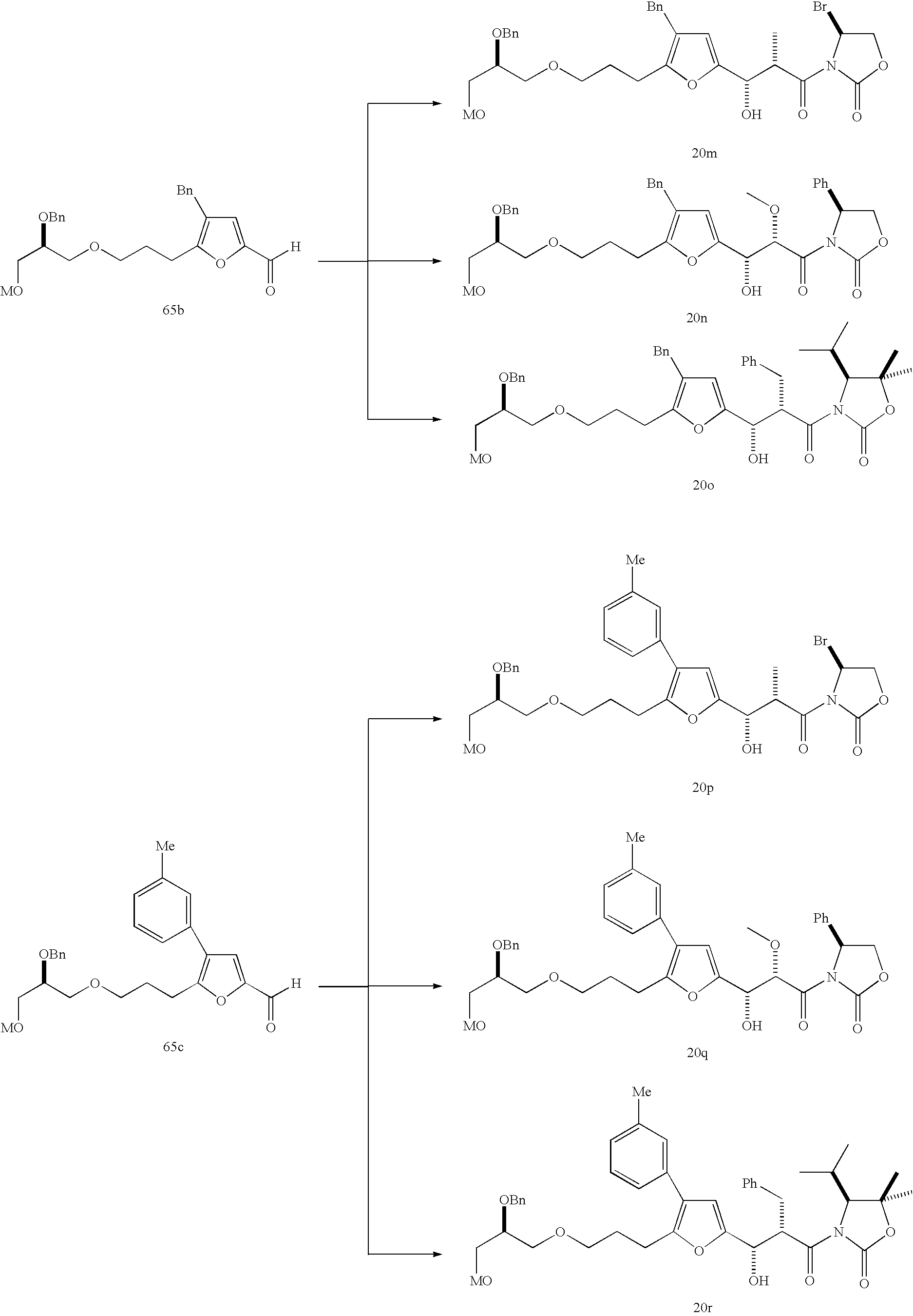

Aldol coupling of Building Block #2 (BB₂). The six pools of light yellow resin from Step 2 (2, 11, 12, and 65a-65c) were then each split into 3 equal portions (18 pools of ˜60 mg each). Each of these 18 portions was then subjected to an aldol coupling reaction with one of the three acyl oxazolidinones BB₂A, BB₂B, or BB₂C. Specifically, in 18 parallel reactions, 2, 11, 12, and 65a-65c were transformed to 20a-r using the same protocols described previously for the transformation of 3→6, 11→13, and 12→15. For the transformation of 65a-65c→201, 20n, and 20r, reactions were maintained at −78° C. for 72 h, −26° C. for 28 h, and 0° C. for 2 h to promote full conversion.

Step 4. Acetylation of Aldol Adducts (σ₂)

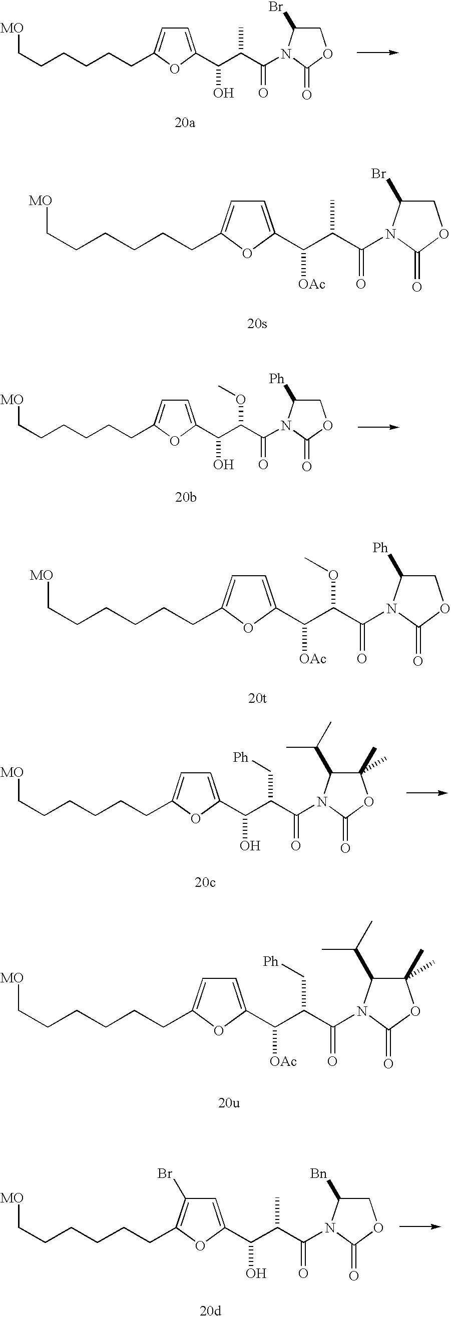

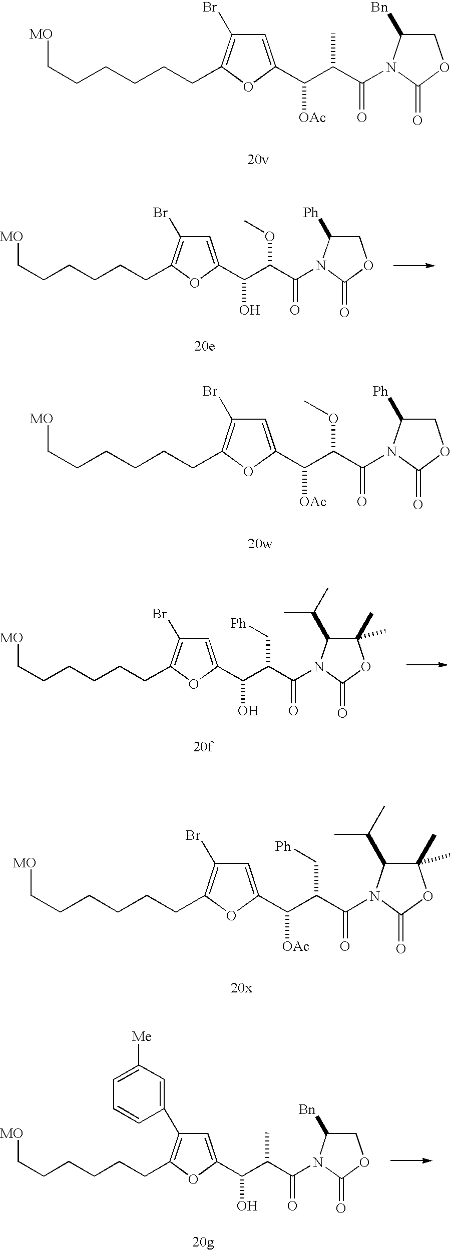

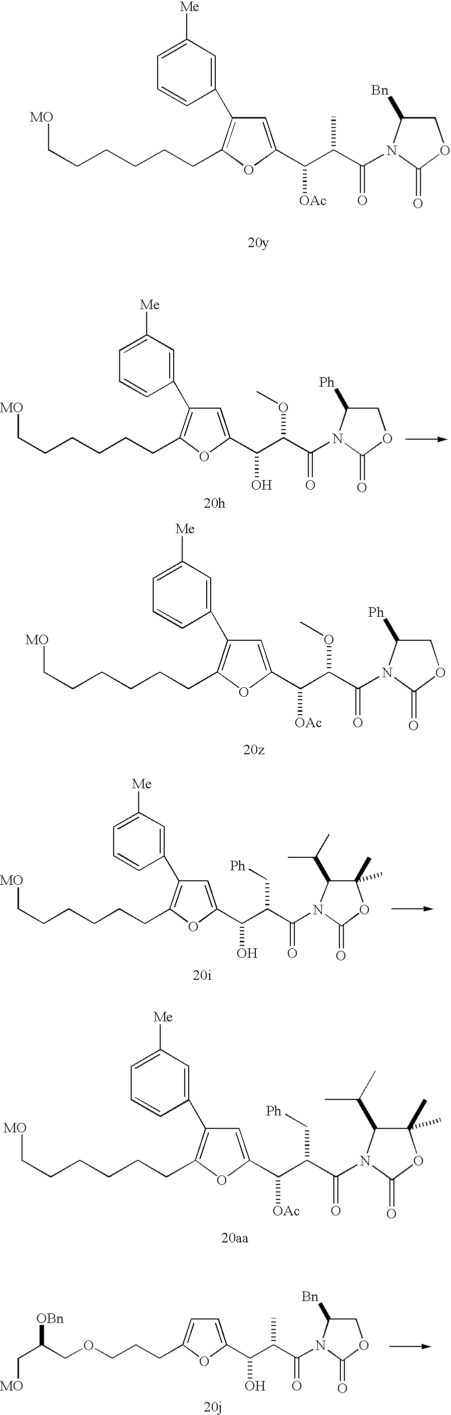

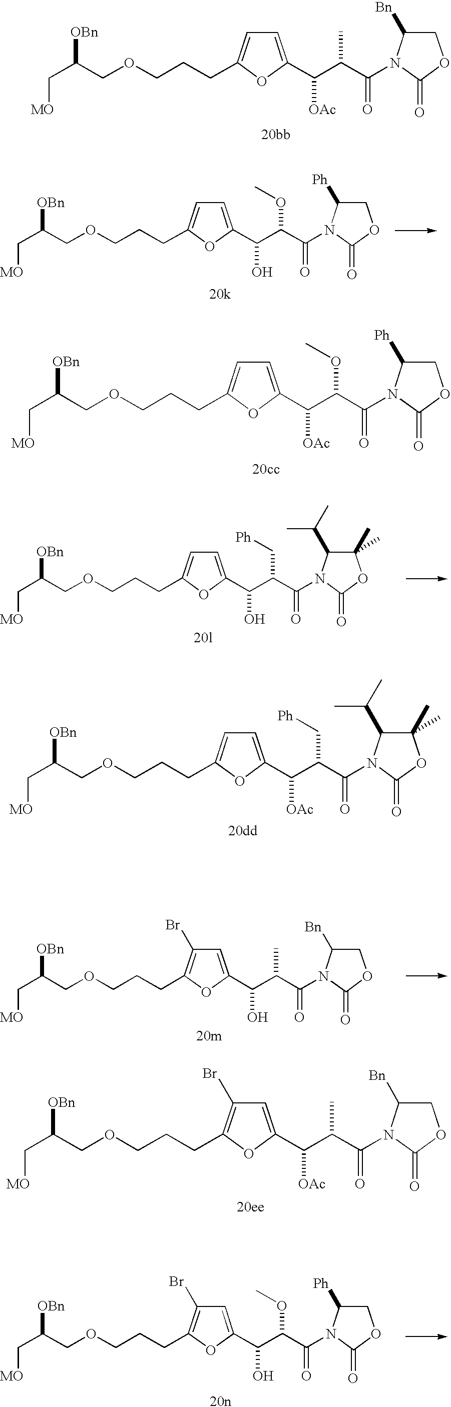

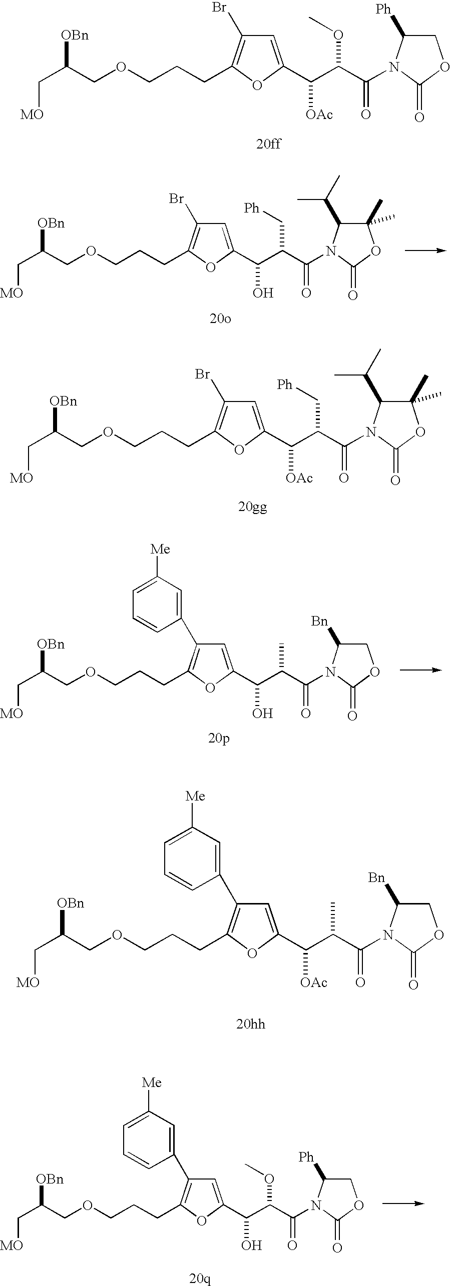

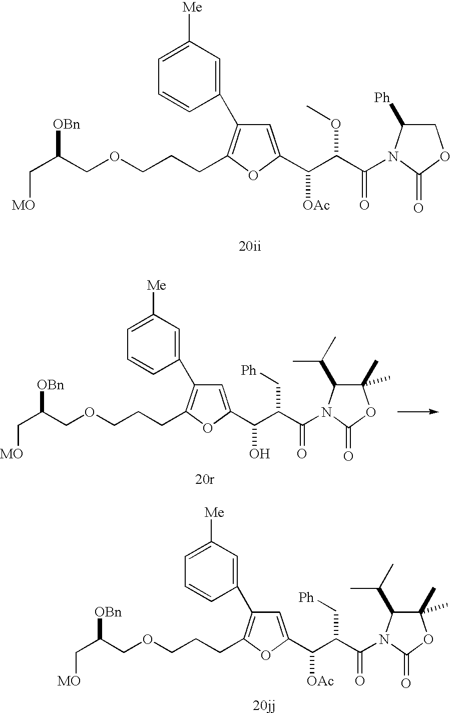

Step 4. 18 portions of light yellow resin from Step 3 (20a-r, ˜60 mg each) were then each divided into two equal portions; one of these portions was subjected to an acetylation reaction using the same protocols described previously for the transformation of 6→7, 13→14, and 15 →16, and the other portion was not acetylated yielding 20a-jj.

TABLE 3
Results of Steps 3 & 4

| | | | | | | % Purity | HRMS | | |
|---|---|---|---|---|---|---|---|---|---|
| No. | BB₁ | σ₁ | BB₂ | σ₂ | ¹H NMR | LCMS, 214 nm | Ionization | Calculated | Observed |
| 20a | A | H | A | H | ✓ | 86 | ES+ | (M + NH₄⁺) 447.2495 | 447.2497 |
| 20b | A | H | B | H | ✓ | 89 | ES+ | (M + Na⁺) 454.1842 | 454.1857 |
| 20c | A | H | C | H | ✓ | >90 | ES+ | (M + Na⁺) 508.2675 | 508.2670 |
| 20d | A | Br | A | H | ✓ | >90 | ES+ | (M + Na⁺) 530.1154 | 530.1169 |
| 20c | A | Br | B | H | ✓ | >90 | ES+ | (M + Na⁺) 532.0947 | 532.0940 |
| 20f | A | Br | C | H | ✓ | 90 | ES+ | (M + NH₄⁺) 581.2226 | 581.2242 |
| 20g | A | m-MePh | A | H | ✓ | >90 | ES+ | (M + NH₄⁺) 537.2964 | 537.2972 |
| 20h | A | m-MePh | B | H | ✓ | 89 | ES+ | (M + NH₄⁺) 539.2757 | 539.2750 |
| 20i | A | m-MePh | C | H | ✓ | 72 | ES+ | (M + NH₄⁺) 594.3591 | 593.3590 |
| 20j | B | H | A | H | ✓ | 81 | ES+ | (M + Na⁺) 573.2417 | 574.2401 |
| 20k | B | H | B | H | ✓ | 81 | ES+ | (M + Na⁺) 576.2210 | 576.2219 |
| 20l | B | H | C | H | ✓ | 81 | ES+ | (M + Na⁺) 630.3043 | 630.3035 |
| 20m | B | Br | A | H | ✓ | 71 | ES+ | (M + Na⁺) 652.1522 | 652.1506 |
| 20n | B | Br | B | H | ✓ | 71 | ES+ | (M + Na⁺) 654.1315 | 654.1289 |
| 20o | B | Br | C | H | ✓ | 79 | ES+ | (M + Na⁺) 708.2148 | 708.2156 |
| 20p | B | m-MePh | A | H | ✓ | 76 | ES+ | (M + Na⁺) 664.2886 | 664.2890 |
| 20q | B | m-MePh | B | H | ✓ | 82 | ES+ | (M + Na⁺) 666.2757 | 666.2742 |
| 20r | B | m-MePh | C | H | ✓ | >90 | ES+ | (M + Na⁺) 720.3512 | 720.3529 |
| 20s | A | H | A | Ac | ✓ | >90 | ES+ | (M + Na⁺) 494.2155 | 494.2169 |
| 20t | A | H | B | Ac | ✓ | 86 | ES+ | (M + Na⁺) 496.1947 | 496.1951 |
| 20u | A | H | C | Ac | ✓ | >90 | ES+ | (M + Na⁺) 550.2781 | 550.2798 |
| 20v | A | Br | A | Ac | ✓ | >90 | ES+ | (M + Na⁺) 572.1260 | 572.1277 |
| 20w | A | Br | B | Ac | ✓ | >90 | ES+ | (M + Na⁺) 574.1052 | 574.1057 |
| 20x | A | Br | C | Ac | ✓ | >90 | ES+ | (M + Na⁺) 628.1886 | 628.1874 |
| 20y | A | m-MePh | A | Ac | ✓ | >90 | ES+ | (M + Na⁺) 584.2624 | 584.2609 |
| 20z | A | m-MePh | B | Ac | ✓ | >90 | ES+ | (M + Na⁺) 586.2417 | 586.2419 |
| 20aa | A | m-MePh | C | Ac | ✓ | 75 | ES+ | (M + Na⁺) 640.3250 | 640.3244 |
| 20bb | B | H | A | Ac | ✓ | 77 | ES+ | (M + Na⁺) 616.2523 | 616.2524 |
| 20cc | B | H | B | Ac | ✓ | 81 | ES+ | (M + Na⁺) 618.2315 | 618.2334 |
| 20dd | B | H | C | Ac | ✓ | 76 | ES+ | (M + Na⁺) 672.3149 | 672.3134 |
| 20ee | B | Br | A | Ac | ✓ | 71 | ES+ | (M + Na⁺) 694.1628 | 694.1645 |
| 20ff | B | Br | B | Ac | ✓ | 67 | ES+ | (M + Na⁺) 696.1420 | 696.1391 |
| 20gg | B | Br | C | Ac | ✓ | 73 | ES+ | (M + Na⁺) 750.2254 | 750.2261 |
| 20hh | B | m-MePh | A | Ac | ✓ | 87 | ES+ | (M + Na⁺) 706.2992 | 706.3015 |
| 20ii | B | m-MePh | B | Ac | ✓ | >90 | ES+ | (M + Na⁺) 708.2785 | 708.2781 |
| 20jj | B | m-MePh | C | Ac | ✓ | >90 | ES+ | (M + Na⁺) 762.3618 | 763.3609 |

✓: ¹H NMR spectrum consistent with anticipated structure

Step 5. NBS and PPTS-mediated transformation of 20a-jj into a complete, combinatorial matrix of molecular skeletons, each derivatized with a complete, combinatorial matrix of building blocks.

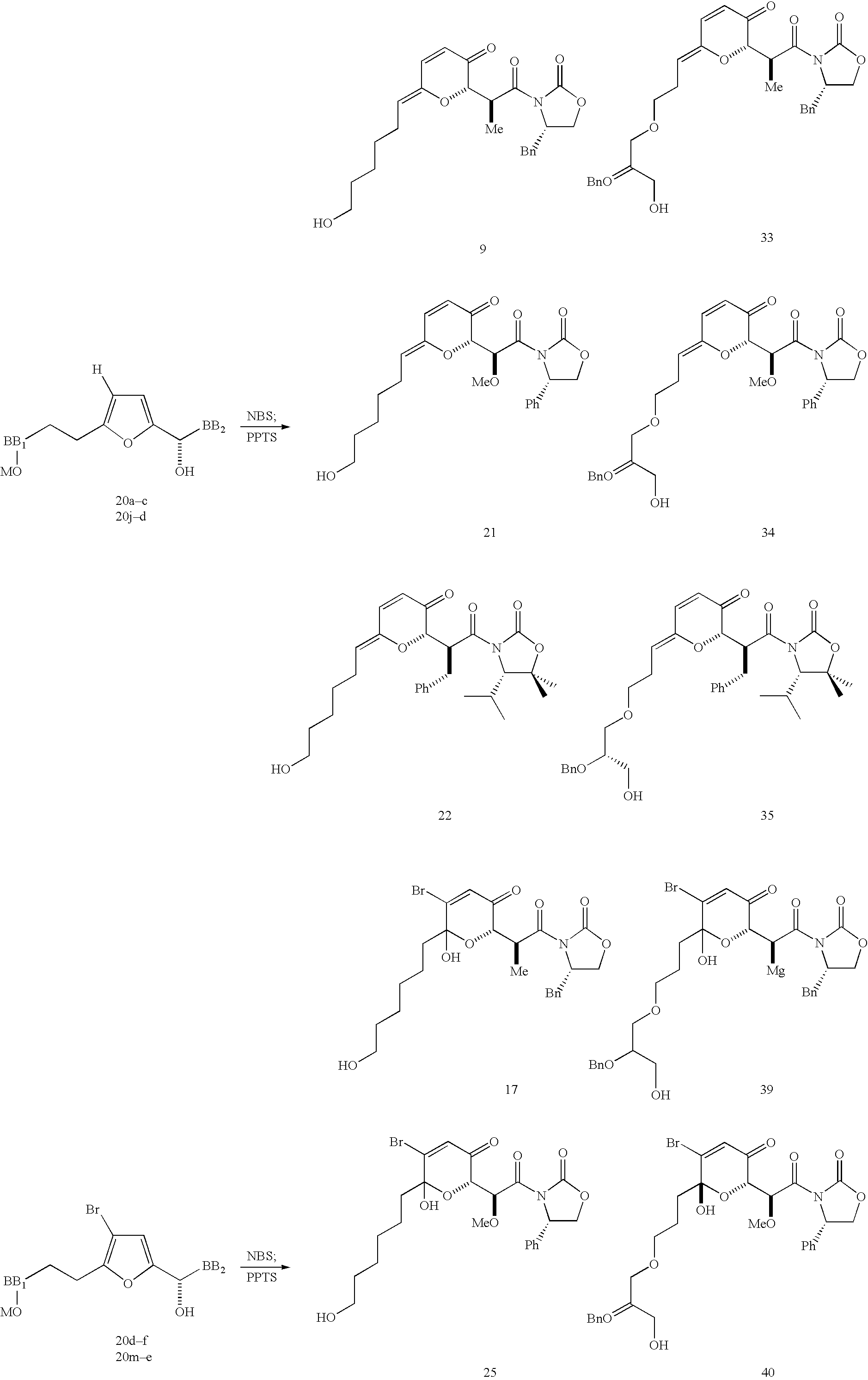

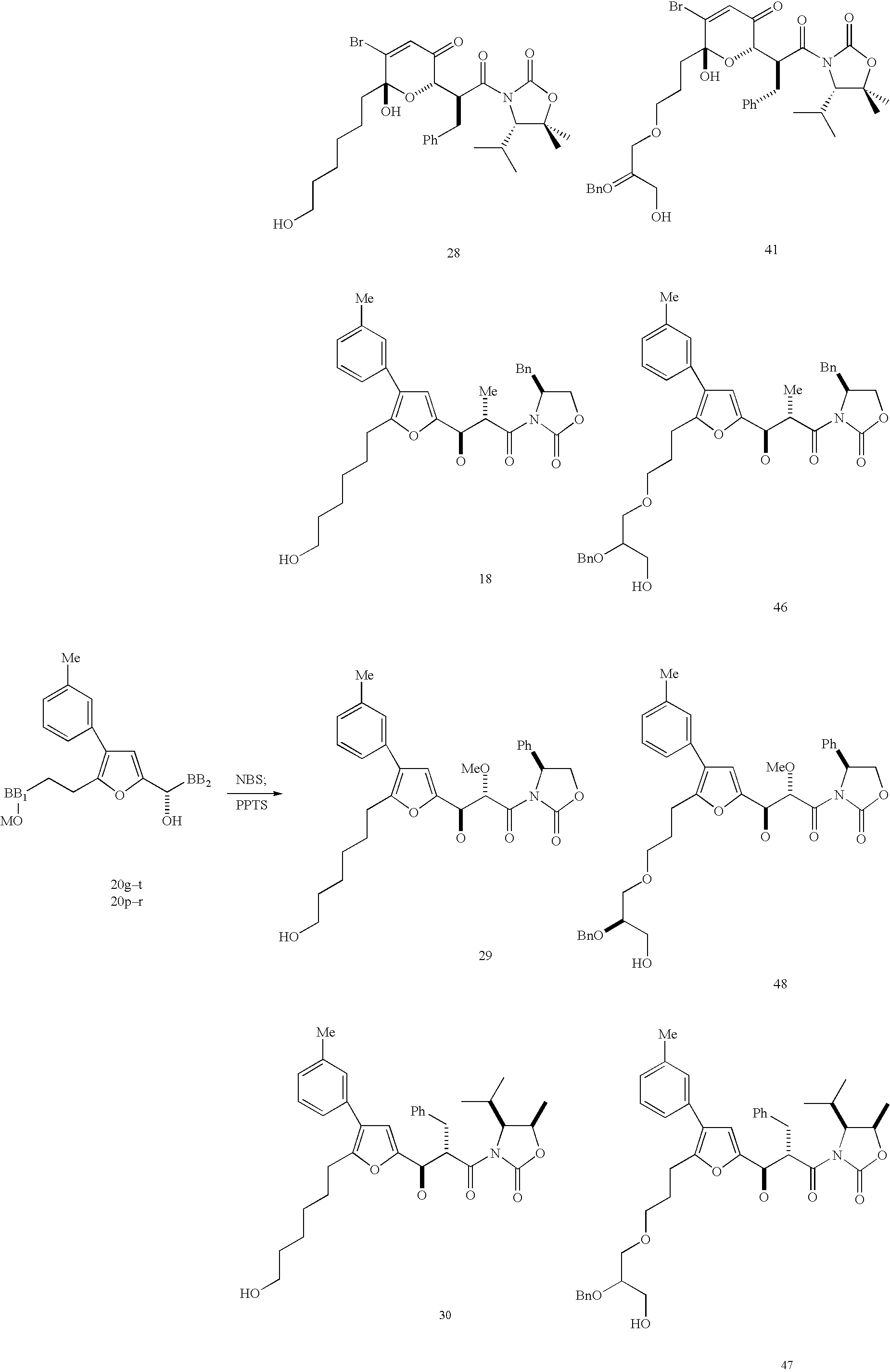

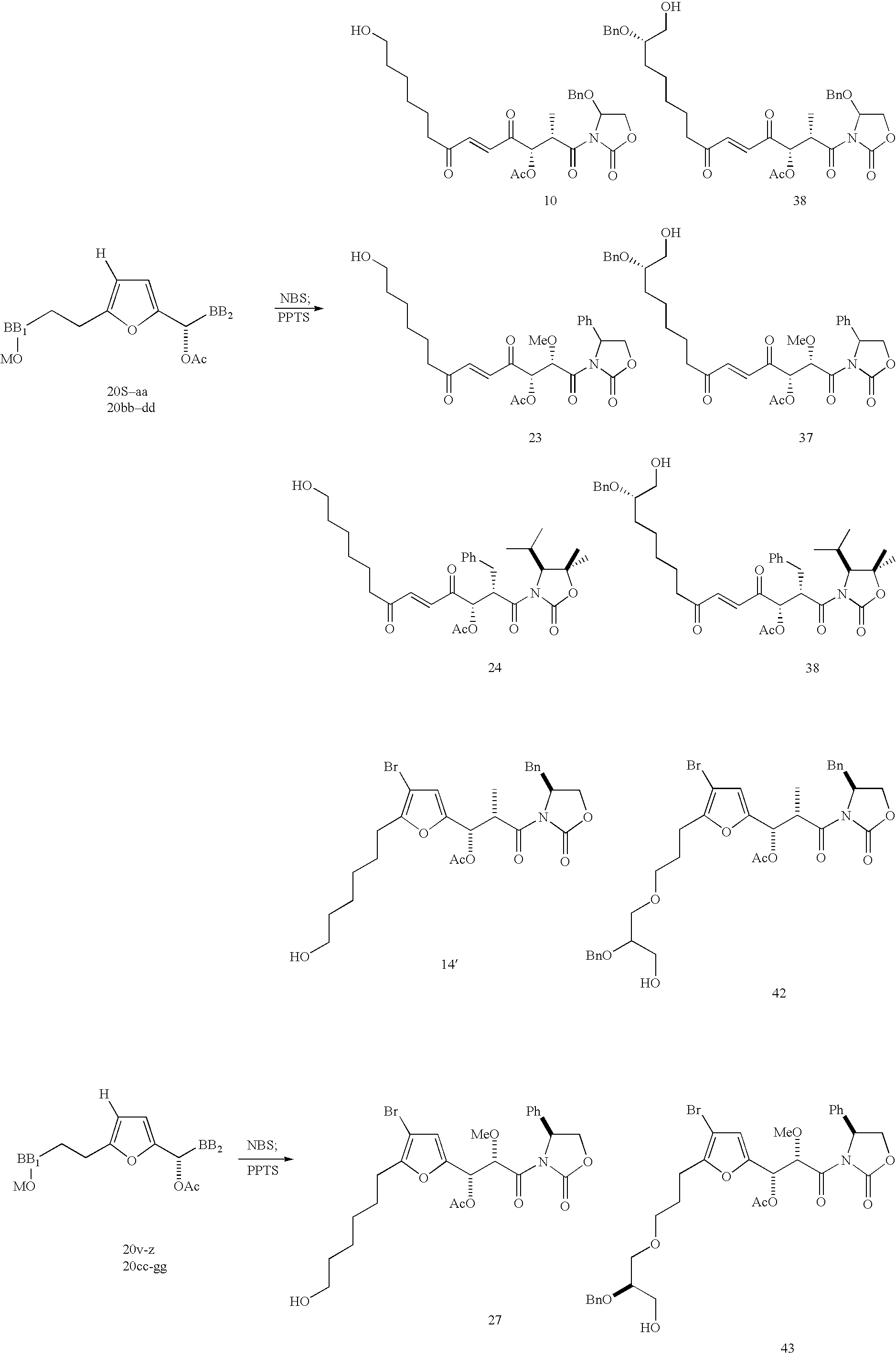

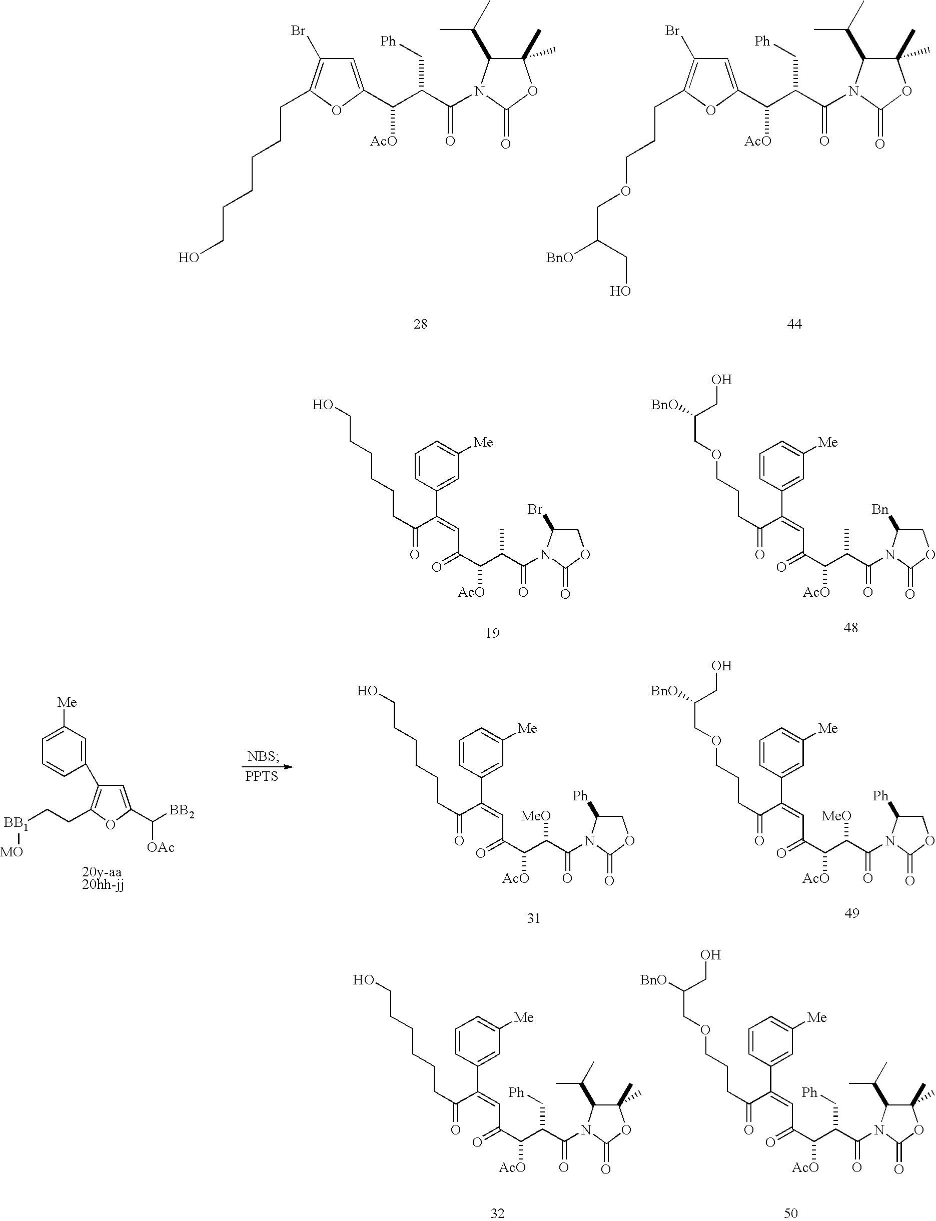

NBS and PPTS-mediated transformations. In 36 parallel reactions, each substrate 20a-jj was subjected to the samne reaction conditions (NBS/THF at rt for 1 h; PPTS/CH₂Cl₂ at 40-45° C. for 20 h) using the protocol described previously for the transforrnation of 5→8.

TABLE 4
Results of Step 5

| | | | | | | % Purity | HRMS | | |
|---|---|---|---|---|---|---|---|---|---|
| No. | BB₁ | σ₁ | BB₂ | σ₂ | ¹H NMR | LCMS, 214 nm | Ionization | Calculated | Observed |
| 9 | A | H | A | H | ✓ | 83 | ES+ | (M + H⁺) 428.2703 | 428.2061 |
| 21 | A | H | B | H | ✓ | >70 | ES+ | (M + Na⁺) 452.1685 | 452.1700 |
| 22 | A | H | C | H | ✓ | >80 | ES+ | (M + H⁺) 484.2699 | 484.2699 |
| 10 | A | H | A | Ac | ✓ | >90 | ES+ | (M + H⁺) 488.2284 | 488.2275 |
| 23 | A | H | B | Ac | ✓ | 85 | ES+ | (M + NH₄⁺) 507.2343 | 507.2358 |
| 24 | A | H | C | Ac | ✓ | 78 | ES+ | (M + NH₄⁺) 561.3176 | 561.3162 |
| 17 | A | Br | A | H | ✓ | >90 (10:1 e.r.) | ES+ | (M + Na⁺) 546.1103 | 546.1086 |
| 25 | A | Br | B | H | ✓ | >90 (1:1 e.r.) | ES+ | (M + Na⁺) 548.0896 | 548.0895 |
| 26 | A | Br | C | H | ✓ | 89 (8:1 e.r) | ES+ | (M + Na⁺) 602.1729 | 602.1733 |
| 14′ | A | Br | A | Ac | ✓ | >90 | ES+ | (M + Na⁺) 572.1260 | 572.1288 |
| 27 | A | Br | B | Ac | ✓ | >90 | ES+ | (M + Na⁺) 574.1052 | 574.1038 |
| 28 | A | Br | C | Ac | ✓ | >90 | ES+ | (M + NH₄⁺) 623.2332 | 623.2353 |
| 18 | A | m-MePh | A | H | ✓ | 80 | ES+ | (M + H⁺) 518.2542 | 518.2532 |
| 29 | A | m-MePh | B | H | ✓ | 52 | ES+ | (M + Na⁺) 542.2155 | 542.2152 |
| 30 | A | m-MePh | C | H | ✓ | 71 | ES+ | (M + H⁺) 574.3168 | 574.3163 |
| 19 | A | m-MePh | A | Ac | ✓ | 76 | ES+ | (M + NH₄⁺) 595.3019 | 595.3034 |
| 31 | A | m-MePh | B | Ac | ✓ | 44 | ES+ | (M + NH₄⁺) 597.2812 | 597.2803 |
| 32 | A | m-MePh | C | Ac | ✓ | 56 | ES+ | (M + Na⁺) 656.3199 | 656.3177 |
| 33 | B | H | A | H | ✓ | 27 | ES+ | (M + H⁺) 550.2441 | 550.2437 |
| 34 | B | H | B | H | ✓ | 21 | ES+ | (M + H⁺) 552.2233 | 552.2233 |
| 35 | B | H | C | H | ✓ | 28 | ES+ | (M + H⁺) 606.3067 | 606.3066 |
| 36 | B | H | A | Ac | ✓ | 70 | ES+ | (M + NH₄⁺) 627.2918 | 627.2930 |
| 37 | B | H | B | Ac | ✓ | 70 | ES+ | (M + H⁺) 612.2445 | 612.2455 |
| 38 | B | H | C | Ac | ✓ | 59 | ES+ | (M + H⁺) 666.3278 | 666.3286 |
| 39 | B | Br | A | H | ✓ | 74 (8:1 e.r) | ES+ | (M + NH₄⁺) 663.1917 | 663.1911 |
| 40 | B | Br | B | H | ✓ | 72 (3:1 e.r) | ES+ | (M + Na⁺) 670.1264 | 670.1268 |
| 41 | B | Br | C | H | ✓ | 68 (8:1 e.r) | ES+ | (M + NH₄⁺+) 719.2543 | 719.2547 |
| 42 | B | Br | A | Ac | ✓ | 76 | ES+ | (M + NH₄⁺) 689.2074 | 689.2081 |
| 43 | B | Br | B | Ac | ✓ | >90 | ES+ | (M + NH₄⁺) 691.1866 | 691.1869 |
| 44 | B | Br | C | Ac | ✓ | >90 | ES+ | (M + NH₄⁺) 745.2700 | 745.2704 |
| 45 | B | m-MePh | A | H | ✓ | 71 | ES+ | (M + H⁺) 640.2910 | 640.2911 |
| 46 | B | m-MePh | B | H | ✓ | 74 | ES+ | (M + H⁺) 642.2703 | 642.2705 |
| 47 | B | m-MePh | C | H | ✓ | 84 | ES+ | (M + H⁺) 696.3536 | 696.3550 |
| 48 | B | m-MePh | A | Ac | ✓ | 33 | ES+ | (M + NH₄⁺) 717.3387 | 717.3383 |
| 49 | B | m-MePh | B | Ac | ✓ | 27 | ES+ | (M + NH₄⁺) 719.3180 | 719.3179 |
| 50 | B | m-MePh | C | Ac | ✓ | 86 | ES+ | (M + NH₄⁺) 773.4013 | 773.4014 |

✓: ¹H NMR spectrum consistent with anticipated structure, e.r. = epimeric ratio
¹H NMR and LC data (UV trace, λ₂₁₄) for all substrates 20a-jj and products 9-10, 14′, 17-19, and 21-50 are available upon request in the form of Appendix A (73 pages).

V. The potential of this σ-element-based strategy to generate overlapping, combinatorial matrices of molecular skeletons and appended building blocks was realized in the context of a highly efficient, five-step, fully-encoded split-pool synthesis pathway (FIG. 4). Toward this end, we first expanded our collections of candidate building blocks to include the diverse set of seven commercially available, terminal olefin-containing primary alcohols (BB₁A-BB₁G) and 15 acyl oxazolidinone coupling partners shown in FIG. 4A (BB₂AS-BB₂OS—a complete matrix of five commercially available, non-racemic, chiral oxazolidinones and three different acyl side chains). The 15 enantiomeric acyl oxazolidinones (BB₂AR-BB₂OR) were also prepared, allowing us to take advantage of reagent-based stereocontrol to generate both sets of possible enantiomeric or diastereomeric (when BB₁ is chiral) aldol adducts.

Screening for BB#1 (BB₁)

The 13 commercially available compounds shown in Scheme 7, each containing both a hydroxyl group and a terminal olefin, were screened for both effective loading onto macrobeads and subsequent B-alkyl Suzuki coupling with one or more of the following: 5-bromofuraldehyde, 4,5-dibromofuraldehyde, and 4-m-MePh-5-bromofuraldehyde. All reactions were run on ˜25 mg of macrobeads.

Scheme 7. Collection of potential building blocks included in screen for BB#1

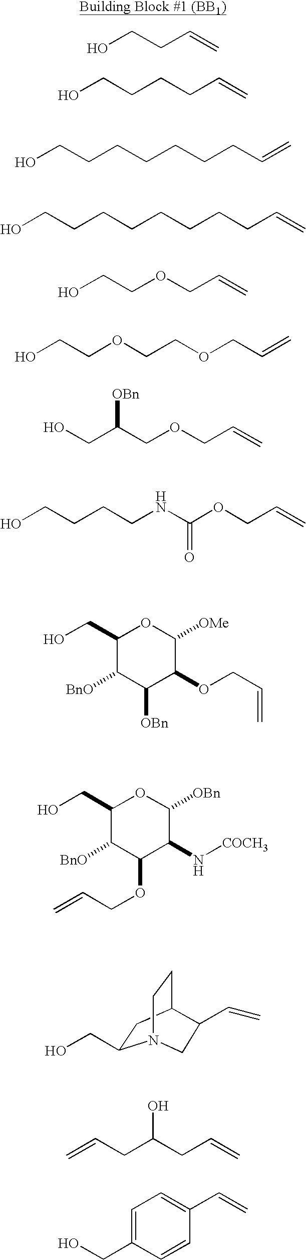

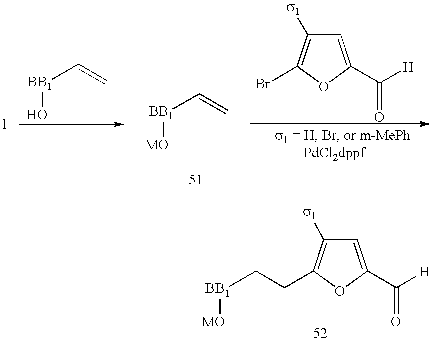

51 3-[Diisopropyl(p-methoxyphenyl)silyl]propyl functionalized beads 1 (25 mg, estimated loading ˜1.3 meq Si/g, ˜0.0325 meq.) in a 2 mL polypropylene tube at rt under Ar were allowed to swell in CH₂Cl₂ (˜10 ml) for 10 min. The colorless beads were then filtered and again washed with CH₂Cl₂ (˜10 mL×10 min.), and then resuspended in a 2.5% (v/v) solution of TMSC₁ in CH₂Cl₂ (˜10 mL) for 30 min. The beads were again filtered and washed thrice with CH₂Cl₂ (5 min each) and then suspended in a 3% (v/v) solution of trifluoromethanesulfonic acid in CH₂Cl₂ (0.575 mL, 0.195 mmol) for 20 min during which the reaction tube was shaken periodically and the beads turned orange. After filtration, the orange-colored beads were again thrice washed with CH₂Cl₂ and then resuspended in a minimum volume of CH₂Cl₂ (˜0.2 mL). Freshly distilled 2,6-lutidine was then added (30.3 uL, 0.26 mmol) resulting in bead discoloration followed by building block #1 (0.26 mmol). The resulting colorless reaction mixture was then shaken manually and let stand at rt for 12 h. The beads were then filtered, washed with CH₂Cl₂ (5×5 mL×5 min. each), and the solvent was removed under Ar flow followed by residual solvent removal in vacuo to yield resin 51 loaded with candidates for building block #1.

52 Macrobeads loaded with candidates for building block #1 51 (˜0.0325 meq.) were washed with THF (2×3 mL×10 min each) at rt and then resuspended in THF (0.750 ML). A 0.5M solution of 9-BBN in THF (0.5 mL, 0.25 mmol) was then added and the resulting mixture was let stand at rt for 5 h (with periodic manual agitation every hour). The reaction solution was then removed via cannula and the colorless resin was washed thoroughly with THF (5×5 mL×10 min each). To the resin was then added PdCl₂dppf (1 mg, 0.00125 mmol) via cannula as a suspension in THF (0.125 mL), one of the following three furaldehyde coupling partners: 5-bromofuraldehyde (21.9 mg, 0.125 mmol), 4,5-dibromofuraldehyde (31.7 mg, 0.125 mmol), or 4-m-MePh-5-bromofuraldehyde (33.1 mg, 0.125 mmol) via cannula as a solution in THF (0.188 mL), a 2M solution of NaOH (31 μL, 0.0625 mmol). The resulting orange reaction mixture was sealed under a cloud of Ar and heated at 60-65° C. with periodic manual agitation for 24-28 h (reaction mixture turned dark brown). The yellow/orange resin was then isolated by filtration and washed as follows, 4×(5×THF, 5×H₂O, 5×THF, THF/H₂O: 3/1×30 min), 5×THF, THF×30 min, 5×CH₂Cl₂, CH₂Cl₂×30 min, and the residual solvent was removed in vacuo to yield product resin 52. 5 mg of this resin was then treated with HF/Pyridine cleavage conditions (see General Methods) and the crude product residue was analyzed by ¹H NMR, LCMS, and HRMS.

This building block screen led to the identification of seven building blocks shown in Scheme 8 (abbreviated alphabetically BB₁A, BB₁B, BB₁C, etc.). The results for these building blocks are shown in Table 5.

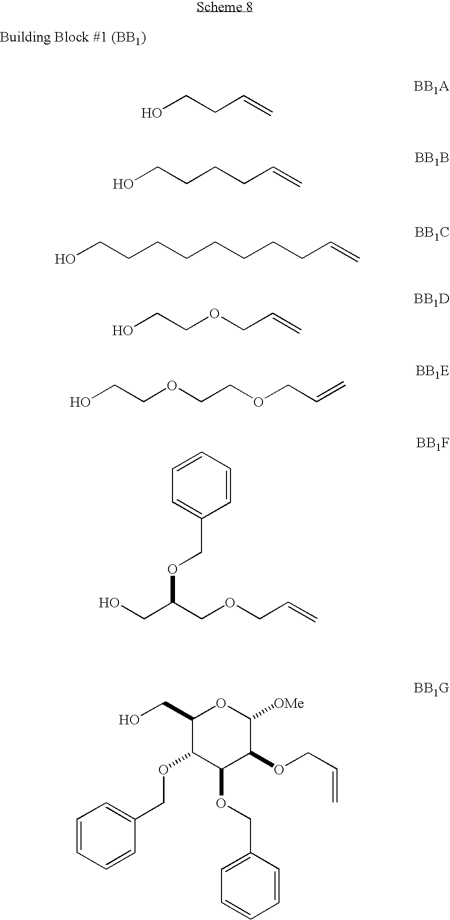

TABLE 5
Results for building block #1

| | | | % Purity | % Purity | HRMS | | |
|---|---|---|---|---|---|---|---|
| BB₁ | σ₁ | ¹H NMR | LCMS, λ₂₈₀ | LCMS, λ₂₁₄ | Ionization | Calculated | Observed |
| BB₁A | H | ✓ | >95 | — | EI+ | (m/z) 168.0786 | 168.0785 |
| BB₁B | H | ✓ | >85 | — | ES+ | (M + H⁺) 197.1177 | 197.1177 |
| BB₁C | H | ✓ | >95 | — | EI+ | (m/z) 252.1725 | 252.1723 |
| BB₁D | H | ✓ | >95 | — | EI+ | (m/z) 198.0892 | 198.0893 |
| BB₁E | H | ✓ | >95 | — | EI+ | (m/z) 242.1154 | 242.1152 |
| BB₁F | H | ✓ | 93 | — | EI+ | (m/z) 318.1467 | 318.1464 |
| BB₁G | H | ✓ | >95 | — | ES+ | (M + Na⁺) 533.2151 | 533.2145 |
| BB₁A | Br | ✓ | >95 | — | ES+ | (M + H⁺) 246.9970 | 246.9969 |
| BB₁B | Br | ✓ | — | >95 | ES+ | (M + H⁺) 397.0650 | 397.0645 |
| BB₁C | Br | ✓ | >90 | — | ES+ | (M + H⁺) 331.0909 | 331.0906 |
| BB₁D | Br | ✓ | >95 | — | ES+ | (M + H⁺) 277.0075 | 277.0066 |
| BB₁E | Br | ✓ | >95 | — | ES+ | (M + H⁺) 321.0337 | 321.0326 |
| BB₁F | Br | ✓ | >95 | — | ES+ | (M + H⁺) 397.0650 | 387.0643 |
| BB₁G | Br | ✓ | 84 | — | ES+ | (M + Na⁺) 611.1256 | 611.1246 |
| BB₁A | m-MeAr | ✓ | — | 90 | ES+ | (M + H⁺) 259.1134 | 259.1340 |
| BB₁B | m-MeAr | ✓ | — | >95 | ES+ | (M + H⁺) 287.1647 | 287.1647 |
| BB₁C | m-MeAr | ✓ | — | >95 | ES+ | (M + H⁺) 343.2273 | 343.2272 |
| BB₁D | m-MeAr | ✓ | — | 92 | ES+ | (M + H⁺) 289.1440 | 289.1430 |
| BB₁E | m-MeAr | ✓ | — | 89 | ES+ | (M + H⁺) 333.1702 | 333.1709 |
| BB₁F | m-MeAr | ✓ | — | 83 | ES+ | (M + H⁺) 409.2015 | 409.2009 |
| BB₁G | m-MeAr | ✓ | — | 80 | ES+ | (M + Na⁺) 623.2621 | 623.2615 |

✓ = ¹H NMR spectrum consistent with anticipated structure

Screening for Building Block #2 (BB₂)

We then screened a variety of commercially available, nonracemic chiral oxazolidinones combined with diverse acyl side chains for efficient coupling with macrobead-bound 5-(6-hydroxyhexyl)-furaldehyde. We first synthesized a diverse set of eight chiral oxazolidinones coupled to various acyl side chains (shown in Scheme 9), and tested them for efficient aldol coupling. Tolerance for diverse oxazolidinones was noted and the three most effective acyl side chains from those tested were identified and used in a second round of screening, in which a 5×3 matrix of commercially available oxazolidinones and acyl side chains were synthesized (shown in Scheme 10) and tested. These 15 building blocks (BB₂AS-BB₂OS) were found to be effective coupling partners in the Evans aldol reaction. The 15 enantiomeric acyl oxazolidinones (BB₂AR-BB₂OR) were also prepared, allowing us to take advantage of reagent-based stereocontrol to generate both sets of possible enantiomeric or diastereomeric (when BB₁ is chiral) aldol adducts.

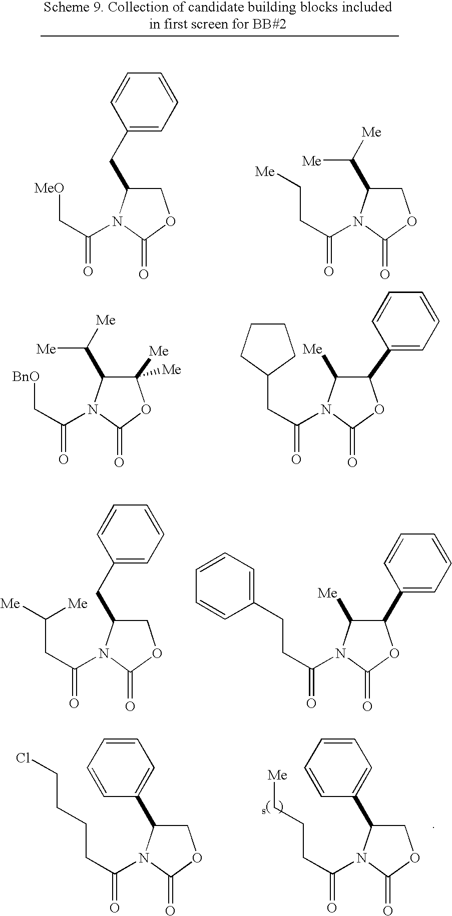

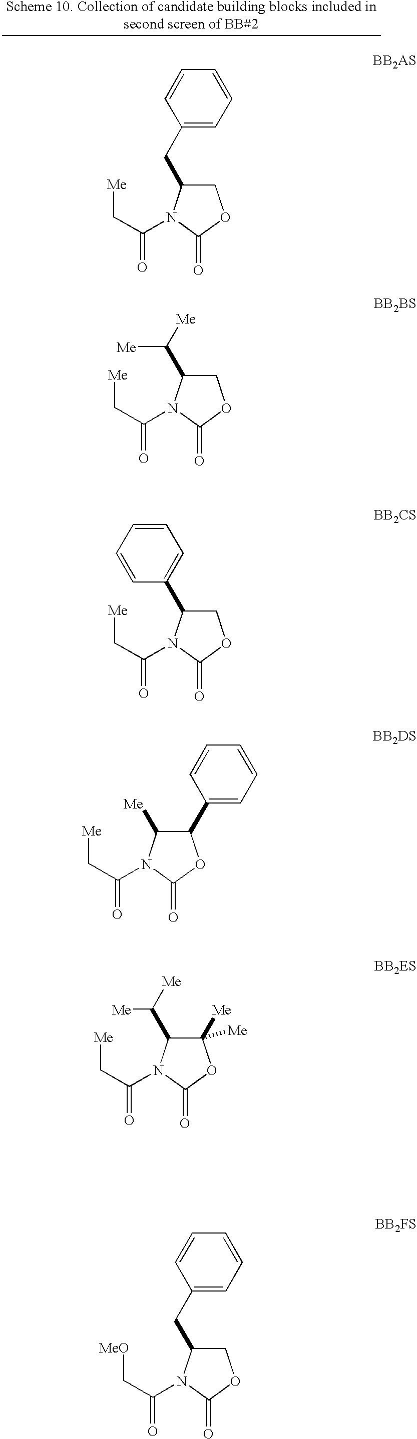

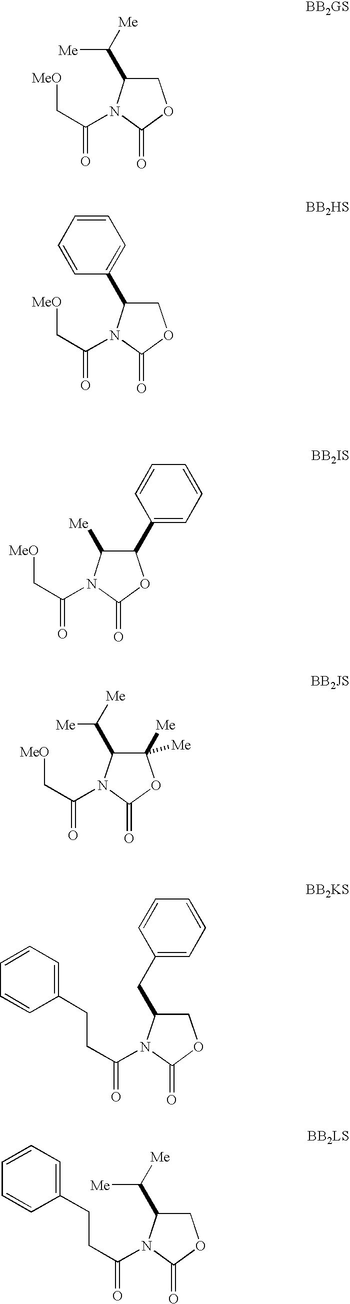

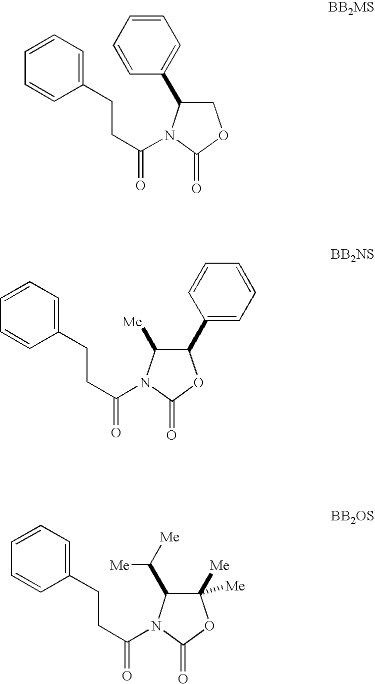

Building block #2 compounds are classified as R or S by the orientation of the 4′-substituent on the oxazolidinone ring.

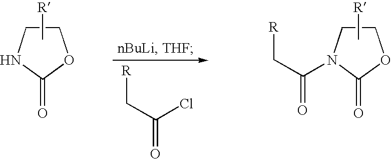

Synthesis of acyl oxazolidinones. A stirred solution of oxazolidinone (1 g.) in anhydrous THF (0.2 M in oxazolidinone) was cooled to −78° C. for 15 minutes. nBu-Li (1.1 equiv.) was slowly added and the mixture was stirred for 15 minutes. The appropriate acid chloride (1.1 equiv.) was then added by syringe and the mixture stirred for another 30 minutes. The mixture was then warmed to rt over 45 minutes, quenched with NH₄C₁ (4 mL), and the THF was removed with rotary evaporation. The resulting slurry was then extracted with CH₂C₁ (2×5 mL), and the combined organic fractions were washed with 2 M NaOH (aq.) (5 mL) and brine (5 mL), dried over sodium sulfate, and concentrated in vacuo. The product was then purified via flash chromatography (SiO₂, hexanes/ethyl acetate), azeotropically dried with benzene, and stored under Argon for further use. The average chemical yield of the syntheses was roughly 85%.

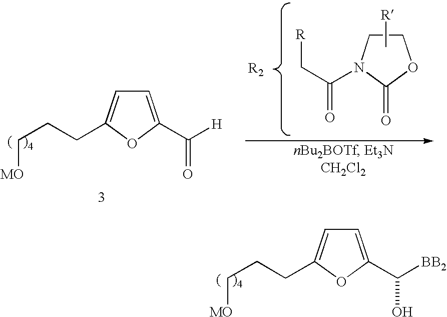

Screening of acyl oxazolidinones Yellow-orange macrobeads 3 (25 mg) were washed with CH₂Cl₂ (3×1 mL×10 min each) at rt, and then cooled to −78° C. In a separate vessel, to a stirred solution of acyl oxazolidinone (0.125 mmol) in CH₂Cl₂ (0.5 mL) at 0° C. was added a 1M solution of dibutylboron triflate in CH₂Cl₂ (131 μL, 0.13 1 mmol) followed by triethylamine (21 μL, 0.150 mmol). The resulting enolate solution was cooled to −78° C. and then transferred rapidly via cannula to the vessel containing 3. The resulting mixture was sealed under a cloud of Ar and maintained at −78° C. for 48 h, −26° C. for 24 h, and 0° C. for 1 h (with periodic manual agitation about once every 8 h). The reaction was then quenched with the addition of pH7 phosphate buffer (500 μL), MeOH (500 μL), and 30% aq. H₂O₂ (333 μL), and the resulting mixture was tumbled at 4° C. for 12-15 h. Resin was then isolated by filtration and washed as follows: 5×CH₂Cl₂, 5×DMF, 5×THF, 5×CH₂Cl₂, CH₂Cl₂×1 h, 5×DMF, DMF×1 h, 5×THF, THF×1 h, 5×CH₂Cl₂, CH₂Cl₂×30 min, 5×anhydrous CH₂Cl₂, anhydrous CH₂Cl₂×30 min, and residual solvent was removed in vacuo to yield the product resin. 5 mg of this product resin was then treated with HF/Pyridine cleavage conditions (see General Methods), and the crude product residue was analyzed by ¹H NMR, LCMS, and HRMS. The results are summarized in (Table 6).

TABLE 6
Table of BB2 results

| | % Conversion | d.r. | % Purity | HRMS | | |
|---|---|---|---|---|---|---|
| BB2 | ¹H NMR | ¹H NMR | LCMS, λ₂₁₄ | Ionization | Calculated | Found |
| BB₂AS | >95 | 20:1 | 86 | ES+ | (M + NH₄⁺) 447.2495 | 447.2497 |
| BB₂BS | >95 | 20:1 | 92 | ES+ | (M + Na⁺) 404.2049 | 404.2049 |
| BB₂CS | >95 | 20:1 | 91 | ES+ | (M + Na⁺) 438.1893 | 438.1899 |
| BB₂DS | >95 | 20:1 | >95 | ES+ | (M + Na⁺) 452.2049 | 452.2058 |
| BB₂ES | >90 | 8:1 | >95 | ES+ | (M + NH₄⁺) 427.2808 | 427.2805 |
| BB₂FS | >95 | 10:1 | 92 | ES+ | (M + Na⁺) 468.1998 | 468.2001 |
| BB₂GS | >95 | 12:1 | 86 | ES+ | (M + Na⁺) 420.1998 | 420.2002 |
| BB₂HS | >95 | 11:1 | 83 | ES+ | (M + Na⁺) 454.1842 | 454.1857 |
| BB₂IS | >95 | 16:1 | 77 | ES+ | (M + Na⁺) 468.1998 | 468.1985 |
| BB₂JS | >95 | 20:1 | 97 | ES+ | (M + Na⁺) 448.2311 | 448.2314 |
| BB₂KS | >95 | 20:1 | 97 | ES+ | (M + Na⁺) 528.2362 | 528.2366 |
| BB₂LS | >95 | 10:1 | 72 | ES+ | (M + Na⁺) 480.2362 | 480.2371 |
| BB₂MS | >85 | 7:1 | 90 | ES+ | (M + Na⁺) 514.2206 | 514.2219 |
| BB₂NS | >90 | 9:1 | 93 | ES+ | (M + NH₄⁺) 523.2808 | 523.2816 |
| BB₂O | >85 | 9:1 | 96 | ES+ | (M + Na⁺) 508.2675 | 508.2670 |

¹H NMR and LC data for all building blocks selected for use in the split-pool synthesis are available upon request in the form of Appendix B (33 pages).

[image]

Step 1. Coupling of Building Block #1 (BB₁)

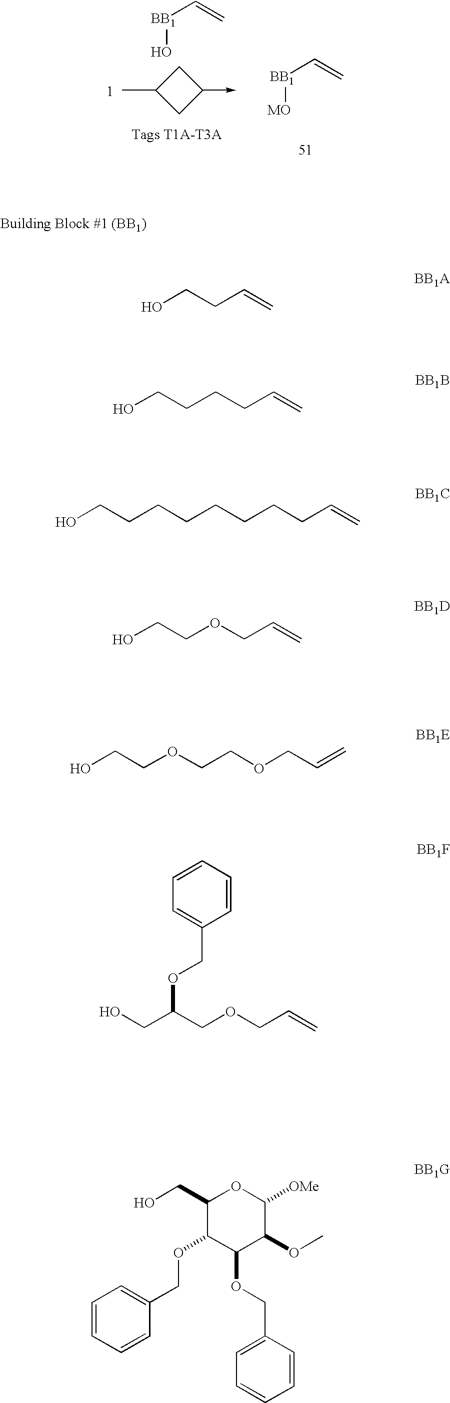

Coupling of Building Block #1 (BB₁). A single pool of 3-[Diisopropyl(p-methoxyphenyl)silyl]-propyl functionalized beads 1 (2 g) was split evenly into seven portions (286 mg each), and each was subjected to a loading reaction with a unique BB#l as described below:

51 3-[Diisopropyl(p-methoxyphenyl)silyl]propyl functionalized beads 1 (286 mg per reaction) in a 10 mL polypropylene tube at rt under Ar were allowed to swell in CH₂Cl₂ (7 ml) for 10 min. The colorless beads were then filtered and again washed with CH₂Cl₂ (7 mL×10 min.), and then resuspended in a 2.5% (v/v) solution of TMSC₁ in CH₂Cl₂ (7 mL) for 30 min. The beads were again filtered and washed thrice with CH₂Cl₂ (5 min each) and then suspended in a 3% (v/v) solution of trifluoromethanesulfonic acid in CH₂Cl₂ (6.6 mL) for 20 min during which time the reaction tube was shaken periodically and the beads turned orange. After filtration, the orange-colored beads were again thrice washed with CH₂Cl₂ and then resuspended in a minimum volume of CH₂Cl₂ (˜1 mL). Freshly distilled 2,6-lutidine was then added (346 uL, addition resulted in bead discoloration) followed by building block #1:

TABLE 7
BB#1 used in split-pool synthesis

| Building | | formula weight | density | volume | |
|---|---|---|---|---|---|
| Block | mol | (g/mol) | (g/mL) | (uL) | mass (g) |
| BB₁A | 0.002974 | 72.11 | 0.85 | 252 | |
| BB₁B | 0.002974 | 100.16 | 0.834 | 357 | |
| BB₁C | 0.002974 | 156.27 | 0.876 | 531 | |
| BB₁D | 0.002974 | 102.13 | 0.955 | 318 | |
| BB₁E | 0.002237 | 146.68 | 1.01 | 325 | |
| BB₁F | 0.002974 | 222.28 | ˜1 | 661 | 0.661 |
| BB₁G | 0.002974 | 414.49 | n/a | | 1.23* |

*dissolved in 300 uL CH₂Cl₂

The resulting colorless reaction mixtures were then shaken manually and let stand at rt for 16 h. The beads were then filtered, washed with CH₂Cl₂ (5×7 mL×20 min. each), and the solvent was removed under Ar flow followed by residual solvent removal in vacuo to yield seven portions of resin 51, each loaded with a unique building block #1.

Tagging for Building Block #1 (BB₁). See reference 19 for detailed report of tagging procedures—H. E. Blackwell and coworkers, Chem. Biol. 8, 1167 (2001). Each of the seven portions of resin 51 loaded with BB#1 were then subjected to a unique encoding reaction. A freshly prepared solution of one or more tags (see Table 8, each tag 4.4 mM in 4.76 mL CH₂Cl₂) was individually prepared for each reaction. The resin 51 (˜286 mg/rxn) was then added to the solution of tags, placed under an Argon cloud, capped and sealed with parafilm, and allowed to rotate gently for 1 h. To this mixture was then added a freshly prepared solution of rhodium triphenylacetate (4.4 mg/mL, 4.76 mL), and the vial was sealed under Ar, wrapped in aluminum foil to prevent exposure to light, and allowed to tumble gently for 15 h. The resin was then isolated by filtration and washed as follows: 2×(5×CH₂Cl₂, CH₂Cl₂×15 min.), 3×(5×THF, THF×2 h), 5×anhydrous CH₂Cl₂, anhydrous CH₂Cl₂×15 min. The solvent was then removed under Ar flow for 1 h followed by residual solvent removal in vacuo to yield seven portions of resin 51 loaded with building block #1 and chemically encoded with polychlorinated aromatic tags T1A-T3A.

TABLE 8
Encoding scheme for building block #1

| | T1A | T2A | T3A | T4A | T5A | T6A | T7A | T8A | T9A | T10A | T11A | T13A |
|---|---|---|---|---|---|---|---|---|---|---|---|---|
| BB₁A | | | 1 | | | | | | | | | |
| BB₁B | | 1 | | | | | | | | | | |
| BB₁C | | 1 | 1 | | | | | | | | | |
| BB₁D | 1 | | | | | | | | | | | |
| BB₁E | 1 | | 1 | | | | | | | | | |
| BB₁F | 1 | 1 | | | | | | | | | | |
| BB₁G | 1 | 1 | 1 | | | | | | | | | |

Two macrobeads from each of the seven portions were removed and subjected to the standard HF-Pyridine-mediated compound cleavage conditions (see General Information), and the individual macrobeads and/or a portion of the solution of cleaved compounds were subsequently subjected to the standard CAN-mediated tag cleavage reaction (see reference 19). After confirming tagging scheme, the seven portions of dry resin 52 were then pooled together in a single polypropylene tube, swollen in anh. THF, tumbled for 30 min, and then the solvent was removed under Ar flow followed by residual solvent removel in vacuo.

Step 2. Suzuki Coupling of Skeletal Information Element #1 (σ₁)

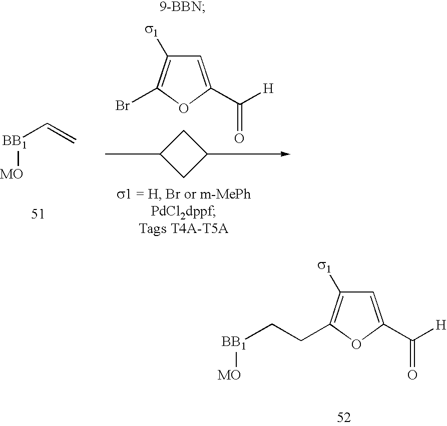

Suzuki coupling of Skeletal Information Element #1 (σ₁) A single pool of resin 51 was split evenly into three portions (672 mg each), and each was subjected to a coupling reaction with a unique a, as described below:

Colorless beads 51 (672 mg) were washed with THF (2×15 mL×10 min each) at rt and then resuspended in 20.2 mL THF. A 0.5M solution of 9-BBN in THF (13.4 mL, 6.72 mmol) was then added and the resulting mixture was manually agitated and let stand at rt for 5 h. The reaction solution was then removed via cannula and the colorless resin was washed thoroughly with THF (5×15 mL×10 min each). To the resin was then added solid PdCl₂dppf(σ_{0.1}□H: 8.2 mg, 0.0101 mmol; σ₁□Br: 13.7 mg, 0.0168 mmol; σ_{0.1}□Ar: 8.2 mg, 0.0101 mmol), and one of the following three 5-bromofuraldehydes:

TABLE 9
σ-elements #1 used in split-pool synthesis

| | | formula weight | |
|---|---|---|---|
| Skeletal information element (σ) | mmol | (g/mol) | mass (g) |
| σ₁A, 5-Bromofuraldehyde | 3.36 | 174.99 | 0.5880 |
| σ₁B, 4,5-Dibromofuraldehyde | 3.36 | 253.88 | 0.8530 |
| σ₁C, 4-m-MePh-5- | 3.36 | 265.1 | 0.8907 |
| bromofuraldehyde | | | |

via cannula as a solution in THF (8.4 mL), and a 1M solution of NaOH (1.68 mL, 1.68 mmol). The resulting orange reaction mixture was sealed under a cloud of Ar and heated at 65° C. with periodic manual agitation for 20 h (each reaction mixture turned dark brown). The yellow/orange resin was then isolated by filtration and washed as follows, 5×THF, 5×H2O, 5×THF, THF/H₂O: 3/1×2 h, 5×THF, 3×H₂O, 5×THF, THF/H₂O: 3/1×45min, 5×THF, THF/H₂O: 3/1×45 min, 5×THF, THF×20 min, 5×CH₂Cl₂, CH₂Cl₂×20 min, 5×anh. CH₂Cl₂, anh. CH₂Cl₂×20 min, and then the solvent was removed under Ar flow followed by residual solvent removal in vacuo to yield three portions of yellow/orange product resin 52.

Tagging for Skeletal Information Element #1 (σ₁). Each of the three product portions 52 was then subjected to a unique encoding reaction. A freshly prepared solution of one or more tags (see Table 10, each tag 4.4 mM in 11.1 mL CH₂Cl₂) was individually prepared for each reaction. The resin 52 (>672 mg/rxn) was then added to the solution of tags, placed under an Argon cloud, capped and sealed with parafilm, and allowed to rotate gently for 1 h. To this mixture was then added a freshly prepared solution of rhodium triphenylacetate (4.4 mg./mL, 11.1 mL), and the vial was sealed under Ar, wrapped in aluminum foil to prevent exposure to light, and allowed to tumble gently for 15 h. The resin was then isolated by filtration and washed as follows: 2×(5×CH₂Cl₂, CH₂Cl₂×15 min.), 3×(5×THF, THF×2 h), 5×anh. THF, anh. THF×1 h, 5×anh. CH₂Cl₂, anh. CH₂Cl₂×20 min, and the solvent was removed under Ar flow followed by residual solvent removal in vacuo to yield three portions of resin 52, collectively representing all combinations of building block #1 and σ-element #1, with each combination chemically encoded with polychlorinated aromatic tags.

TABLE 10
Encoding scheme for skeletal information element #1 (σ₁)

| | T1A | T2A | T3A | T4A | T5A | T6A | T7A | T8A | T9A | T10A | T11A | T13A |
|---|---|---|---|---|---|---|---|---|---|---|---|---|
| σ₁A (H) | | | | | 1 | | | | | | | |
| σ₁B (Br) | | | | 1 | | | | | | | | |
| σ₁C (Ar) | | | | 1 | 1 | | | | | | | |

10 individual macrobeads were removed from each portion 52 and subjected to the standard HF-Pyridine cleavage conditions. The cleaved product from all 30 individual macrobeads was analyzed by LCMS, and the polychlorinated tags remaining on each macrobead were then cleaved and analyzed by GC (data not shown). The three pools of dry resin 52 were then pooled together in a single polypropylene tube, swollen in anh. CH₂Cl₂, tumbled for 30 min, and then the solvent was removed under Ar flow followed by residual solvent removal in vacuo.

Step 3. Evans Aldol Coupling of Building Block #2 (BB₂)

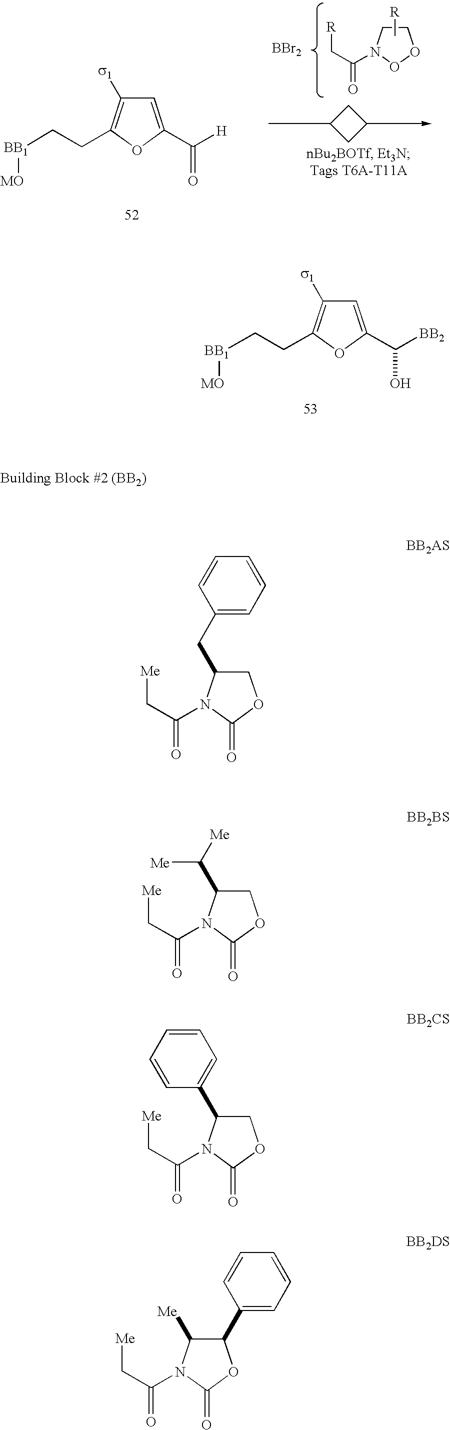

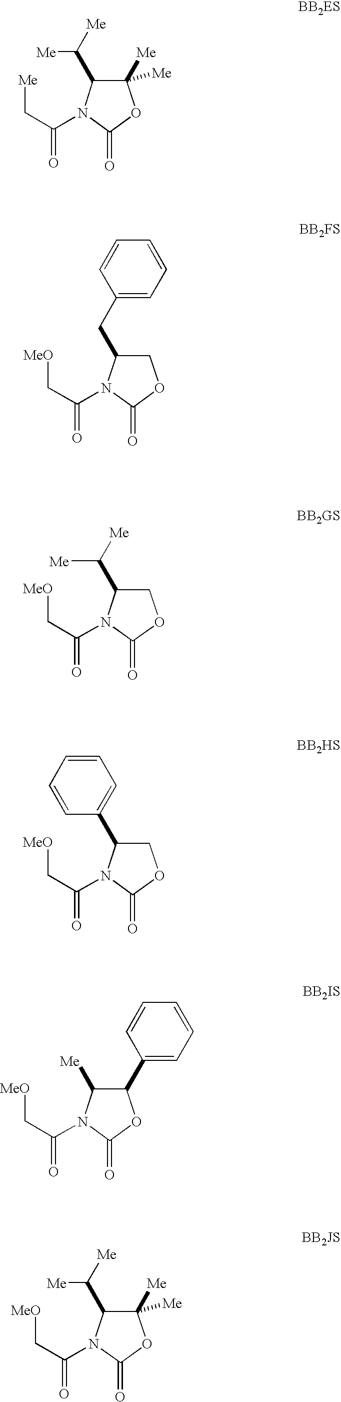

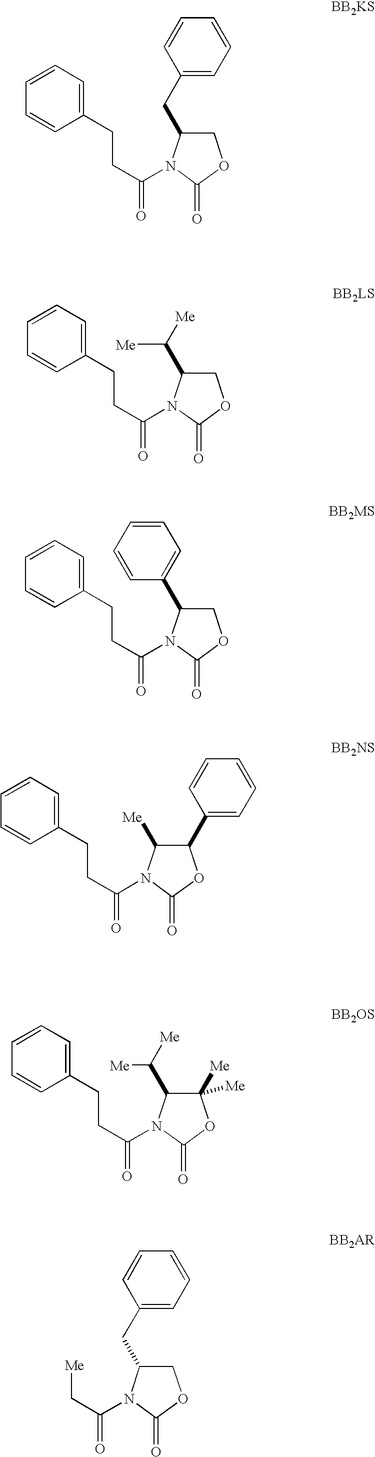

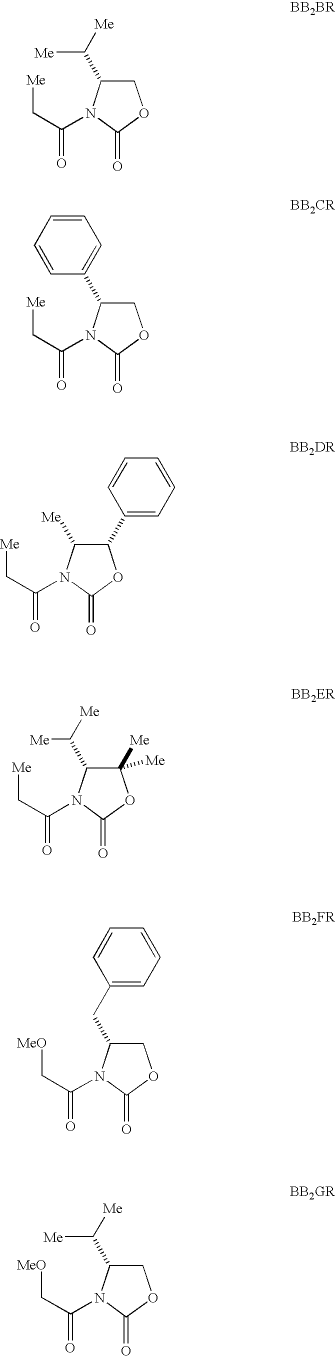

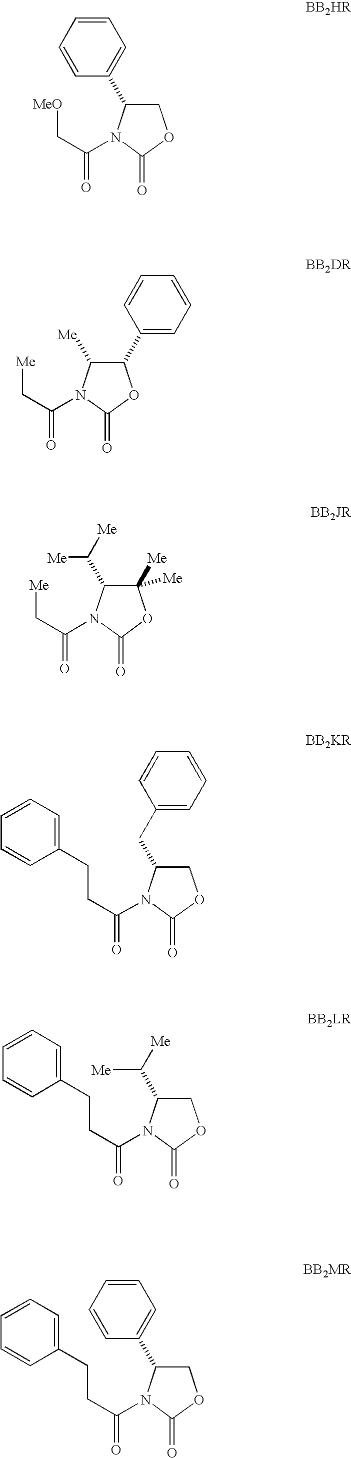

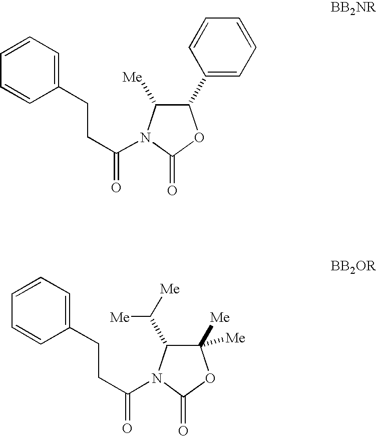

Aldol coupling of Building Block #2 (BB₂). The pooled resin 52 from Step 2 was then split into 30 equal portions (73.5 mg each) and each was subjected to an aldol coupling reaction with a unique BB#2. Resin 52 (73.5 mg) was washed with CH₂Cl₂ (2×3 mL×10 min each) at rt, and then cooled to −78° C. In a separate vessel, to a stirred solution of acyl o oxazolidinone (0.75 mmol, each was azeotropically dried from benzene just prior to reaction, see Table 11):

TABLE 11
BB#2 used in split-pool synthesis

| BB#2 | mmol | FW (g/mol) | mass (g) |
|---|---|---|---|
| BB₂AS | 0.75 | 233.26 | 0.175 |
| BB₂BS | 0.75 | 185.22 | 0.1389 |
| BB₂CS | 0.75 | 219.24 | 0.1644 |
| BB₂DS | 0.75 | 233.26 | 0.175 |
| BB₂ES | 0.75 | 213.27 | 0.16 |
| BB₂FS | 0.75 | 249.26 | 0.1869 |
| BB₂GS | 0.75 | 201.22 | 0.1509 |
| BB₂HS | 0.75 | 235.24 | 0.1764 |
| BB₂IS | 0.75 | 249.26 | 0.1869 |
| BB₂JS | 0.75 | 229.27 | 0.172 |
| BB₂KS | 0.75 | 309.36 | 0.232 |
| BB₂LS | 0.75 | 261.32 | 0.196 |
| BB₂MS | 0.75 | 295.33 | 0.2215 |
| BB₂NS | 0.75 | 309.36 | 0.232 |
| BB₂OS | 0.75 | 289.37 | 0.217 |
| BB₂AR | 0.75 | 233.27 | 0.175 |
| BB₂BR | 0.75 | 185.22 | 0.1389 |
| BB₂CR | 0.75 | 219.24 | 0.1644 |
| BB₂DR | 0.75 | 233.26 | 0.175 |
| BB₂ER | 0.75 | 213.27 | 0.16 |
| BB₂FR | 0.75 | 249.26 | 0.1869 |
| BB₂GR | 0.75 | 201.22 | 0.1509 |
| BB₂HR | 0.75 | 235.24 | 0.1764 |
| BB₂IR | 0.75 | 249.26 | 0.1869 |
| BB₂JR | 0.75 | 229.27 | 0.172 |
| BB₂KR | 0.75 | 309.36 | 0.232 |
| BB₂LR | 0.75 | 261.32 | 0.196 |
| BB₂MR | 0.75 | 295.33 | 0.2215 |
| BB₂NR | 0.75 | 309.36 | 0.232 |
| BB₂OR | 0.75 | 289.37 | 0.217 |

in CH₂Cl₂ (3 mL) at 0° C. was added a 1 M solution of dibutylboron triflate in CH₂Cl₂ (0.788 mL, 0.788 mmol) followed by triethylamine (0.125 mL, 0.900 mmol). The resulting enolate solution was cooled to −78° C. and then transferred rapidly via cannula to the vessel containing 52. The resulting mixture was sealed under a cloud of Ar and maintained at −78 ° C. for 48 h (72 h for BB₂MS, BB₂OS, BB₂MR, BB₂NR, and BB₂OR) −26° C. for 24 h, and 0° C. for 2 h (with periodic manual agitation about once every 8 h). The reaction was then quenched with the addition of pH7 phosphate buffer (3 mL), MeOH (3 mL), and 30% aq. H₂O₂ (2 mL), and the resulting mixture was tumbled at 4° C. for 12-15 h. Resin was then isolated by filtration and washed as follows: 5×CH₂Cl₂, 5×DMF, 5×THF, 5×CH₂Cl₂, CH₂Cl₂×1 h, 5×DMF, DMF×1 h, 5×THF, THF×1 h, 5×CH₂Cl₂, CH₂Cl₂×30 min, 5×anhydrous CH₂Cl₂, anhydrous CH₂Cl₂×30 min, and the solvent was removed under Ar flow followed by residual solvent removal in vacuo to yield yellow product resin 53.

Tagging for building block #2. Each of the 30 portions of product resin 53 loaded with BB2 was then subjected to a unique encoding reaction. A freshly prepared solution of one or more tags (see Table 12, each tag 4.4 mM in 1.1 mL CH₂Cl₂) was individually prepared for each reaction. The resin 53 (>73.5 mg/rxn) was then added to the solution of tags, placed under an Argon cloud, capped and sealed with parafilm, and allowed to rotate gently for 1 h. To this mixture was then added a freshly prepared solution of rhodium triphenylacetate (4.4 mg./mL, 1.1 mL), and the vial was sealed under Ar, wrapped in aluminum foil to prevent exposure to light, and allowed to tumble gently for 15 h. The resin was then isolated by filtration and washed as follows: 2×(5×CH₂Cl₂, CH₂Cl₂×15 min.), 3×(5×THF, THF×2 h), 5×anh. THF, anh. THF×1 h, 5×anh. CH₂Cl₂, anh. CH₂Cl₂×20 min, and the solvent was removed under Ar flow followed by residual solvent removal in vacuo to yield 30 portions of resin 53 representing all combinations of building block #1, σ-element #1, and building block #2, with each combination chemically encoded with polychlorinated aromatic tags.

TABLE 12
Encoding strategy for building block #2

| | T1A | T2A | T3A | T4A | T5A | T6A | T7A | T8A | T9A | T10A | T11A | T13A |
|---|---|---|---|---|---|---|---|---|---|---|---|---|
| BB₂AS | | | | | | | | | 1 | 1 | | |
| BB₂BS | | | | | | | | 1 | | 1 | | |
| BB₂CS | | | | | | | | 1 | 1 | 1 | | |
| BB₂DS | | | | | | | 1 | | | 1 | | |
| BB₂ES | | | | | | | 1 | | 1 | 1 | | |
| BB₂FS | | | | | | | 1 | 1 | | 1 | | |
| BB₂GS | | | | | | | 1 | 1 | 1 | 1 | | |
| BB₂HS | | | | | | 1 | | | | 1 | | |
| BB₂IS | | | | | | 1 | | | 1 | 1 | | |
| BB₂JS | | | | | | 1 | | 1 | | 1 | | |
| BB₂KS | | | | | | 1 | | 1 | 1 | 1 | | |
| BB₂LS | | | | | | 1 | 1 | | | 1 | | |
| BB₂MS | | | | | | 1 | 1 | | 1 | 1 | | |
| BB₂NS | | | | | | 1 | 1 | 1 | | 1 | | |
| BB₂OS | | | | | | 1 | 1 | 1 | 1 | 1 | | |
| BB₂AR | | | | | | | | | 1 | | 1 | |
| BB₂BR | | | | | | | | 1 | | | 1 | |
| BB₂CR | | | | | | | | 1 | 1 | | 1 | |
| BB₂DR | | | | | | | 1 | | | | 1 | |
| BB₂ER | | | | | | | 1 | | 1 | | 1 | |
| BB₂FR | | | | | | | 1 | 1 | | | 1 | |
| BB₂GR | | | | | | | 1 | 1 | 1 | | 1 | |
| BB₂HR | | | | | | 1 | | | | | 1 | |
| BB₂IR | | | | | | 1 | | | 1 | | 1 | |
| BB₂JR | | | | | | 1 | | 1 | | | 1 | |
| BB₂KR | | | | | | 1 | | 1 | 1 | | 1 | |
| BB₂LR | | | | | | 1 | 1 | | | | 1 | |
| BB₂MR | | | | | | 1 | 1 | | 1 | | 1 | |
| BB₂NR | | | | | | 1 | 1 | 1 | | | 1 | |
| BB₂OR | | | | | | 1 | 1 | 1 | 1 | | 1 | |

Two individual macrobeads were removed from each portion of product resin 53 and subjected to the standard HF-Pyridine cleavage conditions. The cleaved product from each of these 60 individual macrobeads was analyzed by LCMS, and the polychlorinated tags remaining on each macrobead were then cleaved and analyzed by GC. The results are presented below in Table 13. The 30 portions of dry resin 53 were then pooled together in a single polypropylene tube and well-mixed.

TABLE 13
Results of Step 3

| | | | | | | | | Mass spec |
|---|---|---|---|---|---|---|---|---|
| | Structure encoded | | | % Purity by | | | | consistent with |
| | by chemical tags | | | LCMS analysis | ES + Mass spec | | | tag-encoded |
| Macrobead | BB1 | σ1 | BB2 | (λ = 214 nm) | Ion | Calculated | Observed | structure |
| 53a | A | C | AS | >90 | M + Na⁺ | 514 | 514 | ✓ |
| 53b | E | B | AS | >90 | M + Na⁺ | 576 | 576 | ✓ |
| 53c | A | C | BS | >90 | M + Na⁺ | 466 | 466 | ✓ |
| 53d | E | B | BS | >90 | M + Na⁺ | 528 | 528 | ✓ |
| 53e | B | A | CS | >90 | M + Na⁺ | 438 | 438 | ✓ |
| 53f | B | C | CS | 90 | M + Na⁺ | 528 | 528 | ✓ |
| 53g | D | A | DS | >90 | M + Na⁺ | 454 | 454 | ✓ |
| 53h | A | C | DS | >90 | M + Na⁺ | 514 | 514 | ✓ |
| 53i | G | A | ES | 67 | M + Na⁺ | 746 | 746 | ✓ |
| 53j | A | B | ES | >90 | M + Na⁺ | 482 | 482 | ✓ |
| 53k | C | C | ES | 88 | M + Na⁺ | 614 | 614 | ✓ |
| 53l | G | B | ES | 55 | M + NH₄⁺ | 855 | 855 | ✓ |
| 53m | G | A | GS | 56 | M + Na⁺ | 734 | 734 | ✓ |
| 53n | A | A | GS | >90 | M + Na⁺ | 392 | 392 | ✓ |
| 53o | C | A | ES | >90 | M + Na⁺ | 510 | 510 | ✓ |
| 53p | B | C | ES | 88 | M + Na⁺ | 544 | 544 | ✓ |
| 53q | D | C | IS | 87 | M + Na⁺ | 560 | 560 | ✓ |
| 53r | F | C | IS | 88 | M + Na⁺ | 680 | 680 | ✓ |
| 53s | C | B | JS | >90 | M + Na⁺ | 582 | 582 | ✓ |
| 53t | D | C | JS | >90 | M + Na⁺ | 540 | 540 | ✓ |
| 53u | E | C | KS | >90 | M + Na⁺ | 664 | 664 | ✓ |
| 53v | C | A | KS | >90 | M + Na⁺ | 584 | 584 | ✓ |
| 53w | E | C | LS | 85 | M + Na⁺ | 616 | 616 | ✓ |
| 53x | C | A | LS | 90 | M + Na⁺ | 536 | 536 | ✓ |
| 53y | G | B | MS | 58 | M + NH₄⁺ | 901 | 901 | ✓ |
| 53z | G | A | MS | 74 | M + NH₄⁺ | 823 | 823 | ✓ |
| 53aa | G | C | NS | ND | M + NH₄⁺ | 927 | 927 | ✓ |
| 53bb | E | C | NS | 82 | M + Na⁺ | 664 | 664 | ✓ |
| 53cc | E | A | OS | 91 | M + Na⁺ | 554 | 554 | ✓ |
| 53dd | F | A | OS | 66 | M + Na⁺ | 630 | 630 | ✓ |
| 53ee | B | C | AR | 86 | M + Na⁺ | 542 | 542 | ✓ |
| 53ff | E | B | AR | >90 | M + Na⁺ | 576 | 576 | ✓ |
| 53gg | F | C | BR | 83 | M + Na⁺ | 616 | 616 | ✓ |
| 53hh | G | A | BR | 78 | M + Na⁺ | 718 | 718 | ✓ |
| 53ii | E | A | CR | >90 | M + Na⁺ | 484 | 484 | ✓ |
| 53jj | D | C | CR | 84 | M + Na⁺ | 530 | 430 | ✓ |
| 53kk | G | A | DR | 86 | M + NH₄⁺ | 761 | 761 | ✓ |
| 53ll | G | A | DR | 81 | M + NH₄⁺ | 761 | 761 | ✓ |
| 53mm | D | C | ER | 71 | M + Na⁺ | 524 | 524 | ✓ |
| 53nn | A | B | ER | 75 | M + Na⁺ | 482 | 482 | ✓ |
| 53oo | E | C | FR | 89 | M + Na⁺ | 604 | 604 | ✓ |
| 53pp | C | A | FR | >90 | M + Na⁺ | 524 | 524 | ✓ |
| 53qq | ND | ND | ND | 89 | ND | ND | ND | — |
| 53rr | C | A | GR | >90 | M + Na⁺ | 476 | 476 | ✓ |
| 53ss | F | B | HR | >90 | M + Na⁺ | 654 | 654 | ✓ |
| 53tt | ND | ND | ND | >90 | ND | ND | ND | — |
| 53uu | A | B | IR | >90 | M + Na⁺ | 518 | 518 | ✓ |
| 53vv | F | B | IR | >90 | M + Na⁺ | 668 | 668 | ✓ |
| 53ww | F | B | JR | 90 | M + Na⁺ | 648 | 648 | ✓ |
| 53xx | G | B | JR | 61 | M + NH₄⁺ | 835 | 835 | ✓ |
| 53yy | A | B | KR | >90 | M + Na⁺ | 578 | 578 | ✓ |
| 53zz | B | C | KR | 67 | M + Na⁺ | 618 | 618 | ✓ |
| 53aaa | G | A | LR | 56 | M + NH₄⁺ | 789 | 789 | ✓ |
| 53bbb | C | C | LR | 62 | M + Na⁺ | 626 | 626 | ✓ |
| 53ccc | G | C | MR | 69 | M + NH₄⁺ | 913 | 913 | ✓ |
| 53ddd | C | C | MR | 67 | M + Na⁺ | 660 | 660 | ✓ |
| 53eee | B | B | NR | >90 | M + Na⁺ | 606 | 606 | ✓ |
| 53fff | G | B | NR | 63 | M + NH₄⁺ | 915 | 915 | ✓ |
| 53ggg | G | B | OR | 36 | M + NH₄⁺ | 895 | 895 | ✓ |
| 53hhh | A | C | OR | 58 | M + Na⁺ | 570 | 570 | ✓ |

Step 4. +/−Acetylation of Aldol Adducts (σ₂)

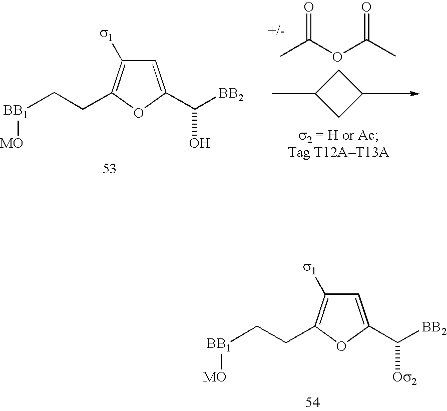

+/−Acetylation of aldol adducts (σ₂). The pooled collection of macrobeads 53 from Step 3 (2.157 g, 0.19 mg/bead, 11,170 beads) was then split evenly into two portions (1.08 g each). One portion was subjected to acetylation and the other portion was not. For the acetylation reaction, an oven-dried 120 mL sealed tube apparatus (ChemGlass) was charged with resin 53 and flushed with Ar stream for 10 minutes. The resin was then washed with anhydrous CH₂Cl₂ (2×50 mL×10 min each) at rt under Ar (washings removed by cannula) and then resuspended in CH₂Cl₂ (55 mL). To this mixture was then added i-Pr₂NEt (3.8 mL, 0.022 mol), DMAP (134 mg, 0.0011 mol), and finally acetic anhydride (1.04 mL, 0.011 mol) with manual agitation of the reaction solution following each addition. The resulting mixture was sealed under a blanket of Ar, the sealed tube was covered with aluminum foil, and the reaction mixture was tumbled at rt for 28 h. Resin was then isolated by filtration into a 20 polypropylene tube and washed as follows: 5×CH₂Cl₂, 5×THF, 5×CH₂Cl₂, CH₂Cl₂×45 min, 5×THF, THF×45 min, 5×CH₂Cl₂, CH₂Cl_{2×}45 min, 5×anh. CH₂Cl₂, anh. CH₂Cl₂×20 min, and then the solvent was removed under argon flow followed by residual solvent removal in vacuo.

Tagging for +/−acetylation of aldol adducts (σ₂). The product resin from this acetylation reaction was then added to a freshly prepared solution of tag T13A in CH₂Cl₂ (16.7 mL, 4.4 mM). The resulting mixture was sealed under an argon cloud and allowed to rotate gently for 1 h. Then, a freshly prepared solution of rhodium triphenylphosphate (16.66 mL., 4.4 mg./mL.) was added to the mixture of tags and resin. This vial was then sealed under an argon cloud, capped and sealed with parafilm, wrapped in aluminum foil to prevent exposure to light, and allowed to rotate gently for 15 h. The resin was then isolated by filtration and washed as follows: 2×(5×CH₂Cl₂, CH₂Cl₂×15 min.), 3×(5×THF, THF×2 h), 5×anh. THF, anh. anh. THF×45 min, 5×anh. CH₂Cl₂, anh. CH₂Cl₂×20 min, and the solvent was removed under Ar flow followed by residual solvent removal in vacuo to yield two portions of resin 54 representing all combinations of building block #1, σ-element #1, and building block #2, and σ-element #2, with each combination chemically encoded with polychlorinated aromatic tags.

TABLE 14
Encoding Strategy for σ-element #2

| | T1A | T2A | T3A | T4A | T5A | T6A | T7A | T8A | T9A | T10A | T11A | T13A |
|---|---|---|---|---|---|---|---|---|---|---|---|---|
| σ₂A (H) | | | | | | | | | | | | |
| σ₂B (Ac) | | | | | | | | | | | | 1 |

The compound and chemical tags were then cleaved from 60 individual macrobeads 54 (30 from each portion) and analyzed by LCMS and GC, respectively. These data were found to be consistent for 60/60 (100%) of these macrobeads, and the compounds cleaved from 55/60 (92%) of these macrobeads were determined to be ≧70% pure by LCMS analysis.

TABLE 15
Results of Step 4

| | | | | | | | | | | Mass spec |
|---|---|---|---|---|---|---|---|---|---|---|
| | Structure encoded | | | | LCMS analysis | | | | | consistent with |
| | by chemical tags | | | | (λ = 214 nm) | | ES + Mass Spec | | | tag-encoded |
| Macrobead | BB1 | σ1 | BB2 | σ2 | Purity(%) | t_{R} (min) | Ion | Calculated | Observed | structure |
| 54a | F | C | HS | A | >90 | 9.36 | M + NH₄⁺ | 661 | 661 | ✓ |
| 54b | G | B | KS | A | 75 | 11.89 | M + NH₄⁺ | 915 | 915 | ✓ |
| 54c | D | C | CR | A | 84 | 8.15 | M + NH₄⁺ | 525 | 525 | ✓ |
| 54d | F | C | ES | A | >90 | 10.10 | M + NH₄⁺ | 639 | 639 | ✓ |
| 54e | A | C | OS | A | 71 | 9.88 | M + NH₄⁺ | 565 | 565 | ✓ |
| 54f | A | B | BS | A | 87 | 6.98 | M + Na⁺ | 454 | 454 | ✓ |
| 54g | C | C | JS | A | >90 | 9.12 | M + NH₄⁺ | 499 | 499 | ✓ |
| 54h | C | B | KR | A | >70 | 11.54 | M + Na⁺ | 662 | 662 | ✓ |
| 54i | C | A | AS | A | 88 | 9.55 | M + Na⁺ | 508 | 508 | ✓ |
| 54j | E | A | DR | A | 90 | 6.70 | M + Na⁺ | 498 | 498 | ✓ |
| 54k | F | A | LS | A | >90 | 9.08 | M + Na⁺ | 602 | 602 | ✓ |
| 54l | E | A | JR | A | 87 | 5.43 | M + NH₄⁺ | 489 | 489 | ✓ |
| 54m | C | A | BS | A | 89 | 9.14 | M + Na⁺ | 460 | 460 | ✓ |
| 54n | F | A | GS | A | 72 | 7.15 | M + NH₄⁺ | 537 | 537 | ✓ |
| 54o | G | B | AR | A | 76 | 11.06 | M + NH₄⁺ | 839 | 839 | ✓ |
| 54p | F | B | HR | A | 78 | 8.43 | M + Na⁺ | 654 | 654 | ✓ |
| 54q | A | A | OR | A | >90 | 8.07 | M + Na⁺ | 480 | 480 | ✓ |
| 54r | G | B | JS | A | 87 | 10.37 | M + NH₄⁺ | 835 | 835 | ✓ |
| 54s | B | A | DS | A | 87 | 7.99 | M + Na⁺ | 452 | 452 | ✓ |
| 54t | A | B | KR | A | 86 | 8.97 | M + NH₄⁺ | 573 | 573 | ✓ |
| 54u | D | B | BR | A | 89 | 6.59 | M + Na⁺ | 484 | 484 | ✓ |
| 54v | B | C | FR | A | 91 | 8.79 | M + NH₄⁺ | 553 | 553 | ✓ |
| 54w | F | B | BR | A | 74 | 8.76 | M + Na⁺ | 604 | 604 | ✓ |
| 54x | E | A | DR | A | >90 | 6.66 | M + NH₄⁺ | 493 | 493 | ✓ |
| 54y | C | H | OR | A | >70 | 10.79 | M + Na⁺ | 564 | 564 | ✓ |
| 54z | C | A | MR | A | <70 | 10.34 | M + Na⁺ | 570 | 570 | ✓ |
| 54aa | B | C | KR | A | <70 | 10.44 | M + NH₄⁺ | 613 | 613 | ✓ |
| 54bb | G | C | HS | A | 88 | 10.74 | M + NH₄⁺ | 853 | 853 | ✓ |
| 54cc | F | H | KR | A | 80 | 9.48 | M + NH₄⁺ | 645 | 645 | ✓ |
| 54dd | A | C | OS | A | 68 | 9.83 | M + NH₄⁺ | 565 | 565 | ✓ |
| 54ee | E | B | HR | B | >90 | 7.50 | M + NH₄⁺ | 615 | 615 | ✓ |
| 54ff | F | C | HK | B | 85 | 12.59 | M + NH₄⁺ | 111 | 111 | ✓ |
| 54gg | C | A | KS | B | >90 | 11.76 | M + NH₄⁺ | 621 | 621 | ✓ |
| 54hh | B | B | OS | B | >70 | 10.85 | M + NH₄⁺ | 623 | 623 | ✓ |
| 54ii | E | B | OR | B | 69 | 9.56 | M + NH₄⁺ | 669 | 669 | ✓ |
| 54jj | D | B | MS | B | 88 | 9.35 | M + NH₄⁺ | 631 | 631 | ✓ |
| 54kk | C | A | JR | B | >90 | 10.28 | M + NH₄⁺ | 541 | 541 | ✓ |
| 54ll | G | A | IS | B | >90 | 10.71 | M + NH₄⁺ | 819 | 819 | ✓ |
| 54mm | B | A | MS | B | 90 | 9.57 | M + NH₄⁺ | 551 | 551 | ✓ |
| 54nn | G | B | JR | B | 71 | 11.43 | M + NH₄⁺ | 877 | 877 | ✓ |
| 54oo | A | B | AR | B | >90 | 8.95 | M + NH₄⁺ | 539 | 539 | ✓ |
| 54pp | A | C | GS | B | 77 | 8.78 | M + NH₄⁺ | 519 | 519 | ✓ |
| 54qq | A | C | NR | B | 88 | 11.27 | M + NH₄⁺ | 627 | 627 | ✓ |
| 54rr | B | C | JS | B | >90 | 10.07 | M + NH₄⁺ | 575 | 575 | ✓ |
| 54ss | B | A | JS | B | >90 | 8.27 | M + NH₄⁺ | 485 | 485 | ✓ |
| 54tt | B | C | FR | B | >90 | 9.88 | M + NH₄⁺ | 595 | 595 | ✓ |
| 54uu | A | B | MR | B | >90 | 9.66 | M + NH₄⁺ | 601 | 601 | ✓ |
| 54vv | B | A | OS | B | >90 | 9.95 | M + NH₄⁺ | 545 | 545 | ✓ |
| 54ww | C | A | MS | B | >90 | 11.31 | M + NH₄⁺ | 607 | 607 | ✓ |
| 54xx | A | C | JS | B | >90 | 9.39 | M + NH₄⁺ | 547 | 547 | ✓ |
| 54yy | D | A | JS | B | >90 | 7.01 | M + NH₄⁺ | 487 | 487 | ✓ |
| 54zz | A | C | AS | B | >90 | 9.87 | M + NH₄⁺ | 551 | 551 | ✓ |
| 54aaa | D | A | HR | B | >90 | 6.62 | M + NH₄⁺ | 493 | 493 | ✓ |
| 54bbb | E | C | HR | B | >90 | 8.56 | M + NH₄⁺ | 627 | 627 | ✓ |
| 54ccc | C | B | CS | B | <70 | 11.12 | M + Na⁺ | 614 | 614 | ✓ |
| 54ddd | F | B | LS | B | 73 | 10.83 | M + NH₄⁺ | 717 | 717 | ✓ |
| 54eee | D | B | LS | B | >90 | 9.20 | M + NH₄⁺ | 597 | 597 | ✓ |
| 54fff | A | A | ES | B | >90 | 8.06 | M + Na⁺ | 446 | 446 | ✓ |
| 54ggg | D | A | ES | B | >90 | 7.71 | M + NH₄⁺ | 471 | 471 | ✓ |
| 54hhh | D | C | JR | B | 87 | 9.08 | M + NH₄⁺ | 577 | 577 | ✓ |

The two portions of light brown product resin 54, representing all possible combinations of BB₁, σ₁, BB₂, and σ₂ in both enantiomeric/diasteromeric forms, were then pooled together in a single polypropylene tube and well mixed.

Step 5. NBS and PPTS-mediated Transformation of Pooled Substrates 54 into 1260 Products Representing a Complete, Combinatorial Matrix of Molecular Skeletons, Each Derivatized With a Complete, Combinatorial Matrix of Building Blocks in Both Enantiomeric/diastereomeric Forms

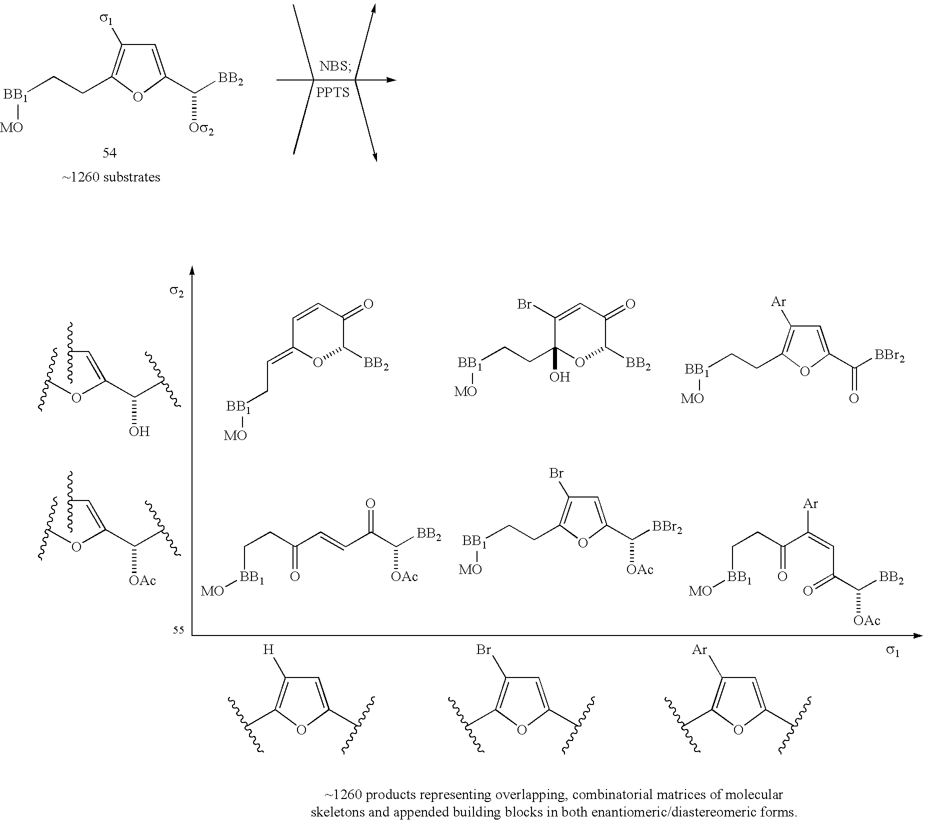

Experimental: A 120 mL sealed tube apparatus (Chemglass) was charged with THF (64 mL), H₂O (16 mL, THF and H₂O were mixed to homogeneity), and macrobead-bound substrates 54 (853 mg, ˜5.2 macrobeads/mg, ˜4410 macrobeads, multiplicative factor=3.5; substrate macrobeads were light brown) at rt under ambient. The resulting mixture was agitated manually for 2 min and then let stand at rt under ambient for 10 minutes. To this mixture was then added NaHCO₃ (3.06 g, 36 mmol) and NaOAc (1.48 g, 18 mmol) and the resulting mixture let stand at rt for 10 minutes with periodic manual agitation (2 layers formed). To this mixture was then added NBS (2.136 g, 12 mmol) and the resulting yellow tinted reaction mixture was sealed under ambient and manually agitated. The flask was immediately wrapped in aluminum foil and then tumbled at rt for 1 h (the reaction solution turned dark yellow/yellow-orange). The resin was then isolated by filtration into a 20 mL polypropylene tube using THF and H₂O (macrobeads were light yellow) and then washed as follows: 5×THF, 5×H₂O, 5×THF, THF/H₂O: 3/1×1 h, 5×THF, THF×1 h, 5×CH₂Cl₂, CH₂Cl₂×30 min, 5×anhydrous CH₂Cl₂, anhydrous CH₂Cl₂×30 min, 5×anhydrous CH₂Cl₂×2 min each, and then the solvent was removed under Ar flow followed by residual solvent removal in vacuo (1 h). An oven-dried, 350 mL sealed tube apparatus (Chemglass) was then charged at rt under a cloud of Ar with anh. CH₂Cl₂ (200 mL) and pyridinium p-toluenesulfonate (37.7 mg, 0.15 mmol, 0.00075M in CH₂Cl₂). The flask was then sealed and manually agitated to make a clear, colorless solution. The resin was added, the flask was sealed under a cloud of Ar, and the reaction mixture was warmed to 40-45° C. and maintained at that temperature for 20 h with periodic manual agitation every 4-8 h. Resin was then isolated by filtration into a 20 mL polypropylene tube (using THF and a glass funnel to transfer resin) and then washed as follows: 5×THF, 5×H₂O, 5×THF, THF/dil. aq. NaHCO₃ (sat. aq. NaHCO₃/H₂O: 1/2): 1/1×1 h, 5×THF, 5×H₂O, 5×THF, THF/dilute aq. NH₄Cl (sat. aq. NH₄Cl/H₂O: 1/2): 1/1×1 h, 5×THF, 5×H₂O, 5×THF, THF/H₂O: 3/1×45 min, 5×THF, THF×45 min, 5×CH₂Cl₂, CH₂Cl₂×30 min, 5×anh. CH₂Cl₂, anh. CH₂Cl₂×30 min, and 5×anhydrous CH₂Cl₂×2 min each. Solvent was then removed under Ar flow followed by residual solvent removal in vacuo to yield a collection of macrobead-bound products 55 representing a complete, combinatorial (3×2=6) matrix of molecular skeletons, each derivatized with a complete, combinatorial (7×15=105) matrix of building blocks in both enantiomeric/diastereomeric forms (6×15×2=1260).

The compound and chemical tags were cleaved from 120 individual product macrobeads 55 and then analyzed by LCMS and GC, respectively. The LCMS data were consistent with the formation of the functionalized skeleton encoded by the corresponding chemical tags in 120 out of 120 cases (100%). Moreover, 84/120 (70%) of these compounds were determined to be ≧70% pure by LCMS analysis.

TABLE 16
Results of step 5

| | | | | | | | | | | Mass Spec |
|---|---|---|---|---|---|---|---|---|---|---|
| | Structure encoded by | | | | LCMS analysis | | | | | consistent with |
| | chemical tags | | | | (λ = 214 nm) | | ES + Mass Spec | | | tag-encoded |
| Macrobead | BB1 | σ1 | BB2 | σ2 | Purity (%) | t_{R} (min) | Ion | Calculated | Observed | structure |
| 55a | F | B | DR | B | 37 | 10.61 | M + NH₄⁺ | 689 | 689 | ✓ |
| 55b | E | A | AR | B | >90 | 6.98 | M + NH₄⁺ | 551 | 551 | ✓ |
| 55c | B | C | KS | B | 65 | 10.39 | M + NH₄⁺ | 671 | 671 | ✓ |
| 55d | D | A | JR | A | 21 | 6.06 | M + H⁺ | 426 | 426 | ✓ |
| 55e | E | A | FR | B | >90 | 6.38 | M + NH₄⁺ | 567 | 567 | ✓ |
| 55f | G | B | DS | A | 49 | 10.98 | M + NH₄⁺ | 855 | 855 | ✓ |
| 55g | D | A | LS | B | >90 | 7.75 | M + NH₄⁺ | 535 | 535 | ✓ |
| 55h | B | A | IR | B | >90 | 7.78 | M + NH₄⁺ | 521 | 521 | ✓ |
| 55i | B | A | IR | A | >90 | 7.67 | M + H⁺ | 444 | 444 | ✓ |
| 55j | F | C | OR | B | 43 | 11.43 | M + NH₄⁺ | 773 | 773 | ✓ |
| 55k | G | A | PR | A | 82 | 9.91 | M + NH₄⁺ | 775 | 775 | ✓ |
| 55l | D | B | MR | A | 79 | 8.06 | M + NH₄⁺ | 605 | 605 | ✓ |
| 55m | B | B | BR | A | >90 | 7.45 | M + Na⁺ | 498 | 498 | ✓ |
| 55n | A | B | FS | B | >90 | 8.31 | M + Na⁺ | 560 | 560 | ✓ |
| 55o | C | B | NS | A | <70 | 11.7 | M + Na⁺ | 678 | 678 | ✓ |
| 55p | G | B | MS | A | 47 | 11.32 | M + NH₄⁺ | 917 | 917 | ✓ |
| 55q | F | B | NR | A | 74 | 10.32 | M + NH₄⁺ | 739 | 739 | ✓ |
| 55r | B | A | CR | A | >90 | 7.73 | M + H⁺ | 414 | 414 | ✓ |
| 55s | B | A | FS | A | >90 | 7.26 | M + H⁺ | 444 | 444 | ✓ |
| 55t | G | A | BR | A | 68 | 10.10 | M + NH₄⁺ | 711 | 711 | ✓ |
| 55u | D | C | HS | B | 71 | 7.81 | M + H⁺ | 582 | 582 | ✓ |
| 55v | D | C | ER | A | 54 | 9.28 | M + H⁺ | 500 | 500 | ✓ |
| 55w | A | B | KS | B | >90 | 9.99 | M + NH₄⁺ | 615 | 615 | ✓ |
| 55x | A | C | HS | A | 49 | 8.76 | M + H⁺ | 492 | 492 | ✓ |
| 55y | B | C | LR | B | 32 | 10.12 | M + NH₄⁺ | 623 | 623 | ✓ |
| 55z | E | B | CS | A | >90 | 6.58 | M + NH₄⁺ | 573 | 573 | ✓ |
| 55aa | B | B | KR | B | >90 | 10.63 | M + NH₄⁺ | 643 | 643 | ✓ |
| 55bb | C | A | CR | A | >90 | 9.72 | M + H⁺ | 470 | 470 | ✓ |
| 55cc | E | A | JR | A | 51 | 6.06 | M + H⁺ | 470 | 470 | ✓ |
| 55dd | F | A | GS | A | 60 | 7.54 | M + H⁺ | 518 | 518 | ✓ |
| 55ee | B | A | NR | A | >90 | 9.74 | M + H⁺ | 504 | 504 | ✓ |
| 55ff | A | C | OS | A | 3 | 10.73 | M + H⁺ | 546 | 546 | ✓ |
| 55gg | B | A | LS | A | >90 | 8.81 | M + H⁺ | 456 | 456 | ✓ |
| 55hh | C | A | FR | B | >90 | 9.26 | M + NH₄⁺ | 577 | 577 | ✓ |
| 55ii | B | A | FS | A | >90 | 7.27 | M + H⁺ | 444 | 444 | ✓ |
| 55jj | C | B | OS | A | >90 | 11.71 | M + Na⁺ | 658 | 658 | ✓ |
| 55kk | G | B | FR | A | 65 | 10.07 | M + NH₄⁺ | 871 | 871 | ✓ |
| 55ll | A | C | NS | A | 84 | 10.86 | M + H⁺ | 566 | 566 | ✓ |
| 55mm | D | B | IS | A | 78 | 6.69 | M + NH₄⁺ | 559 | 559 | ✓ |
| 55nn | E | B | IR | B | <70 | 8.12 | M + NH₄⁺ | 629 | 629 | ✓ |
| 55oo | A | B | NR | B | >90 | 10.36 | M + NH₄⁺ | 615 | 615 | ✓ |
| 55pp | E | B | LS | A | 87 | 7.75 | M + NH₄⁺ | 615 | 615 | ✓ |
| 55qq | B | B | LS | B | >90 | 10.22 | M + Na⁺ | 600 | 600 | ✓ |
| 55rr | F | B | BS | A | 64 | 8.38 | M + NH₄⁺ | 615 | 615 | ✓ |
| 55ss | C | A | DS | A | >90 | 10.54 | M + H⁺ | 484 | 484 | ✓ |
| 55tt | C | A | ER | B | >70 | 10.14 | M + H⁺ | 524 | 524 | ✓ |
| 55uu = 18 | B | C | AS | A | >90 | 10.06 | M + H⁺ | 518 | 518 | ✓ |
| 55vv | D | C | LS | A | >90 | 9.87 | M + H⁺ | 548 | 548 | ✓ |
| 55ww | F | A | HR | B | >90 | 7.98 | M + NH₄⁺ | 629 | 629 | ✓ |
| 55xx | G | C | LS | A | ND | >12.5 | M + NH₄⁺ | 888 | 888 | ✓ |
| 55yy | C | C | JS | B | 49 | 10.92 | M + H⁺ | 630 | 630 | ✓ |
| 55zz | E | B | HR | B | >70 | 7.51 | M + NH₄⁺ | 615 | 615 | ✓ |
| 55aaa | E | C | AR | A | 79 | 8.96 | M + H⁺ | 564 | 564 | ✓ |
| 55bbb | A | B | MS | A | >70 | 8.24 | M + Na⁺ | 580 | 580 | ✓ |
| 55ccc | D | C | LR | A | 61 | 9.83 | M + H⁺ | 548 | 548 | ✓ |
| 55ddd | C | A | IS | A | >90 | 9.40 | M + H⁺ | 500 | 500 | ✓ |
| 55eee | D | A | HS | B | >90 | 5.97 | M + NH₄⁺ | 509 | 509 | ✓ |
| 55fff | B | A | LR | B | >70 | 8.69 | M + Na⁺ | 538 | 538 | ✓ |
| 55ggg | A | C | GR | B | 41 | 8.95 | M + Na⁺ | 540 | 540 | ✓ |
| 55hhh | F | A | JR | A | 43 | 8.38 | M + H⁺ | 546 | 546 | ✓ |
| 55iii | A | C | NR | A | >90 | 10.95 | M + H⁺ | 566 | 566 | ✓ |
| 55jjj | A | C | LS | A | >90 | 10.10 | M + H⁺ | 518 | 518 | ✓ |
| 55kkk | B | A | AR | B | >90 | 8.04 | M + NH₄⁺ | 505 | 505 | ✓ |
| 55lll | C | C | MS | B | ND | 12.07 | M + NH₄⁺ | 713 | 713 | ✓ |
| 55mmm | E | C | AS | A | 78 | 8.97 | M + H⁺ | 564 | 564 | ✓ |
| 55nnn | C | C | NS | A | ND | >12.5 | M + H⁺ | 650 | 650 | ✓ |
| 55ooo | C | B | DR | A | >90 | 10.60 | M + Na⁺ | 602 | 602 | ✓ |
| 55ppp | B | B | GR | B | >90 | 8.55 | M + Na⁺ | 540 | 540 | ✓ |
| 55qqq | F | A | MR | A | 70 | 9.63 | M + H⁺ | 612 | 612 | ✓ |
| 55rrr | A | A | LR | A | >90 | 7.98 | M + H⁺ | 428 | 428 | ✓ |
| 55sss | E | A | OS | A | 81 | 8.30 | M + H⁺ | 530 | 530 | ✓ |
| 55ttt | E | C | BR | A | 72 | 8.41 | M + H⁺ | 516 | 516 | ✓ |
| 55uuu | G | B | NS | A | 37 | 12.08 | M + NH₄⁺ | 931 | 931 | ✓ |
| 55vvv | F | C | KR | A | 82 | 11.78 | M + H⁺ | 716 | 716 | ✓ |
| 55www | A | B | MS | A | >90 | 8.24 | M + Na⁺ | 580 | 580 | ✓ |
| 55xxx | A | C | AR | A | >90 | 8.61 | M + H⁺ | 476 | 476 | ✓ |
| 55yyy | F | B | IS | B | 79 | 9.96 | M + NH₄⁺ | 705 | 705 | ✓ |
| 55zzz | A | C | GS | A | 42 | 8.50 | M + H⁺ | 458 | 458 | ✓ |
| 55aaaa | G | C | FS | A | 38 | 11.78 | M + NH₄⁺ | 865 | 865 | ✓ |
| 55bbbb | G | C | CS | B | 85 | 11.21 | M + NH₄⁺ | 895 | 895 | ✓ |
| 55cccc | D | A | OR | A | 72 | 8.42 | M + H⁺ | 486 | 486 | ✓ |
| 55dddd | C | B | HR | A | >70 | 9.04 | M + Na⁺ | 604 | 604 | ✓ |
| 55eeee | D | C | JS | B | 80 | 8.29 | M + H⁺ | 576 | 576 | ✓ |
| 55ffff | G | A | NS | A | 75 | 11.43 | M + NH₄⁺ | 835 | 835 | ✓ |
| 55gggg | F | A | FS | B | >90 | 8.30 | M + NH₄⁺ | 643 | 643 | ✓ |
| 55hhhh | E | A | AS | B | >90 | 6.93 | M + H⁺ | 534 | 534 | ✓ |
| 55iiii | E | C | HS | A | 66 | 8.28 | M + H++ | 566 | 566 | ✓ |
| 55jjjj | F | C | AS | A | 69 | 10.67 | M + H⁺ | 640 | 640 | ✓ |
| 55kkkk | E | B | FS | A | 81 | 6.28 | M + NH₄⁺ | 603 | 603 | ✓ |
| 55llll | D | B | AR | A | 90 | 7.00 | M + NH₄⁺ | 543 | 543 | ✓ |
| 55mmmm | C | B | JR | A | >90 | 9.60 | M + Na⁺ | 598 | 598 | ✓ |
| 55nnnn | C | A | CS | A | >90 | 9.71 | M + H⁺ | 470 | 470 | ✓ |
| 55oooo | B | C | DS | B | 73 | 9.87 | M + NH₄⁺ | 595 | 595 | ✓ |
| 55pppp | B | A | OR | B | <70 | 9.4 | M + Na⁺ | 566 | 566 | ✓ |
| 55qqqq | A | A | FS | A | >90 | 6.35 | M + H⁺ | 416 | 416 | ✓ |
| 55rrrr | E | A | BS | A | >90 | 5.95 | M + H⁺ | 426 | 426 | ✓ |
| 55ssss | C | B | FS | B | >90 | 10.90 | M + Na⁺ | 644 | 644 | ✓ |
| 55tttt | F | C | KR | B | 79 | 10.95 | M + NH₄⁺ | 793 | 793 | ✓ |
| 55uuuu | C | C | HR | A | 73 | 11.05 | M + H⁺ | 576 | 576 | ✓ |
| 55vvvv | C | B | AR | B | >90 | 11.58 | M + Na⁺ | 628 | 628 | ✓ |
| 55wwww | F | C | DS | B | >90 | 10.47 | M + NH₄⁺ | 717 | 717 | ✓ |
| 55xxxx | C | B | BS | A | 69 | 9.62 | M + Na⁺ | 554 | 554 | ✓ |
| 55yyyy | F | A | MR | B | 85 | 9.49 | M + NH₄⁺ | 689 | 689 | ✓ |
| 55zzzz | F | A | DR | A | 69 | 8.90 | M + H⁺ | 550 | 550 | ✓ |
| 55aaaaa | E | B | LS | A | 89 | 7.74 | M + NH₄⁺ | 615 | 615 | ✓ |
| 55bbbbb | B | B | HS | B | >90 | 8.76 | M + Na⁺ | 574 | 574 | ✓ |
| 55ccccc | F | B | MS | B | >90 | 10.79 | M + NH₄⁺ | 751 | 751 | ✓ |
| 55ddddd | D | A | JS | B | >90 | 6.42 | M + Na⁺ | 508 | 508 | ✓ |
| 55eeeee | G | A | HR | B | 80 | 9.59 | M + NH₄⁺ | 821 | 821 | ✓ |
| 55fffff | D | B | NS | A | >90 | 8.76 | M + Na⁺ | 624 | 624 | ✓ |
| 55ggggg | C | C | KR | B | ND | >12.5 | M + Na⁺ | 732 | 732 | ✓ |
| 55hhhhh | E | A | FR | B | >90 | 6.33 | M + NH₄⁺ | 567 | 567 | ✓ |
| 55iiiii | C | C | LR | A | ND | >12.5 | M + H⁺ | 602 | 602 | ✓ |
| 55jjjjj | D | A | NR | A | 65 | 8.71 | M + H⁺ | 506 | 506 | ✓ |
| 55kkkkk | D | C | OS | A | 76 | 10.49 | M + H⁺ | 576 | 576 | ✓ |
| 55lllll | A | A | ER | A | >90 | 7.48 | M + H⁺ | 380 | 380 | ✓ |
| 55mmmmm | A | C | NR | B | 35 | 11.80 | M + Na⁺ | 648 | 648 | ✓ |
| 55nnnnn | C | C | IS | A | >90 | 11.86 | M + H⁺ | 590 | 590 | ✓ |
| 55ooooo | F | A | KR | A | 71 | 9.90 | M + H⁺ | 626 | 626 | ✓ |
| 55ppppp | A | C | AR | A | >90 | 9.44 | M + H⁺ | 490 | 490 | ✓ |

Chemical structures, HPLC traces (UV/λ₂₁₄ and MS/ES⁺ total ion current) and mass spectroscopic data (ES⁺) are available for all 60 ‘substrates’ 54 and all 120 ‘products’ 55 in the form of Appendix C (181 pages).

### Example 3

### Skeletal Diversity of a Branched Pathway: Efficient Synthesis of 29,400 Discrete, Polycyclic Compounds and Their Arraying into Stock Solutions

Diversity-oriented synthesis (DOS) aims to synthesize efficiently complex, small molecules broadly distributed in multidimensional descriptor space (Schreiber, S. L. Science 2000, 287, 1964-196; incorporated herein by reference). Such collections (Dolle, R. E. J. Comb. Chem. 2001, 3, 477-517; incorporated herein by reference) are key to chemical genetics, where small molecules are used to explore biology and medicine systematically (Schreiber, S. L. Bioorg. Med. Chem. 1998, 6, 1127-1152; Mitchison, T. J. Chem. Biol. 1994, 1, 3-6; each of which is incorporated herein by reference). Skeletal diversity in DOS has proven to be especially challenging. Here, we report a branching DOS pathway that yields 29,400 discrete compounds comprising ten distinct polycyclic skeletons (Here we define compounds with different: (1) numbers or sizes of rings, (2) ring fusion stereochemistry, or (3) degree of ring fusion saturation as having “different skeleton”.).

The six-step, stereoselective synthesis, which affords products having a central skeleton with between two and four rings and up to six stereocenters, has been achieved using an inexpensive and accessible, one bead-one stock solution technology platform (Blackwell, H. E.; Perez, L.; Stavenger, R. A.; Tallarico, J. A.; Eatough, E. C.; Foley, M. A.; Schreiber, S. L. Chem. Biol. 2001, 8, 1167-1182; Clemons, P. A.; Koehler, A. N.; Wagner, B. K.; Sprigings, T. G.; Spring, D. R.; King, R. W.; Schreiber, S. L.; Foley, M. A. Chem. Biol. 2001, 8, 1183-1195; each of which is incorporated herein by reference). The pathway builds on the report by Fallis and co-workers on the use of consecutive Diels-Alder reactions (Woo, S.; Squires, N.; Fallis, A. G. Org. Lett. 1999, 1, 573-575; incorporated herein by reference). We have adapted their reported triene synthesis and subsequent complexity-generating reactions to phenolic aldehyde-loaded macrobeads, and discovered a set of dienophiles that react only once with the Fallis-type trienes. The latter observation provides a branch point to the pathway, where diene products are formed from a single Diels-Alder cycloaddition and monoene products are formed from consecutive Diels-Alder reactions involving either the same or different dienophiles (FIG. 19). An important feature of the branched pathway is that the diastereoselection observed in the original report has been extended to reaction sequences involving different dienophiles.

To optimize the yield and purity of the library members, potential building blocks for the library were tested individually as follows. In separate reaction vessels, 64 hydroxyaldehydes were loaded onto macrobeads through silylation of their hydroxyl groups with the previously described macrobead-alkylsilyl triflate (illustrated with the silylation-loading of vanillin 1 in FIG. 18) (Tallarico, J. A.; Depew, K. D.; Pelish, H. E.; Westwood, N. J.; Lindsley, C. W.; Shair, M. D.; Schreiber, S. L.; Foley, M. A. J Comb. Chem. 2001, 3, 312-318; incorporated herein by reference). Each macrobead-loaded aldehyde was separately reacted with indium dust (for indium-mediated allylation of resin-bound aldehydes with sonication: Cavallaro, C. L.; Herpin, T.; MuGuinness, B. F.; Shimshock, Y. C.; Dolle, R. E. Tetrahedron Lett. 1999, 40, 2711-2714; incorporated herein by reference) and 5-bromo-1,3-pentadiene (Prevost, C.; Miginiac, P.; Miginiac-Groizeleau, L. Bull. Soc. Chim. Fr. 1964, 2485-2492; incorporated herein by reference) in DMF, which provided the γ-addition product (2 in the illustrated case with vanillin). Mesylation followed by elimination using DBU furnished the cross-conjugated triene (c.f., 3). After cleavage with HF-py and analysis of the purity of the triene products by ¹H NMR, 40 of the original 64 hydroxyaldehydes (FIG. 19 top) were found to yield a single identifiable compound. These 40 aldehydes were used in the DOS pathway described below.

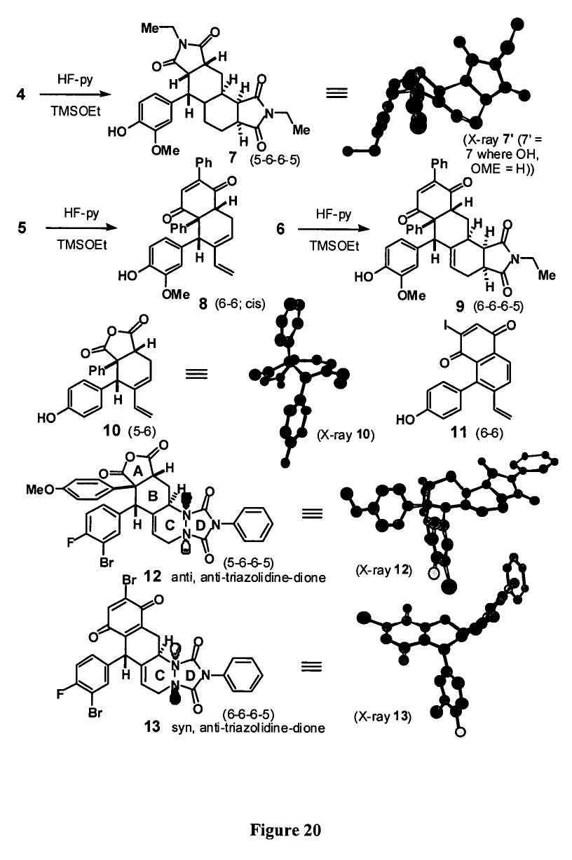

Macrobead-loaded triene 3 (FIG. 18) was used to assess the reactivity and stereoselectivity of 53 disubstituted- and 44 tri- or tetrasubstituted cyclic dienophiles. In earlier pathway-development studies, we had ascertained that non-cyclic dienophiles (acyclic dienophiles tested: trans-β-nitrostyrene, dimethyl maleate, and dimethyl fumarate). afforded stereoisomeric mixtures of double cycloadducts, whereas cyclic dienophiles yielded products stereoselectively. Spectroscopic analyses of single and double cycloadducts, including X-ray crystallography in five cases, verified that the selectivity reported by Fallis and co-workers was general (HF-py-mediated cleavage of macrobead-loaded 4 resulting from 100 mg of 3-[diisopropyl(p-methoxyphenyl)silyl]propyl functionalized macrobeads yielded 32 mg (0.71 mmol/g of beads, 109 nmol/bead) of the tetracyclic product 7 (FIG. 20) (single diastereomer and 95% pure by ¹H NMR)).

An important pattern of reactivity was uncovered using 3: disubstituted dienophiles underwent double cycloaddition (c.f., 4), whereas tri- or tetrasubstituted dienophiles underwent mono cycloaddition (c.f., 5). Using the criterion of single isomer-formation (c.f., 4) in high purity from triene 3, 41 (of 53) disubstituted dienophiles (FIG. 19 middle) were selected for use in the DOS pathway. Representative members of mono-cycloadduct dienes (c.f., 5) were found to undergo stereoselective Diels-Alder reactions with a second dienophile to yield tetracycles derived from two different dienophiles (c.f., 6). Using the criteria of efficient, single isomer-production of both single and double cycloadducts (c.f., 5 and 6), 22 (of 44) tri- or tetrasubstituted dienophiles (FIG. 19 bottom) were selected for use in the DOS pathway. These dienophiles, which “interrupt” the double Diels-Alder process, provide a key skeleton-diversifying branch in the DOS pathway. Combinations of the selected skeletal building blocks are calculated to produce a maximum of 29,400 distinct compounds (800 dienes (40 aldehydes×20 dienophiles), 2640 tetracycles from interrupted D-A with 1,2,4-triazoline-3,5-diones (40 aldehydes×22 dienophiles×3 disubstituted dienophiles), 24,320 tetracycles from interrupted D-A with maleimides (40 aldehydes×16 dienophiles×38 disubstituted dienophiles), and 1640 tetracycles from consecutive D-A (40 aldehydes×41 disubstituted dienophiles)).

Approximately 88,200 macrobeads (Burgess, K.; Liaw, A. I.; Wang, N. J. Med. Chem. 1994, 37, 2985-2987; incorporated herein by reference) were divided into 40 equal portions and loaded with the 40 aldehydes described above in separate reaction vessels. The individual vessels of aldehyde-loaded macrobeads (c.f., 1) were tagged with diazo-based electrophoretic reporters using a binary code (Ohlmeyer, M. J. H.; Swanson, R. N.; Dillard, L. W.; Reader, J. C.; Asouline, G.; Kobayashi, R.; Wigler, M.; Still, W. C. Proc. Natl. Acad. Sci. U.S.A. 1993, 90, 10922-10926; Nestler, H. P.; Bartlett, P. A.; Still, W. C. J. Org. Chem. 1994, 59, 4723-4724; each of which is incorporated herein by reference), pooled, and converted to triene-loaded macrobeads as described above (c.f., 3). The tagged and pooled triene-containing macrobeads were divided into 23 portions. One portion was recombined later with the dienes prepared below for the consecutive Diels-Alder cycloaddition (c.f., 3 to 4). Twenty-two portions were reacted individually, using optimized conditions, with 22 dienophiles (FIG. 19 bottom). Each segregated collection of macrobeads was tagged using additional reporters. The 22 vessels containing single cycloadducts (c.f., 5) were pooled and subsequently divided into 4×41 portions (the 41 portions were grouped into 4 sets since we found that the subsequent Diels-Alder reactions fell into four different optimal reaction conditions depending on the reactivity of the second dienophile). A 42^{nd} portion was set aside to be combined with the collection of tetracyclic compounds, thus ensuring the presence of bicyclic dienes (c.f., 5) in the final collection of products. The 4×41 vessels were treated individually with the 41 dienophiles (using 4 different conditions) that undergo the second cycloaddition (FIG. 19 middle) and tagged using additional reporters. The pooled, 88,200 encoded macrobeads serve to segregate a high percentage of the theoretical 29,400 compounds prior to automated preparation of stock solutions.

Quality control efforts during the pathway development phase of this research identified the reaction partners expected to undergo efficient and predictable outcomes, but they also revealed reactivity patterns that further diversified the skeletons of the products of this DOS pathway (FIG. 20 and 21). Whereas macrobead-bound trienes (c.f., 3) reacted with tri- and tetrasubstituted dienophiles to yield the expected bicycles of structural types S1 and S2 (verified in 10 and 8), they reacted with halogenated dienophiles to yield structural types S3, S9, and S10. These latter compounds result from cycloadditions followed by dehydrohalogenation—S3 (verified in 11) by dehydroiodination and S9-10 (verified in 13) by dehydrobromination (dehydrohalogenation was facilitated with strontium carbonate (Pearlman, B. A.; McNamara, J. M.; Hasan, I.; Hatakeyama, S.; Sekizaki, H.; Kishi, Y. J. Am. Chem. Soc. 1981, 103, 4248-4251; incorporated herein by reference) when maleimides were used as the second dienophile). Macrobead-bound dienes of S1 react with maleimides to yield the expected tetracycle of S4 (verified in 7′), but they react with 4-phenyl-1,2,4-triazoline-3,5-dione (and presumably related dienophiles) to yield products having anti, anti- and syn, anti-trans-fused C-D ring junctions as in S5 and S10 (verified in 12 and 13). Extending these observations to the possible combinations of dienophile building blocks suggests that at least 10 different skeletons (Blackwell, H. E.; Perez, L.; Stavenger, R. A.; Tallarico, J. A.; Eatough, E. C.; Foley, M. A.; Schreiber, S. L. Chem. Biol. 2001, 8, 1167-1182; Clemons, P. A.; Koehler, A. N.; Wagner, B. K.; Sprigings, T. G.; Spring, D. R.; King, R. W.; Schreiber, S. L.; Foley, M. A. Chem. Biol. 2001, 8, 1183-1195; each of which is incorporated herein by reference) will be represented among the 29,400 anticipated products.

Our first step in analyzing purity and identity of these products entailed the random selection of fifty macrobeads from the final pool. Products were eluted from the macrobeads with HF-py (and then TMSOEt), diluted to 10 mM stock solutions (DMF), and analyzed by LC/MS and stock solution decoding (Stavenger, R. A.; Schreiber, S. L. Angew. Chem., Int. Ed. 2001, 40, 3417-3421; Blackwell, H. E.; Perez, L.; Schreiber, S. L. Angew. Chem., Int. Ed. 2001, 40, 3421-3425; each of which is incorporated herein by reference). These data revealed acceptable levels of purity and structures consistent with expectations. Our second step in post-synthesis quality control was performed following both full arraying of all macrobeads and automated stock solution preparation.

The 88,200 individual macrobeads were first arrayed into 384-well microtiter plates using a vacuum-based bead arrayer to entrain 352 beads in an equal number of wells (two columns of wells from each plate were left empty to accommodate controls used in subsequent assays) (Blackwell, H. E.; Perez, L.; Stavenger, R. A.; Tallarico, J. A.; Eatough, E. C.; Foley, M. A.; Schreiber, S. L. Chem. Biol. 2001, 8, 1167-1182; incorporated herein by reference). Microtiter plates containing one bead per well were then subjected to a robotic cleavage process, in which each well was treated with 20 μl HF-py cocktail (5% HF-py, 5% py in THF) delivered using a ceramic pump. After 300 min at room temperature, each cleavage reaction was quenched with 20 μl TMSOEt (TMSOEt was used as the quenching reagent instead of previously reported TMSOMe to minimize cross-contamination in the 386-well microtiter plate) for 30 min, evaporated and eluted from beads with three 30 μl DMF washes. DMF eluates were pooled into fresh 384-well “mother plates,” each of which was mapped into five “daughter plates” by volumetric transfer using a Hydra384 syringe-array robot (50% of stock solution for cell-based assays, 20% for small molecule microarrays 2×10% for compound archiving, and 10% for chemical analysis) (Clemons, P. A.; Koehler, A. N.; Wagner, B. K.; Sprigings, T. G.; Spring, D. R.; King, R. W.; Schreiber, S. L.; Foley, M. A. Chem. Biol. 2001, 8, 1183-1195; incorporated herein by reference).

Currently, 150 microtiter plates (52,800 single compound-containing stock solutions, approximately 2 theoretical copies) have been arrayed, and 61 microtiter plates (21,472 compounds, 73% of a theoretical copy) have been formatted into “daughter plates”. For post-automated formatting, quality control (QC) analysis, we again used LC/MS and stock solution decoding (Blackwell, H. E.; Perez, L.; Schreiber, S. L. Angew. Chem., Int. Ed. 2001, 40, 3421-3425; incorporated herein by reference). The structures of 88 out of 100 samples were inferred successfully by LC/MS and GC decoding. The structures of the remaining 12 were inferred by GC decoding, but could not be confirmed by LC/MS.

Analysis of the purity of resulting stock solutions and their performance in both protein-binding and phenotypic assays has revealed that the overall process is sufficient for identifying novel small molecules having specific and potent protein-binding and cellular activities.

Experimentals

General. All commercially available materials were used without further purification unless otherwise noted. All solvents were dispensed from a solvent purification system wherein solvents are passed through packed columns (THF, Et₂O, CH₃CN, and CH₂Cl₂: dry neutral alumina; hexane, benzene, and toluene: dry neutral alumina and Q5 reactant; DMF: activated molecular sieves). All reactions were performed under dry N₂ unless otherwise indicated. Solution phase reactions were monitored by analytical thin-layer chromatography performed using indicated solvent on E. Merck silica gel 60 F₂₅₄ plates (0.25 mm). Compounds were visualized by staining the plates with a cerium sulfate-ammonium molybdate solution followed by heating. Flash column chromatography was performed using the indicated solvent on E. Merck silica gel 60 (40-63 m). Yields refer to chromatographically and spectroscopically pure compounds except as otherwise noted. Infrared spectra were recorded either as a thin film on NaCl plates or as a KBr disk on a Nicolet 5PC FT-IR spectrometer with internal referencing. Absorption maxima (vₘₐₓ) are reported in wavenumbers (cm⁻¹). NMR (¹H, ¹³C) spectra were recorded on Varian Mercury400 (400 MHz for ¹H), and Varian Unity/Inova500 (500 MHz for ¹H, ¹³C) spectrometers. Chemical shifts (δ_{H}) are quoted in ppm and referenced to CDCl₃ (¹H-NMR, 7.26; ¹³C-NMR, 77.0, center line). Low resolution mass spectra were obtained with JEOL AX-505H, SX-102A (CI/EI), Micromass Platform II and LCT (APCI/ES/LCMS) spectrometers. Only molecular ions, fractions from molecular ions and other major peaks are reported. High resolution mass spectra were obtained with Micromass LCT (ES) spectrometer, and reported mass values are within the error limits of ±5 ppm mass unit. X-ray crystallographic data were collected using a Bruker SMART CCD (charge coupled device) based diffractometer equipped with LT-2 low-temperature apparatus operating at 213 K.

Experimental Procedures (Solution-phase):

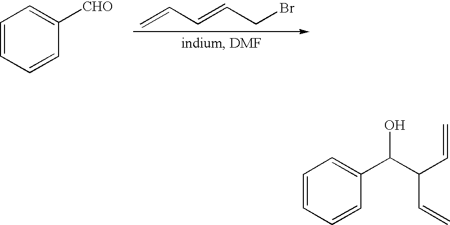

Synthesis of bishomoallylic alcohol, 1-Phenyl-2-vinylbut-3-en-1-ol. This reaction was slightly modified from previous reported procedure (see ref. 5, 7). Indium powder (100 mesh, Aldrich, 282 mg, 2.46 mmol) was added in portions to a mixture of 5-bromo-1,3-pentadiene (657 mg, 4.47 mmol) and benzaldehyde (227 μL, 2.23 mmol) in DMF (2.23 mL) at 0° C. The resulting mixture was stirred for 5 h as the temperature of the ice bath slowly rose to 10° C. The reaction was diluted with CH₂Cl₂ (15 mL) and then added to diethyl ether (190 mL). The resulting turbid mixture was filtered through a pad of silica gel. The silica gel was washed with additional ether and the filtrate was concentrated. The product (344 mg, 89%) was purified by flash column chromatography on silica gel using 10:1 hexane/ethyl acetate: R_{f} 0.25 (10:1 hexanes:EtOAc); ¹H NMR (500 MHz, CDCl₃) δ 7.36-7.27 (m, 5H), 5.86 (ddd, J=17.5, 11.0, 8.5 Hz, 1H), 5.70 (ddd, J=17.0, 10.5, 7.0 Hz, 1H), 5.25 (dd, J=10.0, 1.5 Hz, 1H), 5.19 (d, J=17.0 Hz, 1H), 5.06 (d, J=10.5 Hz, 1H), 5.02 (d, J=17.0 Hz, 1H), 4.59 (dd, J=7.0, 2.5 Hz, 1H), 3.11 (q, J=7.5 Hz, 1H), 2.30 (br s, 1H); ¹³C NMR (125 MHz, CDCl₃) δ 141.7, 136.8, 136.7, 128.1, 127.6, 126.8, 118.2, 117.0, 76.1, 56.1; FT-IR (thin film) 3420, 3078, 3029, 2878, 2360, 2337, 1635, 1453, 1416, 1302, 1194, 1038, 999, 918, 721, 700 cm⁻¹; HRMS (CI, NH₃) calcd for C₁₂H₁₈NO 192.1388 m/z (M+NH₄)⁺; observed 192.1383 (2.6 ppm error).

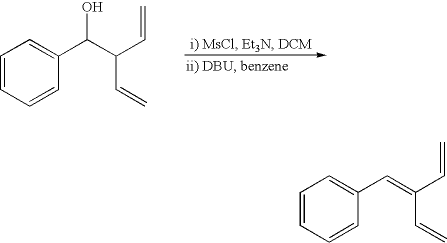

Synthesis of triene, (2-Vinylbuta-1,3-dienyl)benzene. To a solution of the bishomoallylic alcohol (2.28 g, 13.1 mmol) and triethylamine (2.74 mL, 19.7 mmol) in CH₂Cl₂ (100 mL) at −50° C. was added mesyl chloride (1.35 mL, 17.5 mmol) dropwise. The resulting mixture was allowed to warm to −30° C. over 45 min. The reaction was poured into 1:1 saturated NaHCO₃/H₂O (100 mL). The aqueous layer was extracted thrice with diethyl ether (100 mL). Combined organic solution was dried over Na₂SO₄/MgSO₄ and concentrated. A solution of the crude mesylate in dry benzene (100 mL) was treated with 1,8-diazabicyclo[5.4.0]undec-7-ene (2.4 mL, 15.7 mmol) and gently heated at 44° C. for 3 h. Flash column chromatography of the concentrated crude mixture employing hexane furnished the triene (1.89 g, 93%): R_{f} 0.59 (50:1 hexanes:EtOAc); ¹H NMR (500 MHz, CDCl₃) δ 7.89-7.32 (m, 4H), 7.26-7.23 (m, 1H), 6.72 (dd, J=17.5, 11.0 Hz, 1H), 6.66 (s, 1H), 6.57 (ddd, J=17.0, 11.0, 1.0 Hz, 1H), 5.55 (dd, J=17.0, 1.5 Hz, 1H), 5.46 (dd, J=17.5, 1.0 Hz, 1H), 5.36 (dt, J=11.5, 1.5 Hz, 1H), 5.22 (dd, J=11.0, 1.0 Hz, 1H); ¹³C NMR (125 MHz, CDCl₃) δ 137.9, 137.8, 137.1, 133.5, 129.7, 129.5, 128.1, 127.1, 118.3, 116.1; IR (thin film) 3853, 3747, 3673, 3084, 2361, 2338, 1699, 1651, 1558, 1540, 989, 912, 695 cm⁻¹; HRMS (EI) calcd for C₁₂H₁₂ 156.0939 m/z (M)⁺; observed 156.0941 (1.3 ppm error).

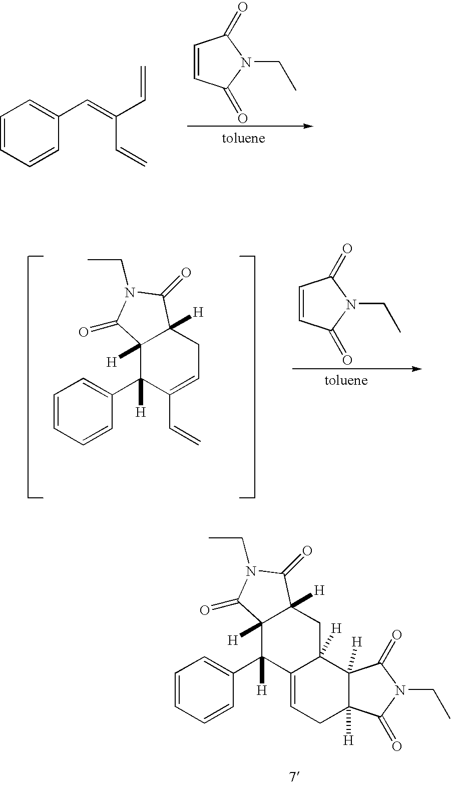

Synthesis of tetracyclic compound, 2,8-Diethyl-6-phenyl-3α,4,6,6α,9α,10,10α,10β-octahydro-isoindolo[5,6ε]isoindole-1,3,7,9-tetraone (7′). N-ethylmaleimide (24.3 mg, 0.194 mmol) was added to a solution of the triene (30.4 mg, 0.194 mmol) in toluene (490 μL) at room temperature and the resulting solution was stirred overnight. Thin layer chromatography indicated three spots corresponding to the starting triene, diene, and the tetracyclic product with the complete consumption of N-ethylmaleimide. More N-ethylmaleimide (36.4 mg, 0.291 mmol) was added and the reaction was continued for an additional day. Flash column chromatography (50:1 CH₂Cl₂:MeOH) of the crude concentrate of the reaction provided the desired tetracyclic product 7′ as a single diastereomer by ¹H NMR (75 mg, 95%): R_{f} 0.35 (50:1 CH₂Cl₂:MeOH); ¹H NMR (500 MHz, CDCl₃) δ 7.35-7.26 (m, 5H), 5.48 (br dd, J=6.5, 3.0 Hz, 1H), 3.86 (br s, 1H), 3.54 (q, J=7.0 Hz, 2H), 3.44 (qd, J=7.0, 1.5 Hz, 2H), 3.34 (dd, J=5.5, 2.0 Hz, 1H), 3.32 (td, J=9.0, 5.0 Hz, 1H), 3.10-3.03 (m, 3H), 2.66 (dd, J=16.0, 8.0 Hz, 1H), 2.46 (ddd, J=14.0, 4.5, 1.5 Hz, 1H), 2.26 (br dd, J=13.0, 2.0 Hz, 1H), 1.99 (br dt, J=15.5, 3.5 Hz, 1H), 1.09 (t, J=7.0 Hz, 3H), 1.02 (t, J=7.5 Hz, 3H); ¹³C NMR (125 MHz, CDCl₃) δ 179.0, 178.9, 177.6, 177.1, 139.8, 135.8, 130.4, 127.9, 127.4, 122.1, 45.7, 43.6, 43.2, 40.5, 39.6, 34.0, 33.7, 24.9, 23.3, 13.1; IR (thin film) 3853, 3747, 2978, 2942, 2361, 1694, 1405, 1349, 1227, 1130, 1034, 916, 731, 421 cm⁻¹; HRMS (TOF ES) calcd for C₂₄H₂₇N₂O₄, 407.1971 m/z (M+H)⁺; observed 407.1964 (1.7 ppm error). X-ray crystallographic data for this compound are shown as sls34t.

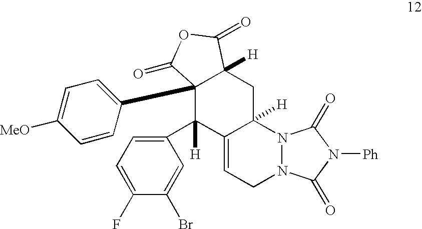

6-(3-Bromo-4-fluorophenyl)-6α-(4-methoxyphenyl)-2-phenyl-4,6,6α,9α,10,10-hexahydro-8-oxa-2,3α,10β-triazadicyclopenta[α,γ]naphthalene-1,3,7,9-tetraone (12): R_{f} 0.31 (50:1 CH₂Cl₂:MeOH); ¹H NMR (500 MHz, CDCl₃) δ 7.53-7.27 (m, 4H), 7.40 (tt, J=7.0, 2.0 Hz, 1H), 7.32 (dd, J=6.5, 2.0 Hz, 1H), 7.20(d, J=8.5 Hz, 2H), 7.09 (dq, J=9.0, 2.5 Hz, 1H), 7.01 (t, J=8.5 Hz, 1H), 6.88 (d, J=9.5 Hz, 2H), 5.62 (br s, 1H), 4.64 (br d, J=12.5 Hz, 1H), 4.25 (dq, J=14.0, 3.0 Hz, 1H), 4.15 (s, 1H), 4.01 (dq, J=17.0, 2.5 Hz, 1H), 3.92 (d, J=6.5 Hz, 1H), 3.84 (dd, J=14.0, 4.5 Hz, 1H), 3.81 (s, 3H), 2.39 (td, J=13.5, 7.5 Hz, 1H); ¹³C NMR (125 MHz, CDCl₃) δ 171.1, 170.7, 159.7, 153.1, 151.8, 135.8, 133.5, 132.4, 130.7, 130.5, 129.2, 128.4, 127.6, 125.4, 120.8, 114.6, 56.1, 55.4, 52.3, 46.9, 43.1, 26.5; FT-IR (thin film) 3853, 2747, 2673, 2361, 2338, 1782, 1715, 1702, 1507, 1419, 1256, 731, 421 cm⁻¹; HRMS (TOF ES) calcd for C₃₁H₂₄BrFN₃O₆, 632.0832 m/z (M+H)⁺; observed 632.0828 (0.6 ppm error). X-ray crystallographic data for this compound are shown as sls55t.

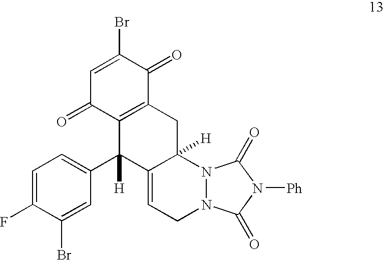

9-Bromo-6-(3-bromo-4-fluorophenyl)-2-phenyl-4,6,11,11α-tetrahydro-2,3α,11β-triaza-cyclopenta[α]anthracene-1,3,7,10-tetraone (13): R_{f} 0.41 (50:1 CH₂Cl₂:MeOH); ¹H NMR (500 MHz, CDCl₃) δ 7.51-5.46 (m, 4H), 7.39 (br t, J=7.0 Hz, 1H), 7.35 (dd, J=6.5, 2.0 Hz, 1H), 7.29 (s, 1H), 7.08 (t, J=8.5 Hz, 1H), 7.04 (dq, J=9.0, 2.5 Hz, 1H), 6.13 (s, 1H), 4.86 (s, 1H), 4.55 (t, J=8.0 Hz, 1H), 4.34 (dd, J=16.5, 4.5 Hz, 1H), 4.06 (d, J=16.5 Hz, 1H), 3.75 (dd, J=18.5, 6.5 Hz, 1H), 2.58 (ddd, J=19.0, 10.0, 1.5 Hz, 1H); ¹³C NMR (125 MHz, CDCl₃) δ 182.5, 178.2, 152.6, 151.5, 141.5, 140.8, 138.1, 137.6, 137.0, 133.5, 132.1, 130.7, 132.1, 130.7, 129.2, 128.4, 127.5, 127.4, 125.4, 117.1, 116.9, 116.3, 48.1, 45.8, 43.3, 29.4; FT-IR (thin film) 3853, 3747, 2361, 2338, 1713, 1651, 1494, 1419, 1301, 1246, 1135, 764 cm⁻¹; HRMS (TOF ES) calcd for C₂₇H₁₇Br₂FN₃O₄, 613.9539 m/z (M+H+2)⁺; observed 613.9551 (1.9 ppm error, most intense isotope ion). X-ray crystallographic data for this compound are shown as sls50t.

Experimental Procedures (Solid-phase): Synthetic route-validation on solid-support.

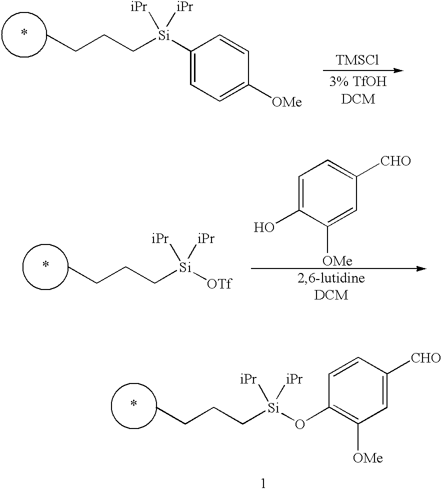

Loading hydroxyaldehyde. 3-[Diisopropyl(p-methoxyphenyl)silyl]propyl functionalized beads (500 mg) in a 40 mL vial was flushed with nitrogen for a few minutes and allowed to swell in CH₂Cl₂ (5 mL) for 15 min. Beads were then treated with chlorotrimethylsilane (180 μL, 2.84 mmol) for 15 min. The beads were filtered and washed thrice with CH₂Cl₂ (2 min each time) and suspended in a 3% (v/v) solution of trifluoromethanesulfonic acid (12.6 mL, 4.26 mmol) in CH₂Cl₂ for 15 min during which the reaction vial was shaken periodically. The beads were filtered and washed thrice with CH₂Cl₂ (2 min. each time) and left suspended in CH₂Cl₂ (2 mL). Freshly distilled 2,6-lutidine (682 μL, 5.68 mmol) was added followed by vanillin (324 mg, 2.13 mmol) solution in CH₂Cl₂ (3 mL). The vial was then shaken for 4 h. The beads were then filtered, suspended and rinsed with CH₂Cl₂ (4×5 min each time), and dried under high vacuum overnight. The loaded beads weighed 525.8 mg, one fifth of which (105.2 mg) was swelled in THF (2 mL) for 10 min and treated with HF-py (100 μL) and pyridine (100 μL). The reaction Eppendorf® tube was tumbled for 2 h. Ethoxytrimethylsilane (1 mL) was added and the Eppendorf® tube was tumbled for another hour. The beads were filtered and washed. Collected filtrate was concentrated and azeotroped twice with CH₃CN to remove pyridine. The crude yield was 23.0 mg (1.51 mmol/g of loading, >95% pure by ¹H NMR): R_{f} 0.53 (20:1 CH₂Cl₂:MeOH); ¹H NMR (500 MHz, CDCl₃) δ 9.82 (s, 1H), 7.43-7.41 (m, 2H), 7.04 (d, J=8.5 Hz, 1H), 6.33 (br s, 1H), 3.96 (s, 3H); ¹³C NMR (125 MHz, CDCl₃) δ 190.9, 151.7, 147.1, 129.8, 127.5, 114.4, 108.7, 56.1; FT-IR (thin film) 3240, 2921, 2849, 2361, 1670, 1652, 1576, 1558, 1507, 1457, 1433, 1265, 1149, 1118 cm⁻¹; LRMS (TOF ES) calcd for C₈H₉O₃ 153.06 m/z (M+H)⁺; observed 153.05.

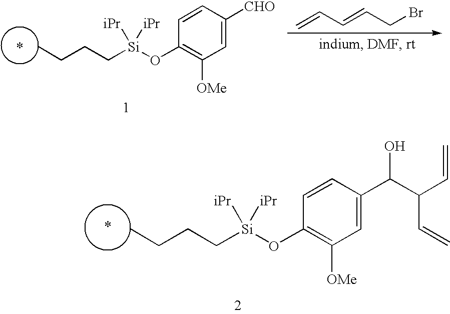

Synthesis of bishomoallylic alcohol, 4-(1-Hydroxy-2-vinyl-but-3-enyl)-2-methoxy-phenol (2): Beads 1 (420.6 mg, resulting from 400 mg of p-methoxyphenylsilane beads) were swelled in DMF (1.5 mL) for 15 min. 5-Bromo-1,3-pentadiene (825 mg, 5.86 mmol) and indium powder (Aldrich, −100 mesh, 326 mg, 2.84 mmol) were added to the reaction vial, which was shaken at room temperature for 24 h. The beads were washed with tumbling using DMF (2×30 min), THF (30 min), and CH₂Cl₂ (3×20 min) and dried under high vacuum overnight. The beads 2 weighed 455.1 mg. A quarter of the beads were cleaved as above employing HF-py. The crude yield for the cleaved alcohol was 17.9 mg (0.81 mmol/g, 54% yield). Crude ¹H NMR indicated some pentadienylation of the alkoxide intermediate. This phenomenon occurs with molar ratio higher than 2:1 5-bromo-1,3-pentadiene/indium in this reaction, therefore, it is important to use a 1.8:1 molar ratio of penta-2,4-dienyl bromide/indium. O-alkylation was also facilitated by strictly anhydrous reaction condition. For this reaction, anhydrous DMF was used as solvent, but the reaction was performed under open air: R_{f} 0.51 (1:1 hexanes:EtOAc); ¹H NMR (500 MHz, CDCl₃) δ 6.87 (s, 1H), 6.86 (d, J=5.5 Hz, 1H), 6.79 (dd, J=7.5, 1.5 Hz, 1H), 5.86 (ddd, J=17.5, 10.0, 8.0 Hz, 1H), 5.66 (ddd J=17.5, 10.0, 7.0 Hz, 1H), 5.61 (br s, 1H) 5.25 (d, J=11.0 Hz, 1H), 5.20 (d, J=17.0 Hz, 1H), 5.04 (d, J=11.0 Hz, 1H), 5.00 (d, J=17.0 Hz, 1H), 4.50 (d, J=8.0 Hz, 1H), 3.89 (s, 3H), 3.70 (q, J=7.5 Hz, 1H), 2.19 (br s, 1H); ¹³C NMR (125 MHz, CDCl₃) δ 146.4, 145.1, 137.0, 136.8, 133.8, 120.1, 118.3, 116.9, 113.8, 109.1, 76.1, 56.2, 55.9; FT-IR (thin film) 3853, 3747, 3649, 3445, 2361, 2338, 1651, 1558, 1472, 1270, 668 cm⁻¹; HRMS (TOF ES) calcd for C₁₃H₁₅O₂, 203.1072 m/z (M−H₂O+H)⁺; observed 203.1064 (3.9 ppm error, only dehydrated compound was observed even under low energy).

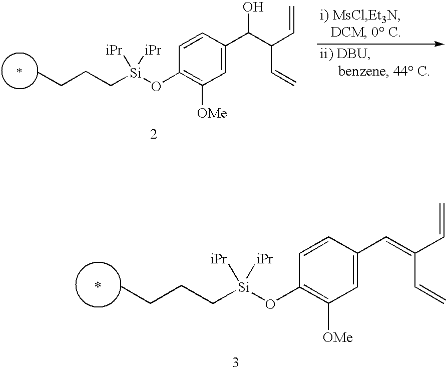

Synthesis of triene, 2-Methoxy-4-(2-vinylbuta-1,3-dienyl)-phenol (3): Beads 2 (341.3 mg, resulting from 300 mg ofp-methoxyphenylsilane beads) were swelled in CH₂Cl₂ (4 mL) and cooled to 0° C. Triethylamine (608 μL, 4.36 mmol) and methanesulfonyl chloride (300 μL, 3.88 mmol) were added and the reaction was allowed to persist for 3 h at 0° C. The reaction solution was removed and the beads were washed with CH₂Cl₂ (4×5 min each time) and suspended in benzene for 30 min. After removal of benzene, the beads were put under vacuum for 30 min and re-suspended in benzene (4 mL). 1,8-Diazabicyclo[5.4.0]undec-7-ene (652 μL, 4.36 mmol) was added and the resulting reaction was heated at 44° C. for 12 h. The beads were washed with CH₂Cl₂ (4×2 h each time) and put under vacuum overnight. The triene beads 3 weighed 324.3 mg. One third of the beads (108.1 mg) were cleaved to afford the cleaved triene (19.8 mg, 0.98 mmol/g, 65% over three steps): R_{f} 0.50 (3:1 hexanes:EtOAc); ¹H NMR (500 MHz, CDCl₃) δ 6.92-6.87 (m, 3H), 6.70 (dd, J=17.5, 11.5 Hz, 1H), 6.57 (s, 1H), 6.53 (dd, J=17.0, 10.5 Hz, 1H), 5.65 (s, 1H), 5.50 (dd, J=17.0, 1.0 Hz, 1H), 5.43 (dd, J =18.5, 1.5 Hz, 1H), 5.35 (d, J=11.0 Hz, 1H), 5.17 (d, J=11.0 Hz, 1H), 3.88 (s, 3H); ¹³C NMR (125 MHz, CDCl₃) δ 145.0, 138.0, 136.4, 133.8, 129.5, 123.4, 118.0, 115.4, 114.1, 112.1, 55.9; FT-IR (thin film) 3853, 3747, 2922, 2850, 2361, 2338, 1509, 1269, 1206,911,420 cm⁻¹; HRMS (TOF ES) calcd for C₁₃H₁₅O₂, 203.1072 m/z (M+H)⁺; observed 203.1070 (0.9 ppm error)

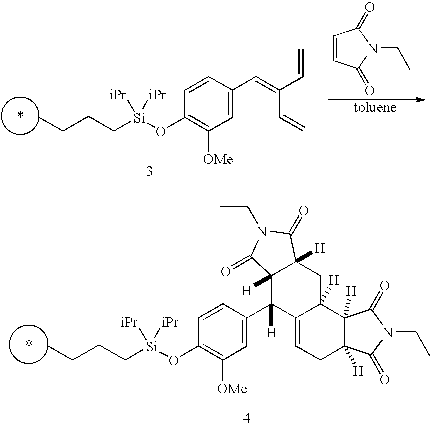

Synthesis of tetracyclic compound, 2,8-Diethyl-6-(4-hydroxy-3-methoxyphenyl)-3α,4,6,6α,9α,10,10α, 10β-octahydro-isoindolo[5,6-ε]isoindole-1,3,7,9-tetraone (7) via 4. Two sets of beads with immobilized triene 3 (108.1 mg each) were swollen in toluene (1.5 mL) for 15 min, followed by the addition of N-ethylmaleimide (178 mg, 1.42 mmol). One vial was agitated for 24 h and the other 72 h, after which beads were washed using CH₂Cl₂ (3×20 min) and dried under high vacuum overnight. Upon cleavage of the product using HF-py, the tetracyclic compound 7 (28.4 mg, 0.63 mmol/g, 42% overall for 1 d of the Diels-Alder reaction; 32 mg, 0.71 mmol/g, 47% overall for 3 d). The crude product was subjected for LC/MS to measure the yield of 1-day reaction sample (84.7%) and 3-day reaction sample (81.5%). Mass results were satisfactory in both run. Crude ¹H NMR indicated single diastereomeric product. The major contaminant based on ¹H NMR was the byproduct which was originated from pentadienylation of the alkoxide intermediate. The crude samples were purified by flash column chromatography (50:1 CH₂Cl₂:MeOH) to yield 7 (26 mg, 76.9%) as a clear oil: R_{f} 0.25 (50:1, CH₂Cl₂:MeOH); ¹H NMR (500 MHz, CDCl₃); δ 7.11(d, J=2.0 Hz, 1H), 6.82 (d, J=8.0 Hz, 1H), 6.73 (dd, J=8.0, 2.0 Hz, 1H), 5.61 (s, 1H), 5.46 (dd, J=7.5, 2.5 Hz, 1H), 3.87 (s, 3H), 3.57 (s, 1H), 3.55 (qd, J=7.5, 2.5 Hz, 2H), 3.44 (qd, J=7.0, 1.5 Hz, 2H), 3.34 (dd, J=6.5, 2.5 Hz, 1H), 3.27 (dd, J=9.0, 5.0 Hz, 1H), 3.05 (m, 3H), 2.66 (dd, J=15.0, 7.5 Hz, 1H), 2.45 (ddd, J=2.0, 5.0, 14 Hz, 1H), 2.24 (dd, J=2.0, 13.0 Hz, 1H), 2.00 (m, 1H), 1.10 (t, J=7.5 Hz, 3H), 1.02 (t, J=7.0 Hz, 3H); ¹³C NMR (125 MHz, CDCl₃) δ 179.3, 179.2, 177.9, 177.7, 146.2, 145.1, 140.8, 128.0, 123.7, 122.0, 114.1, 113.7, 56.2, 45.9, 44.3, 43.4, 40.9, 39.8, 34.3, 33.95, 33.91, 25.2, 23.69, 13.41, 13.37; FT-IR (thin film): 3051, 2987, 2681, 2516, 2405, 2358, 2335, 2301, 2251, 1696, 1417, 1265, 909 cm⁻¹; HRMS (TOF ES) calcd for C₂₅H₂₉N₂O₆ 453.2025 m/z (M+H)⁺; observed 453.2030 (1.1 ppm error).

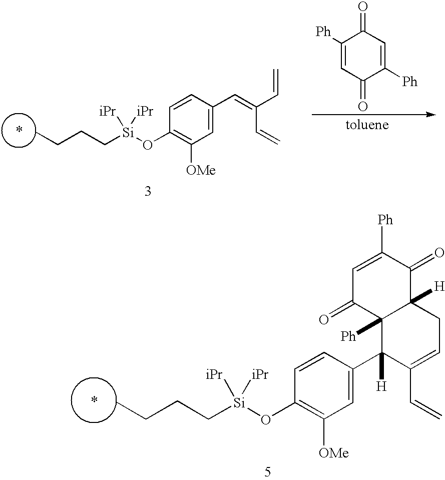

Synthesis of diene, 5-(4-Hydroxy-3-methoxy-phenyl)-2,4α-diphenyl-6-vinyl-4α,5,8,8α-tetrahydro[1,4]naphthoquinone (8) via 5: R_{f} 0.34 (CH₂Cl₂); ¹H NMR (500 MHz, CDCl₃) δ 7.39-7.33 (m, 4H), 7.32-7.30 (m, 2H), 7.28-7.26 (m, 2H), 7.00 (s, 1H), 6.98 (s, 1H), 6.73 (d, J=8.0 Hz, 1H), 6.46 (dd, J=8.5, 2.0 Hz, 1H), 6.40 (s, 1H), 6.33 (s, 1H), 6.31 (dd, J=17.5, 10.5 Hz, 1H), 6.04 (t, J=3.5 Hz, 1H), 5.41 (s, 1H), 4.99 (d, J=17.5 Hz, 1H), 4.95 (d, J=11.0 Hz, 1H), 4.50 (s, 1H), 3.58 (d, J=7.0 Hz, 1H), 3.54 (s, 3H), 3.32 (dd, J=20.0, 4.5 Hz, 1H), 1.76 (dd, J=20.0, 7.0 Hz, 1H); ¹³C NMR (125 MHz, CDCl₃) δ 202.9, 197.0, 149.4, 146.4, 144.9, 142.7, 137.3, 136.2, 135.1, 133.0, 130.0, 129.8, 129.7, 129.2, 128.9, 128.6, 128.3, 128.2, 128.1, 127.7, 127.2, 126.8, 123.5, 123.0, 114.4, 113.4, 112.8, 61.6, 55.5, 52.0, 45.4, 21.1; IR (thin film) 3853, 3747, 3673, 3445, 2361, 1771, 1698, 1651, 1558, 1507, 1457, 1272 cm⁻¹; HRMS (TOF ES) calcd for C₃₁H₂₇O₄, 463.1909 m/z (M+H)⁺; observed 463.1912 (0.6 ppm error).

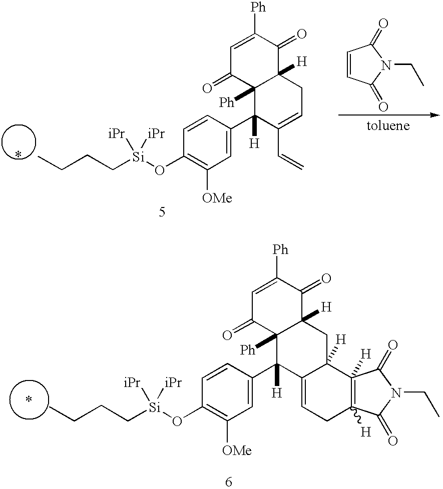

Synthesis of Tetracyclic compound, 2-Ethyl-6-(4-hydroxy-3-methoxyphenyl)-6α,9-diphenyl-3α, 4,6,6α,10α,11,11α,11β-octahydronaphtho[2,3-e]isoindole-1,3,7,10-tetraone (9) via 6: R_{f} 0.23 (50:1 CH₂Cl₂:MeOH); ¹H NMR (500 MHz, CDCl₃) δ 7.38-7.30 (m, 4H), 7.19 (dd, J=7.5, 1.5 Hz, 2H), 7.15-7.11 (m, 3H), 6.94 (dd, J=7.0, 1.5 Hz, 2H), 6.62 (s, 1H), 6.61 (s, 1H), 6.56 (d, J=8.5 Hz, 1H), 6.37 (d, J=8.5 Hz, 1H), 5.54 (dd, J=4.5, 3.0 Hz, 1H), 5.41 (s, 1H), 4.20 (dd, J=9.0, 6.5 Hz, 1H), 3.64 (s, 3H), 3.53-3.44 (m, 2H), 3.16 (dd, J=9.0, 5.5 Hz, 1H), 3.10 (t, J=7.5 Hz, 1H), 2.88 (m, 1H), 2.71 (t, J=8.0 Hz 1H), 2.68 (t, J=6.5 Hz, 1H), 2.52 (dt, J=15.0, 9.5 Hz, 1H), 2.19 (m, 1H), 1.06 (t, J=7.5 Hz, 3H); ¹³C NMR (125 MHz, CDCl₃) δ 201.3, 198.5, 179.2, 178.2, 147.0, 145.7, 144.2, 141.3, 140.5, 137.2, 132.7, 130.8, 129.9, 128.9, 128.5, 128.3, 127.5, 127.2, 124.7, 123.5, 114.3, 113.5, 62.2, 56.1, 55.7, 55.1, 45.6, 40.3, 33.6, 31.9, 25.4, 25.1, 13.1; IR (thin film) 2937, 2360, 1695, 1516, 1227, 910 cm⁻¹; HRMS (TOF ES) calcd for C₃₇H₃₄NO₆, 588.2386 m/z (M+H)⁺; observed 588.2371 (2.5 ppm error)

Validation of Building Blocks B and C: Scaffold Diversity.

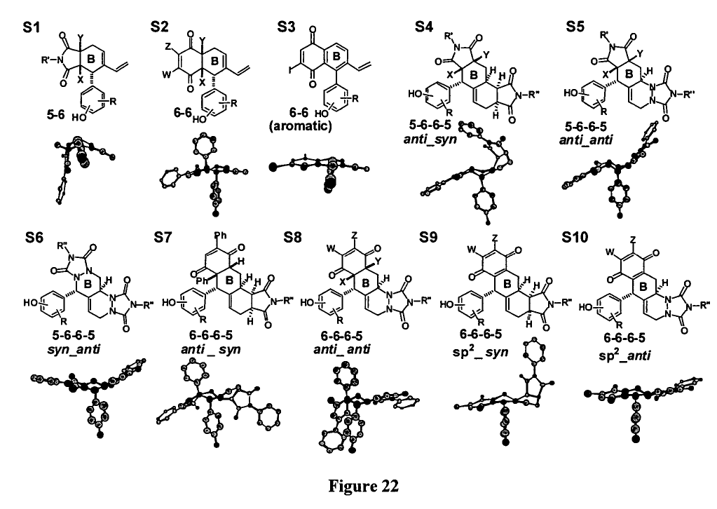

The reaction pathway developed using solution-phase chemistry was translated into the 500-600 micron polystyrene macrobead-supported chemistry platform. All the Diels-Alder reactions on solid-phase were optimized (time, temperature) until there was complete consumption of triene (for diene formation and for consecutive Diels-Alder reaction) or diene (for the construction of tetracycles via interrupted consecutive Diels-Alder reaction) judged by TLC and ¹H NMR of cleaved crude product. Our quality control efforts during the pathway development phase of this research not only identified the reaction partners that are expected to undergo efficient and predictable cycloaddition, but they also revealed reactivity patterns that further diversified the skeletons of the products of this DOS pathway (FIG. 22). For example, triene 3 yielded the expected skeletons S1-2, S4, and S7, verified in bicycle 7, tetracycle 8-9 and the related bicycle 10. On the other hand, halogenated benzoquinone derivatives (building blocks B2-B4) provided different skeletons when allowed to react with trienes. ¹H NMR and LCMS data showed that diene derived from 2,6-diiodo-P-benzoquinone aromatized upon cleavage employing HF-py, providing scaffold S3. Evidently, it exists as diene before cleavage since the macrobead-supported dienes undergo Diels-Alder and concomitant dehydroiodination either with 4-phenyl-1,2,4-triazoline-3,5-dione or N-phenylmaleimide in the presence of SrCO₃ to produce tetracyclic products such as S9 and S10. Building blocks B3-4 produce S2 as a bicyclic-scaffold and S9 and S10 via a [4+2]-dehydrohalogenation-[4+2] cycloaddition sequence. Further structural diversity resulted from the dienophile 4-phenyl-1,2,4-triazoline-3,5-dione (and presumably related dienophiles), which yielded products having a trans-fused C-D ring junction as well as a trans-fused A-B ring junction, such as S5, S6, S8, and S10. Projection of these observations to the possible combinations of dienophile building blocks provides 29,400 anticipated products incorporating 10 unique scaffolds (vide infra).

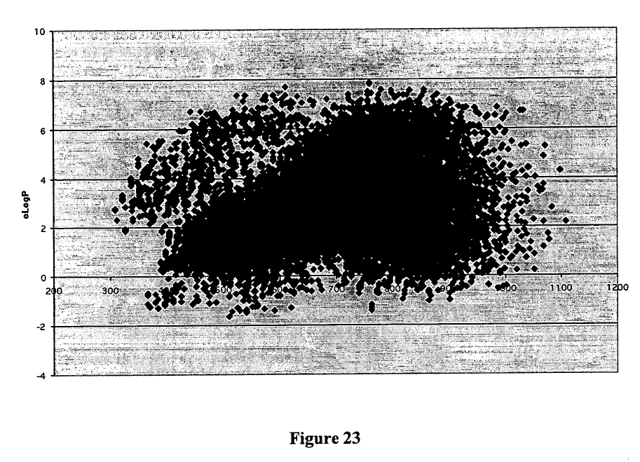

In addition to the scaffold diversity comprising 10 discrete core structures obtained via a single synthetic route, the use of diverse building blocks significantly increases the diversity of the Diels-Alder polycyclic library. Through cheminformatic efforts, we have initiated efforts to describe the multidimensional chemical descriptor space occupied by the synthetic compounds derived from the reported pathway. Using the software package QsarIS (SciVision), we generated basic quantitative information on the polycyclic library. This library has members with molecular weight ranging from 308.4 to 1107.5 (average 687.03); clogP distribution from −1.597 to 7.860 (average 2.728); number of hydrogen bond acceptors from 3 to 25 (average 11.46); and number of hydrogen bond donor from 1 to 5 (average 1.55). In FIG. 23, we demonstrate the distribution of library member using two key molecular descriptors, molecular weight and calculated partition coefficient (clogP). The ranges for calculated logP values and molecular weight suggested that the diverse structures and efficiently filled the molecular descriptor space.

TABLE 1
Quality assessment of dienes formed by the “interrupted” Diels-Alder reaction of
macrobead-supported triene 3 with building blocks B. (Diene arising from BBB2 is
aromatized, which was confirmed by ¹H NMR and MS: see S3 in FIG. 22. Dienes derived
from building blocks B4 and B5 appear as dienes illustrated by S2 in FIG. 22 in their ¹H
NMR, yet the observed masses correspond to M-H₂Br (AP+) indicating aromatization in the
mass spectrometer. Dienes derived from 3,4-dichloromaleimdes-BBB11-14 are evidently
aromatized in the mass spectrometer: molecular ion peak were M-H₂Cl₂ (AP−) and M-HCl₂
(AP+). Their ¹H NMR illustrated dienes of scaffold S1.) Dienes with complex data were
excluded from the members of the library. Purity was based on integration of the peaks in
the chromatograph from TIC of diode array, AP−, or AP+. Common impurity peaks
throughout many samples were excluded from integration.

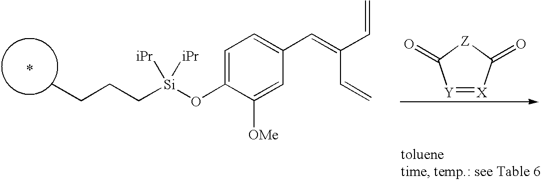

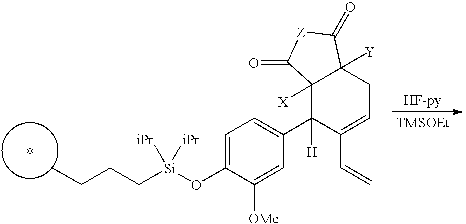

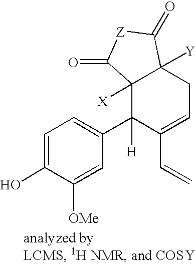

| | | | observed mass | | purity | | | |
|---|---|---|---|---|---|---|---|---|
| BBB | name | exact mass | AP− | AP+ | AP− | diode array | AP+ | ¹H NMR, COSY |
| 1 | 2,5-diphenyl-p-benzoquinone | 462.18 | 461.93 | 463.75 | | >90% | | single diastereomer |
| 2 | 2,6-diiodo-p-benzoquinone | 431.99 | | 432.6 | | | 90% | single diastereomer |
| 3 | 2,5-dibromo-1,4-benzoquinone | 384 | — | — | | N/A | | complex |
| 4 | 2-bromo-5-methyl-1,4-benzoquinone | 320.1 | | 320.9 | | | 70% | single diastereomer |
| 5 | 2-bromo-6-methyl-p-benzoquinone | 320.1 | | 321.56 | | 48% | | complex |
| 6 | 2,6-dimethylbenzoquinone(p-xyloquinone) | 338.15 | | 338.9 | | | 90% | single diastereomer |
| 7 | 2,5-dimethylbenzoquinone | 338.15 | | 339.3 | | | 90% | single diastereomer |
| 8 | trimethylquinone | 352.17 | | 352.8 | | | 80% | single diastereomer |
| 9 | duroquinone(tetramethyl-1,4-benzoquinone) | 366.18 | | 367.3 | | | 90% | single diastereomer |
| 10 | tetrachloro-1,4-benzoquinone | 445.96 | | 446.8 | | | 90% | single diastereomer |
| 11 | N-bromomethyl-2,3-dichloromaleimide | 387.01 | 386.75 | | >90% | 55% | | single diastereomer |
| 12 | 3,4-dichloro-1-methyl-2,5-dihydro-1H- | 309.1 | 308.77 | 310.1 | 92% | 87% | >90% | single diastereomer |
| | pyrrole-2,5-dione | | | | | | | |
| 13 | 2,3-dichloro-N-4-fluorophenylmaleimide | 389.11 | | 390.2 | | | 90% | single diastereomer |
| 14 | N-(pentafluorophenyl)dichloromaleimide | 461.07 | 460.85 | | 90% | 76% | | single diastereomer |
| 15 | 3-(4-Chlorophenyl)-1(4-fluoroanilino)maleimide | 518.14 | | 520.06 | | 81% | 86% | single diastereomer |
| 16 | 3-(4-Chlorophenyl)-1-[[3-chloro-5-(trifluoromethyl)- | 603.09 | 602.92 | 604.74 | | 46% | 73% | single diastereomer |
| | 2-pyridyl]amino]maleimide | | | | | | | |
| 17 | 3-phenyl-1-(4-toluidino)-1H-pyrrole-2,5-dione | 480.2 | | 481.2 | | | 90% | single diastereomer |
| 18 | 1-(2,4-dichloroanilino)-3-(4-methoxyphenyl)-1H- | 564.12 | | 565.34 | | 82% | 90% | single diastereomer |
| | pyrrole-2,5-dione | | | | | | | |
| 19 | 1-(3-chlorophenyl)-3-phenyl-2,5-dihydro-1H- | 485.14 | | 486.1 | | | >90% | single diastereomer |
| | pyrrole-2,5-dione | | | | | | | |
| 20 | 2-methyl-N-phenylmaleimide | 389.16 | | 390.71 | | 51% | 69% | single diastereomer + triene |
| 21 | 1-(3-chloro-4-fluorophenyl)-3-methyl-2,5-dihydro- | 441.11 | 440.89 | 442.43 | 73% | >90% | 44% | single diastereomer |
| | 1H-pyrrole-2,5-dione | | | | | | | |
| 22 | 1-[3-(5-chloro-3-methylbenzo[B]thiophen-2-yl)-1- | 573.15 | 572.98 | 574.77 | | | 90% | single diastereomer |
| | methyl-1H-pyrazol-5-yl]-3-methyl-2,5-dihydro-1H- | | | | | | | |
| | pyrrole-2,5-dione | | | | | | | |

TABLE 2
Quality assessment of tetracycles formed by the “interrupted” Diels-Alder reaction
of macrobead-supported triene 3 with building blocks B followed by the second [4 + 2]
reaction with N-phenyltriazolinedione. (Building blocks B1 or B6-B10 with triazolinedione
provide scaffold S8, B2-B5 with triazolinedione furnish S10, and B11-B22 with the
triazolinediones supply S5.) Purity was based on integration of the peaks in the
chromatograph from TIC of diode array, AP−, or AP+. Common impurity peaks throughout
many samples were excluded from integration.

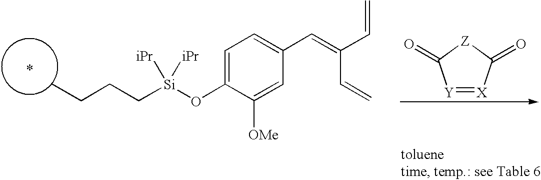

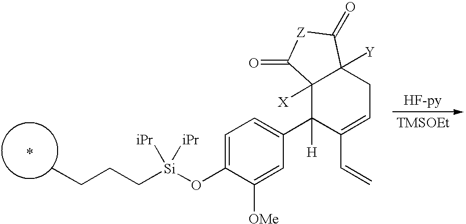

| | | | observed mass | | purity | | |
|---|---|---|---|---|---|---|---|
| BBB | name | exact mass | AP− | AP+ | diode array | AP+ | ¹H NMR, COSY |
| 1 | 2,5-diphenyl-p-benzoquinone | 637.22 | | 637.8 | | 85% | single diastereomer |
| 2 | 2,6-diiodo-p-benzoquinone | 609.04 | | 609.6 | | >90% | single diastereomer |
| 3 | 2,5-dibromo-1,4-benzoquinone | 561.05 | | 563.7 | | 90% | single diastereomer |
| 4 | 2-bromo-5-methyl-1,4-benzoquinone | 497.16 | | 497.9 | | 85% | single diastereomer |
| 5 | 2-bromo-6-methyl-p-benzoquinone | 497.16 | | 498.96 | complex | | single diastereomer |
| 6 | 2,6-dimethylbenzoquinone(p-xyloquinone) | 513.19 | 513 | 515.16 | 51% | 63% | single diastereomer |
| 7 | 2,5-dimethylbenzoquinone | 513.19 | | 515.1 | 66% | 83% | single diastereomer |
| 8 | trimethylquinone | 527.21 | | 527.8 | | >90% | single diastereomer |
| 9 | duroquinone(tetramethyl-1,4-benzoquinone) | 541.22 | | 541.6 | | 80% | single diastereomer |
| 10 | tetrachloro-1,4-benzoquinone | 621 | | 623.6 | | 85% | single diastereomer |
| 11 | N-bromomethyl-2,3-dichloromaleimide | 634 | | 634.6 | | 90% | single diastereomer |
| 12 | 3,4-dichloro-1-methyl-2,5-dihydro-1H-pyrrole-2,5-dione | 556.09 | | 556.7 | | >90% | single diastereomer |
| 13 | 2,3-dichloro-N-4-fluorophenylmaleimide | 636.1 | | 636.7 | | 85% | single diastereomer |
| 14 | N-(pentafluorophenyl)dichloromaleimide | 708.06 | | 710.2 | | 90% | single diastereomer |
| 15 | 3-(4-Chlorophenyl)-1(4-fluoroanilino)maleimide | 693.18 | | 693.8 | | 85% | single diastereomer |
| 16 | 3-(4-Chlorophenyl)-1-[[3-chloro-5-(trifluoromethyl)- | 778.13 | | 778.7 | | >90% | single diastereomer |
| | 2-pyridyl]amino]maleimide | | | | | | |
| 17 | 3-phenyl-1-(4-toluidino)-1H-pyrrole-2,5-dione | 655.24 | | 656.1 | | 85% | single diastereomer |
| 18 | 1-(2,4-dichloroanilino)-3-(4-methoxyphenyl)-1H- | 739.16 | | 739.6 | | >90% | single diastereomer |
| | pyrrole-2,5-dione | | | | | | |
| 19 | 1-(3-chlorophenyl)-3-phenyl-2,5-dihydro-1H- | 662.19 | | 661.1 | | >90% | single diastereomer |
| | pyrrole-2,5-dione | | | | | | |
| 20 | 2-methyl-N-phenylmaleimide | 564.2 | | 565.83 | 36% | 72% | single diastereomer |
| 21 | 1-(3-chloro-4-fluorophenyl)-3-methyl-2,5-dihydro- | 616.15 | | 616.6 | | 90% | single diastereomer |
| | 1H-pyrrole-2,5-dione | | | | | | |
| 22 | 1-[3-(5-chloro-3-methylbenzo[B]thiophen-2-yl)-1- | 748.19 | 748.13 | 749.97 | | 90% | single diastereomer |
| | methyl-1H-pyrazol-5-yl]-3-methyl-2,5-dihydro-1H- | | | | | | |
| | pyrrole-2,5-dione | | | | | | |

TABLE 3
Quality assessment of tetracycles formed by the “interrupted” Diels-Alder reaction
of macrobead-supported triene 3 with building blocks B followed by the second [4 + 2]
reaction with N-phenylmaleimide. (Building block B1 and maleimide combination provides
scaffold S7, B2-B5 plus maleimides under the influence of SrCO₃ furnish S9, and B11-B22
with maleimides supply S4. Dienes derived from p-xyloquinone, 2,5-dimethylbenzoquinone,
trimethylquinone, duroquinone, and tetrachloro-p-benzoquinone were not completely
consumed after 4 days of reflux in toluene in the presence of N-phenylmaleimide. The
condition was too harsh and the beads started to break.) Purity was based on integration of
the peaks in the chromatograph from TIC of diode array, AP−, or AP+. Common impurity
peaks throughout many samples were excluded from integration.

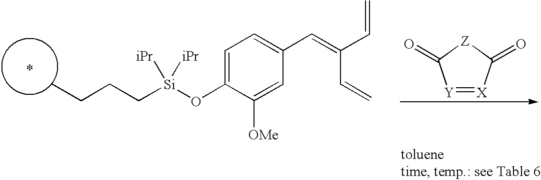

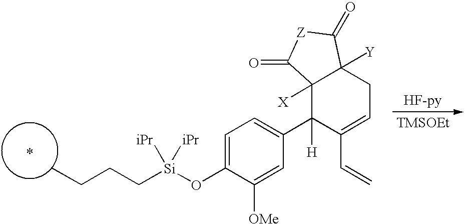

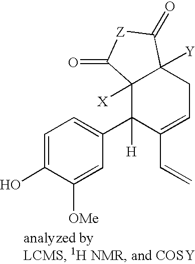

| | | | observed mass | | purity | | | |
|---|---|---|---|---|---|---|---|---|
| BBB | name | exact mass | AP− | AP+ | AP− | diode array | AP+ | ¹H NMR, COSY |
| 1 | 2,5-diphenyl-p-benzoquinone | 635.23 | | 636 | | | 90% | single diastereomer |
| 2 | 2,6-diiodo-p-benzoquinone | 607.05 | | 607.9 | | | 85% | single diastereomer |
| 3 | 2,5-dibromo-1,4-benzoquinone | 559.06 | | | complex | | | complex |
| 4 | 2-bromo-5-methyl-1,4-benzoquinone | 495.17 | | 496 | | | 85% | single diastereomer |
| 5 | 2-bromo-6-methyl-p-benzoquinone | 495.17 | | 496.23 | | 39% | 45% | single diastereomer |
| 6 | 2,6-dimethylbenzoquinone(p-xyloquinone) | | | | | | | N/A |
| 7 | 2,5-dimethylbenzoquinone | | | | | | | N/A |
| 8 | trimethylquinone | | | | | | | N/A |
| 9 | duroquinone(tetramethyl-1,4-benzoquinone) | | | | | | | N/A |
| 10 | tetrachloro-1,4-benzoquinone | | | | | | | N/A |
| 11 | N-bromomethyl-2,3-dichloromaleimide | 632.01 | | 633.1 | 66% | 40% | | single diastereomer |
| 12 | 3,4-dichloro-1-methyl-2,5-dihydro-1H-pyrrole-2,5- | 554.1 | | 554.7 | | | 90% | single diastereomer |
| | dione | | | | | | | |
| 13 | 2,3-dichloro-N-4-fluorophenylmaleimide | 564.17 | | 565 | | | 75% | single diastereomer |
| 14 | N-(pentafluorophenyl)dichloromaleimide | 636.13 | | 637 | | | >90% | single diastereomer |
| 15 | 3-(4-Chlorophenyl)-1(4-fluoroanilino)maleimide | 691.19 | | 692.1 | | | 90% | single diastereomer |
| 16 | 3-(4-Chlorophenyl)-1-[[3-chloro-5-(trifluoromethyl)- | 776.14 | | 770 | | | 90% | single diastereomer |
| | 2-pyridyl]amino]maleimide | | | | | | | |
| 17 | 3-phenyl-1-(4-toluidino)-1H-pyrrole-2,5-dione | 653.25 | | 654.1 | | | 90% | single diastereomer |
| 18 | 1-(2,4-dichloroanilino)-3-(4-methoxyphenyl)-1H- | 737.17 | | 737.7 | | | 85% | single diastereomer |
| | pyrrole-2,5-dione | | | | | | | |
| 19 | 1-(3-chlorophenyl)-3-phenyl-2,5-dihydro-1H- | 660.2 | | 658.4 | | | 90% | single diastereomer |
| | pyrrole-2,5-dione | | | | | | | |
| 20 | 2-methyl-N-phenylmaleimide | 562.21 | | | | | | single diastereomer |
| 21 | 1-(3-chloro-4-fluorophenyl)-3-methyl-2,5-dihydro- | 614.16 | 614 | 616.13 | 87% | 75% | 74% | single diastereomer |
| | 1H-pyrrole-2,5-dione | | | | | | | |
| 22 | 1-[3-(5-chloro-3-methylbenzo[B]thiophen-2-yl)-1- | 746.2 | 746.14 | 747.85 | | | 90% | single diastereomer |
| | methyl-1H-pyrazol-5-yl]-3-methyl-2,5-dihydro-1H- | | | | | | | |
| | pyrrole-2,5-dione | | | | | | | |

TABLE 4
Quality assessment of tetracycles formed by the consecutive Diels-Alder reaction
of macrobead-supported triene 3 with building blocks C. (Building blocks C1-C2 provide
scaffold S6 and C4-C41, S4 with X = Y = H. The molecular ion peak for BBC39 arose
from the oxidation of phenyl boronic acid to phenol under the ionization condition of the
mass spectrometer. Three tetracycles derived from β-(4-hydroxyphenyl)-ethylmaleimide,
maleimide, and N-carbamoylmaleimide gave <50% purity. However, they were included in
the final library synthesis since they display unique functionality and their ¹H NMR and
COSY spectra displayed single diastereomeric products.) Purity was based on integration of
the peaks in the chromatograph from TIC of diode array, AP−, or AP+. Common impurity
peaks throughout many samples were excluded from integration.

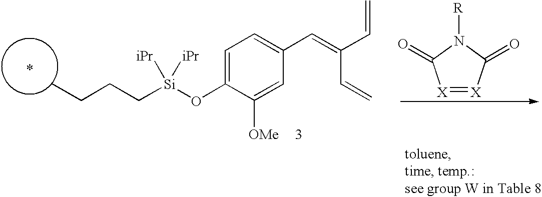

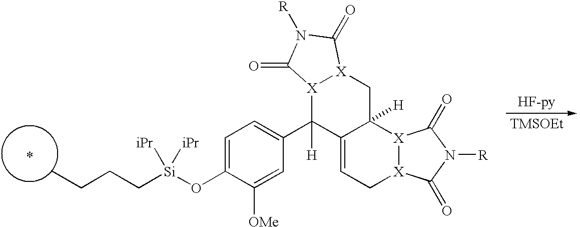

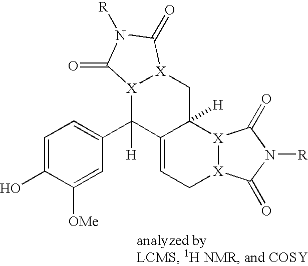

| | | | observed mass | | Purity | | | |
|---|---|---|---|---|---|---|---|---|
| BBC | name | exact ms | AP− | AP+ | AP− | | AP+ | ¹H NMR and COSY |
| 1 | 4-methyl-1,2,4-triazoline-3,5-dione | 428.14 | | 429 | | | | single diastereomer |
| 2 | 4-phenyl-1,2,4-triazoline-3,5-dione | 552.18 | | 552.9 | | | | single diastereomer |
| 3 | DMEQ-TAD | 892.31 | 892.3 | 893.3 | | >95% | | single diastereomer |
| 4 | N-methylmaleimide | 424.16 | | 425.34 | | >95% | | single diastereomer |
| 5 | N-ethylmaleimide | 452.19 | | 453.93 | 86% | 57% | 81% | single diastereomer |
| 6 | N-(n-propyl)maleimide | 480.23 | | 481.55 | | 81% | | single diastereomer |
| 7 | N-benzylmaleimide | 576.23 | | 578.09 | | 95% | | single diastereomer |
| 8 | 2-thienylmethyl maleimide | 588.14 | 587.86 | 590.03 | | >95% | | single diastereomer |
| 9 | N-phenylmaleimide | 548.19 | | 548.9 | | | | single diastereomer |
| 10 | N-(4-ethylphenyl)maleimide | 604.26 | 603.84 | 605.66 | | >95% | | single diastereomer |
| 11 | N-(4-vinylphenyl)maleimide | 600.23 | 599.84 | 601.57 | | >95% | | single diastereomer |
| 12 | 1-[3,5-bis(trifluoromethyl)phenyl]-1H-pyrrole-2,5-dione | 820.14 | 819.91 | 820.84 | | 94% | | single diastereomer |
| 13 | N-methoxycarbonylmaleimide | 512.14 | | 512.9 | | | | single diastereomer |
| 14 | N-cyclohexylmaleimide | 560.29 | | 561.89 | | 91% | | single diastereomer |
| 15 | N-(4-methyl-3-chlorophenyl)maleimide | 644.15 | 643.96 | 645.88 | | 86% | | single diastereomer |
| 16 | N-(4-chlorophenyl)maleimide | 616.12 | 615.92 | 618.13 | | >95% | | single diastereomer |
| 17 | N-(4-bromophenyl)maleimide | 704.02 | | 706.6 | | | | single diastereomer |
| 18 | N-(4-iodophenyl)maleimide | 799.99 | | 800.6 | | | | single diastereomer |
| 19 | N-hydroxymaleimide | 428.12 | 427.89 | — | | >95% | | single diastereomer |
| 20 | N-tert-butylmaleimide | 508.26 | | 509.48 | | 76% | | single diastereomer |
| 21 | beta-(4-hydroxyphenyl)ethylmaleimide | 636.25 | 636.15 | 638.26 | | 33% | | single diastereomer |
| 22 | N-[4-2-benzoxazolyl)phenyl]maleimide | 782.24 | 782.02 | 783.31 | | 85% | | single diastereomer |
| 23 | 2,5-dimethoxystilbene-4′-maleimide | 872.33 | 872.24 | 873.97 | | >95% | | single diastereomer |
| 24 | N-(4-acetylphenyl)maleimide | 632.22 | 632.04 | 633.95 | | >95% | | single diastereomer |
| 25 | 4-(N-maleimido)benzophenone | 756.25 | 756.18 | 758.15 | | 90% | | single diastereomer |
| 26 | 1-(1-benzylpiperidin-4-yl)-1H-pyrrole-2,5-dione | 742.37 | | 744.26 | | 78% | | single diastereomer |
| 27 | N-(3-nitrophenyl)maleimide | 638.16 | 638.05 | 640.07 | | >95% | | single diastereomer |
| 28 | N-(4-nitrophenyl)maleimide | 638.16 | 638.03 | — | | >95% | | single diastereomer |
| 29 | N-(4-dimethylamino-3,5-dinitrophenyl)maleimide | 814.22 | 814.1 | 815.1 | | >95% | | single diastereomer |
| 30 | maleimide | 396.13 | 395.1 | 397.1 | | — | | single diastereomer |
| 31 | N-(4-anilinophenyl)maleimide | 729.27 | | 730.9 | | | >95% | single diastereomer |
| 32 | BIONET 9H-912 | 812.14 | | 812.8 | | | >95% | single diastereomer |
| 33 | N-carbamoylmaleimide | 482.14 | — | — | | complex | | single diastereomer |
| 34 | 3-maleimidopropionic acid | 540.17 | | 540.9 | | | | single diastereomer |
| 35 | 4-maleimidobutyric acid | 568.21 | | 569 | | | | single diastereomer |
| 36 | 6-maleimidocaproic acid | 624.27 | 623.6 | — | 90% | — | | single diastereomer |
| 37 | 4-dimethylaminophenylazophenyl-4′-maleimide | 842.35 | | 844.2 | | >95% | | single diastereomer |
| 38 | 1-(4-morpholinophenyl)-2,5-dihydro-1H-pyrrole- | 718.3 | | 720.22 | | 38% | 82% | single diastereomer |
| | 2,5-dione | | | | | | | |
| 39 | 3-maleimidophenyl boronic acid | 580.18 | 580.06 | 581.94 | | 58% | 74% | single diastereomer |
| 40 | 3-N-maleimidobenzoic acid | 636.17 | — | 637.3 | | 90% | | single diastereomer |
| 41 | N-(4-carboxy-3-hydroxyphenyl)maleimide | 668.16 | 668.1 | 670.24 | | 88% | | single diastereomer |

Experimental Procedures (Library Synthesis):

Loading of hydroxyaldehydes. The p-methoxyphenylsilane beads (400 mg per one hydroxyaldehyde, 1.25 mmol/g of silane, ICCB batch Mx-12) were added to reaction vials which were capped with septa and flushed with dry nitrogen for 5 min. CH₂Cl₂ (4 mL) was added and in 15 min followed by chlorotrimethylsilane (141 μL, 1.11 mmol) and the beads were left suspended for 15 min. After filtered and washed twice with CH₂Cl₂ under nitrogen, beads were suspended in 3% (v/v) solution of triflic acid (9.8 mL, 3.34 mmol) in CH₂Cl₂ for 15 min during which time the vials were shaken periodically. Beads turned reddish orange. The beads were filtered and washed once with CH₂Cl₂ and left suspended in CH₂Cl₂ (1.2 mL). Freshly distilled 2,6-lutidine (518 μL, 4.45 mmol) was added to the beads. Upon decoloration of the reddish orange beads to pale yellow color, solutions of hydroxyaldehydes (Table 5, 1.67 mmol) in CH₂Cl₂ (2 mL) were added. For hydroxyaldehydes that are not soluble in CH₂Cl₂, the triflic acid solution was not washed away; Simply, more 2,6-lutidine (583 μL, 5.00 mmol) was added and solid hydroxyaldehydes (1.67 mmol) were quickly added into the reaction media. Reaction vials were agitated for 3 h. The beads were then filtered, washed with CH₂Cl₂ (4×30 min) and dried under high vacuum overnight. Through the building block screening, 40 out of the original 64 hydroxyaldehyde were found to yield a single identifiable compound. Twelve hydroxyaldehyde manifested low level of loading and twelve different hydroxyaldehydes underwent incomplete conversion to the triene.

Tagging for the building blocks A. Each tag solution (3 mL, 18.9 mM in CH₂Cl₂) was added to the hydroxyaldehyde-loaded beads from the above procedure (hydroxyaldehyde list in Table 5). The beads in tag solution were allowed to stand for 45 min at room temperature. A solution of rhodium triphenylacetate (3 mL, 100 mg/30 mL of CH₂Cl₂) was added to this mixture, which was shaken for 4 h at room temperature. The resulting 40 fractions were then filtered separately and washed with CH₂Cl₂ (2×1 h) to make sure that there was no cross-exposure of different batches of beads to other tags. All batches were combined after preliminary washing and washed more rigorously with THF (overnight) and CH₂Cl₂ (2×1 h), and dried under high vacuum overnight.

TABLE 5
Building blocks A used in the library synthesis and their encoding strategy

| Building | | | | | | | | | | | | | | | | | | |
|---|---|---|---|---|---|---|---|---|---|---|---|---|---|---|---|---|---|---|
| Block | name | A1 | A2 | A3 | A4 | A5 | A6 | A7 | A8 | A9 | A10 | A11 | A12 | A13 | B3 | B4 | B5 | B6 |
| BBA 1 | salicylaldehyde | | | | | | 1 | | | | | | | | | | | |
| BBA 2 | 3-fluoro-2-hydroxybenzaldehyde | | | | | 1 | | | | | | | | | | | | |
| BBA 3 | o-vanillin | | | | | 1 | 1 | | | | | | | | | | | |
| BBA 4 | 3-ethoxysalicylaldehyde | | | | 1 | | | | | | | | | | | | | |
| BBA 5 | 2-hydroxy-4-methoxybenzaldehyde | | | | 1 | | 1 | | | | | | | | | | | |
| BBA 6 | 4,6-dimethoxysalicylaldehyde | | | | 1 | 1 | | | | | | | | | | | | |
| BBA 7 | 5-bromosalicylaldehyde | | | | 1 | 1 | 1 | | | | | | | | | | | |
| BBA 8 | 5-chlorosalicylaldehyde | | | 1 | | | | | | | | | | | | | | |
| BBA 9 | 2-hydroxy-5-methoxybenzaldehyde | | | 1 | | | 1 | | | | | | | | | | | |
| BBA 10 | 5-bromo-3-methoxysalicylaldehyde | | | 1 | | 1 | | | | | | | | | | | | |
| BBA 11 | 3-methyl-2-hydroxybenzaldehyde | | | 1 | | 1 | 1 | | | | | | | | | | | |
| BBA 12 | 5-(trifluoromethoxy)-salicylaldehyde | | | 1 | 1 | | | | | | | | | | | | | |
| BBA 13 | 3-allylsalicylaldehyde | | | 1 | 1 | | 1 | | | | | | | | | | | |
| BBA 14 | 4-benzyloxy-2-hydroxybenzaldehyde | | | 1 | 1 | 1 | | | | | | | | | | | | |
| BBA 15 | 6-methoxysalicylaldehyde | | | 1 | 1 | 1 | 1 | | | | | | | | | | | |
| BBA 16 | 3-hydroxybenzaldehyde | | 1 | | | | | | | | | | | | | | | |
| BBA 17 | 4-methoxy-3-hydroxybenzaldehyde | | 1 | | | | 1 | | | | | | | | | | | |
| BBA 18 | 4-hydroxybenzaldehyde | | 1 | | | 1 | | | | | | | | | | | | |
| BBA 19 | 3-fluoro-4-hydroxybenzaldehyde | | 1 | | | 1 | 1 | | | | | | | | | | | |
| BBA 20 | 6-chloro-4-hydroxybenzaldehyde | | 1 | | 1 | | | | | | | | | | | | | |
| BBA 21 | 3-methyl-4-hydroxybenzaldehyde | | 1 | | 1 | | 1 | | | | | | | | | | | |
| BBA 22 | 3,5-dimethy-4-hydroxybenzaldehyde | | 1 | | 1 | 1 | | | | | | | | | | | | |
| BBA 23 | 6-methoxy-4-hydroxybenzaldehyde | | 1 | | 1 | 1 | 1 | | | | | | | | | | | |
| BBA 24 | 3-ethoxy-4-hydroxybenzaldehyde | | 1 | 1 | | | | | | | | | | | | | | |
| BBA 25 | 2,6-dimethoxy-4-hydroxybenzaldehyde | | 1 | 1 | | | 1 | | | | | | | | | | | |
| BBA 26 | 3,5-dimethoxy-4-hydroxybenzaldehyde | | 1 | 1 | | 1 | | | | | | | | | | | | |
| BBA 27 | 5-chloro-3-methoxy-4- | | 1 | 1 | | 1 | 1 | | | | | | | | | | | |
| | hydroxybenzaldehyde | | | | | | | | | | | | | | | | | |
| BBA 28 | 5-bromo-3-methoxy-4- | | 1 | 1 | 1 | | | | | | | | | | | | | |
| | hydroxybenzaldehyde | | | | | | | | | | | | | | | | | |
| BBA 29 | 5-iodovanillin | | 1 | 1 | 1 | | 1 | | | | | | | | | | | |
| BBA 30 | 3-methoxy-4-hydroxybenzaldehyde | | 1 | 1 | 1 | 1 | | | | | | | | | | | | |
| BBA 31 | 2,3-dibromo-5-methoxy-4- | | 1 | 1 | 1 | 1 | 1 | | | | | | | | | | | |
| | hydroxybenzaldehyde | | | | | | | | | | | | | | | | | |
| BBA 32 | 4-hydroxy-3-methoxycinnamaldehyde | 1 | | | | | | | | | | | | | | | | |
| BBA 33 | 3,5-dimethoxy-4- | 1 | | | | | 1 | | | | | | | | | | | |
| | hydroxycinnamaldehyde | | | | | | | | | | | | | | | | | |
| BBA 34 | 5-hydroxymethyl-2-furaldehyde | 1 | | | | 1 | | | | | | | | | | | | |
| BBA 35 | 2-(2-hydroxyethoxy)benzaldehyde | 1 | | | | 1 | 1 | | | | | | | | | | | |
| BBA 36 | 4-(2-hydroxyethoxy)benzaldehyde | 1 | | | 1 | | | | | | | | | | | | | |
| BBA 37 | 2-hydroxy-1-naphthaldehyde | 1 | | | 1 | | 1 | | | | | | | | | | | |
| BBA 38 | 1-hydroxy-2-naphthaldehyde | 1 | | | 1 | 1 | | | | | | | | | | | | |
| BBA 39 | 4-hydroxy-1-naphthaldehyde | 1 | | | 1 | 1 | 1 | | | | | | | | | | | |
| BBA 40 | 6-hydroxychromen-3- | 1 | | 1 | | | | | | | | | | | | | | |
| | carboxaldehyde | | | | | | | | | | | | | | | | | |

Indium-mediated Barbier-type reaction. DMF, THF, and H₂O are commonly used solvents for reactions involving allyl indium and penta-2,4-dienyl indiums. H₂O was not tested since polystyrene has poor swelling properties in water. The same reaction in THF yielded 19% of the desired product 2. What we found in this Barbier-type reaction was that it is important to use a 1.8:1 molar ratio of penta-2,4-dienyl bromide/indium. When 2:1 molar ratio of 5-bromo-1,3-pentadiene/indium was used, side products presumably arising from O-alkylation contaminated the reaction. O-alkylation was also facilitated by strictly anhydrous condition. Anhydrous DMF was used as solvent for the reaction, but the reaction was performed under open air. In principle, beads after being tagged can be pooled and the following reaction can be run on one large scale. The indium-mediated Barbier-type reaction, though, generates heat during the formation of the organometallic reagent after initiation. Inefficient dissipation of this heat in large reaction scales (over 1 g of beads) leads to side-reactions. Therefore, the following reaction was run in the scale indicated. In the event, the beads from above (corresponding to 400 mg ofp-methoxyphenylsilane beads before loading) were swelled in DMF (3.6 mL) for 15 min, and 5-bromo-1,3-pentadiene (400 μL, 4.0 mmol) and indium (259 mg, 2.27 mmol) were added. The reaction vials were capped and shaken for 24 h at room temperature. The beads were then filtered, washed using DMF (2×1 h), THF (2×2 h), and CH₂Cl₂ (2×1 h), and dried under high vacuum overnight.

Dehydration. The beads from above were combined and divided into 20 fractions for the ease of operation. Again, a very large-scale reaction (over 1 g of beads) was not successfully accomplished and 40 fractions of beads were combined to 20 fractions of approximately 800 mg of beads. The originally reported dehydration under Mitsunobu-type conditions provided the triene 3 in 10% (Ph₃P, DEAD, benzene, room temperature) and in 73% (n-Bu₃P, DIAD, PhOH, CH₂Cl₂, room temperature) yield, respectively. Burgess reagent and Martin's sulfurane only yielded 12% and 6% of the desired product 3. The optimized dehydration condition is following. Beads loaded with bishomoallylic alcohols (corresponding to approximately 800 mg ofp-methoxyphenylsilane beads before loading) were swollen in CH₂Cl₂ (12 mL) for 15 min and cooled to −5° C. Triethylamine (1.60 mL, 11.5 mmol) and MsCl (792 μL, 10.2 mmol) were added and the reaction was allowed to persist for 4 h at below −5° C. Beads were filtered, washed with CH₂Cl₂ three times, and suspended in benzene for 15 min. After removal of benzene, beads were re-suspended in benzene (8 mL) and treated with 1,8-Diazabicyclo[5.4.0]undec-7-ene (1.7 mL, 11.5 mmol). The reaction was allowed to continue for 12 h at 37° C. The beads were filtered, washed with CH₂Cl₂ (2×1 h), and dried under vacuum overnight.

Diels-Alder cycloaddition employing tri- or tetrasubstituted dienophiles. The encoded beads loaded with trienes from above were pooled and split into 23 fractions. The relative amount of each fraction was 42:41:4:3=15 fractions (to be reacted with building blocks B1, 2, 4, and 11-22): 2 fractions (to be reacted with building block B5 and to be left as trienes to be reacted with 41 building block C): 5 fractions (to be reacted with building block B6-10): 1 fraction (to be reacted with building block B3).¹ Beads (837.3 mg, 817.3 mg, 79.7 mg, and 59.8 mg) were swollen in toluene (9.3 mL, 9.1 mL, 0.9 mL, and 0.8 mL) and treated with building blocks B (2.59 mmol, 2.53 mmol, 0.25 mmol, and 0.18 mmol, for the exact amount used, see Table 6) for time at temperature as shown in Table 6. After that, beads were filtered, washed using THF (2×2 h), and CH₂Cl₂ (2×1 h), and dried under high vacuum overnight.

TABLE 6
Reaction conditions for building blocks B used in the library synthesis.

| BBB | name | mol. wt. | amount used | | rxn condition |
|---|---|---|---|---|---|
| 1 | 2,5-diphenyl-p-benzoquinone | 260.3 | 674.3 | mg | 2 d, 37° C. |
| 2 | 2,6-diiodo-p-benzoquinone | 359.89 | 932.3 | mg | 1 d, RT |
| 3 | 2,5-dibromo-1,4-benzoquinone | 265.89 | 49.2 | mg | 2 d, RT |
| 4 | 2-bromo-5-methyl-1,4-benzoquinone | 201.02 | 520.8 | mg | 2 d, RT |
| 5 | 2-bromo-6-methyl-p-benzoquinone | 201.02 | 508.4 | mg | 2 d, RT |
| 6 | 2,6-dimethylbenzoquinone (p-xyloquinone) | 136.15 | 33.6 | mg | 2 d, 55° C. |
| 7 | 2,5-dimethylbenzoquinone | 136.15 | 33.6 | mg | 2 d, 55° C. |
| 8 | trimethylquinone | 150.18 | 37.1 | mg | 1 d, 100° C. |
| 9 | duroquinone (tetramethyl-1,4-benzoquinone) | 164.2 | 40.5 | mg | 4 d, 100° C. |
| 10 | tetrachloro-1,4-benzoquinone | 245.88 | 60.7 | mg | 1 d, 100° C. |
| 11 | N-bromomethyl-2,3-dichloromaleimide | 258.88 | 670.7 | mg | 2 d, 83° C. |
| 12 | 3,4-dichloro-1-methyl-2,5-dihydro-1H-pyrrole-2,5-dione | 179.99 | 466.2 | mg | 1 d, 100° C. |
| 13 | fluoroimide standard (2,3-dichloro-N-4-fluorophenylmaleimide) | 260.05 | 673.7 | mg | 4 d, 100° C. |
| 14 | N-(pentafluorophenyl)dichloromaleimide | 332.02 | 860.1 | mg | 4 d, 83° C. |
| 15 | 3-(4-Chlorophenyl)-1(4-fluoroanilino)maleimide | 316.72 | 820.5 | mg | 4 d, RT |
| 16 | 3-(4-Chlorophenyl)-1-[[3-chloro-5-(trifluoromethyl)-2-pyridyl]amino]maleimide | 402.16 | 1.042 | g | 2 d, RT |
| 17 | 3-phenyl-1-(4-toluidino)-1H-pyrrole-2,5-dione | 278.31 | 721.0 | mg | 4 d, RT |
| 18 | 1-(2,4-dichloroanilino)-3-(4-methoxyphenyl)-1H-pyrrole-2,5-dione | 363.2 | 940.9 | mg | 4 d, 37° C. |
| 19 | 1-(3-chlorophenyl)-3-phenyl-2,5-dihydro-1H-pyrrole-2,5-dione | 283.71 | 735.0 | mg | 4 d, 55° C. |
| 20 | 2-methyl-N-phenylmaleimide | 187.02 | 484.5 | mg | 6 d, 55° C. |
| 21 | 1-(3-chloro-4-fluorophenyl)-3-methyl-2,5-dihydro-1H-pyrrole-2,5-dione | 239.63 | 620.8 | mg | 5 d, 55° C. |
| | 1-[3-(5-chloro-3-methylbenzo[B]thiophen-2-yl)-1-methyl-1H-pyrazol-5-yl]-3- | | | | |
| 22 | methyl- | 371.85 | 963.3 | mg | 4 d, 83° C. |
| (Dienes derived from 15 dienophiles [out of 22 selected building block B (BBB)] indicated ≧90% purity (LC-MS), 2; ≧80%, | | | | | |
| # and 3; ≧70%. Dienes derived from 2,5-dibromo-p-benzoquinone (B1) and 2-bromo-6-methyl-p-benzoquinone (B5) were not included in the | | | | | |
| # final library since they showed <50% purity. All 20 dienes appeared as single diastereomers in¹H NMR and COSY. All 22 | | | | | |
| # tetracycles formed upon treatment of twenty-two dienes with 4-phenyl-1,2,4-triazoline-3,5-dione manifested purity (LC-MS) of ≧90% | | | | | |
| # (for 11 tetracycles), ≧80% (for 8 tetracycles), and ≧70% (for 3 tetracycles). All 22 tetracycles appeared as single diastereomers | | | | | |
| # in ¹H NMR and COSY. Only fifteen tetracyclic products derived from N-phenylmaleimide were included in the final library. Eight | | | | | |
| # tetracycles (made out of 15 selected BBB) displayed ≧90% purity (LC-MS), 3; ≧80%, and 5; ≧70%. All 15 tetracycles appeared as | | | | | |
| # single diastereomers in ¹H NMR and COSY. Tetracyclic compounds derived from diene derived from 2,5-dibromo-p-benzoquinone (B5) | | | | | |
| # were excluded from the final library synthesis based on their poor analytical data. Dienes derived from p-xyloquinone, | | | | | |
| # 2,5-dimethylbenzoquinone, trimethylquinone, duroquinone, and tetrachloro-p-benzoquinone were not completely consumed after 4 days of | | | | | |
| # reflux in toluene. The condition was too harsh and the beads started to break.) | | | | | |

Tagging for the building blocks B. Each tag solution (4 mL for building block B1, 2, 4, 5, and 11-22 and 0.38 mL for building blocks B3 and 610, 13.2 mM in CH₂Cl₂) was added to the diene-loaded beads as described in Table 7. The beads in tag solution were allowed to stand for 45 min at room temperature. A solution of rhodium triphenylacetate (4 mL for building blocks B1, 2, 4, 5, and 11-22 and 0.38 mL for building block B3 and 6-10, 100 mg/20 mL of CH₂Cl₂) was added to this mixture, which was shaken for 4 h at room temperature. The resulting 22 fractions were filtered separately and washed with CH₂Cl₂ (2×1 h) to make sure that there was no cross-exposure of different batches of beads to other tag solutions. All batches combined, washed with THF (overnight) and CH₂Cl₂ (2×1 h), and dried under high vacuum overnight.

TABLE 7
Encoding strategy for building blocks B.

| Building | | | | | | | | | | | | | | | | | | |
|---|---|---|---|---|---|---|---|---|---|---|---|---|---|---|---|---|---|---|
| Block | name | A1 | A2 | A3 | A4 | A5 | A6 | A7 | A8 | A9 | A10 | A11 | A12 | A13 | B3 | B4 | B5 | B6 |
| BBB 1 | 2,5-diphenyl-p-benzoquinone | | | | | | | | | | | 1 | | | | | | |
| BBB 2 | 2,6-diiodo-p-benzoquinone | | | | | | | | | | 1 | | | | | | | |
| BBB 3 | 2,5-dibromo-1,4-benzoquinone | | | | | | | | | | 1 | 1 | | | | | | |
| BBB 4 | 2-bromo-5-methyl-1,4- | | | | | | | | | 1 | | | | | | | | |
| | benzoquinone | | | | | | | | | | | | | | | | | |
| BBB 5 | 2-bromo-6-methyl-p- | | | | | | | | | 1 | | 1 | | | | | | |
| | benzoquinone | | | | | | | | | | | | | | | | | |
| BBB 6 | 2,6-dimethylbenzoquinone | | | | | | | | | 1 | 1 | | | | | | | |
| | (p-xyloquinone) | | | | | | | | | | | | | | | | | |
| BBB 7 | 2,5-dimethylbenzoquinone | | | | | | | | | 1 | 1 | 1 | | | | | | |
| BBB 8 | trimethylquinone | | | | | | | | 1 | | | | | | | | | |
| BBB 9 | duroquinone (tetramethyl- | | | | | | | | 1 | | | 1 | | | | | | |
| | 1,4-benzoquinone) | | | | | | | | | | | | | | | | | |
| BBB 10 | tetrachloro-1,4-benzoquinone | | | | | | | | 1 | | 1 | 1 | | | | | | |
| BBB 11 | N-bromomethyl-2,3- | | | | | | | | 1 | 1 | | | | | | | | |
| | dichloromaleimide | | | | | | | | | | | | | | | | | |
| BBB 12 | 3,4-dichloro-1-methyl-2,5- | | | | | | | | 1 | 1 | | 1 | | | | | | |
| | dihydro-1H-pyrrole-2,5-dione | | | | | | | | | | | | | | | | | |
| BBB 13 | fluoroimide standard (2,3-dichloro- | | | | | | | | 1 | 1 | 1 | | | | | | | |
| | N-4-fluorophenylmaleimide) | | | | | | | | | | | | | | | | | |
| BBB 14 | N-(pentafluorophenyl) | | | | | | | | 1 | 1 | 1 | 1 | | | | | | |
| | dichloromaleimide | | | | | | | | | | | | | | | | | |
| BBB 15 | 3-(4-Chlorophenyl)-1(4- | | | | | | | 1 | | | | | | | | | | |
| | fluoroanilino)maleimide | | | | | | | | | | | | | | | | | |
| BBB 16 | 3-(4-Chlorophenyl)-1-[[3- | | | | | | | 1 | | | | 1 | | | | | | |
| | chloro-5-(trifluoromethyl)-2- | | | | | | | | | | | | | | | | | |
| | pyridyl]amino]maleimide | | | | | | | | | | | | | | | | | |
| BBB 17 | 3-phenyl-1-(4-toluidino)- | | | | | | | 1 | | | 1 | | | | | | | |
| | 1H-pyrrole-2,5-dione | | | | | | | | | | | | | | | | | |
| BBB 18 | 1-(2,4-dichloroanilino)-3-(4-methoxy- | | | | | | | 1 | | | 1 | 1 | | | | | | |
| | phenyl)-1H-pyrrole-2,5-dione | | | | | | | | | | | | | | | | | |
| BBB 19 | 1-(3-chlorophenyl)-3-phenyl-2,5- | | | | | | | 1 | | 1 | | | | | | | | |
| | dihydro-1H-pyrrole-2,5-dione | | | | | | | | | | | | | | | | | |
| BBB 20 | 2-methyl-N-phenylmaleimide | | | | | | | 1 | | 1 | | 1 | | | | | | |
| BBB 21 | 1-(3-chloro-4-fluorophenyl)- | | | | | | | 1 | | 1 | 1 | | | | | | | |
| | 3-methyl-2,5-dihydro- | | | | | | | | | | | | | | | | | |
| | 1H-pyrrole-2,5-dione | | | | | | | | | | | | | | | | | |
| BBB 22 | 1-[3-(5-chloro-3- | | | | | | | 1 | | 1 | 1 | 1 | | | | | | |
| | methylbenzo[B]thiophen-2-yl)-1- | | | | | | | | | | | | | | | | | |
| | methyl-1H-pyrazol-5-yl]-3-methyl | | | | | | | | | | | | | | | | | |

Diels-Alder cycloaddition employing disubstituted dienophiles. The building block screening efforts revealed that some cyclic dienophiles other than maleic anhydride, maleimides, triazolinediones, and benzoquinones provided mixture of diastereomers (e.g., cyclopent-2-ene-1,4-dione, 2-cyclohexene-1-one, and 1-nitrocyclohexene). Unreacted triene was recovered after several days of reflux in toluene with others (e.g., 5-nitrouracil, 4,4-dimethoxy-2,5-cyclohexadien-1-one, and 1,4-benzoquinone dioxime). After the selection of 41 out of original 53 disubstituted dienophiles, the condition for second Diels-Alder reaction was studied. Different dienes generated above undergo Diels-Alder reaction at different conditions with a given disubstituted cyclic dienophile (building block C). For instance, dienes formed from building blocks B2, B4, and B5 need the assistance of SrCO₃ for dehydrohalogenation when treated with maleimide derivatives (building blocks C4-C41). Table 8 shows the reaction condition chart with corresponding reaction partners. In practice, beads loaded with dienes derived from BBB1, BBB11, BBB14-BBB18 were pooled. One out of forty two portions of the pooled beads was set aside to be combined with the final pool of tetracycle-loaded beads to ensure the presence of bicycles as members of the library. The rest was combined with beads loaded with trienes. The resulting pool of beads was split into 41 fractions to be treated with building blocks C (group W). Beads loaded with dienes derived from BBB12, BBB13, BBB19-BBB22 were pooled and split into 42 fractions, one of which was set aside and the rest was to be allowed to react with 41 disubstituted dienophiles (group X). Beads loaded with dienes derived from BBB2 and BBB4 were combined and one forty-second of the pooled beads was set aside to be added to the final collection of the library. The rest was combined with beads loaded with BBB5 derived-diene and split into 41 fractions to be treated with building blocks C (group Y). Beads loaded with dienes derived from BBB6-BBB10 were pooled and split into 4 fractions (group Z), one of which was set aside. The remaining three fractions were to be allowed to react with three triazolinediones (building blocks C1, C2, and C3).

In the event three fractions from four groups above were combined with each other, soaked in toluene (5.1 mL), and treated with triazolinediones (1.70 mmol, 2 equiv., for the exact amount used, see Table 8) since all dienes and triene react with triazolinediones under the same condition (2 days at room temperature). 38 Fractions from group W were swollen in toluene (1.8 mL), treated with 38 maleimde derivatives (0.593 mmol, 3 equiv., for the exact amount used, see Table 8), and allowed to react under the conditions given in Table 8. 38 Fractions from group X were swollen in toluene (1.4 mL), treated with 38 building blocks C (0.444 mmol, 3 equiv., for the exact amount used, see Table 8) to be left under the conditions shown in Table 8. 33 Fractions from group Y were swollen in toluene (0.7 mL) and 5 fractions from group Y were swollen in THF (0.7 mL). All 38 fractions of swollen beads from group Y were then treated with SrCO₃ (22 mg, 0.15 mmol, 2 equiv.) and maleimides (0.222 mmol, 3 equiv., for the exact amount used, see Table 8) for 2 days at 50 ° C. Beads were then filtered, washed using, THF (2×2 h), and CH₂Cl₂ (2×1 h), and dried under high vacuum overnight.

TABLE 8
Reaction condition for building blocks C used in the library synthesis

| | | | | triene + | | triene + | | | | | |
|---|---|---|---|---|---|---|---|---|---|---|---|
| | | | | 22 dienes | | 7 dienes | | 6 dienes | | 3 dienes | |
| | | triene | | (W, X, Y, Z) | | (group W) | | (group X) | | (group Y) | |
| | | dienes | | used | rxn | used | rxn | used | rxn | used | rxn |
| BBC | name | mol. wt. | density | amount | condition | amount | condition | amount | condition | amount | condition |
| 1 | 4-methyl-1,2,4- | 113.08 | | 192.5 | 2 d, | | | | | | |
| | triazoline-3,5-dione | | | mg | RT | | | | | | |
| 2 | 4-phenyl-1,2,4- | 175.15 | | 298.2 | 2 d, | | | | | | |
| | triazoline-3,5-dione | | | mg | RT | | | | | | |
| 3 | DMEQ-TAD | 345.31 | | 587.8 | 2 d, | | | | | | |
| | | | | mg | RT | | | | | | |
| 4 | N-methylmaleimide | 111.1 | | | | 65.8 | 5 d, | 49.3 | 5 d, | 24.7 | 2 d, |
| | | | | | | mg | RT | mg | 37° C. | mg | 50° C. |
| 5 | N-ethylmaleimide | 125.13 | | | | 74.1 | 5 d, | 55.6 | 5 d, | 27.8 | 2 d, |
| | | | | | | mg | RT | mg | 37° C. | mg | 50° C. |
| 6 | N-(n-propyl)maleimide | 139.15 | | | | 82.5 | 5 d, | 61.8 | 5 d, | 30.9 | 2 d, |
| | | | | | | mg | RT | mg | 37° C. | mg | 50° C. |
| 7 | N-benzylmaleimide | 187.2 | | | | 111.0 | 5 d, | 83.1 | 5 d, | 41.6 | 2 d, |
| | | | | | | mg | RT | mg | 37° C. | mg | 50° C. |
| 8 | 2-thienylmethyl | 193.23 | | | | 114.6 | 5 d, | 85.8 | 5 d, | 42.9 | 2 d, |
| | maleimide | | | | | mg | RT | mg | 37° C. | mg | 50° C. |
| 9 | N-phenylmaleimide | 173.17 | | | | 102.7 | 5 d, | 76.9 | 5 d, | 38.5 | 2 d, |
| | | | | | | mg | RT | mg | 37° C. | mg | 50° C. |
| 10 | N-(4-ethyl- | 201.22 | | | | 119.3 | 5 d, | 89.3 | 5 d, | 44.7 | 2 d, |
| | phenyl)maleimide | | | | | mg | RT | mg | 37° C. | mg | 50° C. |
| 11 | N-(4-vlnylphenyl)maleimide | 199.21 | | | | 118.2 | 5 d, | 88.4 | 5 d, | 44.2 | 2 d, |
| | 1-[3,5-bis(trifluoro- | | | | | mg | RT | mg | 37° C. | mg | 50° C. |
| | methyl)phenyl]- | | | | | | | | | | |
| | 1H-pyrrole- | | | | | | | | | | |
| 12 | 2,5-dione | 309.17 | | | | 183.4 | 5 d, | 137.3 | 5 d, | 68.7 | 2 d, |
| | | | | | | mg | RT | mg | 37° C. | mg | 50° C. |
| 13 | N-methoxy- | 155.11 | | | | 92.0 | 5 d, | 68.9 | 5 d, | 34.5 | 2 d, |
| | carbonylmaleimide | | | | | mg | RT | mg | 37° C. | mg | 50° C. |
| 14 | N-cyclohexyl- | 179.22 | | | | 106.3 | 5 d, | 79.6 | 5 d, | 39.8 | 2 d, |
| | maleimide | | | | | mg | RT | mg | 37° C. | mg | 50° C. |
| 15 | N-(4-methyl-3- | 221.64 | | | | 131.5 | 5 d, | 98.4 | 5 d, | 49.2 | 2 d, |
| | chlorophenyl)maleimide | | | | | mg | RT | mg | 37° C. | mg | 50° C. |
| 16 | N-(4-chloro- | 207.62 | | | | 123.1 | 5 d, | 91.2 | 5 d, | 45.6 | 2 d, |
| | phenyl)maleimide | | | | | mg | RT | mg | 37° C. | mg | 50° C. |
| 17 | N-(4-bromo- | 252.07 | | | | 149.5 | 5 d, | 111.9 | 5 d, | 56.0 | 2 d, |
| | phenyl)maleimide | | | | | mg | RT | mg | 37° C. | mg | 50° C. |
| 18 | N-(4-iodo- | 290.08 | | | | 172.0 | 5 d, | 128.8 | 5 d, | 64.4 | 2 d, |
| | phenyl)maleimide | | | | | mg | RT | mg | 37° C. | mg | 50° C. |
| 19 | N-hydroxy- | 113.07 | | | | 67.1 | 5 d, | 50.2 | 5 d, | 25.1 | 2 d, |
| | maleimide | | | | | mg | RT | mg | 37° C. | mg | 50° C. |
| 20 | N-tert-butyl- | 153.18 | 1.059 | | | 85.8 | 5 d, | 64.2 | 5 d, | 32.1 | 2 d, |
| | maleimide | | | | | ul | 55° C. | ul | 75° C. | mg | 50° C. |
| 21 | beta-(4- | 217.22 | | | | 128.8 | 5 d, | 96.4 | 5 d, | 48.2 | 2 d, |
| | hydroxyphenyl)ethyl- | | | | | mg | 55° C. | mg | 75° C. | mg | 50° C. |
| | maleimide | | | | | | | | | | |
| 22 | N-[4-2-benzo- | 290.28 | | | | 172.1 | 5 d, | 128.9 | 5 d, | 64.5 | 2 d, |
| | xazolyl)phenyl]maleimide | | | | | mg | 55° C. | mg | 75° C. | mg | 50° C. |
| 23 | 2,5-dimethoxystilbene- | 335.36 | | | | 198.9 | 5 d, | 148.9 | 5 d, | 74.5 | 2 d, |
| | 4′-maleimide | | | | | mg | 55° C. | mg | 75° C. | mg | 50° C. |
| 24 | N-(4-acetyl- | 215.21 | | | | 127.6 | 5 d, | 95.6 | 5 d, | 47.8 | 2 d, |
| | phenyl)maleimide | | | | | mg | 55° C. | mg | 75° C. | mg | 50° C. |
| 25 | 4-(N-maleimido)benzo- | 277.3 | | | | 164.5 | 5 d, | 123.1 | 5 d, | 61.6 | 2 d, |
| | phenone | | | | | mg | 55° C. | mg | 75° C. | mg | 50° C. |
| 26 | 1-(1-benzylpiperidin- | 270.33 | | | | 160.3 | 5 d, | 120.0 | 5 d, | 60.0 | 2 d, |
| | 4-yl)-1H-pyrrole- | | | | | mg | 55° C. | mg | 75° C. | mg | 50° C. |
| | 2,5-dione | | | | | | | | | | |
| 27 | N-(3-nitro- | 218.16 | | | | 129.4 | 5 d, | 96.9 | 5 d, | 48.5 | 2 d, |
| | phenyl)maleimide | | | | | mg | 55° C. | mg | 75° C. | mg | 50° C. |
| 28 | N-(4-nitro- | 218.17 | | | | 129.4 | 5 d, | 96.9 | 5 d, | 48.5 | 2 d, |
| | phenyl)maleimide | | | | | mg | 55° C. | mg | 73° C. | mg | 50° C. |
| 29 | N-(4-dimethylamino-3,5- | 306.23 | | | | 181.6 | 5 d, | 136.0 | 5 d, | 68.0 | 2 d, |
| | dinitrophenyl)maleimide | | | | | mg | 55° C. | mg | 75° C. | mg | 50° C. |
| 30 | maleimide | 97.07 | | | | 57.6 | 5 d, | 43.1 | 5 d, | 21.6 | 2 d, |
| | | | | | | mg | 55° C. | mg | 75° C. | mg | 50° C. |
| 31 | N-(4-anilino- | 264.28 | | | | 156.7 | 5 d, | 117.3 | 5 d, | 58.7 | 2 d, |
| | phenyl)maleimide | | | | | mg | 55° C. | mg | 75° C. | mg | 50° C. |
| 32 | BIONET 9H-912 | 305.65 | | | | 181.3 | 5 d, | 135.7 | 5 d, | 67.9 | 2 d, |
| | | | | | | mg | 55° C. | mg | 75° C. | mg | 50° C. |
| 33 | N-carbamoyl- | 140.1 | | | | 83.1 | 5 d, | 62.2 | 5 d, | 31.1 | 2 d, |
| | maleimide | | | | | mg | 55° C. | mg | 75° C. | mg | 50° C. |
| 34 | 3-maleimido- | 169.14 | | | | 100.3 | 5 d, | 75.1 | 5 d, | 37.6 | 2 d, |
| | propionic acid | | | | | mg | 55° C. | mg | 75° C. | mg | 50° C. |
| 35 | 4-maleimido- | 183.17 | | | | 108.6 | 5 d, | 81.3 | 5 d, | 40.7 | 2 d, |
| | butyric acid | | | | | mg | 55° C. | mg | 75° C. | mg | 50° C. |
| 36 | 6-maleimido- | 211.22 | | | | 125.3 | 5 d, | 93.8 | 5 d, | 46.9 | 2 d, |
| | caproic acid | | | | | mg | 55° C. | mg | 75° C. | mg | 50° C. |
| 37 | 4-dimethylamino- | 320.35 | | | | 190.0 | 5 d, | 142.2 | 5 d, | 71.1 | 2 d, |
| | phenylazophenyl- | | | | | mg | 75° C. | mg | 90° C. | mg | 50° C. |
| | 4′-maleimide | | | | | | | | | | |
| 38 | 1-(4-morpholinophenyl)- | 258.28 | | | | 153.2 | 5 d, | 114.7 | 5 d, | 57.4 | 2 d, |
| | 2,5-dihydro-1H-pyrrole- | | | | | mg | 75° C. | mg | 90° C. | mg | 50° C. |
| | 2,5-dione | | | | | | | | | | |
| 39 | 3-maleimidophenyl | 216.98 | | | | 128.7 | 5 d, | 96.3 | 5 d, | 48.2 | 2 d, |
| | boronic acid | | | | | mg | 75° C. | mg | 90° C. | mg | 50° C. |
| 40 | 3-N-maleimido- | 217.2 | | | | 128.8 | 5 d, | 96.4 | 5 d, | 48.2 | 2 d, |
| | benzoic acid | | | | | mg | 75° C. | mg | 90° C. | mg | 50° C. |
| 41 | N-(4-carboxy-3- | 233.2 | | | | 138.3 | 5 d, | 103.5 | 5 d, | 51.8 | 2 d, |
| | hydroxyphenyl)maleimide | | | | | mg | 75° C. | mg | 90° C. | mg | 50° C. |

(Purity ascertained by LC-MS was ≧90% (for 29 tetracycles), ≧80% (for 6 tetracycles), and ≧70% (for 3 tetracycles). Three tetracycles derived from β-(4-hydroxyphenyl)ethylmaleimide, maleimide, and N-carbamoylmaleimide demonstrated <50% purity. However, they were included in the final library synthesis since they display unique functionality and their ¹H NMR and COSY spectra displayed single diastereomeric products.)

Tagging for the building blocks C. 38 Fractions from group W, X, and Y were combined to be tagged. Each tag solution (3.4 mL for building blocks C1-C3 and 2.5 mL for building block C4-C41, 17.1 mM in CH₂Cl₂) was added to the tetracycle-loaded beads as described in Table 8. The beads in tag solution were allowed to stand for 45 min at room temperature. A solution of rhodium triphenylacetate (2.7 mL for building blocks C1-C3 and 2.0 mL for building blocks C4-C4 1, 100 mg/20 mL of CH₂Cl₂) was added to this mixture, which was shaken for 4 h at room temperature. The resulting 41 fractions were filtered separately and washed with CH₂Cl₂ (2×1 h) to make sure that there was no cross-exposure of different batches of beads to the wrong combination of tag solution. All batches of beads were combined and washed more rigorously with THF (overnight) and CH₂Cl₂ (2×1 h), and dried under high vacuum overnight. All the resulting tetracycle-loaded beads were combined as well as the bicycle-loaded beads to result in a final weight of 20.2674 g of beads. The starting p-methoxyphenylsilane beads weighed 13.0456 g.

TABLE 9
Encoding strategy for building blocks C.

| Building | | | | | | | | | | | | | | | | | | |
|---|---|---|---|---|---|---|---|---|---|---|---|---|---|---|---|---|---|---|
| Block | name | A1 | A2 | A3 | A4 | A5 | A6 | A7 | A8 | A9 | A10 | A11 | A12 | A13 | B3 | B4 | B5 | B6 |
| BBC 1 | 4-methyl-1,2,4-triazoline-3,5-dione | | | | | | | | | | | | | | | | | 1 |
| BBC 2 | 4-phenyl-1,2,4-triazoline-3,5-dione | | | | | | | | | | | | | | | | 1 | |
| BBC 3 | DMEQ-TAD | | | | | | | | | | | | | | | | 1 | 1 |
| BBC 4 | N-methylmaleimide | | | | | | | | | | | | | | | 1 | | |
| BBC 5 | N-ethylmaleimide | | | | | | | | | | | | | | | 1 | | 1 |
| BBC 6 | N-(n-propyl)maleimide | | | | | | | | | | | | | | | 1 | 1 | |
| BBC 7 | N-benzylmaleimide | | | | | | | | | | | | | | | 1 | 1 | 1 |
| BBC 8 | 2-thienylmethyl maleimide | | | | | | | | | | | | | | 1 | | | |
| BBC 9 | N-phenylmaleimide | | | | | | | | | | | | | | 1 | | | 1 |
| BBC 10 | N-(4-ethylphenyl)maleimide | | | | | | | | | | | | | | 1 | | 1 | |
| BBC 11 | N-(4-vlnylphenyl)maleimide | | | | | | | | | | | | | | 1 | | 1 | 1 |
| BBC 12 | 1-[3,5-bis(trifluoromethyl)phenyl]- | | | | | | | | | | | | | | 1 | 1 | | |
| | 1H-pyrrole-2,5-dione | | | | | | | | | | | | | | | | | |
| BBC 13 | N-methoxycarbonylmaleimide | | | | | | | | | | | | | | 1 | 1 | | 1 |
| BBC 14 | N-cyclohexylmaleimide | | | | | | | | | | | | | | 1 | 1 | 1 | |
| BBC 15 | N-(4-methyl-3-chlorophenyl)maleimide | | | | | | | | | | | | | | 1 | 1 | 1 | 1 |
| BBC 16 | N-(4-chlorophenyl)maleimide | | | | | | | | | | | | | 1 | | | | |
| BBC 17 | N-(4-bromophenyl)maleimide | | | | | | | | | | | | | 1 | | | | 1 |
| BBC 18 | N-(4-iodophenyl)maleimide | | | | | | | | | | | | | 1 | | | 1 | |
| BBC 19 | N-hydroxymaleimide | | | | | | | | | | | | | 1 | | | 1 | 1 |
| BBC 20 | N-tert-butylmaleimide | | | | | | | | | | | | | 1 | | 1 | | |
| BBC 21 | beta-(4-hydroxyphenyl)ethylmaleimide | | | | | | | | | | | | | 1 | | 1 | | 1 |
| BBC 22 | N-[4-2-benzoxazolyl)phenyl]maleimide | | | | | | | | | | | | | 1 | | 1 | 1 | |
| BBC 23 | 2,5-dimethoxystilbene-4′-maleimide | | | | | | | | | | | | | 1 | | 1 | 1 | 1 |
| BBC 24 | N-(4-acetylphenyl)maleimide | | | | | | | | | | | | | 1 | 1 | | | |
| BBC 25 | 4-(N-maleimido)benzophenone | | | | | | | | | | | | | 1 | 1 | | | 1 |
| BBC 26 | 1-(1-benzylpiperidin-4-yl)-1H- | | | | | | | | | | | | | 1 | 1 | | 1 | |
| | pyrrole-2,5-dione | | | | | | | | | | | | | | | | | |
| BBC 27 | N-(3-nitrophenyl)maleimide | | | | | | | | | | | | | 1 | 1 | | 1 | 1 |
| BBC 28 | N-(4-nitrophenyl)maleimide | | | | | | | | | | | | | 1 | 1 | 1 | | |
| BBC 29 | N-(4-dimethylamino-3,5- | | | | | | | | | | | | | 1 | 1 | 1 | | 1 |
| | dinitrophenyl)maleimide | | | | | | | | | | | | | | | | | |
| BBC 30 | maleimide | | | | | | | | | | | | | 1 | 1 | 1 | 1 | |
| BBC 31 | N-(4-anilinophenyl)maleimide | | | | | | | | | | | | | 1 | 1 | 1 | 1 | 1 |
| BBC 32 | BIONET 9H-912 | | | | | | | | | | | | 1 | | | | | |
| BBC 33 | N-carbamoylmaleimide | | | | | | | | | | | | 1 | | | | | 1 |
| BBC 34 | 3-maleimidopropionic acid | | | | | | | | | | | | 1 | | | | 1 | |
| BBC 35 | 4-maleimidobutyric acid | | | | | | | | | | | | 1 | | | | 1 | 1 |
| BBC 36 | 6-maleimidocaproic acid | | | | | | | | | | | | 1 | | | 1 | | |
| BBC 37 | 4-dimethylaminophenylazophenyl- | | | | | | | | | | | | 1 | | | 1 | | 1 |
| | 4′-maleimide | | | | | | | | | | | | | | | | | |
| BBC 38 | 1-(4-morpholinophenyl)-2,5- | | | | | | | | | | | | 1 | | | 1 | 1 | |
| | dihydro-1H-pyrrole-2,5-dione | | | | | | | | | | | | | | | | | |
| BBC 39 | 3-maleimidophenyl boronic acid | | | | | | | | | | | | 1 | | | 1 | 1 | 1 |
| BBC 40 | 3-N-maleimidobenzoic acid | | | | | | | | | | | | 1 | | 1 | | | |
| BBC 41 | N-(4-carboxy-3- | | | | | | | | | | | | 1 | | 1 | | | 1 |
| | hydroxyphenyl)maleimide | | | | | | | | | | | | | | | | | |

X-ray Data for Compounds 7′, 10, 12, and 13

Data were collected using a Bruker APEX CCD (charge coupled device) based diffractometer equipped with an LT-3 low-temperature apparatus operating at 213K. A suitable crystal was chosen and mounted on a glass fiber using grease. Data were measured using omega scans of 0.3° per frame for 30 seconds, such that a hemisphere was collected. A total of 1271 frames were collected with a maximum resolution of 0.75 Å. The first 50 frames were recollected at the end of data collection to monitor for decay. Cell parameters were retrieved using SMART software (SMART V 5.054 (NT) Software for the CCD Detector System; Bruker Analytical X-ray Systems, Madison, Wis. (1998)) and refined using SAINT on all observed reflections. Data reduction was performed using the SAINT software (SAINT V 6.02 (NT) Software for the CCD Detector System Bruker Analytical X-ray Systems, Madison, Wis. (2000); incorporated herein by reference) which corrects for Lp and decay. The structures are solved by the direct method using the SHELXS-97 (Sheldrick, G. M. SHELXS-90, Program for the Solution of Crystal Structure, University of Göttingen, Germany, 1990; incorporated herein by reference) program and refined by least squares method on F², SHELXL-97 (Sheldrick, G. M. SHELXL-97, Program for the Refinement of Crystal Structure, University of Göttingen, Germany, 1997; incorporated herein by reference), incorporated in SHELXTL V5.10 (SHELXTL 6.10 (PC/NT-Version), Program library for Structure Solution and Molecular Graphics; Bruker Analytical X-ray Systems, Madison, Wis. (2000); incorporated herein by reference).

The structure was solved in the space group P2₁/c (# 14) by analysis of systematic absences. All non-hydrogen atoms are refined anisotropically. Hydrogens were calculated by geometrical methods and refined as a riding model. The crystal used for the diffraction study showed no decomposition during data collection. All drawing are done at 50% ellipsiods.

[image]

[image]

[image]

[image]

[image]

[image]

[image]

[image]

[image]

[image]

[image]

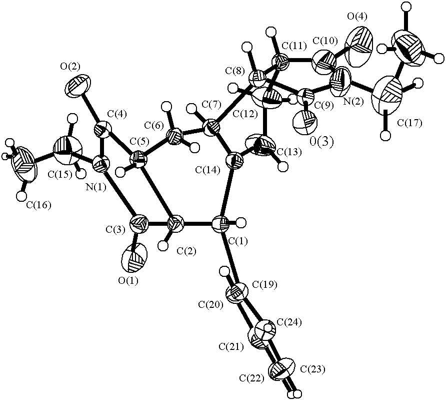

TABLE 1
Crystal data and structure refinement for sls34t.

| Identification code | sls34t | |
|---|---|---|
| Empirical formula | C24 H26 N2 O4 | |
| Formula weight | 406.47 | |
| Temperature | 213(2) K | |
| Wavelength | 0.71073 Å | |
| Crystal system | Monoclinic | |
| Space group | P2(1)/n | |
| Unit cell dimensions | a = 11.4789(6) Å | α = 90°. |
| | b = 11.1983(5) Å | β = 106.559(1)°. |
| | c = 16.7409(8) Å | γ = 90°. |

| | |
|---|---|
| Volume | 2062.7(2) Å³ |
| Z | 4 |
| Density (calculated) | 1.309 Mg/m³ |
| Absorption coefficient | 0.089 mm⁻¹ |
| F(000) | 864 |
| Crystal size | 0.20 × 0.15 × 0.10 mm³ |
| Theta range for data collection | 1.92 to 23.28°. |
| Index ranges | −12 <= h <= 12, −7 <= k <= 12, |
| | −18 <= l <= 17 |
| Reflections collected | 9899 |
| Independent reflections | 2972 [R(int) = 0.0325] |
| Completeness to theta = 23.28° | 99.8% |
| Absorption correction | None |
| Refinement method | Full-matrix least-squares on F² |
| Data/restraints/parameters | 2972/0/271 |
| Goodness-of-fit on F² | 1.055 |
| Final R indices [I > 2sigma(I)] | R1 = 0.0419, wR2 = 0.1098 |
| R indices (all data) | R1 = 0.0495, wR2 = 0.1160 |
| Largest diff. peak and hole | 0.375 and −0.277 e.Å⁻³ |

TABLE 2
Atomic coordinates (×10⁴) and equivalent isotropic displacement
parameters (Å² × 10³) for sls34t. U(eq) is defined as one third
of the trace of the orthogonalized U^{ij} tensor.

| | x | y | z | U(eq) |
|---|---|---|---|---|
| O(1) | 6794(2) | 4225(1) | 4761(1) | 75(1) |
| O(2) | 5496(2) | 5879(1) | 2193(1) | 60(1) |
| O(3) | 5900(1) | 603(1) | 1412(1) | 47(1) |
| O(4) | 2236(2) | −599(1) | 1640(1) | 72(1) |
| N(1) | 5992(1) | 5225(1) | 3541(1) | 41(1) |
| N(2) | 4123(1) | −245(1) | 1471(1) | 43(1) |
| C(1) | 6510(2) | 2160(2) | 3413(1) | 34(1) |
| C(2) | 7048(2) | 3443(2) | 3458(1) | 37(1) |
| C(3) | 6623(2) | 4295(2) | 4018(1) | 45(1) |
| C(4) | 5994(2) | 5161(2) | 2723(1) | 39(1) |
| C(5) | 6698(2) | 4073(2) | 2603(1) | 36(1) |
| C(6) | 5969(2) | 3295(2) | 1882(1) | 37(1) |
| C(7) | 4865(2) | 2768(2) | 2089(1) | 33(1) |
| C(8) | 4148(2) | 1856(2) | 1444(1) | 33(1) |
| C(9) | 4855(2) | 713(2) | 1437(1) | 35(1) |
| C(10) | 3016(2) | 95(2) | 1580(1) | 46(1) |
| C(11) | 2995(2) | 1428(2) | 1650(1) | 36(1) |
| C(12) | 2992(2) | 1776(2) | 2540(1) | 47(1) |
| C(13) | 4246(2) | 1670(2) | 3140(1) | 44(1) |
| C(14) | 5170(2) | 2173(2) | 2940(1) | 34(1) |
| C(15) | 5427(2) | 6204(2) | 3877(2) | 54(1) |
| C(16) | 6203(2) | 7282(3) | 4071(2) | 95(1) |
| C(17) | 4498(2) | −1487(2) | 1427(2) | 64(1) |
| C(18) | 4550(3) | −1814(3) | 554(2) | 95(1) |
| C(19) | 6898(2) | 1486(2) | 4233(1) | 35(1) |
| C(20) | 6318(2) | 1580(2) | 4854(1) | 42(1) |
| C(21) | 6726(2) | 920(2) | 5584(1) | 51(1) |
| C(22) | 7722(2) | 178(2) | 5704(1) | 56(1) |
| C(23) | 8313(2) | 98(2) | 5101(1) | 53(1) |
| C(24) | 7897(2) | 739(2) | 4369(1) | 43(1) |

TABLE 3
Bond lengths [Å] and angles [°] for sls34t.

| | | | |
|---|---|---|---|
| O(2)—C(4) | 1.214(2) | O(1)—C(3) | 1.206(2) |
| O(4)—C(10) | 1.211(2) | O(3)—C(9) | 1.217(2) |
| N(1)—C(3) | 1.384(3) | N(1)—C(4) | 1.373(2) |
| N(2)—C(9) | 1.374(2) | N(1)—C(15) | 1.465(3) |
| N(2)—C(17) | 1.464(3) | N(2)—C(10) | 1.387(3) |
| C(1)—C(19) | 1.517(2) | C(1)—C(14) | 1.516(2) |
| C(2)—C(3) | 1.512(3) | C(1)—C(2) | 1.558(3) |
| C(4)—C(5) | 1.506(3) | C(2)—C(5) | 1.544(2) |
| C(6)—C(7) | 1.525(2) | C(5)—C(6) | 1.529(3) |
| C(7)—C(8) | 1.543(2) | C(7)—C(14) | 1.521(2) |
| C(8)—C(11) | 1.535(2) | C(8)—C(9) | 1.518(2) |
| C(11)—C(12) | 1.542(3) | C(10)—C(11) | 1.498(3) |
| C(13)—C(14) | 1.326(3) | C(12)—C(13) | 1.506(3) |
| C(17)—C(18) | 1.525(4) | C(15)—C(16) | 1.480(3) |
| C(19)—C(20) | 1.390(3) | C(19)—C(24) | 1.384(3) |
| C(21)—C(22) | 1.381(3) | C(20)—C(21) | 1.391(3) |
| C(23)—C(24) | 1.382(3) | C(22)—C(23) | 1.370(3) |
| C(4)—N(1)—C(3) | 112.69(16) | C(4)—N(1)— | 123.31(17) |
| | | C(15) | |
| C(3)—N(1)—C(15) | 123.94(17) | C(9)—N(2)— | 112.70(16) |
| | | C(10) | |
| C(9)—N(2)—C(17) | 123.16(16) | C(10)—N(2)— | 124.10(17) |
| | | C(17) | |
| C(14)—C(1)—C(19) | 118.04(15) | C(14)—C(1)— | 110.19(14) |
| | | C(2) | |
| C(19)—C(1)—C(2) | 113.87(14) | C(3)—C(2)— | 103.99(15) |
| | | C(5) | |
| C(3)—C(2)—C(1) | 114.76(15) | C(5)—C(2)— | 112.46(14) |
| | | C(1) | |
| O(1)—C(3)—N(1) | 123.3(2) | O(1)—C(3)— | 127.71(19) |
| | | C(2) | |
| N(1)—C(3)—C(2) | 109.03(16) | O(2)—C(4)— | 124.10(18) |
| | | N(1) | |
| O(2)—C(4)—C(5) | 126.63(18) | N(1)—C(4)— | 109.27(15) |
| | | C(5) | |
| C(4)—C(5)—C(6) | 111.86(15) | C(4)—C(5)— | 104.65(15) |
| | | C(2) | |
| C(6)—C(5)—C(2) | 114.25(14) | C(7)—C(6)— | 110.16(15) |
| | | C(5) | |
| C(14)—C(7)—C(6) | 113.44(15) | C(14)—C(7)— | 107.46(14) |
| | | C(8) | |
| C(6)—C(7)—C(8) | 114.19(15) | C(9)—C(8)— | 103.67(14) |
| | | C(11) | |
| C(9)—C(8)—C(7) | 112.59(14) | C(11)—C(8)— | 111.81(14) |
| | | C(7) | |
| O(3)—C(9)—N(2) | 122.89(17) | O(3)—C(9)— | 128.32(16) |
| | | C(8) | |
| N(2)—C(9)—C(8) | 108.79(15) | O(4)—C(10)— | 124.13(19) |
| | | N(2) | |
| O(4)—C(10)—C(11) | 127.19(19) | N(2)—C(10)— | 108.62(16) |
| | | C(11) | |
| C(10)—C(11)—C(8) | 105.02(15) | C(10)—C(11)— | 109.36(16) |
| | | C(12) | |
| C(8)—C(11)—C(12) | 112.18(15) | C(13)—C(12)— | 111.09(16) |
| | | C(11) | |
| C(14)—C(13)—C(12) | 118.67(17) | C(13)—C(14)— | 129.05(17) |
| | | C(1) | |
| C(13)—C(14)—C(7) | 115.80(16) | C(1)—C(14)— | 114.97(15) |
| | | C(7) | |
| N(1)—C(15)—C(16) | 112.96(19) | N(2)—C(17)— | 111.7(2) |
| | | C(18) | |
| C(24)—C(19)—C(20) | 118.23(17) | C(24)—C(19)— | 117.77(16) |
| | | C(1) | |
| C(20)—C(19)—C(1) | 124.00(17) | C(19)—C(20)— | 120.3(2) |
| | | C(21) | |
| C(22)—C(21)—C(20) | 120.3(2) | C(23)—C(22)— | 119.7(2) |
| | | C(21) | |
| C(22)—C(23)—C(24) | 120.0(2) | C(23)—C(24)— | 121.4(2) |
| | | C(19) | |

TABLE 4
Anisotropic displacement parameters (Å² × 10³) for sls34t.
The anisotropic displacement factor exponent takes the form:
−2π²[h² a*²U¹¹ + . . . + 2 h k a* b* U¹²]

| | U¹¹ | U²² | U³³ | U²³ | U¹³ | U¹² |
|---|---|---|---|---|---|---|
| O(1) | 134(2) | 52(1) | 34(1) | −1(1) | 15(1) | −2(1) |
| O(2) | 77(1) | 47(1) | 46(1) | 8(1) | 4(1) | 12(1) |
| O(3) | 35(1) | 45(1) | 62(1) | −5(1) | 17(1) | 1(1) |
| O(4) | 52(1) | 48(1) | 125(2) | 16(1) | 40(1) | −6(1) |
| N(1) | 41(1) | 41(1) | 40(1) | −1(1) | 12(1) | −2(1) |
| N(2) | 37(1) | 33(1) | 61(1) | 5(1) | 15(1) | 1(1) |
| C(1) | 33(1) | 36(1) | 32(1) | 1(1) | 9(1) | 1(1) |
| C(2) | 29(1) | 40(1) | 38(1) | 3(1) | 4(1) | −3(1) |
| C(3) | 55(1) | 42(1) | 34(1) | 0(1) | 7(1) | −11(1) |
| C(4) | 36(1) | 39(1) | 37(1) | 3(1) | 5(1) | −5(1) |
| C(5) | 33(1) | 38(1) | 36(1) | 3(1) | 11(1) | −4(1) |
| C(6) | 39(1) | 38(1) | 35(1) | 4(1) | 11(1) | −2(1) |
| C(7) | 33(1) | 33(1) | 33(1) | 1(1) | 9(1) | 1(1) |
| C(8) | 31(1) | 35(1) | 30(1) | 3(1) | 6(1) | 1(1) |
| C(9) | 33(1) | 39(1) | 33(1) | 0(1) | 8(1) | −1(1) |
| C(10) | 37(1) | 44(1) | 56(1) | 9(1) | 13(1) | −1(1) |
| C(11) | 29(1) | 40(1) | 37(1) | 3(1) | 7(1) | 1(1) |
| C(12) | 35(1) | 67(1) | 41(1) | −2(1) | 15(1) | −6(1) |
| C(13) | 41(1) | 58(1) | 34(1) | 3(1) | 12(1) | −8(1) |
| C(14) | 35(1) | 37(1) | 30(1) | −1(1) | 10(1) | −1(1) |
| C(15) | 51(1) | 53(1) | 62(1) | −7(1) | 26(1) | 3(1) |
| C(16) | 57(2) | 71(2) | 154(3) | −55(2) | 27(2) | −6(1) |
| C(17) | 52(1) | 35(1) | 109(2) | 14(1) | 30(1) | 6(1) |
| C(18) | 78(2) | 70(2) | 108(2) | −46(2) | −19(2) | 17(2) |
| C(19) | 36(1) | 33(1) | 33(1) | −1(1) | 6(1) | −4(1) |
| C(20) | 44(1) | 44(1) | 36(1) | 0(1) | 9(1) | −4(1) |
| C(21) | 61(1) | 54(1) | 36(1) | 2(1) | 13(1) | −15(1) |
| C(22) | 66(2) | 47(1) | 45(1) | 16(1) | −1(1) | −8(1) |
| C(23) | 54(1) | 41(1) | 57(1) | 12(1) | 4(1) | 5(1) |
| C(24) | 45(1) | 36(1) | 45(1) | 1(1) | 8(1) | 1(1) |

TABLE 5
Hydrogen coordinates (×10⁴) and isotropic
displacement parameters (Å² × 10³) for sls34t.

| | x | y | z | U(eq) |
|---|---|---|---|---|
| H(1A) | 6897 | 1719 | 3044 | 41 |
| H(2A) | 7945 | 3387 | 3660 | 44 |
| H(5A) | 7451 | 4338 | 2479 | 43 |
| H(6A) | 5703 | 3778 | 1374 | 44 |
| H(6B) | 6485 | 2649 | 1780 | 44 |
| H(7A) | 4306 | 3438 | 2098 | 40 |
| H(8A) | 3923 | 2220 | 882 | 39 |
| H(11A) | 2265 | 1755 | 1240 | 43 |
| H(12A) | 2704 | 2599 | 2541 | 56 |
| H(12B) | 2432 | 1253 | 2723 | 56 |
| H(13A) | 4370 | 1255 | 3646 | 53 |
| H(15A) | 5248 | 5931 | 4385 | 64 |
| H(15B) | 4655 | 6414 | 3470 | 64 |
| H(16A) | 5786 | 7899 | 4290 | 142 |
| H(16B) | 6369 | 7568 | 3568 | 142 |
| H(16C) | 6962 | 7085 | 4483 | 142 |
| H(17A) | 3922 | −2016 | 1587 | 77 |
| H(17B) | 5301 | −1607 | 1824 | 77 |
| H(18A) | 4801 | −2641 | 548 | 142 |
| H(18B) | 5129 | −1301 | 398 | 142 |
| H(18C) | 3752 | −1710 | 161 | 142 |
| H(20A) | 5647 | 2092 | 4780 | 50 |
| H(21A) | 6322 | 980 | 5999 | 61 |
| H(22A) | 7993 | −271 | 6197 | 67 |
| H(23A) | 9002 | −393 | 5185 | 64 |
| H(24A) | 8301 | 667 | 3956 | 51 |

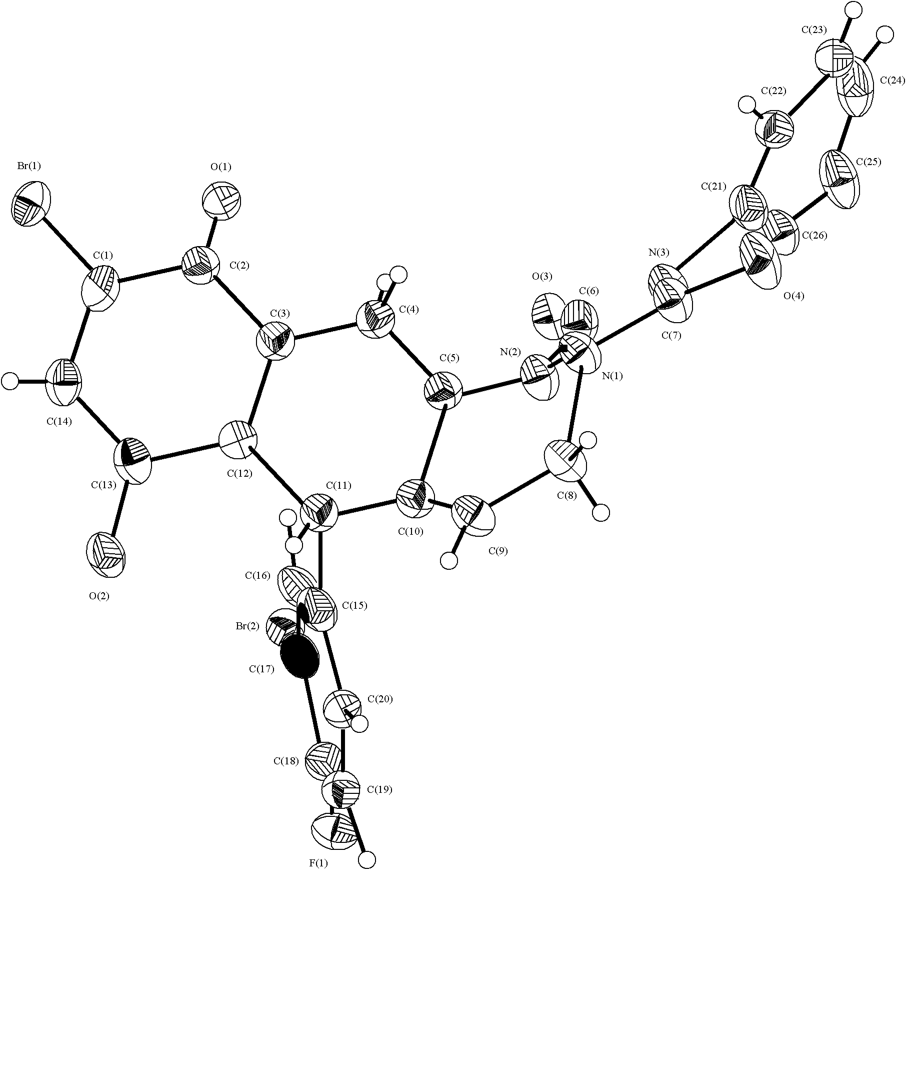 TABLE 1
Crystal data and structure refinement for sls50t.

| Identification code | sls50t | |
|---|---|---|
| Empirical formula | C26 H16 Br2 F N3 O4 | |
| Formula weight | 613.24 | |
| Temperature | 213(2) K | |
| Wavelength | 0.71073 Å | |
| Crystal system | Triclinic | |
| Space group | P-1 | |
| Unit cell dimensions | a = 10.4042(6) Å | α = 87.6570(10)°. |
| | b = 10.9633(7) Å | β = 71.8390(10)°. |
| | c = 11.9333(7) Å | γ = 69.4440(10)°. |

| | |
|---|---|
| Volume | 1207.41(13) Å³ |
| Z | 2 |
| Density (calculated) | 1.687 Mg/m³ |
| Absorption coefficient | 3.404 mm⁻¹ |
| F(000) | 608 |
| Crystal size | 0.10 × 0.08 × 0.08 mm³ |
| Theta range for data collection | 1.80 to 27.93°. |
| Index ranges | −7 <= h <= 13, −13 <= k <= 14, |
| | −15 <= l <= 14 |
| Reflections collected | 7913 |
| Independent reflections | 5030 [R(int) = 0.0592] |
| Completeness to theta = 27.93° | 87.0% |
| Absorption correction | None |
| Refinement method | Full-matrix least-squares on F² |
| Data/restraints/parameters | 5030/0/325 |
| Goodness-of-fit on F² | 1.008 |
| Final R indices [I > 2sigma(I)] | R1 = 0.0466, wR2 = 0.1187 |
| R indices (all data) | R1 = 0.0683, wR2 = 0.1303 |
| Largest diff. peak and hole | 0.969 and −0.681 e.Å⁻³ |

TABLE 2
Atomic coordinates (×10⁴) and equivalent isotropic displacement
parameters (Å² × 10³) for sls50t. U(eq) is defined as one third
of the trace of the orthogonalized U^{ij} tensor.

| | x | y | z | U(eq) |
|---|---|---|---|---|
| Br(1) | −5107(1) | −5925(1) | −3015(1) | 58(1) |
| Br(2) | −267(1) | −2131(1) | −2097(1) | 56(1) |
| O(1) | −2415(3) | −6143(3) | −5041(3) | 59(1) |
| O(2) | −5349(3) | −1176(3) | −3526(3) | 54(1) |
| O(3) | 2264(3) | −5525(3) | −7216(2) | 42(1) |
| O(4) | 1491(4) | −4099(3) | −10708(2) | 70(1) |
| F(1) | −736(3) | 687(3) | −2628(2) | 66(1) |
| N(1) | 271(3) | −3405(3) | −8744(3) | 40(1) |
| N(2) | 675(3) | −3696(3) | −7705(2) | 35(1) |
| N(3) | 2269(3) | −5136(3) | −9154(3) | 42(1) |
| C(1) | −4516(4) | −4577(4) | −3757(3) | 43(1) |
| C(2) | −3113(4) | −5002(4) | −4719(3) | 41(1) |
| C(3) | −2585(4) | −3953(3) | −5293(3) | 34(1) |
| C(4) | −1189(4) | −4433(3) | −6307(3) | 40(1) |
| C(5) | −521(4) | −3383(3) | −6593(3) | 35(1) |
| C(6) | 1802(4) | −4871(4) | −7937(3) | 38(1) |
| C(7) | 1356(4) | −4194(4) | −9665(3) | 46(1) |
| C(8) | −494(4) | −2001(4) | −8800(3) | 42(1) |
| C(9) | −1637(4) | −1496(3) | −7641(3) | 39(1) |
| C(10) | −1667(4) | −2097(3) | −6654(3) | 34(1) |
| C(11) | −2828(4) | −1608(3) | −5474(3) | 35(1) |
| C(12) | −3324(3) | −2692(3) | −4924(3) | 34(1) |
| C(13) | −4702(4) | −2316(4) | −3917(3) | 40(1) |
| C(14) | −5274(4) | −3336(4) | −3407(3) | 44(1) |
| C(15) | −2288(4) | −995(3) | −4669(3) | 34(1) |
| C(16) | −1690(4) | −1703(3) | −3848(3) | 37(1) |
| C(17) | −1147(4) | −1141(4) | −3177(3) | 40(1) |
| C(18) | −1242(4) | 127(4) | −3307(3) | 44(1) |
| C(19) | −1831(4) | 863(4) | −4104(4) | 49(1) |
| C(20) | −2334(4) | 286(4) | −4790(3) | 43(1) |
| C(21) | 3453(4) | −6282(4) | −9749(3) | 45(1) |
| C(22) | 3262(5) | −7073(4) | −10501(4) | 57(1) |
| C(23) | 4386(6) | −8233(5) | −11019(5) | 72(2) |
| C(24) | 5658(7) | −8577(5) | −10779(5) | 84(2) |
| C(25) | 5867(5) | −7759(6) | −10047(5) | 88(2) |
| C(26) | 4736(5) | −6593(5) | −9515(4) | 65(1) |

TABLE 3
Bond lengths [Å] and angles [°] for sls50t.

| | | | |
|---|---|---|---|
| Br(2)—C(17) | 1.898(4) | Br(1)—C(1) | 1.881(4) |
| O(2)—C(13) | 1.222(5) | O(1)—C(2) | 1.211(5) |
| O(4)—C(7) | 1.212(5) | O(3)—C(6) | 1.205(4) |
| N(1)—C(7) | 1.361(5) | F(1)—C(18) | 1.358(5) |
| N(1)—C(8) | 1.473(4) | N(1)—N(2) | 1.420(4) |
| N(2)—C(5) | 1.461(4) | N(2)—C(6) | 1.369(4) |
| N(3)—C(7) | 1.401(5) | N(3)—C(6) | 1.387(5) |
| C(1)—C(14) | 1.317(6) | N(3)—C(21) | 1.429(5) |
| C(2)—C(3) | 1.496(5) | C(1)—C(2) | 1.479(5) |
| C(3)—C(4) | 1.506(5) | C(3)—C(12) | 1.337(5) |
| C(5)—C(10) | 1.510(5) | C(4)—C(5) | 1.520(5) |
| C(9)—C(10) | 1.323(5) | C(8)—C(9) | 1.486(5) |
| C(11)—C(12) | 1.506(5) | C(10)—C(11) | 1.509(5) |
| C(12)—C(13) | 1.491(5) | C(11)—C(15) | 1.531(5) |
| C(15)—C(16) | 1.388(5) | C(13)—C(14) | 1.470(6) |
| C(16)—C(17) | 1.381(5) | C(15)—C(20) | 1.391(5) |
| C(18)—C(19) | 1.373(6) | C(17)—C(18) | 1.365(5) |
| C(21)—C(22) | 1.375(6) | C(19)—C(20) | 1.373(6) |
| C(22)—C(23) | 1.391(6) | C(21)—C(26) | 1.372(6) |
| C(24)—C(25) | 1.390(9) | C(23)—C(24) | 1.361(8) |
| | | C(25)—C(26) | 1.402(7) |
| C(7)—N(1)— | 108.2(3) | C(7)—N(1)— | 124.0(3) |
| N(2) | | C(8) | |
| N(2)—N(1)— | 113.4(3) | C(6)—N(2)— | 107.8(3) |
| C(8) | | N(1) | |
| C(6)—N(2)— | 121.6(3) | N(1)—N(2)— | 115.5(3) |
| C(5) | | C(5) | |
| C(6)—N(3)— | 110.5(3) | C(6)—N(3)— | 122.8(3) |
| C(7) | | C(21) | |
| C(7)—N(3)— | 126.5(3) | C(14)—C(1)— | 122.2(3) |
| C(21) | | C(2) | |
| C(14)—C(1)— | 122.0(3) | C(2)—C(1)— | 115.7(3) |
| Br(1) | | Br(1) | |
| O(1)—C(2)— | 122.2(3) | O(1)—C(2)— | 120.8(3) |
| C(1) | | C(3) | |
| C(1)—C(2)— | 117.0(3) | C(12)—C(3)— | 121.4(3) |
| C(3) | | C(2) | |
| C(12)—C(3)— | 123.8(3) | C(2)—C(3)— | 114.8(3) |
| C(4) | | C(4) | |
| C(3)—C(4)— | 110.7(3) | N(2)—C(5)— | 109.2(3) |
| C(5) | | C(10) | |
| N(2)—C(5)— | 112.9(3) | C(10)—C(5)— | 109.6(3) |
| C(4) | | C(4) | |
| O(3)—C(6)— | 126.1(3) | O(3)—C(6)— | 128.2(3) |
| N(2) | | N(3) | |
| N(2)—C(6)— | 105.7(3) | O(4)—C(7)— | 126.8(4) |
| N(3) | | N(1) | |
| O(4)—C(7)— | 127.7(4) | N(1)—C(7)— | 105.5(3) |
| N(3) | | N(3) | |
| N(1)—C(8)— | 107.9(3) | C(10)—C(9)— | 124.4(3) |
| C(9) | | C(8) | |
| C(9)—C(10)— | 122.9(3) | C(9)—C(10)— | 125.1(3) |
| C(5) | | C(11) | |
| C(5)—C(10)— | 112.1(3) | C(12)—C(11)— | 110.3(3) |
| C(11) | | C(10) | |
| C(12)—C(11)— | 112.5(3) | C(10)—C(11)— | 110.3(3) |
| C(15) | | C(15) | |
| C(3)—C(12)— | 119.6(3) | C(3)—C(12)— | 123.1(3) |
| C(13) | | C(11) | |
| C(13)—C(12)— | 117.3(3) | O(2)—C(13)— | 120.6(3) |
| C(11) | | C(14) | |
| O(2)—C(13)— | 120.4(4) | C(14)—C(13)— | 119.0(3) |
| C(12) | | C(12) | |
| C(1)—C(14)— | 120.5(3) | C(16)—C(15)— | 118.6(4) |
| C(13) | | C(20) | |
| C(16)—C(15)— | 121.7(3) | C(20)—C(15)— | 119.7(3) |
| C(11) | | C(11) | |
| C(17)—C(16)— | 120.2(3) | C(18)—C(17)— | 119.2(4) |
| C(15) | | C(16) | |
| C(18)—C(17)— | 120.6(3) | C(16)—C(17)— | 120.2(3) |
| Br(2) | | Br(2) | |
| F(1)—C(18)— | 119.2(4) | F(1)—C(18)— | 118.5(4) |
| C(17) | | C(19) | |
| C(17)—C(18)— | 122.3(4) | C(18)—C(19)— | 118.0(4) |
| C(19) | | C(20) | |
| C(19)—C(20)— | 121.5(4) | C(22)—C(21)— | 121.6(4) |
| C(15) | | C(26) | |
| C(22)—C(21)— | 119.5(4) | C(26)—C(21)— | 118.8(4) |
| N(3) | | N(3) | |
| C(21)—C(22)— | 119.4(5) | C(24)—C(23)— | 120.0(6) |
| C(23) | | C(22) | |
| C(23)—C(24)— | 120.6(5) | C(24)—C(25)— | 119.6(5) |
| C(25) | | C(26) | |
| C(21)—C(26)— | 118.6(5) | | |
| C(25) | | | |

TABLE 4
Anisotropic displacement parameters (Å² × 10³) for sls50t.
The anisotropic displacement factor exponent takes the form:
−2π²[h² a*²U¹¹ + . . . + 2 h k a* b* U¹²]

| | U¹¹ | U²² | U³³ | U²³ | U¹³ | U¹² |
|---|---|---|---|---|---|---|
| Br(1) | 62(1) | 63(1) | 54(1) | 20(1) | −14(1) | −34(1) |
| Br(2) | 74(1) | 63(1) | 38(1) | 6(1) | −31(1) | −19(1) |
| O(1) | 72(2) | 35(2) | 56(2) | 8(1) | −2(2) | −18(2) |
| O(2) | 44(2) | 44(2) | 53(2) | 6(1) | −8(1) | 3(1) |
| O(3) | 37(1) | 44(2) | 35(1) | 10(1) | −15(1) | −2(1) |
| O(4) | 87(2) | 63(2) | 27(1) | 4(1) | −20(1) | 12(2) |
| F(1) | 80(2) | 66(2) | 64(2) | −7(1) | −26(1) | −35(2) |
| N(1) | 48(2) | 39(2) | 26(2) | 7(1) | −19(1) | −3(1) |
| N(2) | 35(2) | 36(2) | 27(1) | 7(1) | −13(1) | −4(1) |
| N(3) | 43(2) | 42(2) | 29(2) | 5(1) | −10(1) | 0(1) |
| C(1) | 43(2) | 48(2) | 42(2) | 18(2) | −20(2) | −20(2) |
| C(2) | 43(2) | 40(2) | 38(2) | 11(2) | −15(2) | −12(2) |
| C(3) | 38(2) | 31(2) | 33(2) | 6(1) | −15(1) | −7(2) |
| C(4) | 39(2) | 32(2) | 39(2) | 5(2) | −11(2) | −4(2) |
| C(5) | 37(2) | 38(2) | 28(2) | 8(1) | −14(1) | −9(2) |
| C(6) | 34(2) | 40(2) | 35(2) | 9(2) | −11(2) | −10(2) |
| C(7) | 51(2) | 42(2) | 34(2) | 5(2) | −16(2) | −3(2) |
| C(8) | 55(2) | 34(2) | 35(2) | 10(2) | −21(2) | −8(2) |
| C(9) | 48(2) | 26(2) | 35(2) | 4(1) | −19(2) | −1(2) |
| C(10) | 40(2) | 27(2) | 34(2) | 6(1) | −17(2) | −8(2) |
| C(11) | 38(2) | 30(2) | 34(2) | 6(1) | −18(2) | −2(2) |
| C(12) | 33(2) | 35(2) | 31(2) | 8(1) | −16(1) | −6(2) |
| C(13) | 32(2) | 40(2) | 40(2) | 6(2) | −16(2) | 2(2) |
| C(14) | 33(2) | 53(3) | 40(2) | 10(2) | −11(2) | −9(2) |
| C(15) | 34(2) | 28(2) | 31(2) | 4(1) | −8(1) | −2(2) |
| C(16) | 42(2) | 30(2) | 32(2) | 2(1) | −12(2) | −6(2) |
| C(17) | 42(2) | 43(2) | 28(2) | 1(2) | −10(2) | −9(2) |
| C(18) | 46(2) | 44(2) | 39(2) | −9(2) | −10(2) | −15(2) |
| C(19) | 59(2) | 32(2) | 53(2) | 4(2) | −13(2) | −18(2) |
| C(20) | 48(2) | 35(2) | 45(2) | 11(2) | −18(2) | −10(2) |
| C(21) | 47(2) | 39(2) | 34(2) | 9(2) | −5(2) | −4(2) |
| C(22) | 61(3) | 45(3) | 53(3) | 2(2) | −3(2) | −17(2) |
| C(23) | 83(4) | 48(3) | 67(3) | 0(2) | −1(3) | −21(3) |
| C(24) | 80(4) | 54(3) | 67(4) | 6(3) | 11(3) | 6(3) |
| C(25) | 46(3) | 96(5) | 74(4) | 1(3) | −3(3) | 16(3) |
| C(26) | 51(3) | 66(3) | 54(3) | 1(2) | −13(2) | 4(2) |

TABLE 5
Hydrogen coordinates (×10⁴) and isotropic
displacement parameters (Å² × 10³) for sls50t.

| | x | y | z | U(eq) |
|---|---|---|---|---|
| H(4A) | −1377 | −4677 | −7005 | 48 |
| H(4B) | −509 | −5213 | −6098 | 48 |
| H(5A) | −149 | −3284 | −5947 | 42 |
| H(8A) | −930 | −1871 | −9435 | 51 |
| H(8B) | 186 | −1534 | −8963 | 51 |
| H(9A) | −2389 | −697 | −7607 | 46 |
| H(11A) | −3666 | −917 | −5615 | 42 |
| H(14A) | −6188 | −3100 | −2825 | 53 |
| H(16A) | −1655 | −2568 | −3749 | 44 |
| H(19A) | −1889 | 1736 | −4178 | 59 |
| H(20A) | −2719 | 768 | −5355 | 52 |
| H(22A) | 2381 | −6832 | −10663 | 69 |
| H(23A) | 4266 | −8779 | −11535 | 87 |
| H(24A) | 6402 | −9376 | −11110 | 101 |
| H(25A) | 6761 | −7987 | −9910 | 105 |
| H(26A) | 4855 | −6036 | −9009 | 78 |

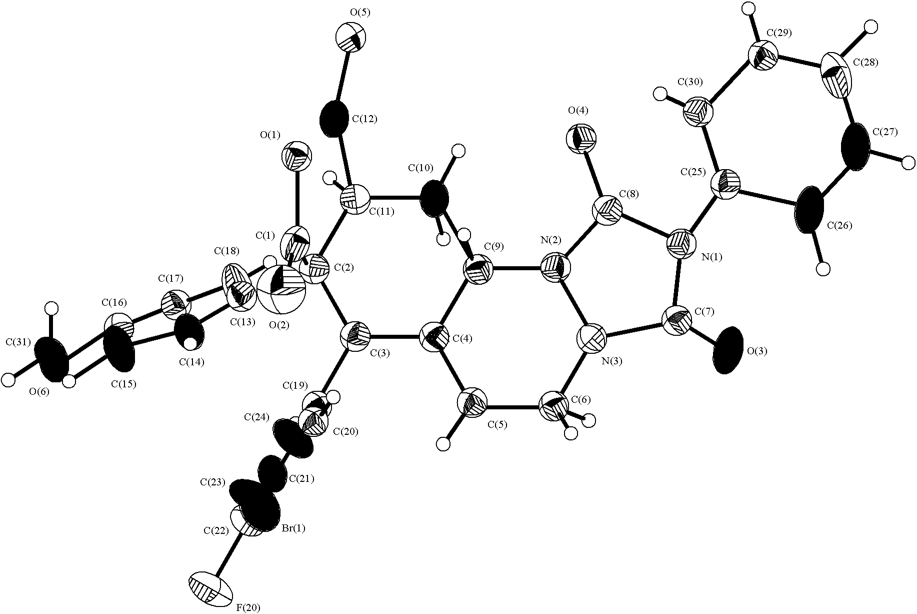 TABLE 1
Crystal data and structure refinement for sls55t.

| Identification code | sls55t | |
|---|---|---|
| Empirical formula | C31.50 H24 Br Cl F N3 O6 | |
| Formula weight | 674.90 | |
| Temperature | 213(2) K | |
| Wavelength | 0.71073 Å | |
| Crystal system | Triclinic | |
| Space group | P-1 | |
| Unit cell dimensions | a = 8.7883(11) Å | α = 89.879(2)°. |
| | b = 10.7793(15) Å | β = 81.922(3)°. |
| | c = 16.657(2) Å | γ = 66.124(2)°. |

| | |
|---|---|
| Volume | 1426.0(3) Å³ |
| Z | 2 |
| Density (calculated) | 1.572 Mg/m³ |
| Absorption coefficient | 1.593 mm⁻¹ |
| F(000) | 686 |
| Crystal size | 0.15 × 0.15 × 0.12 mm³ |
| Theta range for data collection | 1.24 to 25.00°. |
| Index ranges | −10 <= h <= 8, −12 <= k <= 12, |
| | −19 <= l <= 9 |
| Reflections collected | 8038 |
| Independent reflections | 4989 [R(int) = 0.0556] |
| Completeness to theta = 25.00° | 99.0% |
| Absorption correction | None |
| Refinement method | Full-matrix least-squares on F² |
| Data/restraints/parameters | 4989/0/406 |
| Goodness-of-fit on F² | 1.002 |
| Final R indices [I > 2sigma(I)] | R1 = 0.0489, wR2 = 0.1167 |
| R indices (all data) | R1 = 0.0991, wR2 = 0.1361 |
| Largest diff. peak and hole | 0.515 and −0.472 e.Å⁻³ |

TABLE 2
Atomic coordinates (×10⁴) and equivalent isotropic displacement
parameters (Å² × 10³) for sls55t. U(eq) is defined as one
third of the trace of the orthogonalized U^{ij} tensor.

| | x | y | z | U(eq) |
|---|---|---|---|---|
| Br(1) | 1640(1) | 1210(1) | 4437(1) | 64(1) |
| F(20) | 4094(4) | 1626(3) | 5420(2) | 77(1) |
| O(1) | 6192(3) | −861(3) | 707(2) | 38(1) |
| O(2) | 4774(4) | −425(3) | 1971(2) | 45(1) |
| O(3) | 57(3) | 6401(3) | 331(2) | 40(1) |
| O(4) | 5149(3) | 3322(3) | −1053(2) | 35(1) |
| O(5) | 7675(4) | −767(3) | −482(2) | 44(1) |
| O(6) | 11973(4) | −2678(3) | 3497(2) | 59(1) |
| N(1) | 2417(4) | 4965(3) | −599(2) | 30(1) |
| N(2) | 4070(4) | 3944(3) | 313(2) | 30(1) |
| N(3) | 2413(4) | 4716(3) | 720(2) | 32(1) |
| C(1) | 5809(5) | −273(4) | 1496(3) | 33(1) |
| C(2) | 6961(5) | 460(4) | 1605(2) | 30(1) |
| C(3) | 6016(5) | 1931(4) | 2058(2) | 30(1) |
| C(4) | 4666(5) | 2944(4) | 1615(2) | 30(1) |
| C(5) | 3474(5) | 4090(4) | 1976(2) | 34(1) |
| C(6) | 2253(5) | 5187(4) | 1554(2) | 34(1) |
| C(7) | 1441(5) | 5475(4) | 171(3) | 35(1) |
| C(8) | 4018(5) | 3982(4) | −518(2) | 30(1) |
| C(9) | 4940(5) | 2648(4) | 706(2) | 31(1) |
| C(10) | 6813(5) | 2015(4) | 379(2) | 31(1) |
| C(11) | 7631(5) | 587(4) | 707(2) | 30(1) |
| C(12) | 7258(5) | −412(4) | 220(3) | 33(1) |
| C(13) | 8364(5) | −435(4) | 2074(2) | 31(1) |
| C(14) | 8031(5) | −1173(4) | 2722(2) | 40(1) |
| C(15) | 9250(5) | −1895(4) | 3174(3) | 40(1) |
| C(16) | 10841(5) | −1911(4) | 3012(2) | 40(1) |
| C(17) | 11208(5) | −1170(4) | 2389(2) | 39(1) |
| C(18) | 9954(5) | −446(4) | 1930(2) | 36(1) |
| C(19) | 5440(5) | 1900(4) | 2969(2) | 34(1) |
| C(20) | 3988(5) | 1699(4) | 3251(2) | 36(1) |
| C(21) | 3566(5) | 1584(4) | 4068(3) | 42(1) |
| C(22) | 4535(6) | 1724(5) | 4615(3) | 50(1) |
| C(23) | 5948(6) | 1954(5) | 4356(3) | 55(1) |
| C(24) | 6385(6) | 2043(4) | 3527(3) | 43(1) |
| C(25) | 1814(5) | 5328(4) | −1363(2) | 33(1) |
| C(26) | 638(5) | 6643(4) | −1444(3) | 37(1) |
| C(27) | 62(5) | 6996(5) | −2173(3) | 42(1) |
| C(28) | 679(6) | 6062(5) | −2846(3) | 47(1) |
| C(29) | 1844(6) | 4752(5) | −2760(3) | 44(1) |
| C(30) | 2403(5) | 4368(4) | −2019(3) | 41(1) |
| C(31) | 13655(6) | −2790(6) | 3314(3) | 70(2) |
| C(1S) | 1330(20) | 5182(17) | 4563(9) | 94(6) |
| Cl(1) | 2877(4) | 5230(3) | 5101(2) | 86(1) |
| Cl(2) | −240(8) | 4836(5) | 5190(3) | 94(2) |

TABLE 3
Bond lengths [Å] and angles [°] for sls55t.

| | | | |
|---|---|---|---|
| F(20)—C(22) | 1.358(5) | Br(1)—C(21) | 1.914(4) |
| O(1)—C(1) | 1.397(5) | O(1)—C(12) | 1.386(5) |
| O(3)—C(7) | 1.214(5) | O(2)—C(1) | 1.182(5) |
| O(5)—C(12) | 1.190(5) | O(4)—C(8) | 1.212(4) |
| O(6)—C(31) | 1.422(6) | O(6)—C(16) | 1.372(5) |
| N(1)—C(7) | 1.412(5) | N(1)—C(8) | 1.403(5) |
| N(2)—C(8) | 1.391(5) | N(1)—C(25) | 1.439(5) |
| N(2)—C(9) | 1.496(5) | N(2)—N(3) | 1.421(4) |
| N(3)—C(6) | 1.448(5) | N(3)—C(7) | 1.374(5) |
| C(2)—C(13) | 1.538(5) | C(1)—C(2) | 1.544(6) |
| C(2)—C(3) | 1.592(5) | C(2)—C(11) | 1.555(5) |
| C(3)—C(19) | 1.536(5) | C(3)—C(4) | 1.529(5) |
| C(4)—C(9) | 1.512(5) | C(4)—C(5) | 1.328(5) |
| C(9)—C(10) | 1.521(5) | C(5)—C(6) | 1.491(5) |
| C(11)—C(12) | 1.513(6) | C(10)—C(11) | 1.547(5) |
| C(13)—C(14) | 1.407(5) | C(13)—C(18) | 1.380(5) |
| C(15)—C(16) | 1.380(6) | C(14)—C(15) | 1.367(5) |
| C(17)—C(18) | 1.394(5) | C(16)—C(17) | 1.390(6) |
| C(19)—C(20) | 1.397(6) | C(19)—C(24) | 1.377(6) |
| C(21)—C(22) | 1.378(6) | C(20)—C(21) | 1.378(6) |
| C(23)—C(24) | 1.396(6) | C(22)—C(23) | 1.373(7) |
| C(25)—C(26) | 1.396(5) | C(25)—C(30) | 1.395(6) |
| C(27)—C(28) | 1.398(6) | C(26)—C(27) | 1.376(6) |
| C(29)—C(30) | 1.393(6) | C(28)—C(29) | 1.390(6) |
| C(1S)—Cl(1) | 1.744(17) | C(1S)—Cl(2)#1 | 0.999(16) |
| Cl(1)—Cl(2)#1 | 2.461(7) | C(1S)—Cl(2) | 1.776(17) |
| Cl(2)—C(1S)#1 | 0.999(16) | Cl(2)—Cl(2)#1 | 0.865(7) |
| | | Cl(2)—Cl(1)#1 | 2.461(7) |
| C(12)—O(1)—C(1) | 110.8(3) | C(16)—O(6)—C(31) | 117.6(4) |
| C(8)—N(1)—C(7) | 110.7(3) | C(8)—N(1)—C(25) | 124.4(3) |
| C(7)—N(1)—C(25) | 124.7(3) | C(8)—N(2)—N(3) | 107.7(3) |
| C(8)—N(2)—C(9) | 122.2(3) | N(3)—N(2)—C(9) | 112.4(3) |
| C(7)—N(3)—N(2) | 109.1(3) | C(7)—N(3)—C(6) | 123.7(3) |
| N(2)—N(3)—C(6) | 116.8(3) | O(2)—C(1)—O(1) | 120.0(4) |
| O(2)—C(1)—C(2) | 130.1(4) | O(1)—C(1)—C(2) | 109.9(3) |
| C(13)—C(2)—C(1) | 108.9(3) | C(13)—C(2)—C(11) | 112.8(3) |
| C(1)—C(2)—C(11) | 101.5(3) | C(13)—C(2)—C(3) | 109.3(3) |
| C(1)—C(2)—C(3) | 114.6(3) | C(11)—C(2)—C(3) | 109.7(3) |
| C(4)—C(3)—C(19) | 114.2(3) | C(4)—C(3)—C(2) | 114.0(3) |
| C(19)—C(3)—C(2) | 113.4(3) | C(5)—C(4)—C(9) | 121.9(3) |
| C(5)—C(4)—C(3) | 122.8(3) | C(9)—C(4)—C(3) | 114.7(3) |
| C(4)—C(5)—C(6) | 125.0(4) | N(3)—C(6)—C(5) | 109.1(3) |
| O(3)—C(7)—N(3) | 126.2(4) | O(3)—C(7)—N(1) | 128.6(4) |
| N(3)—C(7)—N(1) | 105.2(3) | O(4)—C(8)—N(2) | 126.5(4) |
| O(4)—C(8)—N(1) | 127.9(4) | N(2)—C(8)—N(1) | 105.6(3) |
| N(2)—C(9)—C(4) | 108.9(3) | N(2)—C(9)—C(10) | 110.7(3) |
| C(4)—C(9)—C(10) | 110.2(3) | C(9)—C(10)—C(11) | 109.5(3) |
| C(12)—C(11)—C(10) | 109.0(3) | C(12)—C(11)—C(2) | 104.8(3) |
| C(10)—C(11)—C(2) | 115.4(3) | O(5)—C(12)—O(1) | 120.5(4) |
| O(5)—C(12)—C(11) | 129.6(4) | O(1)—C(12)—C(11) | 109.9(3) |
| C(18)—C(13)—C(14) | 117.1(4) | C(18)—C(13)—C(2) | 121.5(3) |
| C(14)—C(13)—C(2) | 121.1(3) | C(15)—C(14)—C(13) | 121.1(4) |
| C(14)—C(15)—C(16) | 120.9(4) | O(6)—C(16)—C(15) | 116.2(4) |
| O(6)—C(16)—C(17) | 124.2(4) | C(15)—C(16)—C(17) | 119.6(4) |
| C(16)—C(17)—C(18) | 118.7(4) | C(13)—C(18)—C(17) | 122.4(4) |
| C(24)—C(19)—C(20) | 118.7(4) | C(24)—C(19)—C(3) | 119.8(4) |
| C(20)—C(19)—C(3) | 121.5(4) | C(21)—C(20)—C(19) | 120.1(4) |
| C(20)—C(21)—C(22) | 120.2(4) | C(20)—C(21)—Br(1) | 119.6(3) |
| C(22)—C(21)—Br(1) | 120.2(3) | F(20)—C(22)—C(23) | 119.5(4) |
| F(20)—C(22)—C(21) | 119.6(4) | C(23)—C(22)—C(21) | 120.9(4) |
| C(22)—C(23)—C(24) | 118.6(4) | C(19)—C(24)—C(23) | 121.5(4) |
| C(30)—C(25)—C(26) | 120.1(4) | C(30)—C(25)—N(1) | 120.1(4) |
| C(26)—C(25)—N(1) | 119.8(4) | C(27)—C(26)—C(25) | 120.0(4) |
| C(26)—C(27)—C(28) | 120.7(4) | C(29)—C(28)—C(27) | 119.0(4) |
| C(28)—C(29)—C(30) | 121.0(4) | C(29)—C(30)—C(25) | 119.2(4) |
| Cl(2)#1—C(1S)—Cl(1) | 125.4(13) | Cl(2)#1—C(1S)—Cl(2) | 16.4(6) |
| Cl(1)—C(1S)—Cl(2) | 112.2(8) | C(1S)—Cl(1)—Cl(2)#1 | 19.3(5) |
| Cl(2)#1—Cl(2)—C(1S)#1 | 144.5(15) | Cl(2)#1—Cl(2)—C(1S) | 19.1(9) |
| C(1S)#1—Cl(2)—C(1S) | 163.6(6) | Cl(2)#1—Cl(2)—Cl(1)#1 | 113.9(8) |
| C(1S)#1—Cl(2)—Cl(1)#1 | 35.3(10) | C(1S)—Cl(2)—Cl(1)#1 | 131.1(5) |

Symmetry transformations used to generate equivalent atoms:
#1 −x, −y + 1, −z + 1

TABLE 4
Anisotropic displacement parameters (Å² × 10³) for sls55t. The anisotropic
displacement factor exponent takes the form:
−2π²[h²a*²U¹¹ + . . . + 2hka*b*U¹²]

| | U¹¹ | U²² | U³³ | U²³ | U¹³ | U¹² |
|---|---|---|---|---|---|---|
| Br (1) | 57 (1) | 76 (1) | 57 (1) | 23 (1) | 4 (1) | −31 (1) |
| F (20) | 88 (2) | 114 (3) | 32 (2) | 19 (2) | −7 (2) | −45 (2) |
| O (1) | 45 (2) | 33 (2) | 37 (2) | 0 (1) | −5 (1) | −18 (1) |
| O (2) | 45 (2) | 52 (2) | 46 (2) | 3 (2) | 1 (2) | −31 (2) |
| O (3) | 31 (2) | 30 (2) | 49 (2) | 6 (1) | 0 (1) | −5 (1) |
| O (4) | 32 (2) | 36 (2) | 29 (2) | 6 (1) | 1 (1) | −8 (1) |
| O (5) | 55 (2) | 33 (2) | 35 (2) | −1 (1) | −3 (2) | −9 (1) |
| O (6) | 39 (2) | 74 (2) | 55 (2) | 23 (2) | −12 (2) | −14 (2) |
| N (1) | 29 (2) | 26 (2) | 32 (2) | 3 (2) | 0 (2) | −10 (2) |
| N (2) | 31 (2) | 25 (2) | 29 (2) | 4 (2) | 0 (2) | −7 (2) |
| N (3) | 33 (2) | 26 (2) | 32 (2) | 2 (2) | −1 (2) | −8 (2) |
| C (1) | 31 (2) | 28 (2) | 38 (3) | 3 (2) | −7 (2) | −10 (2) |
| C (2) | 27 (2) | 33 (2) | 30 (2) | 4 (2) | 0 (2) | −13 (2) |
| C (3) | 32 (2) | 34 (2) | 29 (2) | 2 (2) | −1 (2) | −19 (2) |
| C (4) | 31 (2) | 33 (2) | 30 (2) | 4 (2) | −5 (2) | −18 (2) |
| C (5) | 38 (3) | 39 (3) | 26 (2) | 3 (2) | −2 (2) | −19 (2) |
| C (6) | 36 (2) | 30 (2) | 31 (2) | −2 (2) | 2 (2) | −13 (2) |
| C (7) | 39 (3) | 28 (2) | 40 (3) | 4 (2) | 0 (2) | −19 (2) |
| C (8) | 33 (2) | 25 (2) | 34 (2) | 6 (2) | −4 (2) | −14 (2) |
| C (9) | 36 (2) | 29 (2) | 31 (2) | 9 (2) | −5 (2) | −17 (2) |
| C (10) | 32 (2) | 29 (2) | 32 (2) | 5 (2) | −4 (2) | −14 (2) |
| C (11) | 25 (2) | 32 (2) | 31 (2) | 4 (2) | 0 (2) | −10 (2) |
| C (12) | 31 (2) | 25 (2) | 35 (3) | 6 (2) | −5 (2) | −3 (2) |
| C (13) | 26 (2) | 33 (2) | 33 (2) | 0 (2) | 2 (2) | −14 (2) |
| C (14) | 36 (3) | 47 (3) | 38 (3) | 9 (2) | −1 (2) | −20 (2) |
| C (15) | 39 (3) | 47 (3) | 38 (3) | 17 (2) | −9 (2) | −19 (2) |
| C (16) | 39 (3) | 41 (3) | 32 (2) | 6 (2) | −9 (2) | −8 (2) |
| C (17) | 31 (2) | 43 (3) | 42 (3) | 5 (2) | −6 (2) | −15 (2) |
| C (18) | 42 (3) | 38 (3) | 31 (2) | 9 (2) | −2 (2) | −20 (2) |
| C (19) | 35 (2) | 33 (2) | 31 (2) | 4 (2) | −4 (2) | −11 (2) |
| C (20) | 35 (2) | 37 (3) | 32 (2) | 7 (2) | −3 (2) | −12 (2) |
| C (21) | 38 (3) | 46 (3) | 39 (3) | 12 (2) | −2 (2) | −14 (2) |
| C (22) | 64 (3) | 56 (3) | 28 (2) | 11 (2) | −7 (2) | −22 (3) |
| C (23) | 63 (3) | 75 (4) | 33 (3) | 7 (2) | −15 (2) | −30 (3) |
| C (24) | 48 (3) | 51 (3) | 34 (3) | 7 (2) | −10 (2) | −24 (2) |
| C (25) | 31 (2) | 34 (2) | 35 (2) | 9 (2) | −2 (2) | −16 (2) |
| C (26) | 36 (3) | 31 (3) | 48 (3) | 5 (2) | −9 (2) | −16 (2) |
| C (27) | 38 (3) | 35 (3) | 59 (3) | 16 (2) | −17 (2) | −18 (2) |
| C (28) | 48 (3) | 51 (3) | 52 (3) | 24 (3) | −21 (2) | −26 (2) |
| C (29) | 47 (3) | 48 (3) | 36 (3) | 3 (2) | −4 (2) | −21 (2) |
| C (30) | 42 (3) | 35 (3) | 42 (3) | 6 (2) | −4 (2) | −13 (2) |
| C (31) | 40 (3) | 95 (4) | 63 (4) | 22 (3) | −18 (3) | −12 (3) |
| C (1S) | 172 (19) | 64 (9) | 65 (10) | 47 (7) | −29 (12) | −65 (13) |
| Cl (1) | 88 (2) | 77 (2) | 71 (2) | 17 (2) | −10 (2) | −12 (2) |
| Cl (2) | 152 (5) | 73 (3) | 75 (5) | 11 (3) | −20 (4) | −63 (3) |

TABLE 5
Hydrogen coordinates (×10⁴) and isotropic displacement
parameters (Å²× 10³) for sls55t.

| | x | y | z | U(eq) |
|---|---|---|---|---|
| H(3A) | 6893 | 2291 | 2035 | 36 |
| H(5A) | 3393 | 4220 | 2541 | 41 |
| H(6A) | 1103 | 5421 | 1831 | 40 |
| H(6B) | 2485 | 6003 | 1567 | 40 |
| H(9A) | 4444 | 2002 | 590 | 37 |
| H(10A) | 7345 | 2593 | 550 | 37 |
| H(10B) | 6981 | 1940 | −216 | 37 |
| H(11A) | 8865 | 303 | 644 | 36 |
| H(14A) | 6953 | −1171 | 2846 | 48 |
| H(15A) | 9000 | −2387 | 3600 | 48 |
| H(17A) | 12279 | −1157 | 2278 | 47 |
| H(18A) | 10202 | 53 | 1507 | 44 |
| H(20A) | 3297 | 1642 | 2883 | 43 |
| H(23A) | 6606 | 2048 | 4731 | 67 |
| H(24A) | 7347 | 2205 | 3344 | 51 |
| H(26A) | 239 | 7287 | −1000 | 45 |
| H(27A) | −754 | 7875 | −2220 | 50 |
| H(28A) | 312 | 6316 | −3349 | 56 |
| H(29A) | 2259 | 4116 | −3208 | 52 |
| H(30A) | 3169 | 3474 | −1962 | 49 |
| H(31A) | 14322 | −3354 | 3699 | 105 |
| H(31B) | 13659 | −1894 | 3348 | 105 |
| H(31C) | 14132 | −3201 | 2768 | 105 |
| H(1SA) | 1892 | 4353 | 4228 | 113 |
| H(1SB) | 1110 | 5912 | 4203 | 113 |

TABLE 6
Torsion angles [°] for sls55t.

| | |
|---|---|
| C(8)—N(2)—N(3)—C(7) | −13.9(4) |
| C(9)—N(2)—N(3)—C(7) | −151.4(3) |
| C(8)—N(2)—N(3)—C(6) | −160.4(3) |
| C(9)—N(2)—N(3)—C(6) | 62.1(4) |
| C(12)—O(1)—C(1)—O(2) | 171.8(4) |
| C(12)—O(1)—C(1)—C(2) | −10.7(4) |
| O(2)—C(1)—C(2)—C(13) | 75.0(5) |
| O(1)—C(1)—C(2)—C(13) | −102.2(3) |
| O(2)—C(1)—C(2)—C(11) | −165.8(4) |
| O(1)—C(1)—C(2)—C(11) | 17.0(4) |
| O(2)—C(1)—C(2)—C(3) | −47.7(6) |
| O(1)—C(1)—C(2)—C(3) | 135.1(3) |
| C(13)—C(2)—C(3)—C(4) | 176.1(3) |
| C(1)—C(2)—C(3)—C(4) | −61.4(4) |
| C(11)—C(2)—C(3)—C(4) | 51.9(4) |
| C(13)—C(2)—C(3)—C(19) | −50.9(4) |
| C(1)—C(2)—C(3)—C(19) | 71.6(4) |
| C(11)—C(2)—C(3)—C(19) | −175.1(3) |
| C(19)—C(3)—C(4)—C(5) | 30.2(5) |
| C(2)—C(3)—C(4)—C(5) | 162.8(4) |
| C(19)—C(3)—C(4)—C(9) | −158.4(3) |
| C(2)—C(3)—C(4)—C(9) | −25.8(5) |
| C(9)—C(4)—C(5)—C(6) | 1.7(6) |
| C(3)—C(4)—C(5)—C(6) | 172.5(4) |
| C(7)—N(3)—C(6)—C(5) | 178.7(3) |
| N(2)—N(3)—C(6)—C(5) | −40.2(4) |
| C(4)—C(5)—C(6)—N(3) | 8.4(6) |
| N(2)—N(3)—C(7)—O(3) | −167.4(4) |
| C(6)—N(3)—C(7)—O(3) | −23.7(6) |
| N(2)—N(3)—C(7)—N(1) | 11.2(4) |
| C(6)—N(3)—C(7)—N(1) | 154.9(3) |
| C(8)—N(1)—C(7)—O(3) | 173.8(4) |
| C(25)—N(1)—C(7)—O(3) | −10.5(6) |
| C(8)—N(1)—C(7)—N(3) | −4.7(4) |
| C(25)—N(1)—C(7)—N(3) | 171.0(3) |
| N(3)—N(2)—C(8)—O(4) | −171.4(4) |
| C(9)—N(2)—C(8)—O(4) | −38.9(6) |
| N(3)—N(2)—C(8)—N(1) | 10.4(4) |
| C(9)—N(2)—C(8)—N(1) | 142.8(3) |
| C(7)—N(1)—C(8)—O(4) | 178.1(4) |
| C(25)—N(1)—C(8)—O(4) | 2.4(6) |
| C(7)—N(1)—C(8)—N(2) | −3.6(4) |
| C(25)—N(1)—C(8)—N(2) | −179.4(3) |
| C(8)—N(2)—C(9)—C(4) | −176.3(3) |
| N(3)—N(2)—C(9)—C(4) | −45.8(4) |
| C(8)—N(2)—C(9)—C(10) | 62.4(5) |
| N(3)—N(2)—C(9)—C(10) | −167.1(3) |
| C(5)—C(4)—C(9)—N(2) | 16.4(5) |
| C(3)—C(4)—C(9)—N(2) | −155.0(3) |
| C(5)—C(4)—C(9)—C(10) | 138.1(4) |
| C(3)—C(4)—C(9)—C(10) | −33.4(4) |
| N(2)—C(9)—C(10)—C(11) | −172.3(3) |
| C(4)—C(9)—C(10)—C(11) | 67.2(4) |
| C(9)—C(10)—C(11)—C(12) | 78.7(4) |
| C(9)—C(10)—C(11)—C(2) | −38.9(5) |
| C(13)—C(2)—C(11)—C(12) | 99.8(4) |
| C(1)—C(2)—C(11)—C(12) | −16.5(4) |
| C(3)—C(2)—C(11)—C(12) | −138.1(3) |
| C(13)—C(2)—C(11)—C(10) | −140.2(3) |
| C(1)—C(2)—C(11)—C(10) | 103.4(4) |
| C(3)—C(2)—C(11)—C(10) | −18.1(5) |
| C(1)—O(1)—C(12)—O(5) | −177.3(3) |
| C(1)—O(1)—C(12)—C(11) | −0.9(4) |
| C(10)—C(11)—C(12)—O(5) | 63.7(5) |
| C(2)—C(11)—C(12)—O(5) | −172.2(4) |
| C(10)—C(11)—C(12)—O(1) | −112.4(3) |
| C(2)—C(11)—C(12)—O(1) | 11.7(4) |
| C(1)—C(2)—C(13)—C(18) | 147.2(4) |
| C(11)—C(2)—C(13)—C(18) | 35.4(5) |
| C(3)—C(2)—C(13)—C(18) | −87.0(4) |
| C(1)—C(2)—C(13)—C(14) | −39.0(5) |
| C(11)—C(2)—C(13)—C(14) | −150.9(4) |
| C(3)—C(2)—C(13)—C(14) | 86.8(4) |
| C(18)—C(13)—C(14)—C(15) | −1.5(6) |
| C(2)—C(13)—C(14)—C(15) | −175.5(4) |
| C(13)—C(14)—C(15)—C(16) | 0.5(7) |
| C(31)—O(6)—C(16)—C(15) | 175.9(4) |
| C(31)—O(6)—C(16)—C(17) | −4.4(6) |
| C(14)—C(15)—C(16)—O(6) | −179.4(4) |
| C(14)—C(15)—C(16)—C(17) | 0.8(7) |
| O(6)—C(16)—C(17)—C(18) | 179.0(4) |
| C(15)—C(16)—C(17)—C(18) | −1.2(6) |
| C(14)—C(13)—C(18)—C(17) | 1.1(6) |
| C(2)—C(13)—C(18)—C(17) | 175.1(4) |
| C(16)—C(17)—C(18)—C(13) | 0.3(6) |
| C(4)—C(3)—C(19)—C(24) | −127.7(4) |
| C(2)—C(3)—C(19)—C(24) | 99.4(4) |
| C(4)—C(3)—C(19)—C(20) | 53.7(5) |
| C(2)—C(3)—C(19)—C(20) | −79.2(5) |
| C(24)—C(19)—C(20)—C(21) | −3.0(6) |
| C(3)—C(19)—C(20)—C(21) | 175.6(4) |
| C(19)—C(20)—C(21)—C(22) | 2.8(6) |
| C(19)—C(20)—C(21)—Br(1) | −176.6(3) |
| C(20)—C(21)—C(22)—F(20) | 179.2(4) |
| Br(1)—C(21)—C(22)—F(20) | −1.4(6) |
| C(20)—C(21)—C(22)—C(23) | −1.3(7) |
| Br(1)—C(21)—C(22)—C(23) | 178.1(4) |
| F(20)—C(22)—C(23)—C(24) | 179.6(4) |
| C(21)—C(22)—C(23)—C(24) | 0.0(7) |
| C(20)—C(19)—C(24)—C(23) | 1.8(7) |
| C(3)—C(19)—C(24)—C(23) | −176.8(4) |
| C(22)—C(23)—C(24)—C(19) | −0.4(7) |
| C(8)—N(1)—C(25)—C(30) | 29.5(6) |
| C(7)—N(1)—C(25)—C(30) | −145.7(4) |
| C(8)—N(1)—C(25)—C(26) | −150.5(4) |
| C(7)—N(1)—C(25)—C(26) | 34.3(6) |
| C(30)—C(25)—C(26)—C(27) | −0.3(6) |
| N(1)—C(25)—C(26)—C(27) | 179.8(4) |
| C(25)—C(26)—C(27)—C(28) | −1.9(6) |
| C(26)—C(27)—C(28)—C(29) | 2.2(7) |
| C(27)—C(28)—C(29)—C(30) | −0.4(7) |
| C(28)—C(29)—C(30)—C(25) | −1.7(7) |
| C(26)—C(25)—C(30)—C(29) | 2.0(6) |
| N(1)—C(25)—C(30)—C(29) | −178.0(4) |
| Cl(2)—C(1S)—Cl(1)—Cl(2)#1 | −11.2(9) |
| Cl(1)—C(1S)—Cl(2)—Cl(2)#1 | 146(3) |
| Cl(2)#1—C(1S)—Cl(2)—C(1S)#1 | 0.000(17) |
| Cl(1)—C(1S)—Cl(2)—C(1S)#1 | 146(3) |
| Cl(2)#1—C(1S)—Cl(2)—Cl(1)#1 | 29(2) |
| Cl(1)—C(1S)—Cl(2)—Cl(1)#1 | 175.1(4) |

Symmetry transformations used to generate equivalent atoms:
#1 −x, −y + 1, −z + 1

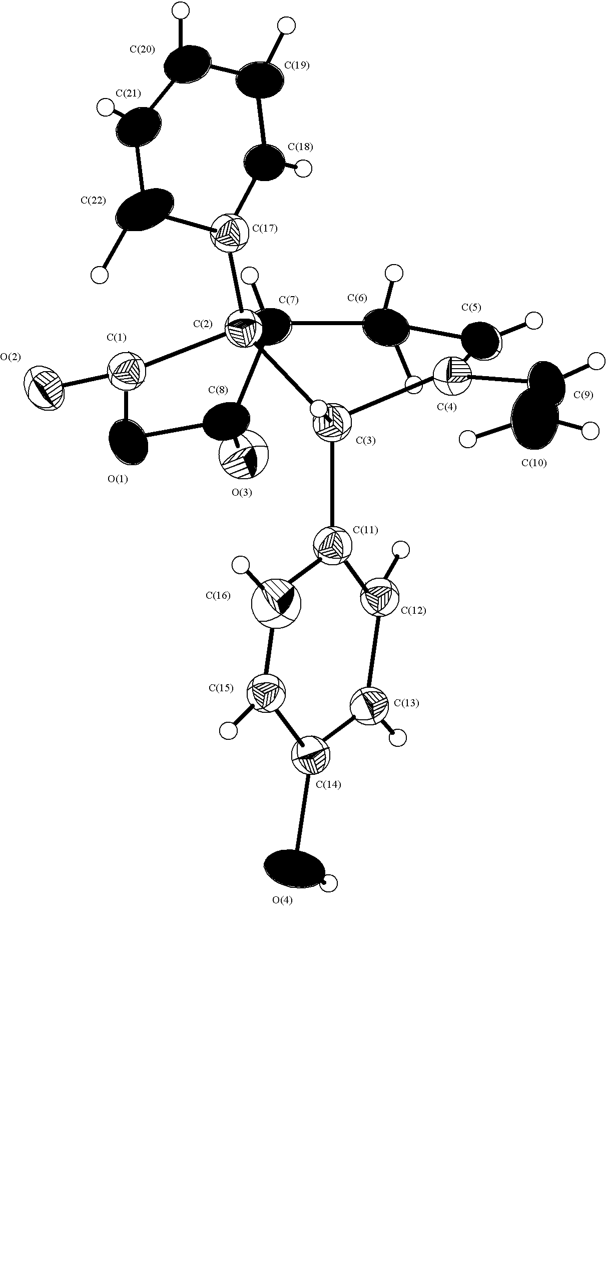 TABLE 1
Crystal data and structure refinement for sls56t.

| Identification code | sls56t | |
|---|---|---|
| Empirical formula | C23 H20 Cl2 O4 | |
| Formula weight | 431.29 | |
| Temperature | 213(2) K | |
| Wavelength | 0.71073 Å | |
| Crystal system | Monoclinic | |
| Space group | P2(1)/n | |
| Unit cell dimensions | a = 9.2454(5) Å | α = 90°. |
| | b = 8.6672(4) Å | β = 95.5260(10)°. |
| | c = 25.6417(15) Å | γ = 90°. |

| | |
|---|---|
| Volume | 2045.16(19) Å³ |
| Z | 4 |
| Density (calculated) | 1.401 Mg/m³ |
| Absorption coefficient | 0.345 mm⁻¹ |
| F(000) | 896 |
| Crystal size | 0.20 × 0.20 × 0.05 mm³ |
| Theta range for data collection | 1.60 to 27.94°. |
| Index ranges | −11 <= h <= 11, −4 <= k <= 11, |
| | −32 <= l <= 31 |
| Reflections collected | 12796 |
| Independent reflections | 4409 [R(int) = 0.0379] |
| Completeness to theta = 27.94° | 89.7% |
| Absorption correction | None |
| Refinement method | Full-matrix least-squares on F² |
| Data/restraints/parameters | 4409/0/262 |
| Goodness-of-fit on F² | 0.968 |
| Final R indices [I > 2sigma(I)] | R1 = 0.0465, wR2 = 0.1206 |
| R indices (all data) | R1 = 0.0769, wR2 = 0.1397 |
| Largest diff. peak and hole | 0.611 and −0.825 e.Å⁻³ |

TABLE 2
Atomic coordinates (×10⁴) and equivalent isotropic displacement
parameters (Å² × 10³) for sls56t. U(eq) is defined as one
third of the trace of the orthogonalized U^{ij} tensor.

| | x | y | z | U(eq) |
|---|---|---|---|---|
| O(1) | −5798(2) | −8628(2) | −2294(1) | 41(1) |
| O(2) | −5217(2) | −10362(2) | −1672(1) | 41(1) |
| O(3) | −6413(2) | −6458(2) | −2738(1) | 55(1) |
| O(4) | 70(2) | −7472(2) | −2514(1) | 36(1) |
| C(1) | −5639(2) | −9088(3) | −1779(1) | 33(1) |
| C(2) | −5992(2) | −7764(2) | −1421(1) | 30(1) |
| C(3) | −4504(2) | −7040(2) | −1187(1) | 29(1) |
| C(4) | −4767(2) | −5433(2) | −984(1) | 33(1) |
| C(5) | −5786(3) | −4524(3) | −1222(1) | 39(1) |
| C(6) | −6780(3) | −4940(3) | −1692(1) | 42(1) |
| C(7) | −6839(2) | −6675(3) | −1814(1) | 35(1) |
| C(8) | −6346(3) | −7121(3) | −2331(1) | 40(1) |
| C(9) | −3820(3) | −4895(3) | −528(1) | 43(1) |
| C(10) | −2719(3) | −5635(3) | −283(1) | 56(1) |
| C(11) | −3285(2) | −7078(2) | −1546(1) | 28(1) |
| C(12) | −3174(2) | −6023(2) | −1951(1) | 30(1) |
| C(13) | −2071(2) | −6127(3) | −2279(1) | 32(1) |
| C(14) | −1043(2) | −7295(2) | −2203(1) | 29(1) |
| C(15) | −1111(2) | −8333(3) | −1799(1) | 33(1) |
| C(16) | −2225(2) | −8217(2) | −1474(1) | 31(1) |
| C(17) | −6823(2) | −8295(3) | −969(1) | 32(1) |
| C(18) | −8050(3) | −7517(3) | −837(1) | 42(1) |
| C(19) | −8740(3) | −7958(4) | −402(1) | 53(1) |
| C(20) | −8221(3) | −9174(4) | −95(1) | 55(1) |
| C(21) | −7019(3) | −9960(3) | −224(1) | 53(1) |
| C(22) | −6317(3) | −9526(3) | −654(1) | 43(1) |
| C(1S) | −730(3) | −3378(4) | −1337(1) | 69(1) |
| Cl(1) | −2305(1) | −2281(1) | −1366(1) | 56(1) |
| Cl(2) | 649(1) | −2633(2) | −902(1) | 106(1) |

TABLE 3
Bond lengths [Å] and angles [°] for sls56t.

| | | | |
|---|---|---|---|
| O(1)—C(8) | 1.400(3) | O(1)—C(1) | 1.374(3) |
| O(3)—C(8) | 1.187(3) | O(2)—C(1) | 1.194(3) |
| C(1)—C(2) | 1.524(3) | O(4)—C(14) | 1.370(3) |
| C(2)—C(7) | 1.539(3) | C(2)—C(17) | 1.523(3) |
| C(3)—C(4) | 1.515(3) | C(2)—C(3) | 1.578(3) |
| C(4)—C(5) | 1.331(3) | C(3)—C(11) | 1.523(3) |
| C(5)—C(6) | 1.488(3) | C(4)—C(9) | 1.468(3) |
| C(7)—C(8) | 1.496(3) | C(6)—C(7) | 1.535(3) |
| C(11)—C(16) | 1.391(3) | C(9)—C(10) | 1.312(4) |
| C(12)—C(13) | 1.385(3) | C(11)—C(12) | 1.395(3) |
| C(14)—C(15) | 1.377(3) | C(13)—C(14) | 1.389(3) |
| C(17)—C(22) | 1.390(3) | C(15)—C(16) | 1.389(3) |
| C(18)—C(19) | 1.392(4) | C(17)—C(18) | 1.389(3) |
| C(20)—C(21) | 1.371(4) | C(19)—C(20) | 1.374(4) |
| C(1S)—Cl(2) | 1.735(3) | C(21)—C(22) | 1.384(4) |
| | | C(1S)—Cl(1) | 1.735(3) |
| C(1)—O(1)—C(8) | 109.95(18) | O(2)—C(1)—O(1) | 119.5(2) |
| O(2)—C(1)—C(2) | 129.9(2) | O(1)—C(1)—C(2) | 110.52(19) |
| C(17)—C(2)—C(1) | 112.59(18) | C(17)—C(2)—C(7) | 114.94(18) |
| C(1)—C(2)—C(7) | 101.14(17) | C(17)—C(2)—C(3) | 108.39(16) |
| C(1)—C(2)—C(3) | 107.42(17) | C(7)—C(2)—C(3) | 112.04(18) |
| C(4)—C(3)—C(11) | 112.36(17) | C(4)—C(3)—C(2) | 109.50(17) |
| C(11)—C(3)—C(2) | 115.47(17) | C(5)—C(4)—C(9) | 121.3(2) |
| C(5)—C(4)—C(3) | 121.1(2) | C(9)—C(4)—C(3) | 117.5(2) |
| C(4)—C(5)—C(6) | 125.7(2) | C(5)—C(6)—C(7) | 114.15(19) |
| C(8)—C(7)—C(6) | 115.2(2) | C(8)—C(7)—C(2) | 104.03(19) |
| C(6)—C(7)—C(2) | 117.53(19) | O(3)—C(8)—O(1) | 120.0(2) |
| O(3)—C(8)—C(7) | 131.3(3) | O(1)—C(8)—C(7) | 108.64(19) |
| C(10)—C(9)—C(4) | 127.1(2) | C(16)—C(11)— | 117.58(19) |
| | | C(12) | |
| C(16)—C(11)—C(3) | 119.20(18) | C(12)—C(11)— | 123.22(19) |
| | | C(3) | |
| C(13)—C(12)—C(11) | 121.3(2) | C(12)—C(13)— | 119.77(19) |
| | | C(14) | |
| O(4)—C(14)—C(15) | 117.20(19) | O(4)—C(14)— | 122.76(19) |
| | | C(13) | |
| C(15)—C(14)—C(13) | 120.0(2) | C(14)—C(15)— | 119.6(2) |
| | | C(16) | |
| C(15)—C(16)—C(11) | 121.7(2) | C(22)—C(17)— | 118.0(2) |
| | | C(18) | |
| C(22)—C(17)—C(2) | 120.5(2) | C(18)—C(17)— | 121.5(2) |
| | | C(2) | |
| C(17)—C(18)—C(19) | 120.7(3) | C(20)—C(19)— | 120.5(3) |
| | | C(18) | |
| C(19)—C(20)—C(21) | 119.3(2) | C(20)—C(21)— | 120.7(3) |
| | | C(22) | |
| C(21)—C(22)—C(17) | 120.8(3) | Cl(2)—C(1S)— | 112.65(18) |
| | | Cl(1) | |

TABLE 4
Anisotropic displacement parameters (Å² × 10³) for sls56t. The anisotropic
displacement factor exponent takes the form:
−2π²[h²a*²U¹¹ + . . . + 2hka*b*U¹²]

| | U¹¹ | U²² | U³³ | U²³ | U¹³ | U¹² |
|---|---|---|---|---|---|---|
| O (1) | 44 (1) | 52 (1) | 28 (1) | −7 (1) | 5 (1) | −6 (1) |
| O (2) | 37 (1) | 41 (1) | 44 (1) | −8 (1) | 6 (1) | 0 (1) |
| O (3) | 58 (1) | 74 (1) | 32 (1) | 9 (1) | 0 (1) | −8 (1) |
| O (4) | 31 (1) | 41 (1) | 38 (1) | 5 (1) | 10 (1) | 3 (1) |
| C (1) | 24 (1) | 45 (1) | 31 (1) | −5 (1) | 3 (1) | −8 (1) |
| C (2) | 26 (1) | 35 (1) | 29 (1) | −2 (1) | 4 (1) | −2 (1) |
| C (3) | 26 (1) | 34 (1) | 26 (1) | 1 (1) | 3 (1) | −2 (1) |
| C (4) | 31 (1) | 35 (1) | 33 (1) | −1 (1) | 10 (1) | 4 (1) |
| C (5) | 39 (1) | 34 (1) | 44 (1) | −2 (1) | 13 (1) | 2 (1) |
| C (6) | 36 (1) | 46 (1) | 43 (1) | 8 (1) | 7 (1) | 10 (1) |
| C (7) | 25 (1) | 49 (1) | 32 (1) | 4 (1) | 1 (1) | 1 (1) |
| C (8) | 33 (1) | 55 (2) | 32 (1) | 1 (1) | 1 (1) | 9 (1) |
| C (9) | 48 (2) | 42 (1) | 39 (1) | −10 (1) | 7 (1) | −7 (1) |
| C (10) | 60 (2) | 59 (2) | 45 (2) | −7 (1) | −14 (1) | −12 (2) |
| C (11) | 24 (1) | 32 (1) | 27 (1) | 2 (1) | 1 (1) | 3 (1) |
| C (12) | 25 (1) | 32 (1) | 34 (1) | 2 (1) | 2 (1) | 2 (1) |
| C (13) | 32 (1) | 33 (1) | 30 (1) | 4 (1) | 5 (1) | −2 (1) |
| C (14) | 23 (1) | 34 (1) | 30 (1) | −2 (1) | 4 (1) | −3 (1) |
| C (15) | 25 (1) | 34 (1) | 39 (1) | 3 (1) | 2 (1) | 3 (1) |
| C (16) | 28 (1) | 33 (1) | 33 (1) | 5 (1) | 2 (1) | −3 (1) |
| C (17) | 27 (1) | 40 (1) | 29 (1) | −5 (1) | 4 (1) | −10 (1) |
| C (18) | 32 (1) | 53 (1) | 42 (1) | −4 (1) | 9 (1) | −2 (1) |
| C (19) | 39 (2) | 75 (2) | 49 (2) | −10 (1) | 20 (1) | −10 (1) |
| C (20) | 48 (2) | 80 (2) | 39 (1) | −1 (1) | 13 (1) | −27 (2) |
| C (21) | 51 (2) | 63 (2) | 44 (2) | 14 (1) | 4 (1) | −13 (1) |
| C (22) | 37 (1) | 51 (1) | 41 (1) | 8 (1) | 6 (1) | −6 (1) |
| C (1S) | 53 (2) | 81 (2) | 73 (2) | −26 (2) | −3 (2) | 6 (2) |
| Cl (1) | 54 (1) | 49 (1) | 62 (1) | 6 (1) | −4 (1) | 4 (1) |
| Cl (2) | 49 (1) | 195 (1) | 73 (1) | −60 (1) | −8 (1) | 14 (1) |

TABLE 5
Hydrogen coordinates (×10⁴) and isotropic displacement
parameters (Å² × 10³) for sls56t.

| | x | y | z | U(eq) |
|---|---|---|---|---|
| H(4A) | 12 | −6793 | −2743 | 54 |
| H(3A) | −4168 | −7671 | −877 | 34 |
| H(5A) | −5888 | −3533 | −1081 | 46 |
| H(6A) | −6467 | −4392 | −1996 | 50 |
| H(6B) | −7761 | −4583 | −1641 | 50 |
| H(7A) | −7874 | −6980 | −1824 | 42 |
| H(9A) | −4024 | −3913 | −397 | 51 |
| H(10A) | −2466 | −6621 | −397 | 67 |
| H(10B) | −2182 | −5180 | 7 | 67 |
| H(12A) | −3862 | −5225 | −2003 | 36 |
| H(13A) | −2017 | −5411 | −2551 | 38 |
| H(15A) | −409 | −9114 | −1743 | 39 |
| H(16A) | −2264 | −8927 | −1199 | 38 |
| H(18A) | −8418 | −6684 | −1044 | 50 |
| H(19A) | −9568 | −7419 | −317 | 64 |
| H(20A) | −8685 | −9464 | 200 | 66 |
| H(21A) | −6668 | −10802 | −19 | 63 |
| H(22A) | −5488 | −10070 | −735 | 52 |
| H(1SA) | −949 | −4431 | −1231 | 83 |
| H(1SB) | −392 | −3428 | −1688 | 83 |

TABLE 6
Torsion angles [°] for sls56t.

| | |
|---|---|
| C(8)—O(1)—C(1)—O(2) | 178.1(2) |
| C(8)—O(1)—C(1)—C(2) | −4.6(2) |
| O(2)—C(1)—C(2)—C(17) | −42.3(3) |
| O(1)—C(1)—C(2)—C(17) | 140.81(18) |
| O(2)—C(1)—C(2)—C(7) | −165.5(2) |
| O(1)—C(1)—C(2)—C(7) | 17.6(2) |
| O(2)—C(1)—C(2)—C(3) | 76.9(3) |
| O(1)—C(1)—C(2)—C(3) | −99.9(2) |
| C(17)—C(2)—C(3)—C(4) | −77.2(2) |
| C(1)—C(2)—C(3)—C(4) | 160.92(17) |
| C(7)—C(2)—C(3)—C(4) | 50.7(2) |
| C(17)—C(2)—C(3)—C(11) | 154.84(18) |
| C(1)—C(2)—C(3)—C(11) | 32.9(2) |
| C(7)—C(2)—C(3)—C(11) | −77.3(2) |
| C(11)—C(3)—C(4)—C(5) | 95.2(2) |
| C(2)—C(3)—C(4)—C(5) | −34.5(3) |
| C(11)—C(3)—C(4)—C(9) | −82.5(2) |
| C(2)—C(3)—C(4)—C(9) | 147.75(19) |
| C(9)—C(4)—C(5)—C(6) | 178.6(2) |
| C(3)—C(4)—C(5)—C(6) | 1.0(4) |
| C(4)—C(5)—C(6)—C(7) | 15.4(3) |
| C(5)—C(6)—C(7)—C(8) | −118.3(2) |
| C(5)—C(6)—C(7)—C(2) | 4.9(3) |
| C(17)—C(2)—C(7)—C(8) | −144.39(19) |
| C(1)—C(2)—C(7)—C(8) | −22.8(2) |
| C(3)—C(2)—C(7)—C(8) | 91.3(2) |
| C(17)—C(2)—C(7)—C(6) | 87.0(2) |
| C(1)—C(2)—C(7)—C(6) | −151.5(2) |
| C(3)—C(2)—C(7)—C(6) | −37.3(3) |
| C(1)—O(1)—C(8)—O(3) | 172.0(2) |
| C(1)—O(1)—C(8)—C(7) | −11.3(2) |
| C(6)—C(7)—C(8)—O(3) | −31.7(4) |
| C(2)—C(7)—C(8)—O(3) | −161.8(3) |
| C(6)—C(7)—C(8)—O(1) | 152.05(19) |
| C(2)—C(7)—C(8)—O(1) | 22.0(2) |
| C(5)—C(4)—C(9)—C(10) | −175.0(3) |
| C(3)—C(4)—C(9)—C(10) | 2.7(4) |
| C(4)—C(3)—C(11)—C(16) | 133.5(2) |
| C(2)—C(3)—C(11)—C(16) | −99.9(2) |
| C(4)—C(3)—C(11)—C(12) | −46.8(3) |
| C(2)—C(3)—C(11)—C(12) | 79.8(2) |
| C(16)—C(11)—C(12)—C(13) | 1.6(3) |
| C(3)—C(11)—C(12)—C(13) | −178.08(19) |
| C(11)—C(12)—C(13)—C(14) | −0.5(3) |
| C(12)—C(13)—C(14)—O(4) | 179.52(19) |
| C(12)—C(13)—C(14)—C(15) | −0.8(3) |
| O(4)—C(14)—C(15)—C(16) | −179.35(19) |
| C(13)—C(14)—C(15)—C(16) | 1.0(3) |
| C(14)—C(15)—C(16)—C(11) | 0.2(3) |
| C(12)—C(11)—C(16)—C(15) | −1.4(3) |
| C(3)—C(11)—C(16)—C(15) | 178.24(19) |
| C(1)—C(2)—C(17)—C(22) | 50.8(3) |
| C(7)—C(2)—C(17)—C(22) | 165.9(2) |
| C(3)—C(2)—C(17)—C(22) | −67.9(3) |
| C(1)—C(2)—C(17)—C(18) | −133.1(2) |
| C(7)—C(2)—C(17)—C(18) | −18.0(3) |
| C(3)—C(2)—C(17)—C(18) | 108.2(2) |
| C(22)—C(17)—C(18)—C(19) | 0.4(4) |
| C(2)—C(17)—C(18)—C(19) | −175.8(2) |
| C(17)—C(18)—C(19)—C(20) | −0.2(4) |
| C(18)—C(19)—C(20)—C(21) | −0.5(4) |
| C(19)—C(20)—C(21)—C(22) | 0.9(4) |
| C(20)—C(21)—C(22)—C(17) | −0.7(4) |
| C(18)—C(17)—C(22)—C(21) | 0.1(4) |
| C(2)—C(17)—C(22)—C(21) | 176.3(2) |

### Example 4

### Biological Testing

Cell and Protein Based Screens

It will be appreciated that the small molecule compounds of the present invention may be screened in any of a variety of biological assays, for example, cell-based assays may be employed. Such cell-based assays generally involve contacting a cell with a compound and detecting any of a number of events, such as binding of the compound to the cell, initiation of a biochemical pathway or physiological change in the cell, changes in cell morphology, initiation or blockage of the cell cycle, etc.

As but one example, once synthesized, the compounds may be arrayed in multiwell plates (e.g., in 384-well plates by a robotic 384 pin arrayer) and assayed for their ability to bind to a particular cell type present in the well. Detection can be carried out , for example, by detecting a tag that is attached to the small molecule. Alternatively, the small molecule may be detected by using a second molecule that has a tag, the second molecule specifically binding the small molecule, e.g., a tagged antibody specific to the small molecule.

Alternatively or additionally, inventive compounds may be studied in assays. In such assays, the compounds are bound to a solid support and then contacted with a protein of interest. The presence or absence of binding between the compound and the protein is then detected. In certain cases, the protein itself is tagged with a molecule that can be detected, e.g., with a fluorescent molecule. Alternatively, the protein is detected by utilizing any immunoassay, such as the ELISA.

For example, a process known as small molecule printing (see, for example, U.S. Ser. No. 09/567,910, filed May 10, 2000; U.S. Ser. No. 10/370,885, filed Feb. 20, 2003; U.S. Ser. No. 60/480,724, filed Jun. 23, 2003; the entire contents of each of which is hereby incorporated by reference) may be utilized to screen proteins that interact with the library compounds. First, a split pool library is arrayed onto beads. The compounds are then cleaved from the beads and prepared in a standard stock solution, such as DMSO. The compounds are then arrayed onto a 384-well stock plate. Next, the compounds are printed onto glass slides, e.g., a glass microscope slides, and the slides are probed with a tagged ligand, e.g., a tagged protein of interest. Binding between a compound and the ligand is then detected by any available means appropriate to the tag being utilized, e.g., via fluorescence.

Although any of the general assay methods described above may identify molecules having biological properties beyond those of the natural product, certain assays are of special interest, including but not limited to those as described below.

Protein Trafficking

Protein trafficking (or vesicle transport) is the general process in eukaryotic cells by which proteins synthesized in the endoplasmic reticulum (ER) are transported via the golgi network to the various compartments in the cell where they will carry out their function. Some proteins are transported through the golgi apparatus all the way to the cell surface where they are secreted (exocytosis). Such proteins include membrane bound receptors or other membrane proteins, neurotransmitters, hormones, and digestive enzymes. The transport process uses a series of transport vesicles that shuttle a protein from one membrane-bound compartment (donor compartment) to another (acceptor compartment) until the protein reaches its proper destination (Rothman et al. Science 272:227-234, 1996; incorporated herein by reference).

The process of vesicle transport begins with the budding of a vesicle out of the donor compartment. The vesicle containing the protein to be transported is surrounded by a protective coat made up of protein subunits recruited from the cytosol. The initial budding and coating processes are controlled by cytosolic GTP-binding proteins (GTPB). When GTP binds and activates the GTPB, the GTP-GTPB complex binds to the donor compartment and initiates the vesicle assembly process. The coated vesicle containing the GTP-GTPB complex detaches from the donor compartment and is transported through the cytosol. During the transport process, the GTP is hydrolyzed to GDP, and the inactivated GTPB dissociates from the transport vesicle and is recycled. At this point, the protective coat of the vesicle becomes unstable and dissociates from the enclosed vesicle. The uncoated vesicle is recognized by its acceptor compartment through exposed surface identifiers (v-SNAREs) which bind with corresponding molecules on the acceptor compartment membrane (t-SNAREs). The transport process ends when the vesicle fuses with the target membrane.

Many of the proteins involved in synaptic vesicle transport have been identified and the biochemical interactions between them have been characterized. Interestingly, many of these proteins are homologous to yeast proteins involved in yeast secretory pathways. In addition, many agents that disrupt the golgi apparatus and interfere with trafficking have been identified, e.g., monensin, bafilomycin, ilimaquinone, retinoic acid, okadaic acid, and nocodazole. Another agent, brefeldin A, is a natural compound that blocks protein secretion by disrupting the structure of the golgi apparatus. The present invention expands the limited pool of molecules presently available that may block protein trafficking.

It will be appreciated that cell-based phenotypic assays are commonly used to identify a block in protein trafficking from the endoplasmic reticulum to the golgi apparatus, or a block in exocytosis. Such phenotypic assays generally involve visualizing the transport lo of an intracellular protein within the cell. For example, a fluorescence immunoassay may be used to assess the location of a protein known to be shuttled from the endoplasmic reticulum to the golgi apparatus or to be exocytosed. Alternatively, cells may be transfected with an expression vector expressing a protein that is known to be trafficked that is a fusion protein with a fluorescent protein, such as green fluorescent protein. The location of the protein within a cell may be assessed by fixing the cell and visualizing the cell using fluorescence microscopy. Such assays are amenable to high-throughput screening via multiplexing, as described below.

Indeed, the present invention identifies certain compounds as potent inhibitors of the movement of a specific cellular protein from the endoplasmic reticulum to the golgi apparatus or as a potent inhibitor of the movement of a specific cellular protein from the golgi apparatus to the plasma membrane. Compounds resembling natural products are capable of dramatically effecting a biological process where a natural product itself may show little or no activity. Thus, the present invention provides compounds that effect protein trafficking and secretion, which may be useful probe reagents for exploring these cellular pathways.

The present example illustrates an effective assay for identifying compounds that effect protein trafficking. The library of compounds described herein may be screened using a cell-based phenotypic assay. The fluorescent fusion protein viral glycoprotein ts045 (VSVG-GFP) was used to monitor the ability of individual library members to block protein trafficking, as described in Presley et al. Nature 389:81-85, 1997, and Scales et al. Cell 19;90(6):1137-48, 1997, each of which is incorporated herein by reference.

Briefly, VSVG from the ts045 mutant strain of vesicular stomatitis virus has been widely used to study membrane transport because of its reversible misfolding and retention in the endoplasmic reticulum at 40° C. and its ability to move out of the endoplasmic reticulum at 32° C. (Kreis et al. Cell 46:929-927, 1986; Beckers et al. Cell 50,1 523-534, 1987; Bergmann et al. Methods Cell Biol. 32:85-110, 1989; each of which is incorporated herein by reference). Green fluorescent protein is attached to the cytoplasmic tail of VSVG. To examine how VSVG-GFP is transported from the endoplasmic reticulum to the golgi and then to the plasma membrane in the presence and absence of compound, cells in the presence and absence of compound are placed on the stage, of a fluorescent microscope warmed to 32° C. and fluorescent images were collected at distance intervals, e.g., every 3.6 seconds. Inhibition of the phenotype at 32° C. is determined by dramatic slow down of VSVG-GFP moving from one compartment to another.

Specifically, VSV-GFP was expressed in BSC1 cells at 40° C. Compounds were added to cells for 1 hour at 40° C. and then the cells were shifted to 32° C. for 2.5 hours before fixing and inspection using the fluorescent microscope. In the absence of compound, at this time point, the VSVG-GFP protein has already moved from the endoplasmic reticulum through the golgi to the plasma membrane. The VSVG^{ts}-GFP fusion protein is an effective exocytosis tracer, its localization being successively detected in the endoplasmic reticulum, the golgi apparatus, and the plasma membrane at 30° C. as protein trafficking proceeds. Compounds are screened on cells expressing the VSVG^{ts}-GFP fusion protein. Disruption of VSVG^{ts}-GFP fusion protein trafficking verified compounds capable of blocking the secretory pathway.

The compounds are arrayed in 384-well plates containing cells as solutions in DMSO. This was accomplished using a robotic 384 pin arrayer, as described above. Cells are then visualized by fluorescence microscopy on a plate reader.

Wound Healing

The present invention further relates to compounds that promote the repair of damaged tissues in animals, particularly in humans, and, more particularly, to the modulation of the healing of wounds in such tissue.

An endless variety of pathological and non-pathological causes results in injury and tissue wounds. A variety of cells have been determined to cooperate in response to injury to repair the damaged tissue and heal the wound. Cells resident in the local tissue participate in the process of wound healing, as do circulating blood cells specifically recruited into the wound itself and the area nearby. Dramatic changes in cellular function are required by both the resident and recruited cells in order to initiate, coordinate, and sustain the complex process of wound healing. Damaged cells and disrupted tissue matrix must be removed, and new cells must be born, grow, and mature to replace those cells that were lost. Finally, the tissue matrix must be resynthesized and remodeled. Even the microvasculature may need to be rebuilt to supply the new tissue with blood flow.

Wound healing is a complex process involving interactions among a variety of different cell types. Among recruited cells, macrophages are considered essential for normal wound healing. Macrophages are a rich source of peptide cytokines, which, as a group, are thought to be integral to the tissue repair responses to local injury. It is well known that individual cytokines can act on more than one cell type and can have more than one effect. Cytokines, especially interferon-alpha (IFN-alpha), IFN-alpha, and IFN-alpha 2b, may also reduce scar formation. These cytokines decrease the proliferation rate of fibroblasts and reduce the rate of collagen and fibronectin synthesis by reducing the production of mRNA. New cytokines continue to be described, and new functions are being attributed to them, as well as to previously described cytokines.

The normal wound repair process consists of three phases—inflammation, proliferation, and remodeling that occur in a predictable series of cellular and biochemical events. Furthermore, wounds are classified according to various criteria: etiology, lasting, morphological characteristics, communications with solid or hollow organs, the degree of contamination, etc. In the last few years many authors use the Color Code Concept, which classifies wounds as red, yellow, and black wounds. Compounds of the present invention may be screened for their effect on any of these phases or criteria.

Stimulation of local wound healing generally includes use of such compounds as antiseptic solutions that disinfect the area and topical antibiotic treatments. In addition, growth factors (e.g., epidermal growth factor (EGF), transforming growth factor-beta (TGF-beta), insulin-like growth factor (IGF), platelet-derived growth factor (PDGF), fibroblast growth factor (FGF), interleukins (ILs), and colony-stimulating factor (CSF)) play a role in many wound healing processes, including cell division, migration, differentiation, protein expression, and enzyme production. Moreover, growth factors have a potential ability to heal wounds by stimulating angiogenesis and cellular proliferation, affecting the production and degradation of the extracellular matrix, and by being chemotactic for inflammatory cells and fibroblasts. Acute wounds contain many growth factors that play a crucial role in the initial phases of wound healing.

Applications of some drugs (antioxidants—asiaticoside, vitamin E and ascorbic acid; calcium D-pantothenate, exogenous fibronectin; antileprosy drugs—oil of hydnocarpus; alcoholic extract of yeast) accelerate wound healing. Thymic peptide thymosin beta 4 (T beta 4R) topically applicated, increases collagen deposition and angiogenesis and stimulates keratinocyte migration. Thymosin alpha 1 (T alpha IR), peptide isolated from the thymus, is a potent chemoattractant which accelerates angiogenesis and wound healing. Furthermore, expression of nitric oxide synthase (NOS) and heat shock proteins (HSP) have an important role in wound healing, as well as the trace elements zinc, copper, manganese.

According to the present invention, compounds that either have the activities of any of the above wound healing agents or modify (enhance or reduce) the activities of the above wound healing agents may be easily identified. Those skilled in the art will appreciate that the availability of the wide variety of wound healing agents indicates that assays for identifying such agents are well known in the art. A typical assay for identifying compounds having would healing activity involves 1) creating a wound, 2) applying a compound to the wound, and 3) after an appropriate amount of time, visualizing the wound to determine the extent of closure, as compared to the extent of closure in the absence of compound. It will be appreciated that wound healing assays may be conducted on cells in vivo or in vitro, and such are provided herein.

The present example illustrates an assay for identifying compounds that are candidate wound healing agents. 5500 BS-C-1 epithelial cells are plated in 384 well clear bottom plates (30 μl total volume). The cells are incubated overnight or until cells form a confluent monolayer. The monolayers are mechanically wounded using a 96 floating pin array, and this procedure is repeated 4 times for one 384 well plate. Immediately after wounding 40 nl of compound is pin transferred into plates. The plate is then incubated for 7 hrs to allow cells to migrate in to heal the wound. The cells are then fixed using 4% paraformaldehyde and stained with rhodamine phalloidin to image actin and hoechst to image the nuclei. The plate is imaged with a 4× objective using the automated microscope from Image1. Each image is visually inspected and the extent of wound healing categorized. Certain inventive compounds are identified as having interesting phenotypes and specifically have been found to affect the would healing, or cell migration; that is the compounds affect migration into the wound, affect cell density, affect migration/adhesion of the cells such that they pile upon each other, affect morphology along the front of migration, or affect morphology along the front of migration.

Identification of Antimicrobials

Antimicrobial agents, such as antibiotics, have been effective tools in the treatment of infectious diseases during the last half century. From the time that antibiotic therapy was first developed to the late 1980s, there was almost complete control over bacterial infections in developed countries. The emergence of resistant bacteria, especially during the late 1980s and early 1990s, is changing this situation. The increase in antibiotic resistant strains has been particularly common in major hospitals and care centers. The consequences of the increase in resistant strains include higher morbidity and mortality, longer patient hospitalization, and an increase in treatment costs. (Murray, New Engl. J Med. 330:1229-1230, 1994; incorporated herein by reference).

Many different bacterial populations that are resistant to many antibiotics have been identified over the past twenty-five years. These populations include opportunistic and virulent pathogens that were previously susceptible to antibiotic treatment. Resistant opportunistic pathogens are particularly problematic for debilitated or immunocompromised patients. The development of tolerance and resistance in virulent pathogens poses a significant threat to the ability to treat disease in all patients, compromised as well as noncompromised.

One major factor that has contributed to the increase in the number of resistance strains is the over-use and/or inappropriate administration of antimicrobials in the treatment arena. Newly acquired resistance is generally due to the relatively rapid mutation rate in bacteria. Another contributing factor is the ability of many microorganisms to exchange genetic material that confers resistance, e.g., exchanging of resistance plasmids (R plasmids) or resistance transposons.

For example, following years of use to treat various infections and diseases, penicillin resistance has become increasingly widespread in the microbial populations that were previously susceptible to the action of these drugs. Some microorganisms produce β-lactamase, an enzyme that destroys the antimicrobial itself, while some microorganisms have undergone genetic changes that result in alterations to the cell receptors known as the penicillin-binding proteins, such that penicillin no longer effectively binds to the receptors. As but another example, other organisms have evolved in a manner that prevents the lysis of cells to which the drug has bound. The drug therefore inhibits the growth of the cell, but does not kill the cell. This appears to contribute to the relapse of disease following premature discontinuation of treatment, as some of the cell remain viable and may begin growing once the antimicrobial is removed from their environment.

The first report of penicillin resistance occurred in Australia in 1967. Since this initial report, additional penicillin resistant strains have been reported worldwide. In addition, strains having resistance to numerous other antibiotics have also been reported, including chloramphenicol, erythromycin, tetracycline, clindamycin, rifampin, methicillin, and sulfamethoxazole-trimethoprim.

Infections by naturally resistant opportunistic or virulent pathogens are difficult to treat with current antibiotics. There is an urgent medical need for new antibiotic molecules which can override the mechanisms of resistance and maintain the level of public health we enjoy today.

The compounds of the present invention may be screened for antimicrobial activity. Those skilled in the art will appreciate that any compound that inhibits the division of a microbial cell, e.g., yeast, fungi, bacteria, and the like, may be identified, and such assays are well known in the art. Bacterial cells divide by first initiating DNA replication. At the end of the bacterial cell cycle, the chromosomes segregate and the cells divide by forming a septum that cuts the cells in two, a process known as septation. Many of the proteins that regulate bacterial replication and septation have yet to be identified.

a. Vibrio cholera inhibitors: Certain of the inventive compounds are identified as having an antibacterial effect (either bacteriocidal or bacteriostatic) using the following assay:

An overnight culture of Vibrio cholerae strain M329 is diluted 1:10000 in fresh growth medium. The freshly diluted culture is dispensed in 25 microliter volumes to each well of an appropriate number of 384-well microtiter plates. The compounds and library of compounds as described herein are transferred to corresponding wells in the cell-containing 384 well microtiter plates. The microtiter plates re then incubated for 12-18 hours at 30° C. before being imaged with a CCD camera or a luminescence plate reader. The microtiter lo plates that contain positive hits (dark non luminescent wells) are then welled on and assayed for viable bacteria. Compounds of interest have an IC₅₀ lower than or approximately the same as known antibiotics (e.g., tetracycline) used to treat Vibrio cholerae infections

b. Toxoplams gondii Inhibitors

It will be appreciated that certain of the inventive compounds may demonstrate anti-protozoal activity, more particularly anti-toxoplasma activity.

Protozoa are unicellular eukaryotic microorganisms that lack cell walls and are usually motile and colorless. They are distinguished from algae by their lack of chlorophyll, from fungi by their motility and absence of a cell wall, and from slime molds by their lack of fruiting body formation.

Protozoa are generally classified into four major groups based on their life cycles or mechanisms of motility: the flagellates, the cilliates, the amoeba, and the sporozoa (or apicomplexa). The flagellates are protozoa that employ from one to eight or so flagella for movement. The ciliates employ cilia, which are shorter than flagella and are present in large numbers. Protozoa which move by extending pseudopodia are called amoeba. The fourth major group, the sporozoa or apicomplexa, are non-motile, intracellular parasites (except during their sexual stage) that penetrate host cells by a mechanism involving their characteristic apical complex. Some protozoa do not fit into any of these four groups, such as the non-motile, intracellular microsporidia, which penetrate host cells by an injection mechanism.

Clinically important representatives of the flagellate group include Giardia lamblia, Trichomonas vaginalis, Leishmania spp., and Trypanosoma spp. G. lamblia is a waterborne intestinal parasite which occurs worldwide, causing diarrhea, and other intestinal symptoms. The most commonly used drugs used to treat giardiasis are metronidazole and other members of the 5-nitroimidazoles. Unfortunately, Metronidazole is mutagenic in the Ames test (Vogd et al. Mutation Research 26:483-490 1974; incorporated herein by reference) and has various toxic side effects. In addition, the development of resistance to these drugs in Giardia and other protozoan parasites such as Entamoeba histolytica and Trichomonas vaginalis also limits their effectiveness. Leishmaniasis, a life-threatening disease caused by Leishmania spp., is a major health problem worldwide with an estimated 10-15 million people infected and 400,000 new cases each year. There is currently no satisfactory treatment for leishmaniasis. The treatment of choice is pentavalent antimony in the form of sodium stibogluconate or meglumine antimonate. Both drugs are administered intravenously, have severe adverse side effects, require hospitalization during treatment, and are not always effective (Ouelette and Papadopoulou, Parasitology Today 9:150-153, 1993; incorporated herein by reference). Trypanosoma spp. cause life-threatening diseases in humans, including African sleeping sickness and Chagas disease, as well as a number of important diseases in domestic animals. Leishmania and Trypanosoma are closely-related genera, representing the major pathogens in the kinetoplastid group of protozoa.

The ciliates are generally non pathogenic, except for Balantidium coli which is an intestinal parasite of domestic animals, in particular, swine. Occasionally, B. coli infects humans, producing a severe dysentery.

The amoeba group includes the intestinal parasite Entamoeba histolytica which causes amoebic dysentery and extraintestinal abscesses of organs such as the liver and lung. The most commonly used drug for treating E. histolytica infection is metronidazole. Other free-living amoeba which occasionally cause infections in humans include Acanthamoeba and Naegleria spp.; these infections are typically difficult to treat.

The sporozoa are a large group of protozoa, all of which are obligate parasites. Representative sporozoas are the malaria parasite Plasmodium spp., the human pathogen Cryptosporidium spp., Toxoplasma gondii, and several parasites veterinary importance including Sarcocystis spp., Theileria spp., and Eimeria spp. (causing coccidiosis in fowl and domestic animals). Cryptosporidium parvum is a common cause of intestinal infection leading to self-limited diarrhea, but in the immunocompromized individual C. parvum infection is chronic and life-threatening. There is currently no effective treatment for cryptosporidiosis.

Toxoplasma gondii is the causative agent in toxoplasmosis, an important disease in immunocompromised patients as well as congenitally-infected fetuses. Toxoplasma gondii is also pathogenic to animals, particularly sheep, in which it causes abortion, stillbirth, and fetal mummification. The pathology of toxoplasmosis in its human and animal hosts is a direct result of repeated cycles of host cell invasion, parasite replication, and host cell lysis. In addition, Toxoplasma gondii causes encephalitis, a dangerous life-threatening disease. Toxoplasmic encephalitis is currently treated with a combination of pyrimethamine and sulfadiazine, the side effects of which are frequently so severe as to require discontinuation of the treatment.

Microsporidia are obligate, intracellular pathogens which cause intestinal and systemic infections in immunocompromized patients, as well as economically important infections in fish and invertebrates. Microsporidiosis in patients suffering from acquired immune deficiency syndrome (AIDS) is primarily associated with Encephalitozoon species (including E. intestinalis, E. cuniculi, and E. hellem) and Enterocytozoon bieneusi. Microsporidiosis is a frequent cause of chronic diarrhea in AIDS patients and may also be found outside of the intestine in the eye, biliary tract, nasal sinuses, urinary tract, and respiratory tract.

It will be appreciated that there is an urgent need for new chemotherapeutic agents to combat protozoal parasites that are sufficiently effective, do not have harmful side effects, and are not difficult or expensive to administer. Preferably, the anti-protozoal compounds are active against a broad spectrum of protozoa, while remaining non-toxic to human and other mammalian cells. Current approaches for identifying compounds that are anti-protozoal agents often rely on classical genetic systems, e.g., the identification of temperature sensitive mutants, inducible promoters, and the like.

As will be appreciated by those skilled in the art, the anti-protozoal agents identified may be used in pharmaceutical compositions that may be used for the eradication or inactivation of harmful protozoal parasites. This includes compounds that inhibit the invasion of a cell by protozoal parasites, such as flagellates (Giardia lamblia, Trichomonas vaginalis, Leishmania spp., and Trypanosoma spp. G. lamblia), cilliates (e.g., Balantidium coli), amoebas (e.g., Entamoeba histolytica, Acanthamoeba spp., and Naegleria spp.), and sporozoas (or apicomplexa) (e.g., Plasmodium spp., Cryptosporidium spp., Toxoplasma gondii, Sarcocystis spp., Theileria spp., and Eimeria spp), microsporidia (Encephalitozoon species (including E. intestinalis, E. cuniculi, and E. hellem) and Enterocytozoon bieneus), and the like. The pharmaceutical compositions may thus be utilized as preventative and/or disinfectant agents.

Additionally, it will be appreciated that pharmaceutically acceptable derivatives of the anti-protozoal compounds identified using the assays described herein. Furthermore, the methods of treating animals (e.g., equines, bovines, felines, canines, swine, ovines, birds, insects, and humans) using these anti-protozoal compounds and pharmaceutical compositions thereof, or either of these in combination with one or more additional therapeutic agents as provided, as described in detail herein.

High-throughput assay systems for the identification of anti-protozoal agents are illustrated herein using a protozoa of the apicomplexa family of protozoa, Toxoplasma gondii. As demonstrated by the present example, the ability or inability of the parasite to invade a cell may be determined by detecting the number of parasites on the exterior vs. the interior of a host cell.

The process of host cell invasion by Toxoplasma gondii initiates with the of attachment of the parasite to the host cell membrane. Once attached, the protozoa secretes a cocktail of proteins that initiate degradation of the cell wall. After the cell is permeated, invagination of the host cell begins and is complete when the parasite is entirely engulfed by the host cell. The process of vacuole formation is then initiated within the cell. The process of invasion is then complete and the parasite begins the process of replication inside the cell before it exits the cell and begins the invasion process again in other host cells. The assay described herein may identify compounds capable of inhibiting protozoal infection that can effect any stage of the Toxoplasma life cycle.

Identification of Anti-Protozoal Agents Using Labeled Protozoa

The following protocol is carried out in all wells of a 384 well plate. The media covering a confluent monolayer of host cells is removed and replaced with a previously prepared solution of a test compound in media. The host cells are BSC-1 cells, a monkey kidney cell line (however, any host cell may be used since Toxoplasma gondii can invade essentially any nucleated cell). A solution of T. gondii tachyzoites expressing the yellow fluorescent protein is then added and the host cells and labeled parasites are preincubated with the compound at a temperature at which invasion does not occur (20-22° C.). It will be appreciated that a variety of fluorescent proteins (e.g., green, red, and yellow) are available in the art (see, e.g., Harpur et al. Nat. Biotechnol. 19(2):167-169, 2001; Mizuno et al. Biochemistry 40(8):2502-2510, 2001; Huang et al. Traffic 2(5):345-357, 2001; incorporated herein by reference). After 15 minutes the assay plate is temperature shifted to 37° C., a temperature at which host cell invasion by the parasites occurs in the absence of compound. After 1 hour, excess parasites are removed by repeat rounds of washing. External parasites are immunostained using dye-conjugated anti-SAG1 antibody. The dye is an Alexa dye (red) (Molecular Probes). The cells are then fixed by treating the cells for 30 minutes with formaldehyde/gluteraldehyde solution in Hanks buffer. Those skilled in the art will appreciate that antibodies may be attached to a wide variety of labels available in the art, see for example, U.S. Pat. No. 6,027,890, incorporated herein by reference.

Automated image acquisition and analysis techniques are used to determine the number of invaded parasites. Digital fluorescence images are collected on a fully automated fluorescence microscope having an automated XY stage and a Z-motor that is required for computer controlled auto focusing, and the number of invading vs. external parasites quantitated automatically from the stored images (Metamorph software by Universal Imaging). Positive results from the automated analysis are confirmed, e.g., by manual re-examination of individual wells under the microscope.

In order to quantitate invasion, the number of parasites inside the cell, which are yellow only, are counted. Alternatively, the total number of external parasites (which are both red and yellow) are subtracted from the total number of parasites, both internal and external (which are labeled yellow and red). Compounds that lower the invasion level by 80% or raise it (by 2 fold) compared to control values (cells plus parasites in the absence of test compound) are considered as preliminary hits in this assay to be followed up with secondary screening.

Identification of Anti-protozoal Compounds Using Antibody Detection

The following protocol is carried out in all wells of a 384 well plate and visualized as described above using fluorescence microscopy. The media covering a confluent monolayer of BSC-1 host cells was removed and replaced with a previously prepared solution of a test compound under examination in media. A solution of wild-type T. gondii tachyzoites (that are not labeled) is then added and the host cells and parasites are preincubated with the compound at a temperature at which invasion does not occur (20-22° C.). After 15 minutes the assay plate was temperature shifted to 37° C., a temperature at which host cell invasion by the parasites occurs in the absence of compound. After 1 hour, excess parasites were removed by repeat rounds of washing. External parasites were immunostained using dye-conjugated anti-SAG1 antibodies. The dye is an Alexa dye (red) (Molecular Probes). The cells were then fixed by treating the cells for 30 minutes with formaldehyde/gluteraldehyde solution in Hanks buffer, which permeabilizes the cells. All parasites (internal and external) are then stained with a second SAG1 antibody that is labeled with a green fluorescent label.

Automated image acquisition and analysis techniques were used to determine the number of invaded parasites. In order to quantitate invasion, the number of parasites inside the cell, which are green only, are counted. Alternatively, the total number of external parasites (which are both red and green) are subtracted from the total number of parasites (both internal and external, which are labeled green and red). As noted above, compounds that lower the invasion level by 80% or raise it (by 2 fold) compared to control values (cells plus parasites in the absence of test compound) are considered as preliminary hits in this assay. The SAG1 antibody may be used twice because there is enough SAG1 on the surface of these parasites that you do not saturate all of the sites with the first antibody.

### Other Embodiments

Those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

What is claimed is:

1. A method of synthesizing a collection of chemical compounds, the method comprising steps of:
providing at least two precursor templates that when exposed to certain reaction conditions will generate different molecular skeletons;
providing reagents for generating the different molecular skeletons from the precursor templates; and
contacting the templates with the reagents so as to generate the different molecular skeletons.

2. A method of synthesizing a collection of chemical compounds, the method comprising steps of:
providing at least two precursor templates that when exposed to a common set of reaction conditions will generate different molecular skeletons, whereby the templates encode the molecular skeleton to be formed;
providing reagents for generating the different molecular skeletons from the precursor templates; and
contacting the templates with the reagents so as to generate the different molecular skeletons.

3. A method of synthesizing a collection of chemical compounds, the method comprising steps of:
providing at least two precursor templates that when exposed to different reaction conditions will generate different molecular skeletons, whereby the different molecular skeletons are encoded by the reaction conditions used;
providing reagents for generating the different molecular skeletons from the precursor templates; and
contacting the templates with the reagents so as to generate the different molecular skeletons.

4. The method of claim 1 further comprising steps of:
providing reactants for further derivatizing the molecular skeletons; and
contacting the molecular skeletons with reactants using a split-pool method to generate a collection of chemical compounds.

5. The method of claim 4, wherein the synthesis of each chemical compound is accomplished in less than 10 steps.

6. The method of claim 4, wherein the synthesis of each chemical is accomplished in 3-5 steps.

7. The method of claim 1, wherein the precursor templates include a common core structure.

8. The method of claim 7, wherein the common core structure includes a heterocycle.

9. The method of claim 7, wherein the common core structure includes an aromatic heterocycle.

10. The method of claim 7, wherein the common core structure includes a nitrogen-containing heterocycle.

11. The method of claim 7, wherein the common core structure includes an oxygen-containing heterocycle.

12. The method of claim 7, wherein the common core structure includes a polycyclic system.

13. The method of claim 7, wherein the common core structure includes an unsaturated system.

14. The method of claim 7, wherein the common core structure includes an alkene.

15. The method of claim 7, wherein the common core structure includes an alkyne.

16. The method of claim 1, wherein the precursor templates are furan derivatives.

17. The method of claim 1, wherein generating molecular skeletons comprises creating at least one cyclic structure.

18. The method of claim 1, wherein generating molecular skeletons comprises creating at least two cyclic structures in a molecule.

19. The method of claim 1, wherein generating molecular skeletons comprises opening up of at least one cyclic structure in a molecule.

20. The method of claim 1, wherein generating molecular skeletons comprises opening up at least one cyclic structure and creating at least one cyclic structure in a molecule.

21. The method of claim 1, wherein the reaction conditions for generating molecular skeletons comprise an oxidation.

22. The method of claim 1, wherein the reaction conditions for generating molecular skeletons comprises a reduction.

23. The method of claim 1, wherein the reaction conditions for generating molecular skeletons comprise an acid-catalyzed reaction.

24. The method of claim 1, wherein the reaction conditions for generating molecular skeletons comprise a base-catalyzed reaction.

25. The method of claim 1, wherein the reaction used to generate the molecular skeleton is an Achmatowicz reaction.

26. The method of claim 1, wherein the template is bound to a solid support.

27. A compound of one of the structures: 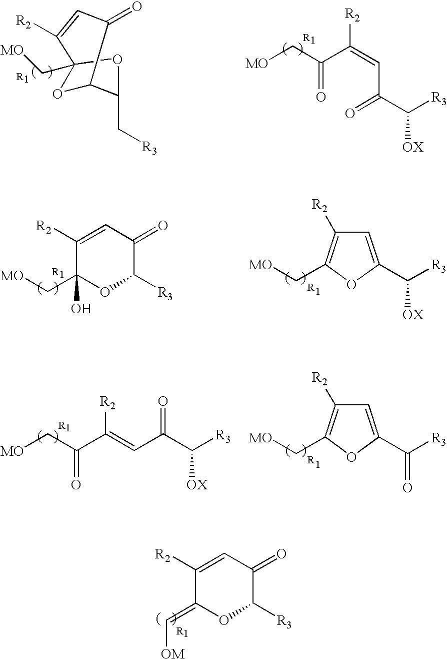
wherein M is a solid support, polymeric support, a hydrogen, a protecting group, a lower alkyl group, or a lower acyl group;
X is a hydrogen, a protecting group, a lower alkyl group, or a lower acyl group;
R¹ is selected from the group consisting of: 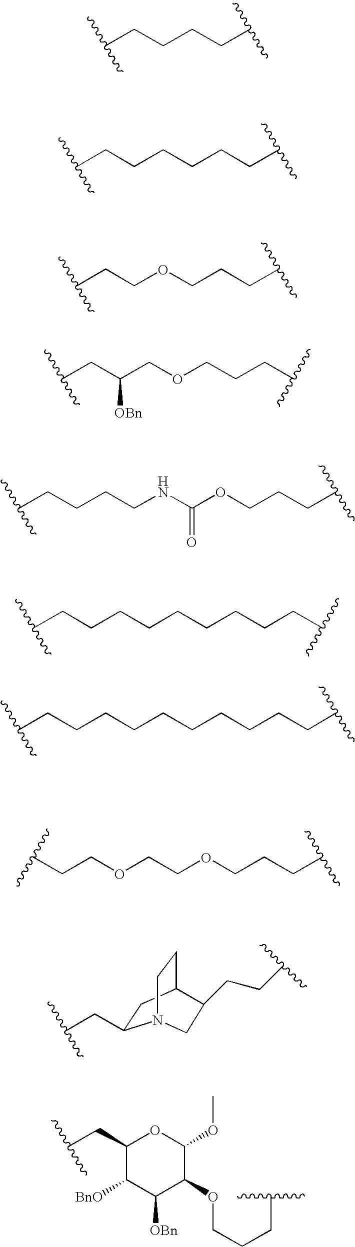
or stereoisomers thereof;
R₂ is selected from the group consisting of: 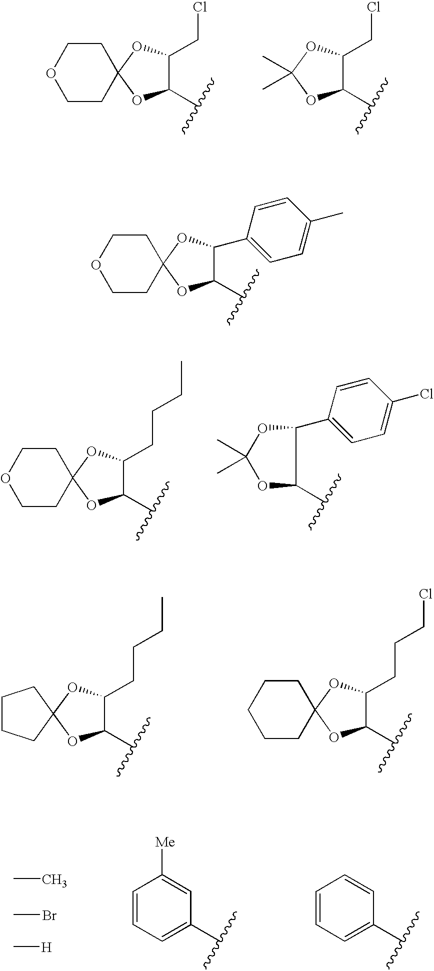
or stereoisomers thereof;
R₃ is selected from the group consisting of: 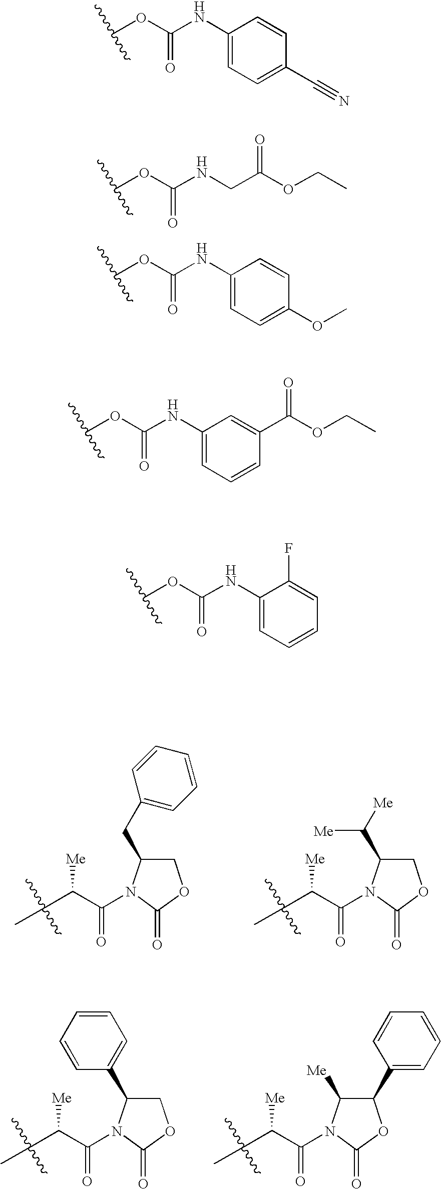 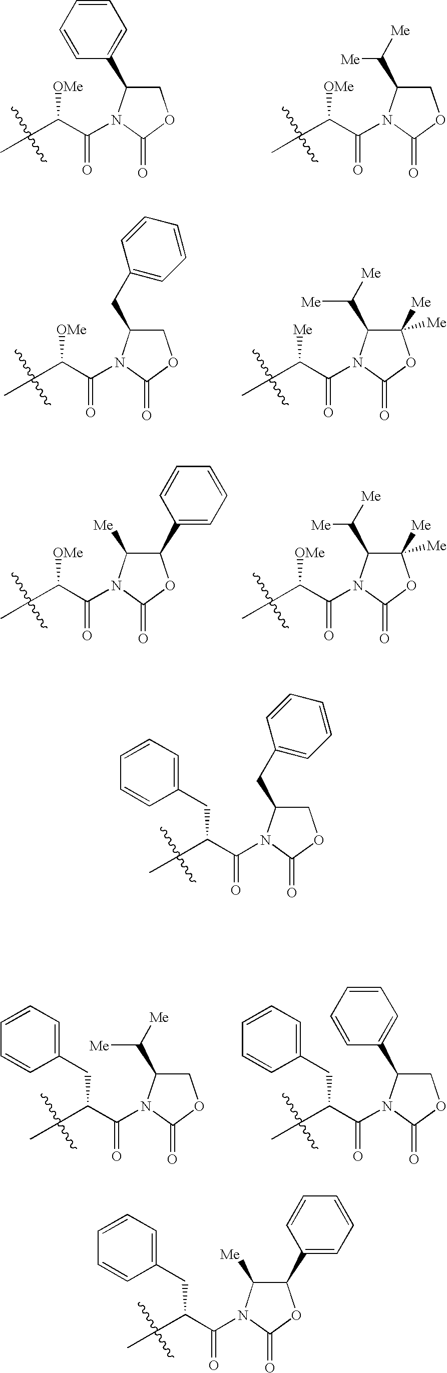 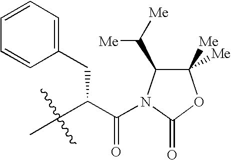
or stereoisomers thereof,

28. The compound of claim 27 of formula: 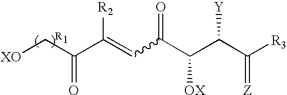
X is independently chosen as a solid support, a polymeric support, a hydrogen, a protecting group, a lower alkyl group, or a lower acyl group;
Z is O, S, CH₂, NH, or alkylamino;
Y is a protected hydroxyl group, hydroxy group, lower alkyl, methyl, lower alkoxy, methoxy, benzyl, or arylalkyl group;
R¹ is selected from the group consisting of: 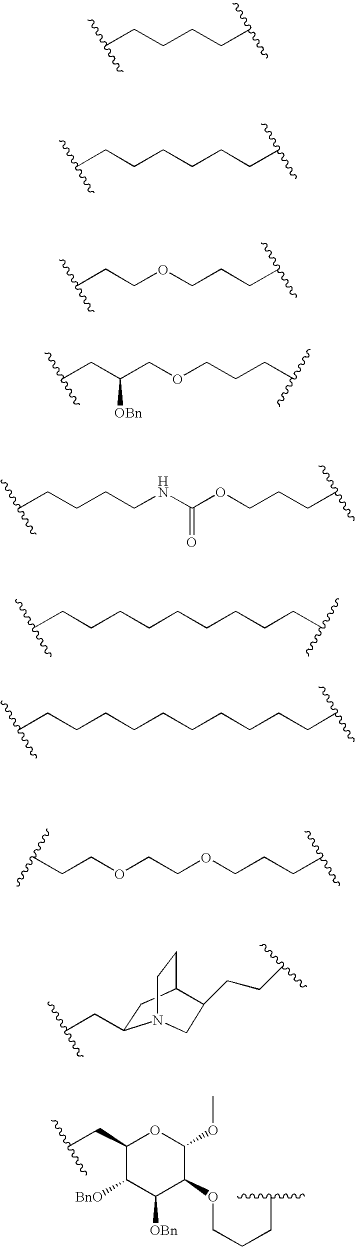
or stereoisomers thereof;
R₂ is selected from the group consisting of: 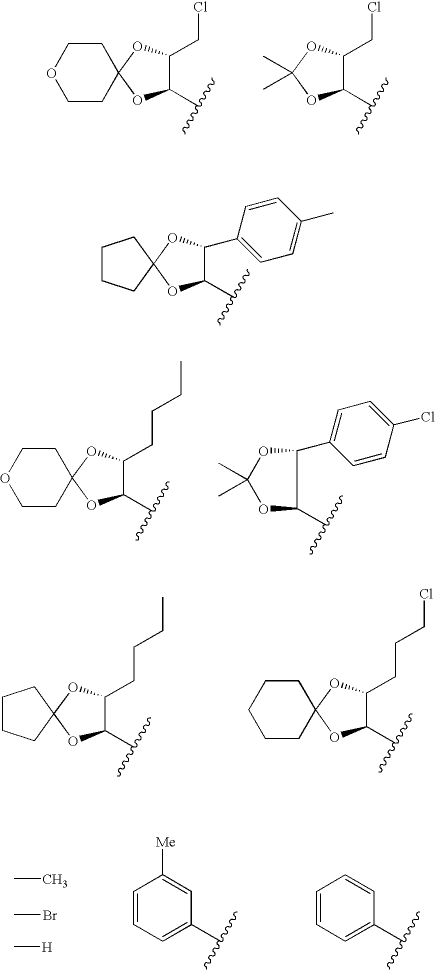
or stereoisomers thereof;
R₃ is selected from the group consisting of: 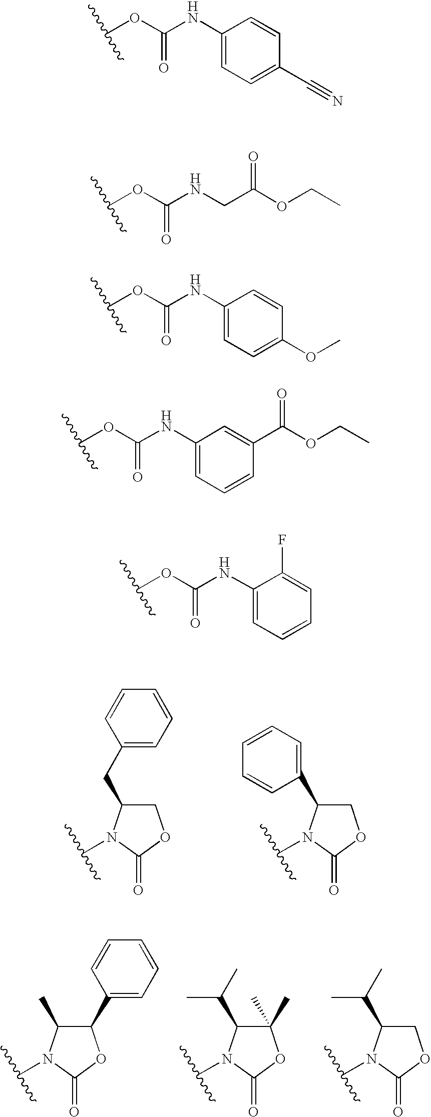
or stereoisomers thereof.

29. The compound of claim 28, wherein the carbon-carbon double bond is in the E configuration.

30. The compound of claim 28, wherein the carbon-carbon double bond is in the Z configuration.

31. The compound of claim 27 of formula: 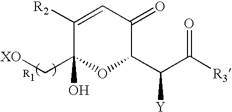
X is chosen as a solid support, a polymeric support, a hydrogen, a protecting group, a lower alkyl group, or a lower acyl group;
Y is a protected hydroxyl group, hydroxy group, lower alkyl, methyl, lower alkoxy, methoxy, benzyl, or arylalkyl group;
R¹ is selected from the group consisting of: 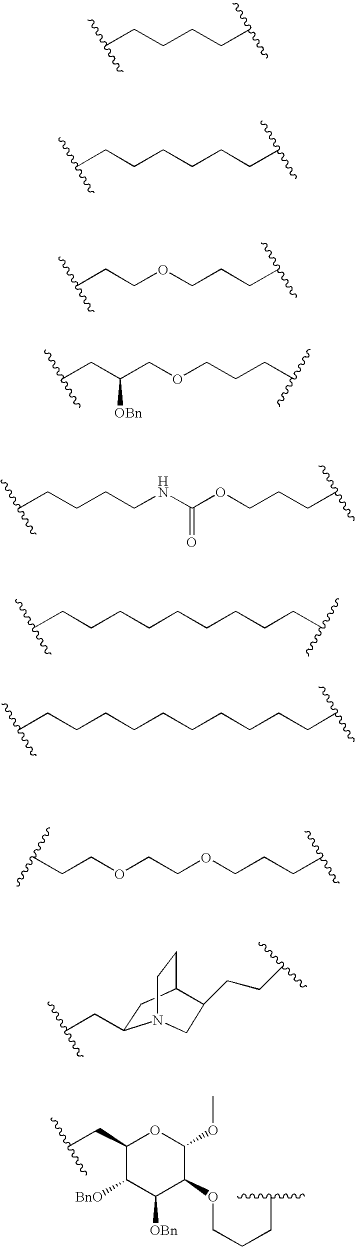
or stereoisomers thereof;
R₂ is selected from the group consisting of: 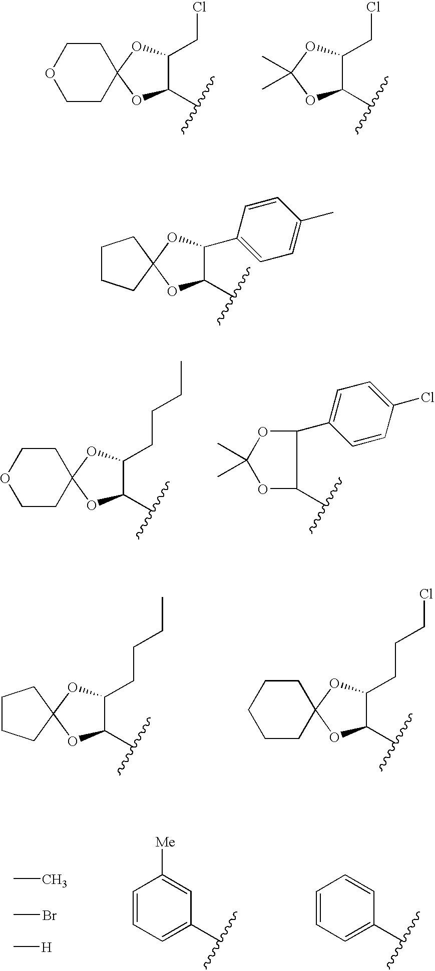
or stereoisomers thereof;
R₃′ is selected from the group consisting of: 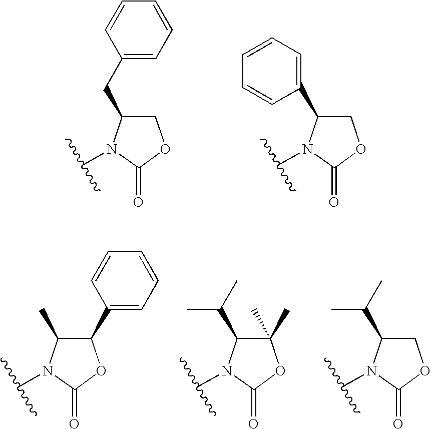
or stereoisomers thereof.

32. The compound of claim 27 of formula: 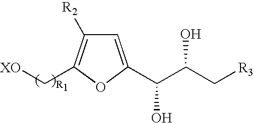
X is a solid support, polymeric support, a hydrogen, a protecting group, a lower alkyl group, or a lower acyl group;
R¹ is selected from the group consisting of: 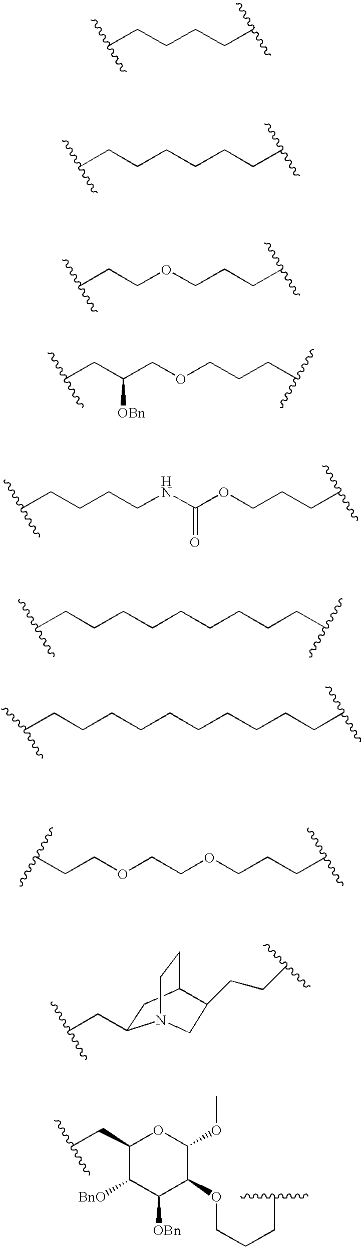
or stereoisomers thereof;
R₂ is selected from the group consisting of: 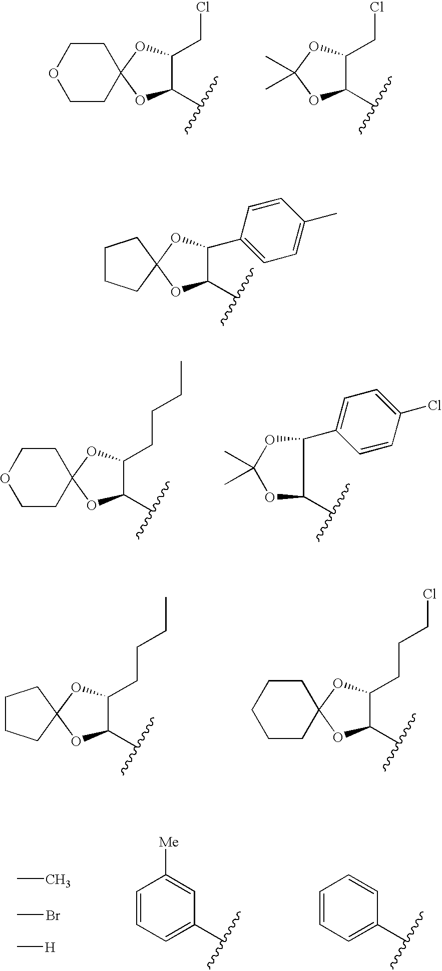
or stereoisomers thereof;
R₃ is selected from the groups consisting of:

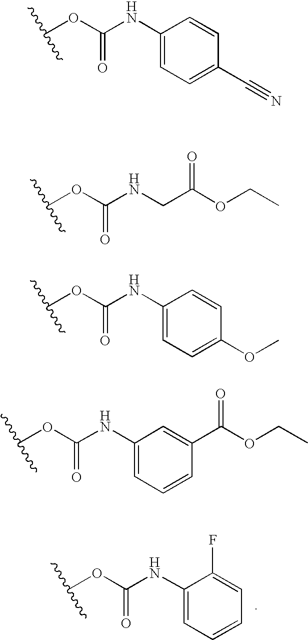

33. The compound of claim 27 of formula: 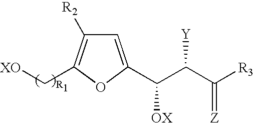
X is independently chosen as a solid support, a polymeric support, a hydrogen, a protecting group, a lower alkyl group, or a lower acyl group;
Z is O, S, CH₂, NH, or alkylamino;
Y is a protected hydroxyl group, hydroxy group, lower alkyl, methyl, lower alkoxy, methoxy, benzyl, or arylalkyl group;
R₁ is selected from the group consisting of: 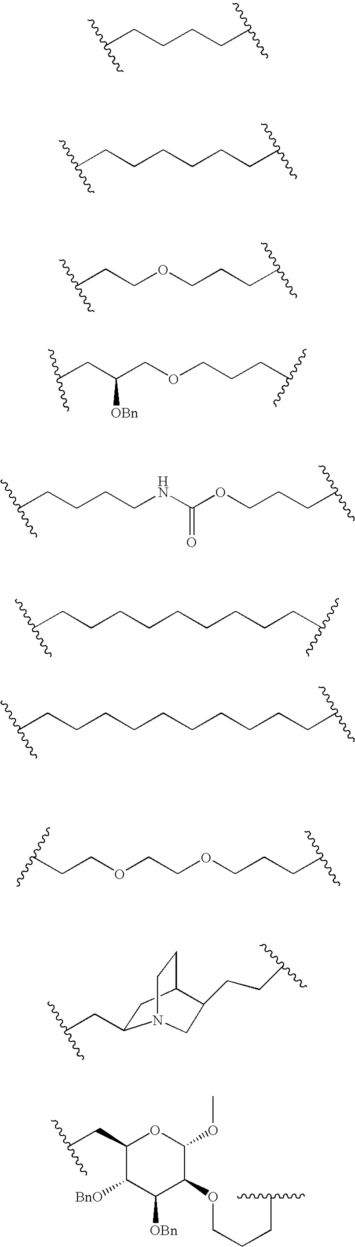
or stereoisomers thereof;
R₂ is selected from the group consisting of: 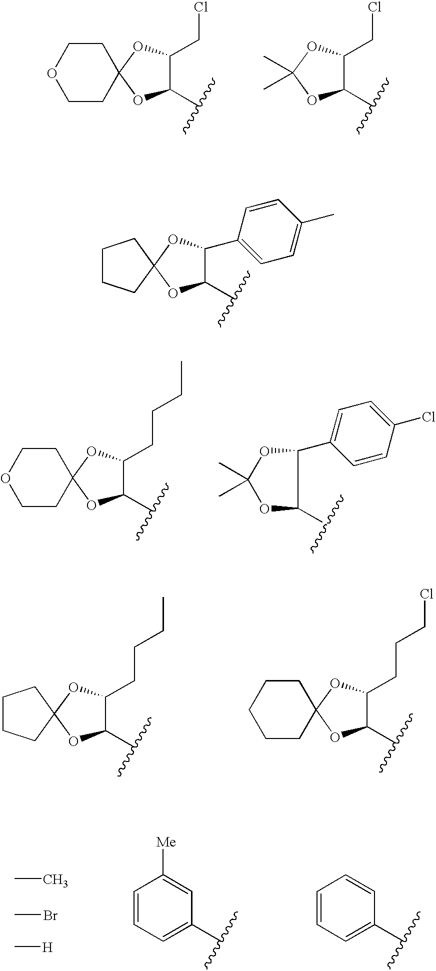
or stereoisomers thereof;
R₃ is selected from the group consisting of: 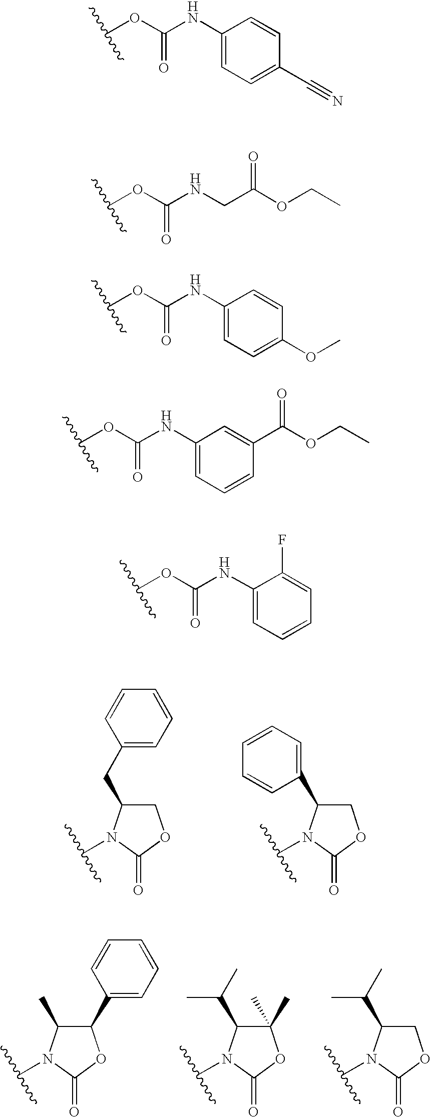
or stereoisomers thereof.

34. The compound of claim 27 of formula: 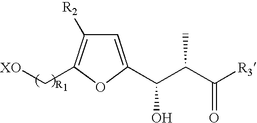
X is a solid support, a hydrogen, a protecting group, a lower alkyl group, or a lower acyl group;
Y is methyl, methoxy, or benzyl;
R₁ is selected from the group consisting of: 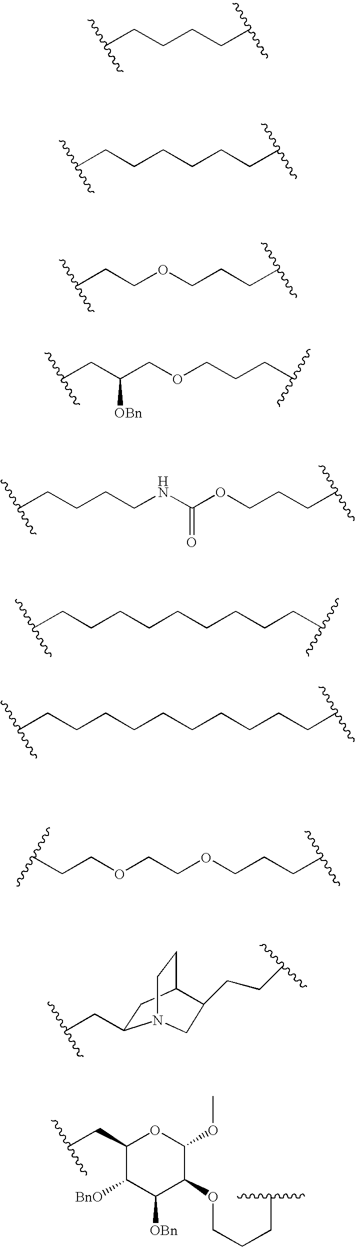
or stereoisomers thereof;
R₂ is selected from the group consisting of: 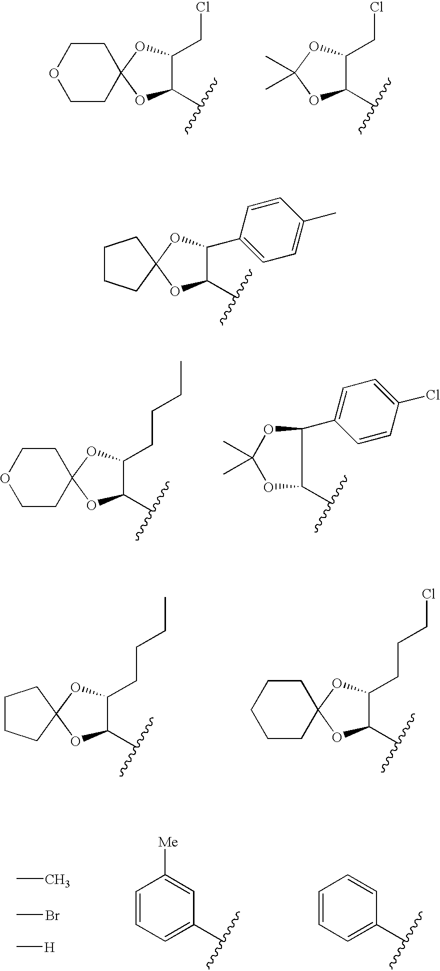
or stereoisomers thereof;
R₃′ is selected from the group consisting of: 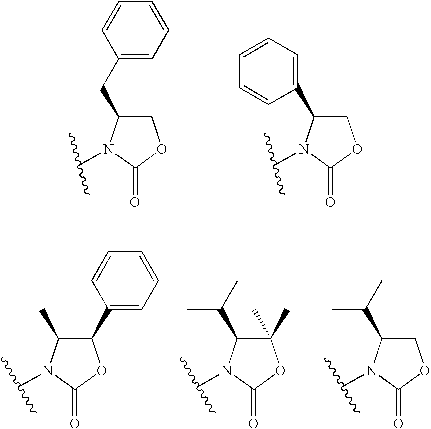
or stereoisomers thereof.

35. The compound of claim 27 of formula: 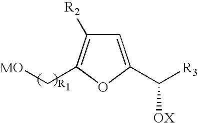
wherein M is a solid support, polymeric support, a hydrogen, a protecting group, a lower alkyl group, or a lower acyl group;
X is a hydrogen, a protecting group, a lower alkyl group, acetyl, or a lower acyl group;
R₁ is selected from the group consisting of: 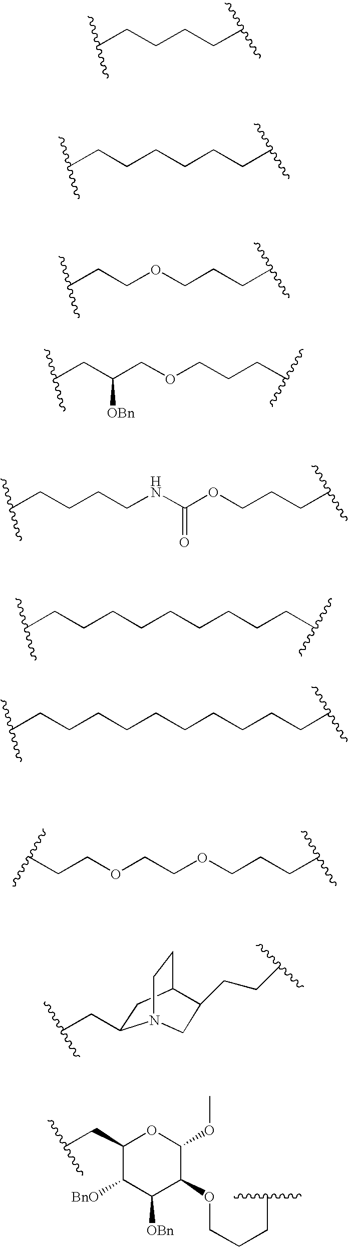
or stereoisomers thereof;
R₂ is selected from the group consisting of: 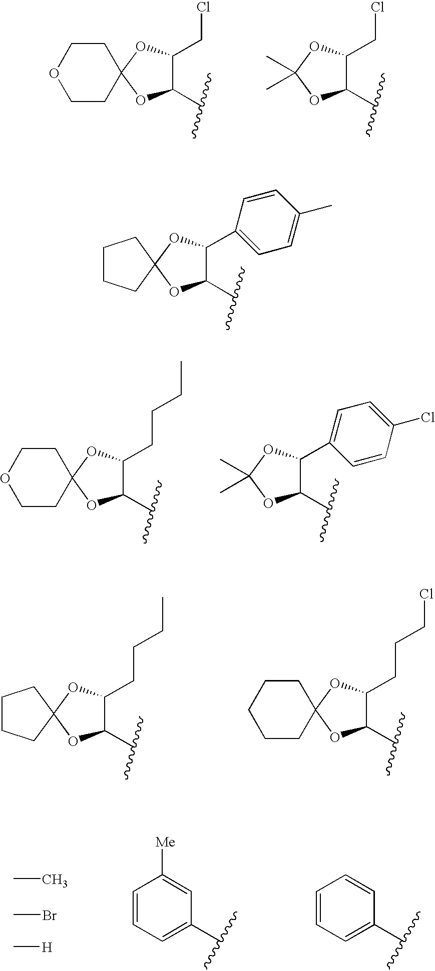
or stereoisomers thereof;
R₃ is selected from the group consisting of: 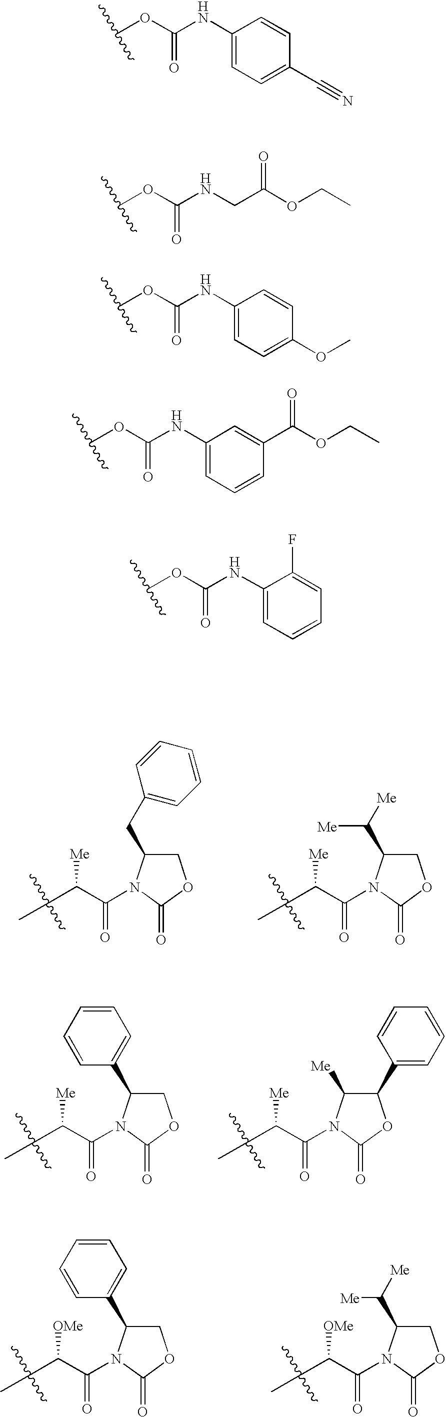 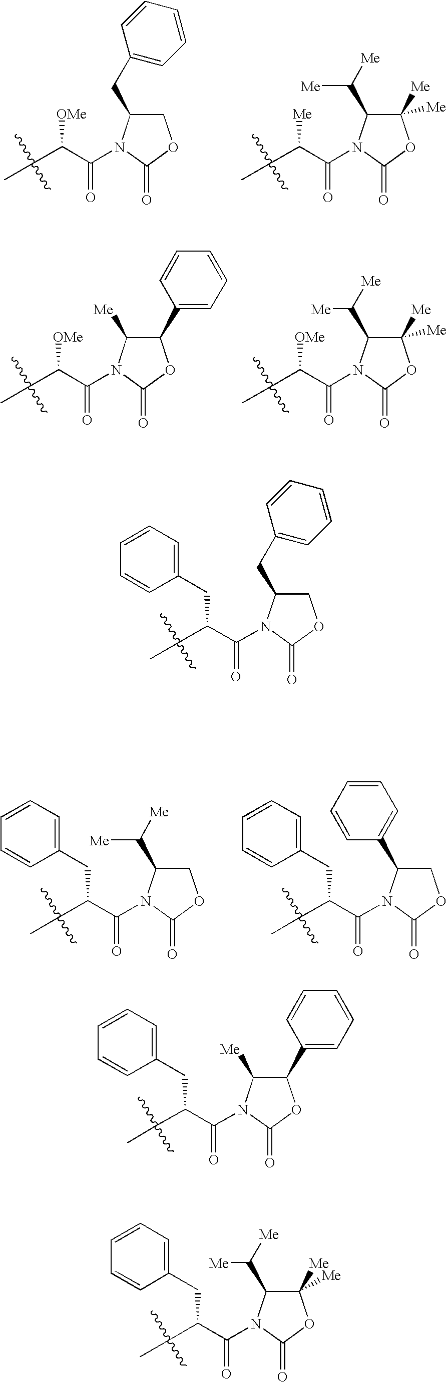
or stereoisomers thereof.

36. A collection of compounds comprising two or more compounds of claim 27.

37. The collection of claim 36, wherein the collection is provided in array format.

38. The collection of claim 36, wherein the collection comprises at least 100 compounds.

39. The collection of claim 36, wherein the collection comprises at least 500 compounds.

40. The collection of claim 36, wherein the collection comprises at least 1,000 compounds.

41. The collection of claim 36, wherein the collection comprises at least 2,000 compounds.

42. The collection of claim 36, wherein the collection comprises at least 3,000 compounds.

43. A compound of one of formula S1 through S10: 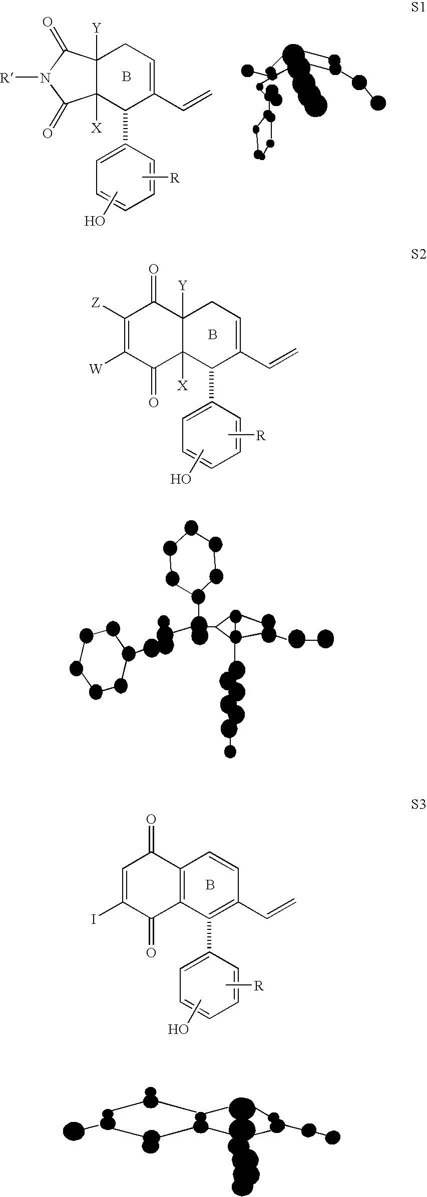 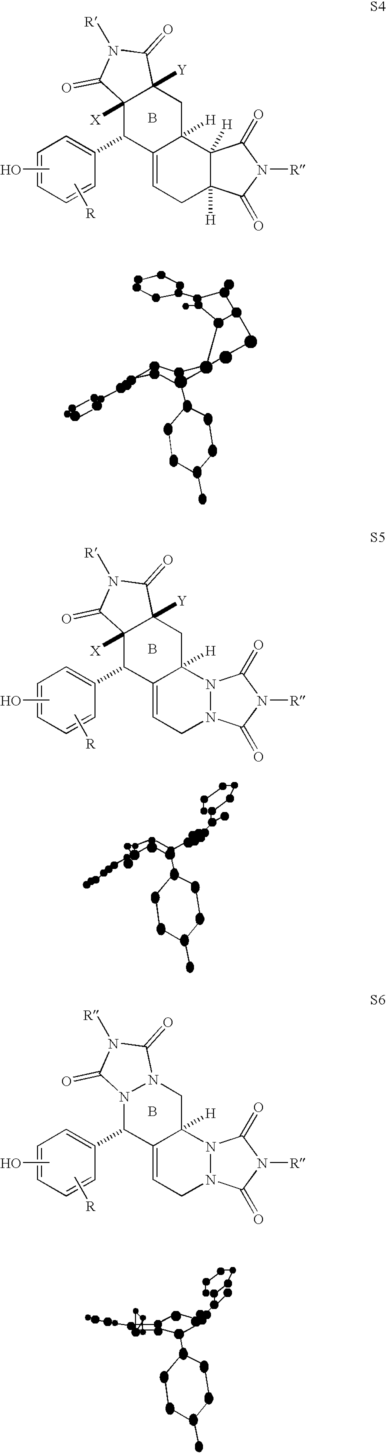 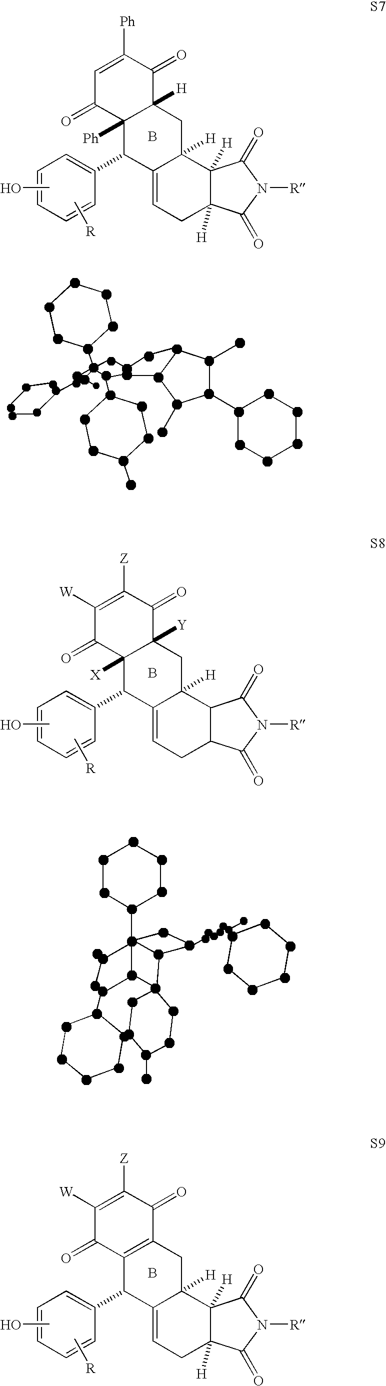 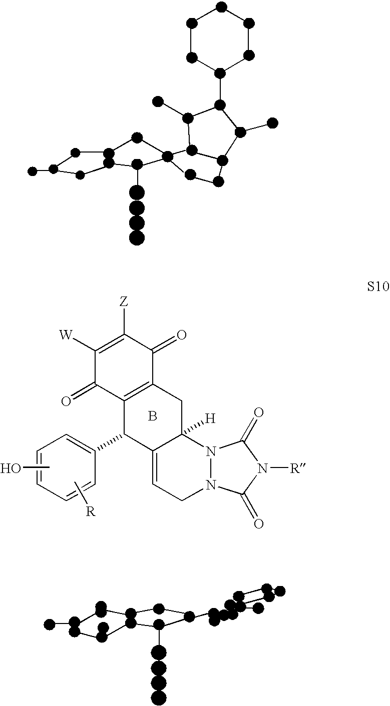
wherein R is hydrogen, halogen, lower alkyl, lower alkoxy, or hydroxy;
n is an integer between 1 and 4;
R′ and R″ are independently hydrogen, aryl, substituted aryl, heteroaryl, substituted heteroaryl, carobcyclic, heterocyclic, acyl, hydroxyl, lower alkyl, or lower alkenyl; and
W, X, Y, and Z are independently hydrogen, lower alkyl, aryl, substituted aryl, heteroaryl, substituted heteroaryl, or halogen.

44. The compound of claim 43, wherein W, X, Y, and Z are each independently selected from the group consisting of hydrogen, methyl, ethyl, propyl, fluorine, bromine, chlorine, iodine, phenyl, and substituted phenyl.

45. The compound of claim 43, wherein each occurrence of R is independently selected from the group consisting of fluorine, chlorine, bromine, iodine, methoxy, ethoxy, benxyloxy, methyl, ethyl, propyl, and allyl.

46. The compound of claim 43, wherein R′ is selected from the group consisting of hydrogen, methyl, ethyl, propyl, tert-butyl, arylalkyl,benzyl, phenyl, substituted phenyl, acyl, cyclohexyl, hydroxy, amino, alkylamino, and dialkylamino.

47. The compound of claim 43, wherein R″ is selected from the group consisting of hydrogen, methyl, phenyl, arylalkyl, and heteroarylalkyl.

48. A collection of compounds comprising two or more compounds of claim 43.

49. A kit comprising precursors templates, reagents for producing molecular skeletons, and reagents for derivatizing the molecular skeletons.

50. The kit of claim 49, wherein the templates are attached to solid supports.

51. The kit of claim 49, wherein the templates are furan derivatives.

52. The kit of claim 49, wherein the reagents for producing molecular skeletons are an oxidation reagent and an acid.

53. A method of screening the collection of compounds of claim 36, the method comprising:
providing the collection of compounds of claim 36;
providing at least one cell;
contacting each of the compounds of the collection with the cell; and
analyzing for any phenotypic or genotypic changes in cell.

54. The method of claim 53 comprising the additional step of cleaving the compound from a solid support.

55. A method of screening the collection of compounds of claim 36, the method comprising:
providing the collection of compounds of claim 36;
providing at least one potential bind partner;
contacting each of the compounds of the collection with each of the binding partners; and
analyzing for binding of the compound with the binding partner.

56. The method of claim 55, wherein the binding partner is a protein.
